SEQUENCE LISTING (858 sequences)

SEQ ID NO: 1 - DNA, 101 bp, Homo sapiens
tgtacctggaactgaaggatggcagatgacaagccaggcagggaggaatgracctggattcctggtgaaggacgtggatatatcttgtggggtataacttg

SEQ ID NO: 2 - DNA, 101 bp, Homo sapiens
ttatttttaagtaaaaaaaaaaaaaagaggaacagaggttatattttttgytatactcaatgagctatctttggataccaataccctatactattcactcc

SEQ ID NO: 3 - DNA, 101 bp, Homo sapiens
gatgttttagtttgctccatgaggaaatagtttccctttttctatttggcrtataaattttcttgctgattataattcttatgtacattacatttttatta

SEQ ID NO: 4 - DNA, 101 bp, Homo sapiens
atagtggtcactggtgacgccaataagagcaacaaaattggaacttttctragcacacaagtgccattgtattaaagagaatatttgctttggatgacaga

SEQ ID NO: 5 - DNA, 101 bp, Homo sapiens
ttatataaactattttgattatgcttcttatatctaaggcagcctgtatarttcatttttttccaaagttagatcctagctgagtagtgaggtatgtacgg

SEQ ID NO: 6 - DNA, 101 bp, Homo sapiens
aaacaatacataacatttcacctttttctaaaacctccaacttttgtctgmtctttgaaaataccaaagtctacctagtctgtgagtacaccccaaattgt

SEQ ID NO: 7 - DNA, 101 bp, Homo sapiens
atgctgccttactctttaagaagatactcatcttcaatagaagaaactgtrtctgtggaacagtaaaaaggccagacactgttcaagatatgtaacacaaa

SEQ ID NO: 8 - DNA, 101 bp, Homo sapiens
atgctgttctctttgagatcctaaattgtttatgagcttggggagctagtyatataatgatgttattttctaaaatgtctgtgtgaacatctttttttttc

SEQ ID NO: 9 - DNA, 101 bp, Homo sapiens
tgattatttttggaaaatgaagtcacatacatatagttctgcatcttgctyttcttacttttcaatatcctgtggaaccctctccaagtcaactggtatag

SEQ ID NO: 10 - DNA, 101 bp, Homo sapiens
atattcaaaataaaaaatttgtaataatcaaagaatatcttctcccatatkagattaatttggcccccaatcaaccacaccacagcctagtaaggtcttaa

SEQ ID NO: 11 - DNA, 101 bp, Homo sapiens
aaaaaaaaactgcacaggcttgaaaactacatttactttacacagctagaygagagaacttgtcatgtcttctaagaacaatgacaatgatacaaactaga

SEQ ID NO: 12 - DNA, 101 bp, Homo sapiens
tacatgtgaggcagacctgaaagacataaatgccagtttccctcttccccmtttggagtgtgtgcttttgatttccctggaggctacatttcctgatctgc

SEQ ID NO: 13 - DNA, 101 bp, Homo sapiens
tctgaagtcagaggttttggatatgaggcagtgctctgacattcactggaytctgaggctcatatgtgtcccagttgagtaatcacaggttccttaaccct

SEQ ID NO: 14 - DNA, 101 bp, Homo sapiens
ctttatggtatggggaaaaggaggatgatgacagcctctgctgcagtgtayaccatccactatggttgctgttcactctcacctccatggagcatcctctg

SEQ ID NO: 15 - DNA, 101 bp, Homo sapiens
gatccaaaagccatgtttgctgtctcagtggggtagatcacggcctagggyatggtctgagtggggcactgcagaagtgagactgacctcaacaactgcat

SEQ ID NO: 16 - DNA, 101 bp, Homo sapiens
tcttattgatcctattttatcagtctcctctactagaatgtgacctccacrtgaatcgggattttttttggtctatttactggctttactttctacattaa

SEQ ID NO: 17 - DNA, 101 bp, Homo sapiens
caagactccgtctcaagcaaacaaacaaaagagcagtgacagcttggttayggttctgtgaattgaaatgctaggcttcccttagggttagttcctccata

SEQ ID NO: 18 - DNA, 101 bp, Homo sapiens
ttttattatcaggccttggcagccacacttcaactttttacaggtaactcrctggaccctcacagtgactcttcaaatgaggttttgcactagacccattt

SEQ ID NO: 19 - DNA, 101 bp, Homo sapiens
tgctggtggaactcactggtcaatattccttttacccatatatagacatcytgtgtcagtgaacttcaaagctgctgattagttttttcctccataatatt

SEQ ID NO: 20 - DNA, 101 bp, Homo sapiens
atgtcaagataagctgattatcctagaatatccaagtgggtccatgatacygagaagcaggaaaatggtaatggaacaatcagtccagacaagccatgcaa

SEQ ID NO: 21 - DNA, 101 bp, Homo sapiens
tgctcaaatctgctctccatacccactagaaaatcctaaaagaaatatagycttaaatacagtttttaggctccatcaccttacctatcctggctgttgtg

SEQ ID NO: 22 - DNA, 101 bp, Homo sapiens
ttccccaggggaaaagtggactgcagaaagacactcactcaccctctctcrtagtggggattcactctcagttcctggtctatcatggtcatataagctgc

SEQ ID NO: 23 - DNA, 101 bp, Homo sapiens
gggcctgatgtatcacaagggtccttataagaaagaagtggggattgaaakatgttatgctttgcagatggaggaaggggccacaaaccaagaaatgccag

SEQ ID NO: 24 - DNA, 101 bp, Homo sapiens
gttgttggtgtaatgaacgtatttaaccttttcctgatagtcaagttcttyctcaatttaggcatcaatctcatctgtgctgtctatggtgattgccttca

SEQ ID NO: 25 - DNA, 101 bp, Homo sapiens
cgcagtgcttctgagagcgggaatccgcgaactggagtcccgtcttccttytggcgtcctgtcttccttttggagctccccctcaaggaccccgggagccc

SEQ ID NO: 26 - DNA, 101 bp, Homo sapiens
aaatgacaggaacacatggacacatagagggaaaaaatagatgctgggacrtacctgaaggtgcaggatgggagaagggtgaggactgagaaactagaaaa

SEQ ID NO: 27 - DNA, 101 bp, Homo sapiens
aaagaagattctcccttttgaaaataatggaactccaggaaagccaaatakgttcaacataattatgagaaagaagtgtgccactgtcagattggcattta

SEQ ID NO: 28 - DNA, 101 bp, Homo sapiens
ggtagatcacaattccatgaagagcaagcaaatatgaatggagttggatgmtaaacagcaaagtgatatttaagtgatcagactacatcacactttttttc

SEQ ID NO: 29 - DNA, 101 bp, Homo sapiens
ccagttgtctcactttttttttttttaccacgtctgtgttcctcatctcayagcaacctggctttaacttccatcccttcacaaaaattacagaagccacc

SEQ ID NO: 30 - DNA, 101 bp, Homo sapiens
ccaagttgtacactttaaatacaatttttgggttaatctaattcctttgcygtttcatgtaaatttttgaattagattgtttacatctataaaaaataagc

SEQ ID NO: 31 - DNA, 101 bp, Homo sapiens
tgcatacagacctaaaatatcgtagttttgaaatgtgcattgagggaaagmtaaggattagcctggtggcataaaatatgggcagcagctggaggtgaagt

SEQ ID NO: 32 - DNA, 101 bp, Homo sapiens
cctctgctcattggcatgccccctgacatctgtttcccctgtctttcactrttggaagtctcagagcctagaaacaattggacacagacatttccaattct

SEQ ID NO: 33 - DNA, 101 bp, Homo sapiens
actgtctctacaaggaaaactataaaacaacaatgaaagttactgaagagracattaaataatagaaagttattccatgctcatgctttgaaaaaattatt

SEQ ID NO: 34 - DNA, 101 bp, Homo sapiens
caggaagcactggaagtagctagcaagaataatagttccttgaggatgggrccgatgctatgcttttttatgatgctccactgaacttacaataattctgt

SEQ ID NO: 35 - DNA, 101 bp, Homo sapiens
ttactttctgggctgatgaaagtgttctggaatcagcagtgatggttgtgyaatcctataagtacataaaccactactttttaaaaagctttgtaaataca

SEQ ID NO: 36 - DNA, 101 bp, Homo sapiens
acctcagttttaccatctttaaattgggtgtaataatgagatctatcttayagctctgtgaagattaaaggagttgaaattggaaatggtaggtgctcaac

SEQ ID NO: 37 - DNA, 101 bp, Homo sapiens
gtttctacattctgaccttgttctgtgctctgcggggctgatctcaatggmaagtgtctctggatttcccttatcccttattggctttgactaactggggg

SEQ ID NO: 38 - DNA, 101 bp, Homo sapiens
tgggttgacatatgagaacatggaagggccatgtaacaggtttagtctagkcagccaagccttttcaaagtgatttctaaattgggtaatggagggtgggt

SEQ ID NO: 39 - DNA, 101 bp, Homo sapiens
aagaaaacaaaaacagactctttcttacagagtaagaggaaaaacagaaartgaaggcaaaaaacaaatgaaaagatgccctttctattttctgaagccag

SEQ ID NO: 40 - DNA, 101 bp, Homo sapiens
aaggcagaacgccagaacaggtggatatgagtcccaagccactgtgctcamtcaccgtgctaattctgcctccctgcagctgctgtggctgataaggagga

SEQ ID NO: 41 - DNA, 101 bp, Homo sapiens
ctaccaacctgccttccttcctgttaacttaatgagctgttagtgctcaayctaatggtgagttcattgtccttatcttatttgacccagcaacagcttgt

SEQ ID NO: 42 - DNA, 101 bp, Homo sapiens
gttgacattcctggtaggaagatccacaaagcatttggtcctattgccagrggtatcatcactggttctgaggagtggaaagagtacacaggcaaggcaga

SEQ ID NO: 43 - DNA, 101 bp, Homo sapiens
caacatcatgtcatttctgtgagcagagccaaacattgttgcctgagagarccccaaggagggcttgaaaagagtttctcatcagcaatctcatactcatt

SEQ ID NO: 44 - DNA, 101 bp, Homo sapiens
ttccccactagagtggaagcatcctgaggacagggacctttgctgctttgytcaccactcaatcatcttgcccagaactgagcttggtacattgtaagatg

SEQ ID NO: 45 - DNA, 101 bp, Homo sapiens
tgactgtatatacaaaggtggaattgctggatcctatgtgttaaacctttracctcttgaagaattggcagactgtttgccaaagtagctgcactaataaa

SEQ ID NO: 46 - DNA, 101 bp, Homo sapiens
tctttttggctattccccttctgtgccctttttgcagaagtaaactctgtkgggaagggtaaatgtgtagcctcaaattcctcattaaggttttattttat

SEQ ID NO: 47 - DNA, 101 bp, Homo sapiens
gctcagtaaatatttgttaaatgaatgaagtgtatgtttctgcaaatgctrtcagaatctcattttatctctctgacaagactgcacctttagtgcaggga

SEQ ID NO: 48 - DNA, 101 bp, Homo sapiens
agagaacagcattgacagagacgattagtttcctcccccgcccccagtccrttggcctctgttgctaataacgcttggttgaggattatattaaaatgagt

SEQ ID NO: 49 - DNA, 101 bp, Homo sapiens
atgcttccagctctgttatttttcttaaaattgcttgggccattcgagctyttttttttgtttaatatgaattttagggtttgagtacattgaagcttttt

SEQ ID NO: 50 - DNA, 101 bp, Homo sapiens
acaaagtttgtgtataatacatgccaagagggtaggaataaaataccattygctgtcaagatatatttctaaacaagtttattaggaaggcagtagcagat

SEQ ID NO: 51 - DNA, 101 bp, Homo sapiens
agtgtttattaatgaactagccatagtaaaattacagcccatttaaacatycctctttgactaacactagtgtctatcccttgccattgcagcaatgatct

SEQ ID NO: 52 - DNA, 101 bp, Homo sapiens
gaggaaaacaatttctcaatccacggttatttctttgttatactaagaacmgtgcccaatacttatggaacaaaataagcctatcatttggacgtctccta

SEQ ID NO: 53 - DNA, 101 bp, Homo sapiens
agaggcaagtgtcagaaattaagcaagtaaacaacagaacactgtgagccrttggtttgtaacatgacagctgcctgtctgtgcctcttactgtgtctgtg

SEQ ID NO: 54 - DNA, 101 bp, Homo sapiens
gagctaggctaaaatcaggacccaagaacctcacctaagatattttacagrgataaaaccattatctattcatttttcaaaatccccctttaatccaaatt

SEQ ID NO: 55 - DNA, 101 bp, Homo sapiens
cctttttcctctctctagaaagggaggatcaccaggaagaaataagtccarattccccatcagttcagtggtatggagtccagagtcagaatataattttt

SEQ ID NO: 56 - DNA, 101 bp, Homo sapiens
cttatatgagctatgaattagcccgaccaccatcactgctactgctactaygccccagactctctgtgctgctgccttgccagcctgctgtgccctgctga

SEQ ID NO: 57 - DNA, 101 bp, Homo sapiens
ggtgtttggcagtgctgttgttcaaaaatatggccaaggcttcttaaatayactgactgttggattcccttccctgcctccactccctcatctgctgaatc

SEQ ID NO: 58 - DNA, 101 bp, Homo sapiens
cttgactaagtggagggtattgtggagtagagcccttctgaataacagcarctaacattctcatagcactaactgcacccctttgaggtaggcggtcttat

SEQ ID NO: 59 - DNA, 101 bp, Homo sapiens
gcaacagagaaaaaaatgttttttgtttattttagcatgtttatttttggyccaagcctttatcaggttggagttggaggctggggaggaagaataacaaa

SEQ ID NO: 60 - DNA, 101 bp, Homo sapiens
ttttaaaaatacaaattaaaaattatctattggacagagccatgtgtagaycttagcctttgcacttgcaaatcaaagctttacaagagatgctctccaaa

SEQ ID NO: 61 - DNA, 101 bp, Homo sapiens
ttaaaaaaacttcatttacaccagaatgatttccgtctgtcactcattgaytttacctctttttttctacctctaattactataaaaatatttgggatggt

SEQ ID NO: 62 - DNA, 101 bp, Homo sapiens
ggcaaaggggttaggtgtcaatgcctggctgatttcctgcattacaaaatktacctcttacttttctgtcttcctgatgttaccccctcttttctttcacc

SEQ ID NO: 63 - DNA, 101 bp, Homo sapiens
tttccctgataaaaaggcatcttgtccacagctgtacttgttttcttattragtgatcctggttatagaacatgtgacttcaggcataaaattctttctac

SEQ ID NO: 64 - DNA, 101 bp, Homo sapiens
aggaaacacaaacttctagaacttttaaattgttaaacatctttgtggaaktaactaccattttcaccaaatctgcaaatcatattccaacaagttgtaaa

SEQ ID NO: 65 - DNA, 101 bp, Homo sapiens
tgtggctgttaagtggtgactgaagtagaatggaggtgaaaataattcaaratggaaagctaaaacaaccgagaggcttggaagctgaagaattccttcat

SEQ ID NO: 66 - DNA, 101 bp, Homo sapiens
cacatacgcatatcctcctcaattttataaagaaatagaagcaccattccrcaccttcatattccacccttaatcattgttaagttggttgcatgtcttcc

SEQ ID NO: 67 - DNA, 101 bp, Homo sapiens
gcaaagagggccagtagttacactgcaccattgtggtgacatcaccctatrtatgtattttttaaataacttgttaatgcatatttccctagctagactaa

SEQ ID NO: 68 - DNA, 101 bp, Homo sapiens
ttttggctgttaggctgtagagactttatgagggtgccaaacttggaagamatattgaaggtagactcaacagaattttcacaatatgaaccctgtgagac

SEQ ID NO: 69 - DNA, 101 bp, Homo sapiens
ctattgtgaggcagggtgtggaaatcgtgattgagatgacaaggcacccarttgtactcatataaagaacactgcttgcgcgtatgattgctgttcaggtc

SEQ ID NO: 70 - DNA, 101 bp, Homo sapiens
tagtatgcttattaaatctgcagatgaatgcatcttgtcaaggaaaatttyctatgttacaactgaatttcttctatttcacatgttgaggtctctttgga

SEQ ID NO: 71 - DNA, 101 bp, Homo sapiens
gacaggtcttctttcctgccagagggagctctgaagacaactagagaattytgggcctgaaatttcaatctagttagaaagaaaaatgaggcaatgatttt

SEQ ID NO: 72 - DNA, 101 bp, Homo sapiens
gacagggcacgtaggaatatggaagtcagaaggacaacacagctctgctaygtcccggttcttggtaactttcttaaccccactatgctttatctttagtt

SEQ ID NO: 73 - DNA, 101 bp, Homo sapiens
tgaggagagttcctgggccaagggctggctggcccatgtgacttttggggkctcaggaggagcctgttgtgttggggagtctctctgctcaggtcctgtgt

SEQ ID NO: 74 - DNA, 101 bp, Homo sapiens
gccccttggctggttcttacccatcagcaagctctgaatgcggtcgtaatrtgtgaagttgtaggtgctgctcgtggaggctgcctcatccctgggcagcg

SEQ ID NO: 75 - DNA, 101 bp, Homo sapiens
tgggcaaattcgctatgcatcaggctgacggcctggaggaagcggcgatcmtgcggggtggccacctgcggcaggtttgcttccagaagaggacacagagt

SEQ ID NO: 76 - DNA, 101 bp, Homo sapiens
gggttcccacccagacagacggactcaagaactcacgcactgcctctgcaycctctgctgccaatgaaaatttaaatgagggcaacaggagatcagagatg

SEQ ID NO: 77 - DNA, 101 bp, Homo sapiens
tgaaatctacaaggtgcctttcatcacgagagctgagcgatgacccctgartgaggagggccaggagcttagtcccatctcagagacagacactgactcag

SEQ ID NO: 78 - DNA, 101 bp, Homo sapiens
tccttgaccccattcgccctcttacaaataatgaggttcagaaggcaggtrcaccagatgggagggagaaacaaaaataaagataaacgaaacaacattta

SEQ ID NO: 79 - DNA, 101 bp, Homo sapiens
gcacttcatttattcaccaaatacctgctttggaaaataattggagtcggrgggagcagcaagaagggtgaaatagggcagtgcagggctcctggattggg

SEQ ID NO: 80 - DNA, 101 bp, Homo sapiens
ttcataggcatgcaagccttcttatgaactaactgcacgtgccagggatcraggttgcacactccttataagaatctaatgcctgatgatctgaggtggga

SEQ ID NO: 81 - DNA, 101 bp, Homo sapiens
atcatggcagaaggcaaaggagaagcaggaaccttcttcataagggggcargacaatgtgagtgccagcagggaaaatgccagattcttataaagccatca

SEQ ID NO: 82 - DNA, 101 bp, Homo sapiens
gctgaactggccatggaaatggcagcctgggcaacaggttcatgaaaacaracttttcacacctggtcctgctctccaggcctgagcgaactccatgtgtt

SEQ ID NO: 83 - DNA, 101 bp, Homo sapiens
ggctcttgtgggacagggctagtggaacctacttgggtgtctccattgcgrgcagaacgtaatagctgtgtgtagaaggtcccactggatgaagggccagt

SEQ ID NO: 84 - DNA, 101 bp, Homo sapiens
tggctggaggaacccaggaacaccctgagcatccatgttcttaatgacaaragagggaacacagatttggcttccctttcttcataagaaaagaaagaaaa

SEQ ID NO: 85 - DNA, 101 bp, Homo sapiens
catgcatatccagaaactacagtaatttacaggggcaaactctgcaactargaaaaggagacagaactgtttccactcaatgcattcctccatcaaagaac

SEQ ID NO: 86 - DNA, 101 bp, Homo sapiens
ttgtgtttctgtgtggctgaaatcgtgtcgtaaagttagaagaaaggctgytgtggggcctgcgttgcttggcagaatgttccttaccttttgatttgcag

SEQ ID NO: 87 - DNA, 101 bp, Homo sapiens
gtgccaagcagagcaggtagttggctaagtttgcctccaggaaagaagtcyctggagagcgagctggttctagaaagctccattattatattcctattgct

SEQ ID NO: 88 - DNA, 101 bp, Homo sapiens
gtcagtggtgatattctctttatcattttcattgtgtccatttgattcttytcacttttctttgtctagctagcagtctatctattttattaatttttttc

SEQ ID NO: 89 - DNA, 101 bp, Homo sapiens
cccatgtaagacacccatgaaacaatgctctggtcataattagtctctaamctttcaaaatgcctgcttcagtgacctcacctgctattgaacacgatgcc

SEQ ID NO: 90 - DNA, 101 bp, Homo sapiens
agccacctctcatttgcatggtggacagctgcggctgacaggcaaacaaaratgtctgcggccatggcagctcctagagaaactcttctctccttactctc

SEQ ID NO: 91 - DNA, 101 bp, Homo sapiens
ctgcgcttcccccagaaagcatgcctgggtgaggggccaggtgacacttcytacgatctggattttaaaatatgtttgcttatgccttcaccctccaccaa

SEQ ID NO: 92 - DNA, 101 bp, Homo sapiens
gcgctcacgggagggcggatgtggagagggcagaggagcaatggtgacctrggaaggtaccctgagcggctacgctaggatctctgttctgcagacttctg

SEQ ID NO: 93 - DNA, 101 bp, Homo sapiens
agggaagcatcagatgtcactggcttgggaaagatattccagaaggaaggracaggttgtacaaagtaaggtaattttgtttggggaagctccagcaggtc

SEQ ID NO: 94 - DNA, 101 bp, Homo sapiens
agttatcagcttattgctattaaaaataacactaaacttttgtttatctamagagtgtcaggtaagcaagtgaacattttgatgcaaaaagaaatcacttt

SEQ ID NO: 95 - DNA, 101 bp, Homo sapiens
ggctgagtaaattaaggtacatctgtattaaggaataaaatgcaactacgraaaatgataaactagatggaggggtgcctatgacactgtaaagtttaaca

SEQ ID NO: 96 - DNA, 101 bp, Homo sapiens
tggctgtgttctgagtgggagtgtcctaagagtgagagttcctagtgaccyaggcagaagttgggttgacacttcttgcaagatttctgatgacctagcct

SEQ ID NO: 97 - DNA, 101 bp, Homo sapiens
ggtctctgtggattcccaaaggaggtttcaaatggagtcattgtaaagacrattcatgatcttagaagtgtctcatgcagtttcctcgtgatggtcttgtt

SEQ ID NO: 98 - DNA, 101 bp, Homo sapiens
caggaatcccaattatggggaaagaagatgagcttctgagactattccgakccacaagatttttcaaattcttcacaatctctgtctcatggatcagagag

SEQ ID NO: 99 - DNA, 101 bp, Homo sapiens
cactgtaccttcgcagcacgaggagaggagagttcgaaaccacaaagctcyttcctttctttcaggagaaagaaaatggaggatgggaacgtcatcagccc

SEQ ID NO: 100 - DNA, 101 bp, Homo sapiens
gggcctcaattttctcagctataatatgggctgacaagagtaaacgacaakagcaaatgagttaatatgtgttgcccctgatgttacagtggataacgatg

SEQ ID NO: 101 - DNA, 101 bp, Homo sapiens
aatcttaaacagtaaagtttcacgaagacaaaaatctttttgatcaatcaygtctcttttacaaagtttacaaggaaagtattcatccctaaaactatttt

SEQ ID NO: 102 - DNA, 101 bp, Homo sapiens
gagttacttatacaaaattacacactaagagatttgtatgtataattgtgkgtacacattcctagtattttcctgatataaaaaaattattcctatataag

SEQ ID NO: 103 - DNA, 101 bp, Homo sapiens
gaaggagtttggatatattccctcttctttaatttttttgaagaatttgartagaattagtgttagttctttacatgtttgttagaattcagctgtgaagc

SEQ ID NO: 104 - DNA, 101 bp, Homo sapiens
agttagtacaggagcggggccaggagagtgctgtcccctcagctccagtgrgtggctgcccatccagagcaagcctgcagcccccacccgcctcctccttt

SEQ ID NO: 105 - DNA, 101 bp, Homo sapiens
tcttgaatgcaggaactattatataaaagcattgcagctcttggtggttgyggcagagacgcagagaaagccagtttgcattgaaggaagggtacagcaga

SEQ ID NO: 106 - DNA, 101 bp, Homo sapiens
tgctatagtacacatagcaaatctgcaaaagtgctagctatcattattatmtgaggcttttgacccagctctcagagaagctggaaatttgcatttttatg

SEQ ID NO: 107 - DNA, 101 bp, Homo sapiens
ggagaatgcataatgaggctgaatgagaattagatgcttaattgaggcctrgaaaagggaaagaaaaagccagacatgtggaatgtgatcagaatgcagct

SEQ ID NO: 108 - DNA, 101 bp, Homo sapiens
acagactgtccttggaatgttggaaagttatttggaaagtccttatgagcytggggcacattcttctgaagagctttcttgattaggaaaatcctgtgctt

SEQ ID NO: 109 - DNA, 101 bp, Homo sapiens
tacacacaaattcatgcccacacccatagacacacatatacatatatacaygcatgtatatgtccgtatagagagctctatgctggaatatacaaaaacat

SEQ ID NO: 110 - DNA, 101 bp, Homo sapiens
gagcttcaggacttcaagtagatcacaaaaaaagtgtggaatttccatttyggtgcagaaggacagcctcaaaacagtcaaggtctcgagcagggaaccca

SEQ ID NO: 111 - DNA, 101 bp, Homo sapiens
gcctggggggtggtaatttgggagccactgaaatgaacttgcaaaaggttktgggactattcatttatctgcagaaggctcagaaatttcattagattctc

SEQ ID NO: 112 - DNA, 101 bp, Homo sapiens
tttgtttttttgtattttcacaataaatatgaaaacagttttaatttaatkattatgaacaaaaaaggatgaaaaccaatagtcagtttctttgtaaaatt

SEQ ID NO: 113 - DNA, 101 bp, Homo sapiens
caccacacaggaagggattttgtctgtcatgttcactgctgtgtccccagyatgctaagtaggggccagggtcaaagtaaatgcttgatgaatctttgccg

SEQ ID NO: 114 - DNA, 101 bp, Homo sapiens
tccccacttcttgcataaagggtagcattcatgagcataccgttctgcacyttgcttttttcatttgtgtcttgaaacctgttccctgttggctaagagag

SEQ ID NO: 115 - DNA, 101 bp, Homo sapiens
gccttggacctgctgggcccagccactggctgtctactggacgatgggctygagggcctgtttgaggatattgacacctctatgtatgacaatgaactttg

SEQ ID NO: 116 - DNA, 101 bp, Homo sapiens
ggccctcatgctgtaaagaagttgagttctggaaactccaagttatcatcrtccaagtttagcaatcccatcagcagcagtaagaggaatgtctccctcct

SEQ ID NO: 117 - DNA, 101 bp, Homo sapiens
aagagtgcataggagttttctaggcagagaaaacaaccctgcaggcgcacrttggctcccattcctggattgagggcgtggccatgaagtctgggtgctgc

SEQ ID NO: 118 - DNA, 101 bp, Homo sapiens
caggaggggtcaacttggagggccaagcaaccaggggtcacatgggcatayggctgagcctggacccatccacctgactactatgctattatagggctccc

SEQ ID NO: 119 - DNA, 101 bp, Homo sapiens
agaagtttctttattgagaatgatattcattagtaggcattcaatgataargacacagcctgattttaaagatttcctttttttttttttttttgcacatg

SEQ ID NO: 120 - DNA, 101 bp, Homo sapiens
ctccaagggcggatggcctgaccgggataagacccgtgaacagatagtaartgtgggtttggcatttggcaggaaatgcttgtggaattcaggaggcaact

SEQ ID NO: 121 - DNA, 101 bp, Homo sapiens
tgtgctcaggcaagattatggagcgagcttggttttgtcctactccatcgyggtcagagtggccccatctgatatgagcgttctgtgagttttttttatta

SEQ ID NO: 122 - DNA, 101 bp, Homo sapiens
gattacaagcgtgagccaccacacctggccttgaggtcacctttgcatgcraaggctgtatactgctaacacctgtgacatctcctgtctgatggtgtcct

SEQ ID NO: 123 - DNA, 101 bp, Homo sapiens
aaatttttcctgtaattgaccaagtagcaaatatattcagctttgctggcygtaaatttcctggcaatgactcagtcctgccgcggcagtgtggttaacag

SEQ ID NO: 124 - DNA, 101 bp, Homo sapiens
tgtcgaaaaacctatcaacaattccttagtttcaccacttcaaaaaatttrttctagtgtcaaatcccacattttaaataaatacagaaatgattttgatg

SEQ ID NO: 125 - DNA, 101 bp, Homo sapiens
gaaggagggatttggagccagggcagacagagcagcatggtgctgggagarcaagaggggcagccagtgataaggagagcacagggagaaccacagcctgg

SEQ ID NO: 126 - DNA, 101 bp, Homo sapiens
gcacattatctatgctgtttgttataggtaatagtttcagcaaactagacmggaaggaaaaaatgcattaagagtgaaggtgaaagagagagcgagagtgt

SEQ ID NO: 127 - DNA, 101 bp, Homo sapiens
acaagatattccctctgatctctggccctctcctccagccctctccaagarggacattgtccttgcctcctatcccagagagctggcaaatattcccctac

SEQ ID NO: 128 - DNA, 101 bp, Homo sapiens
gatttctcctgtgtgggcaagtcacacacaaaactccagaaatacatattyaaaatgctcctagcttccctctgcattagtcacaataacactaaatgctg

SEQ ID NO: 129 - DNA, 101 bp, Homo sapiens
agcaagactccatctcaaaaacaaaaaaggcaaattaaatttatactaacrtcagcaaactagagaatttaatggctcatgtaactacaggtagagatggg

SEQ ID NO: 130 - DNA, 101 bp, Homo sapiens
atagctcctcttttattactcggtcctggggttaacctcaattgtatccayttactcaactagtgtttaatgagttgccatggtgtgcctcgtacttgtga

SEQ ID NO: 131 - DNA, 101 bp, Homo sapiens
tcatagcttcctttgtacctcaaactaagtagcttcatattcctttgctcrtgcaacccaatcatatttgggaagctgcagatgaaaagcatactgacttt

SEQ ID NO: 132 - DNA, 101 bp, Homo sapiens
gggtcatctgacaataaggccacctaaggtccgccagtagtagttgtagaygaactggtgacttctggcatggtcattagggcaattgttaaaacttttat

SEQ ID NO: 133 - DNA, 101 bp, Homo sapiens
tgtttgctgagccttctctgcgctgtgtatagtactcagggaagcttcacrtaagtgtcttccttcactcatgtgttcgctcaggaaatacgtatttactg

SEQ ID NO: 134 - DNA, 101 bp, Homo sapiens
gccatggacattccgggttcccaagtcaggtggggcccagggataagcatytatttttgatcagcacctcaggtaactcctgtcttcaccatagtttgaaa

SEQ ID NO: 135 - DNA, 101 bp, Homo sapiens
tatcttatttattttcaagtcacaccaaaggaaaggcaaggctcagagaartggattaatttgctggaggctacatagtaagcagagggggtgggatatga

SEQ ID NO: 136 - DNA, 101 bp, Homo sapiens
tataagtgtatatgtagaagaaaatgtccggagtctggagacagaaccaakagagagaattagaggttagatttccagtgcttacacagagccagtgttat

SEQ ID NO: 137 - DNA, 101 bp, Homo sapiens
ctgtacaaagtctgaattttgggggaatctgaagagtctcatttaaatatycagctgattaattataagtgtatatgtagaagaaaatgtccggagtctgg

SEQ ID NO: 138 - DNA, 101 bp, Homo sapiens
tcttctcattacttcagaatacagacatccagtgtttaattctgtttgtgrttatctcataattattaagatatattcataactatttgtttattaatcaa

SEQ ID NO: 139 - DNA, 101 bp, Homo sapiens
agaacaaaagtaggtgattgatatagtttggatatttgtcccctcttaatyttatgttggaatgtggttcccaatgttggacatggagcctggtgggagat

SEQ ID NO: 140 - DNA, 101 bp, Homo sapiens
acaggacatgctcaatgtgggctttttttaaattttttttccttctcttgyttttcttttatttctgtgcgattacctgctcctctgtggtttctttattg

SEQ ID NO: 141 - DNA, 101 bp, Homo sapiens
ctgacaggcagaaatatatgccaccccaaaatatgtcagcctaaaagatgycttctcaattgaaggcaattgagaagaagcagatacaagaaaagctctct

SEQ ID NO: 142 - DNA, 101 bp, Homo sapiens
gaggttgataaacatgatggtgaagatgttgagcagttttccttaaaactrgttctcaattcactgctgatttgtggaaatctggcactgtctataccagg

SEQ ID NO: 143 - DNA, 101 bp, Homo sapiens
tacagtgtctagatgtgctagtgtatccagaatggtgcccaagagagaaamgtaggttaggaatatattgagctgacctattttccatacgtaagtatggg

SEQ ID NO: 144 - DNA, 101 bp, Homo sapiens
aatataaaaacatttgacttaagattttctgaggaagcttaagtagtttcrttgaaggctgaactggtttggtcctgaatctcatcctctatggcataatt

SEQ ID NO: 145 - DNA, 101 bp, Homo sapiens
cccaaactctcctttcgatcctttaatctcccttaatcatctcttgaatcygcctcttcctgtctattctcacacactctgttctaacctagaaccacttt

SEQ ID NO: 146 - DNA, 101 bp, Homo sapiens
gaaaagacctcaaatttgctagtaagattcaacgataaatgcaaaatacayacatctacacacacttacttagaagggtagtaagatagacatatttgaca

SEQ ID NO: 147 - DNA, 101 bp, Homo sapiens
atgcccccgtttaacctctgaaaccttgtcattaaactacagggaattaartccaataataaacccttccattgtcaacagaactctcaatgaactgtacc

SEQ ID NO: 148 - DNA, 101 bp, Homo sapiens
gatgattgtagagcataaagaaactaattcacgtaaaacattttcatgtcyaggatacaggtttcaataaatattagtcagaagcatcgtgatcattttgt

SEQ ID NO: 149 - DNA, 101 bp, Homo sapiens
catcgtcactgggttaggtctcaatgtcggcagggctggctgaggctctcrggaggattatctttccttgcctttttccagcttctagaagccaccttcaa

SEQ ID NO: 150 - DNA, 101 bp, Homo sapiens
actgcccgctctccttgccttcatggggccacaactttctgacttctcccrtttgcttttgcagacacctcctcttcctctagatattcttctccagagag

SEQ ID NO: 151 - DNA, 101 bp, Homo sapiens
ggcaagtccagcaagtctacatatttctagtcacatttccttgcctataayttattaatccatttatcaaatatttattgagcacatacttactatcatgt

SEQ ID NO: 152 - DNA, 101 bp, Homo sapiens
cacaggatggaaacaaaatatcatgagggtccagcagtcttcagagcagtrttttttcagctggggacagaaacaccaggaggcttatgaggagtttctag

SEQ ID NO: 153 - DNA, 101 bp, Homo sapiens
ttgatgtcatttgggacaatggcagaaccgtctccttctccaagttctaamaatgaacttagatgactggcaaaacccccagagtgtgaaggcttgtagct

SEQ ID NO: 154 - DNA, 101 bp, Homo sapiens
catgtgacaggaatatactagatgtatctacaagttttcttatgacacagrtattcatgacatcaatctcatgacacaggtagtaggaatatattttaaag

SEQ ID NO: 155 - DNA, 101 bp, Homo sapiens
aactggaactgctggttaatcttgaatcagacaaagagcaccatggacacytcgaggaagtgcccacagcccagcaacaaaagtttctgcagagatttctt

SEQ ID NO: 156 - DNA, 101 bp, Homo sapiens
aagtcaaactatccgtgtttgcagatgacatgatcctatatctagaaaacyccctaatcttagcccagagcttcttaggctcataaacaacttcagcaaag

SEQ ID NO: 157 - DNA, 101 bp, Homo sapiens
ggtggcattatttaaaatgtactaaggtatgactcagtcatcatgctaaarcattattgtaccttatataaacatgactgtaattcgatgttttaaattct

SEQ ID NO: 158 - DNA, 101 bp, Homo sapiens
aaggaaaagtccttctaacttctacagggccaaagcatgcatgtatcataytaatgtcaatcctgtgccagaccctttgtaaaattaagtacttcaaactt

SEQ ID NO: 159 - DNA, 101 bp, Homo sapiens
cctagttggccacagggagggctggtcaactgcaggggcaggcaggggtayacatgacccaggcctagcctggaagtgttctcagcctggtcctgctccgt

SEQ ID NO: 160 - DNA, 101 bp, Homo sapiens
cattttctacaattgtgaaaatcagacaccgcagtaggattagtgtaagcrtcgtggtttctaggtagtcttctctgacacctaggcagaatcagggccct

SEQ ID NO: 161 - DNA, 101 bp, Homo sapiens
gccttcaaagcggcagtggccacccacacagggaactagtgtttgtgagargagaatgaacgttgtttgtaatatgttggtgtgaattgtcagcagagcac

SEQ ID NO: 162 - DNA, 101 bp, Homo sapiens
gctgaaaggtttccatgtggaagcccctgactaccaccaaccagttcaggygagagacctgaatcctttcccccttttctttttaccttttctgaatccta

SEQ ID NO: 163 - DNA, 101 bp, Homo sapiens
atctcaatatatttcaacaatgggaacttctgcggggcacaactcatgtcyacagcctcgtctatgtacagagcccaaagcagcaccactatcagtttggg

SEQ ID NO: 164 - DNA, 101 bp, Homo sapiens
ttctaccaccgtagatccgttttgccttttgtgtctggtttcaatgcatcrtaggtccacgacatccttccacaggtaccggccactcattcctttccttg

SEQ ID NO: 165 - DNA, 101 bp, Homo sapiens
ataggcacatatcggatctcccagcctggtgactcttccgtggtctaatckgaacacctctggcctgccacacctctggccagcctccagttagctgcttt

SEQ ID NO: 166 - DNA, 101 bp, Homo sapiens
tcctagggaacgccctcttctcgctgcggccctggcgtgtgtcgctggatkgtgagggccccactgcattggtctccatgtgctctgccttctcaatgtcc

SEQ ID NO: 167 - DNA, 101 bp, Homo sapiens
agatgggggcagtcctttggcaggggtgctcaagttggtcgattatcccarcggtgccagagcggcagtgatttgtgggtgggcaggctccttccctaggg

SEQ ID NO: 168 - DNA, 101 bp, Homo sapiens
tctgctgcagttcatagggttcttcctgttggtctccataccactcacccraagcatgcgagaagctgcaggggcttgggggcagttggagttcatgtggg

SEQ ID NO: 169 - DNA, 101 bp, Homo sapiens
gatgtatgtgtataaattgcactcatggctctaaaacaaatcagcagaacmcattctagaaaaaatcgcattcaagagatactatactaatagattatgta

SEQ ID NO: 170 - DNA, 101 bp, Homo sapiens
aaaattactcctggcctcagctgcctcatgtctgggtccctccctgccaayagatttgtgatggatatttacacgctggaagtgactgggccatggtctca

SEQ ID NO: 171 - DNA, 101 bp, Homo sapiens
gggagaactacagttcccagaagagtgtgcggaagaagcggcccatgctcycggaagacgctgtggttgagcatcatgggagttgtagtactcctgctgct

SEQ ID NO: 172 - DNA, 101 bp, Homo sapiens
ggccatccgtggggcctgcaggagaacaagtggaatctgcagcatgggacrtctctgcctagagcctgtgcaaacaatggcactgtcctcatcattgaggg

SEQ ID NO: 173 - DNA, 101 bp, Homo sapiens
aaacacaaggaggcaccgaggctgctgtacaagagttggttcctgctcacyccacaaactctacttccacctactgcaaaaggttctgtccttttttttaa

SEQ ID NO: 174 - DNA, 101 bp, Homo sapiens
tgctgaccagggaatacctccccattgaagcctaggccagattccagtccrttttgaccataccccatcatggtattttagagtacacctgaataagatac

SEQ ID NO: 175 - DNA, 101 bp, Homo sapiens
cacgcccccacccgccgcagcccctactcactcttcgtataggagagccaytatgtaggtgagggccaccagcaccgtcaggagcaggcccgtggggctgg

SEQ ID NO: 176 - DNA, 101 bp, Homo sapiens
cagtccccacatttgcattgtccccaaatctaacccaagctgaaagacatyaggcctatcttcttgctttatgcataatggcagatctccagggagggaga

SEQ ID NO: 177 - DNA, 101 bp, Homo sapiens
gccttttcattcccctctttttttaataaaggaaagccaattttaccgggrgtggcaaagtgtctggagaaaacataacatttcttagtttcctttgtagc

SEQ ID NO: 178 - DNA, 101 bp, Homo sapiens
tgtgtgcgttttcctgagtgtgcaggagtacgtgataatttcctgctaggrtggaatgacttccgggtccatgagtgtggaattagggtcagctctgggtt

SEQ ID NO: 179 - DNA, 101 bp, Homo sapiens
cagtttctgaggcccggttctcccccaggggctgggctgcaatcagcaggkactaaatctcactgccaagggcctgggccaaggcatccaactctctgtgc

SEQ ID NO: 180 - DNA, 101 bp, Homo sapiens
ctgaacagcaaacccagaggccattgcagctgcctcggtattctacacccyccttgggtctggaagttgttggaggcaggcataccagactgtttataata

SEQ ID NO: 181 - DNA, 101 bp, Homo sapiens
gtgctctcatcctaatttagggcccctttctgcctagaactctgtagattyccgccgtctgtgtttttccatcatcccagaccctcagctgcaagctcagg

SEQ ID NO: 182 - DNA, 101 bp, Homo sapiens
cccacttgttctgcagagaaagtgagagggaaaggttgctgatcagatgcygctttaaaatgtaatcataagttttggctcagggagagagagagagagag

SEQ ID NO: 183 - DNA, 101 bp, Homo sapiens
gttctagggcctggaccaggggcttacctaaagcccatggtgcctcctccrtctgaatgggagcctccacagccagtaatgagtatccttcctcaaacctg

SEQ ID NO: 184 - DNA, 101 bp, Homo sapiens
agtagtttcgtctctcagaaccttataaaatggataatagagtagtacccmtccgatagggctgttgtcagggacaaggaactaatacccatgaagcactg

SEQ ID NO: 185 - DNA, 101 bp, Homo sapiens
tcagaaaatatttgcacacacattgtctcttctggcccttgaaacattccytgtgtggctgaagaaagtcaatagtggaaccatttaatagataaggacat

SEQ ID NO: 186 - DNA, 101 bp, Homo sapiens
aaaatcttttagttcctaaaaagcacaaacttaaaaaaaaaagggggaaaygaaagggacttcttcaatttggcaaagaacatctacaaaatacctacaga

SEQ ID NO: 187 - DNA, 101 bp, Homo sapiens
atgttttccatgatgagtgggcaacagttaccacccagggctgctccacaragggaatgaactggagacttcacatgtgttcaatttcttgaaagaaaatg

SEQ ID NO: 188 - DNA, 101 bp, Homo sapiens
acacctggggggtgtactcaccttcttcgatgatgcttttcagcatttctrtgtacatgtccttgttgctgggagctgcgctgttcatcttgaagtggggc

SEQ ID NO: 189 - DNA, 101 bp, Homo sapiens
ttaagagatgatttgagaaagaataaatgttgaatgagcatttattatagrgtcgtttatgctacatttgcattttgactctatttctgccatgcaggatg

SEQ ID NO: 190 - DNA, 101 bp, Homo sapiens
gctcatcagctgtagttagtgtatgtgtactttatgtgtggtccaagtcarttctttcagtgtgtcccagggaaaccaaaagattggacgcccctgtgtct

SEQ ID NO: 191 - DNA, 101 bp, Homo sapiens
acctgcagtggactttgagcaagaaatcagcttttatgtgtcaatccaccrgaatttagggctttctcttaattgcagcaaagcctagcccaccgtgagta

SEQ ID NO: 192 - DNA, 101 bp, Homo sapiens
tcatcctattaaggccaggctgcagaggcgttgcgatggagcagagattgrggagggggtacggtgcgagtctctgcaagatgcacagcaaggcagggagt

SEQ ID NO: 193 - DNA, 101 bp, Homo sapiens
gagtgaggtggaaatgtcggtgcagcctgcagctcacctggttgtcactcrcagatcggcctcggaaagctccaggaagttgatttgggatgagccagcca

SEQ ID NO: 194 - DNA, 101 bp, Homo sapiens
ttttctacaaaactaaacactccaaacacaggcacagcaaactgcatttckaaaggttttgtaagttaaacaagccaaggaagttacatggaaaaaaaaaa

SEQ ID NO: 195 - DNA, 101 bp, Homo sapiens
agtgaaaagttattgtgttcacttgaaagtctaactggcctttagaagggytatgcaactagactcaggcttcaagcatagcaagtggcatcaccaacatt

SEQ ID NO: 196 - DNA, 101 bp, Homo sapiens
acatttgaaacagcatgttaaactgtaagtacatcctcaaaatgcagaaayctccattctcatcaagttacatgctcacagtgacagcctgagaaggtaga

SEQ ID NO: 197 - DNA, 101 bp, Homo sapiens
aagctgccttccttcttgaaaaatgttaatgtctccagtagccctaagaartccataggctccattctgttattcaagatgccaaccaatggttttgacct

SEQ ID NO: 198 - DNA, 101 bp, Homo sapiens
ccgagttctggtaccatgactgtgccgttcaccattgttcttcagcacctrgcactgggctggcactcaacaagaacttgctagatcatgaagatgagcaa

SEQ ID NO: 199 - DNA, 101 bp, Homo sapiens
ctctgttagctaaactgaggaaccacaggcagggtggccttgaatttcagkctgaaggacccatcacccaagagtcttggcagcttcctcagcaaagatga

SEQ ID NO: 200 - DNA, 101 bp, Homo sapiens
ccagctgtctaaaaacatatatattttagagtttgttttcccaaataagayctcatacacggttcatccactgtgtttggttattgggtctctcaagctta

SEQ ID NO: 201 - DNA, 101 bp, Homo sapiens
atcacttccaggctaaatgtcacactcagatactcagctgcctacttactrgacacctctactgagatgtctgaattctggaccctcctcccaagccttct

SEQ ID NO: 202 - DNA, 101 bp, Homo sapiens
gggaagctctggagcattttgtgagcaccgtctcggtggatgggaaagccraagtctctgcccgtctcttactggaggcactaaaccccctccctgggttg

SEQ ID NO: 203 - DNA, 101 bp, Homo sapiens
agtcaccaccctggactatagtctgttgattttctacctctattctcttaytaaacttttggatacattccaaagcatcatggtcacttccagttatgaaa

SEQ ID NO: 204 - DNA, 101 bp, Homo sapiens
agcccagagacctctttggaaagattaccaaaccttgttaaaaacagacayccttggggccagacacggtggctcacgcctgtaatcccagcactttggga

SEQ ID NO: 205 - DNA, 101 bp, Homo sapiens
tgaagaaagtttaatgatggatttttgtttaagtatgcattcatccagaaracactttaactgttcttcagagagacatgatgtggactctaactgatgaa

SEQ ID NO: 206 - DNA, 101 bp, Homo sapiens
tcagctatcacaaaaaataaacgcaattctgaagatagcaatagctcataracatcaggtcaaatctgcaaagatgagcattgtcctaggtgctaaggata

SEQ ID NO: 207 - DNA, 101 bp, Homo sapiens
ttaggtaaagcgaaaaatgacagaattacattaacttgacaaatcaacacmgatagcaggaattttttcacacatttattagtaagcaattgtattagtcc

SEQ ID NO: 208 - DNA, 101 bp, Homo sapiens
gagctttaaaaaaaaaaatgcctggactccacccctaaagcttctgatttmattggcccatttgtttaactatcaatgacaatacagagagatgctaaagt

SEQ ID NO: 209 - DNA, 101 bp, Homo sapiens
aatggatgaaaagtaggattggtttgtttgttttcaggaagtgaggcaatygtaaaagggaaaaatgggaaaggcgaaacaagcaggatgtcttttttttt

SEQ ID NO: 210 - DNA, 101 bp, Homo sapiens
tgaagagggctatctgcctattccagactttatttccctggaaacaaaaargaatatgcacaaatcactgtattttggatttgaatattatatttaaaaaa

SEQ ID NO: 211 - DNA, 101 bp, Homo sapiens
aactcttgagcaaggcatcaagagttggtccttaccccacgcttggtacayttcagccacacttaaggtttaccgttccttttctcatgccatttcctcag

SEQ ID NO: 212 - DNA, 101 bp, Homo sapiens
cgtgagacctcatggttgtcttgtcagtcaaatgctctgaaaccccattgyctgaagctctaggttcaaactttgctccttcaggtgttcagagctgcccc

SEQ ID NO: 213 - DNA, 101 bp, Homo sapiens
tggatataagttcctgtttttctgattaatgtgcatgatcagacaagaaarttatatacaggaatcttaaactaatcattgctacagaaaagaatgggaag

SEQ ID NO: 214 - DNA, 101 bp, Homo sapiens
acacagtagtgtaatcctaatctttattgtgttagaaagttcctcaagacrtagatggaagtccataccccaggagaattactcataaaaatgaaatttcc

SEQ ID NO: 215 - DNA, 101 bp, Homo sapiens
ttcgatatgcatttattagcaaagcttctgaaggtgtcgtaagctgaacgygaggcagctgcctctagaagtgagattcacatgcagggtggaaatggtag

SEQ ID NO: 216 - DNA, 101 bp, Homo sapiens
gggccctttaaacatagccttgttttaataattagaccccccaccccagargagagagggaggaaatgaagcaaggcatccaccctcaggtgtaacatcaa

SEQ ID NO: 217 - DNA, 101 bp, Homo sapiens
atgatctgtgccaatactctgttcttcttagcataaaggtgaacagcaccyctgcactgtagcgtgaaagagtggatttgagtcttggctccacgggctcc

SEQ ID NO: 218 - DNA, 101 bp, Homo sapiens
agtagcagcagtttcacaaagactatctcatttattcctttaataatcctrggcaggaaattattagcagtcccattttatagctaagaaaactgaggctc

SEQ ID NO: 219 - DNA, 101 bp, Homo sapiens
tacatgggactaaactgataatggattataatttttatgacttttatttaraatattgctaattctttaatattttattttccagatttaaggaaactttt

SEQ ID NO: 220 - DNA, 101 bp, Homo sapiens
ggtctacgcactgcatcaaaatccaagctcagaaggcaggaaggcatctcycgcttctacattatccaagtggtgttccgaaatgccctggaaattgggtt

SEQ ID NO: 221 - DNA, 101 bp, Homo sapiens
ttattttcctaactccttgttacttcagtttagcaaattttttaaaaagtraaagtataaatatattaagacttttttgtaggggggctctggaatgtgaa

SEQ ID NO: 222 - DNA, 101 bp, Homo sapiens
tggacagccctggggctcctgctcctcccctacacatcaggcttcttcctrtggagctttctgtaccttcccaagccctcaatgaatgcaaaggaaaaaat

SEQ ID NO: 223 - DNA, 101 bp, Homo sapiens
ccaccacatacacagtaaacattctctcttctcagtggttgaagttgttcytgattacagctctcttatctgttctccctttgatttgctgactgatggat

SEQ ID NO: 224 - DNA, 101 bp, Homo sapiens
tgtgcgcatttcttatatcttcaatttataagtgcagaaattgagaatgaraggtctagaattaaacagtccaggattcaggatcttggttctgctactga

SEQ ID NO: 225 - DNA, 101 bp, Homo sapiens
gttgcttttcccaggaggtgtgagcctacctggaggaggcttaggcacagrgatacctgctggaggtctgagcgttggttgagcacctcctgtttgtagga

SEQ ID NO: 226 - DNA, 101 bp, Homo sapiens
caattatcttccatcatcaccctctccccaactggctgccgtttccacctrtgatagatcagtgttacacatgtgcattttccagaactcccagctgtgag

SEQ ID NO: 227 - DNA, 101 bp, Homo sapiens
ctgacatttactatatgccaaaacagggctgtttaaagttcatggtggttycatctactccttctgaggctacttcaaggtagggaggctacttcaaggta

SEQ ID NO: 228 - DNA, 101 bp, Homo sapiens
attctaggaaaagcacctgcagttattaatgcattaaaccagtgttctgamatgactaaatgcattatttctgctgtagaagaaaacgctgaggtgaggcc

SEQ ID NO: 229 - DNA, 101 bp, Homo sapiens
cacacgccaggcatggacgctttccattgttgtcaacaaaaactcatgcarctcaaatacttaaatgaattctcaaacatgtggttcacaattgaaaaaaa

SEQ ID NO: 230 - DNA, 101 bp, Homo sapiens
caactaagatcgtgtgccttgtgttggtggtaaagcaatatcagagccccrgtatggtaattctcaatctaatgcctgtctatgtgatcaggcttctcccc

SEQ ID NO: 231 - DNA, 101 bp, Homo sapiens
gaatttgataaaaacaagaaatagaagcataattatttttgaaaattacarttaaaactgttagaatcagaagcagaaaccattagcagcatagagagggg

SEQ ID NO: 232 - DNA, 101 bp, Homo sapiens
tctttctgagctttctgagctttgcaatccccagctcacccccccaacacrcccccacagtccttcttcccaacagttgccagcccaccctggccataaac

SEQ ID NO: 233 - DNA, 101 bp, Homo sapiens
atgacccactacaacttcacctcatgtatcttgaactttagggatatagcrccatttaaagagactaacctctcttggttcttgtcagtgaaactgggaag

SEQ ID NO: 234 - DNA, 101 bp, Homo sapiens
aaacttaaggtcagatatttcctcgagacatcagaagttaaagcccatgayataatgagtgaaaacatgcatagtaaactgtaaagctgtctacatatgta

SEQ ID NO: 235 - DNA, 101 bp, Homo sapiens
gtgtgttctttttagtttatcctttcatacatatatgtcaagtctccctarctcaattgtaagccctacaatggtaagggctatgttttatgcattttggc

SEQ ID NO: 236 - DNA, 101 bp, Homo sapiens
gcagagaaagacttctaataaaattccctccatatggaaggaaaaggagayatcgggagttacgttaatcatgctcatttcttaacagtgcaaatatcaag

SEQ ID NO: 237 - DNA, 101 bp, Homo sapiens
tccaaatggccaatctggccactccaaagtcccgcttccagactgaggaargggtgttaatgaagattccagcaaacaacagctctgtcctaccaactttt

SEQ ID NO: 238 - DNA, 101 bp, Homo sapiens
agagaactggagacaatgtagtataatattcggatgtacaaagtacaaacyataaagtctattttgttttaataattaacaaaggtgcacctagtacacac

SEQ ID NO: 239 - DNA, 101 bp, Homo sapiens
taagtacatgacattatctaatattggaaataagagtgcaaagccaaatcrtagccgtgtatagcagtgaatgttaggttgtcaggttcattcaaatgaac

SEQ ID NO: 240 - DNA, 101 bp, Homo sapiens
aggttaccgtgtatgtcaaggtcacccaggggaatgacttaggagtcaaaragcatggatcctactgcccactgtggtgtcaagttgctgttcacccttga

SEQ ID NO: 241 - DNA, 101 bp, Homo sapiens
aaattgcacgcaatgcatacaggaacaaagagagggtcaagatggttatcyttcctcctggcttccaacacaacctgctttgtaaaagccccacactgtta

SEQ ID NO: 242 - DNA, 101 bp, Homo sapiens
catgtcaacaacatctttcagaattggttttctttcacgatgtcgtccagytatgaaaacgagcctcacatgaaatatgctccaagccttttgagggcaac

SEQ ID NO: 243 - DNA, 101 bp, Homo sapiens
ctactccctctatgcttgtggtgattcagttgcagaaagacacatctatayttcatagctgtagaaaaattctttttttgtggttgatttcatgtggttta

SEQ ID NO: 244 - DNA, 101 bp, Homo sapiens
agtcaccagctggtgaccttgagcaagtctttagacctctctgagcttttycctcatgtgtaaaatggggacagacggagcccaacccaagatgttcctgt

SEQ ID NO: 245 - DNA, 101 bp, Homo sapiens
gtcagatgttacacaactttgcaatttccaatatgtgaatattaacatagrccaatgacattattacagaagcttactagaaatatattctgctggtcacc

SEQ ID NO: 246 - DNA, 101 bp, Homo sapiens
ctggcccaaatgccagcatttgctctcctgcctatttcccaggccgtggtraggggcttttcctcagggtcttcatggggagagtcaggggatgagtgcct

SEQ ID NO: 247 - DNA, 101 bp, Homo sapiens
agggagaagccagtacagaggccccagctagagtctgaatgaggacgatcmctctcccctgtcctggggagcctggggtcaccttgcagaacaagatggtc

SEQ ID NO: 248 - DNA, 101 bp, Homo sapiens
tctcccattttcctcctttatgctcctgccagttctgcaaatgtgggagtygcccaaggctttgttcatcagccctcttacctaatcacatttcttccaag

SEQ ID NO: 249 - DNA, 101 bp, Homo sapiens
ccaaggcaggcacctcctggtgctgccaaaaggcatcagaccccatgcccygctccttcctcatcctggactagaactgctttggggtggagacgttacct

SEQ ID NO: 250 - DNA, 101 bp, Homo sapiens
atccatttactgaagttatctgacatggctctcgagtcccttctaccccaygactccccttttttccctttatccttgtgaattatctgttgaagaagcca

SEQ ID NO: 251 - DNA, 101 bp, Homo sapiens
taaaaataaaatagttatgctatttacaagacacacctgttgaaataaggyagtgtaaatataaataaaagggtggaatatttatcatgtaaatgccaaaa

SEQ ID NO: 252 - DNA, 101 bp, Homo sapiens
tgtcagattatttaggcccaatccattctgttgattggacctagtataagyggaaggataaagatttctatccctacattaacacattttatgggttgcaa

SEQ ID NO: 253 - DNA, 101 bp, Homo sapiens
tctggagattcagctgaacacctggagagtctattgaggtctttgtccctygtctgttcagaatggcaccaggtactagcactgtataattttcaaaatct

SEQ ID NO: 254 - DNA, 101 bp, Homo sapiens
atacaaaaaagtagcaaaaagtgggatggggaaaataagattagataactrggtaataaccataaacgatgccctttttaagaaatccaattgttgtttat

SEQ ID NO: 255 - DNA, 101 bp, Homo sapiens
ccaaagaccttgttacagtgtttttaggcatggctcactttataaaggtcrtcacagttggccaagctatctggtatttattactcatttgatactcacac

SEQ ID NO: 256 - DNA, 101 bp, Homo sapiens
taagttctagagtgacagtggcttgctcaaggtcatatgtctaattcagtrgttccagggacaattggataatgtctggagacatttttggttgtcacaac

SEQ ID NO: 257 - DNA, 101 bp, Homo sapiens
atagggcattttgattattaaaactgtgaactgcttcctggaagggcaaayagaggtaactttggctgcatgttacaatccacaattcaatttggcatagc

SEQ ID NO: 258 - DNA, 101 bp, Homo sapiens
ctcagctctaaatgcactggtataactgttgccatttctggacatgccacrtgaaatttttcctttgctcatactattcatgcagtttggaattgattccc

SEQ ID NO: 259 - DNA, 101 bp, Homo sapiens
aaggtttaaggaactttcattttattagccagtggttaagtgcctgtgagmgcaatcatcagcaggtgcagtggtagaagataacaagcttcctaataaat

SEQ ID NO: 260 - DNA, 101 bp, Homo sapiens
ccccattttctgggcacaccccaaacatcttccatgggagaaattggtcaygtgagcccatccctgatgcccgaggagggatgggcttgccaaggctcttc

SEQ ID NO: 261 - DNA, 101 bp, Homo sapiens
cctccctgggaatgacaggttctgtttttcccttcaactattttagcacakggagttcacaactcattccagctacaatgggaaatgtttagtcccgactc

SEQ ID NO: 262 - DNA, 101 bp, Homo sapiens
atgaaatggaacaaggaaaaagaaagattagaatacatgtgaaacctctamaatttttaccatatagagcaggaaagaaacataatctaaaccatattttt

SEQ ID NO: 263 - DNA, 101 bp, Homo sapiens
taaccgaaataccctgtgtgtgtgtgtgtacatatgatcgagccagcctcytcagtgccttgcattgctgttaagaggggaagttctaggctaagactttg

SEQ ID NO: 264 - DNA, 101 bp, Homo sapiens
gccttccatttttaagcaaacattttacaagcttgtactcattctctccaygttgtattaagttttatatttgacattgtatttaaagcatttaccatatt

SEQ ID NO: 265 - DNA, 101 bp, Homo sapiens
tgtgaaaaacattgttagcttgaagaatgtgcaaaaacaagctgtgtgccygatttggctttcaggctgtagtttgccaacttgtgacctaggccttgagt

SEQ ID NO: 266 - DNA, 101 bp, Homo sapiens
gagattgtgtcttaaaaagttttgctctctcctcagaacctagctcatttrgtaacttgttattgctgaataaaaaccaatttattgataaatgaatgtca

SEQ ID NO: 267 - DNA, 101 bp, Homo sapiens
atatataacatagatagtattttttcttgtatcttagtgttctgagttcamctttcttcttctcttcttcctgaagtacatacttgaaacctcattcacag

SEQ ID NO: 268 - DNA, 101 bp, Homo sapiens
ttgtggtaggctgcttaataattaattccctcacctcagtttttgaatgtygttctgtttatgcctcagtatcaaaaacaactgagaaaggggccgcagct

SEQ ID NO: 269 - DNA, 101 bp, Homo sapiens
cttaatatttggctctgtgtccccaaccaaatctcaccttgaattgtaatratcctaacgtgtcatgggaggtaaatggtgggaagtaattgaatcatggg

SEQ ID NO: 270 - DNA, 101 bp, Homo sapiens
gatgaaaaggtcctatcttatcatacacctttaccataaacttcccctccygccacccccagaaggaagagctgaggcagtttccaaaggtgcctgacttg

SEQ ID NO: 271 - DNA, 101 bp, Homo sapiens
gcagagcgatggttcagatcccaggcaggaaggagatggatagcaaaagaktttatcacactactcagaattgtgcttaatttaaaacttttaaaatattc

SEQ ID NO: 272 - DNA, 101 bp, Homo sapiens
tttatccaaagaagggaaatcagaatgatgaagagatacttttcctcttayatttttaggtttatcaccttcatattgtcaaagcatgatgccaataacct

SEQ ID NO: 273 - DNA, 101 bp, Homo sapiens
ctctgcaatttgagtttgttgtgttctaaagaggtacaaaaaaacatgcarctggttagcagcatgctccagagacccagaactgccccagaatgatgggt

SEQ ID NO: 274 - DNA, 101 bp, Homo sapiens
gccaatatccaagacagacgttcaattttccaaaaagcccaagaaattctraaaagtggcctcacaaacaggtttttctgaggcttagacaaaaattcaag

SEQ ID NO: 275 - DNA, 101 bp, Homo sapiens
ttggagaaatgttaattcactctctctagtgtcctgaaatggattggatgrtgcagtatgttgtattgcatggctcctaacccaattccagggagtttctt

SEQ ID NO: 276 - DNA, 101 bp, Homo sapiens
gtacttaggcactaattggcatttttcaacatttctgttaatgtagaacaygtctttcgaaccctcaggggccttgctttggagctaatgaaaataaagca

SEQ ID NO: 277 - DNA, 101 bp, Homo sapiens
tttggggatgtggagggaaagcgagctgggagctgagcccagaccagctcyggtaggagtcagaagaatgtgccctgctgccagtctgagggtcaaagtgc

SEQ ID NO: 278 - DNA, 101 bp, Homo sapiens
tggttaatcattcactcaatcatttgataaatatttgccaagaactgtctrtgtgtaaggtacataatagacactcatttatgtgattatgaatccctcta

SEQ ID NO: 279 - DNA, 101 bp, Homo sapiens
acctctcctacattctaaaagaatggcctgaactatccatgagaacatgayatccgaacttgtaaacttatttccctcatcacagcccataaagaattata

SEQ ID NO: 280 - DNA, 101 bp, Homo sapiens
tgcaacttggtaaaaatattttaacttcatatgctacgaatttgattttcyttgtattaactacacatgtaattagatttttttctttccaaatcatcttt

SEQ ID NO: 281 - DNA, 101 bp, Homo sapiens
agagagatccctgtctctcctcttcttataaggctacccatttttatcaarttagtactccatccttatgaccccttttgatttttttttcttttgaaaag

SEQ ID NO: 282 - DNA, 101 bp, Homo sapiens
ttatataaagggatcttacctctctggatggaagagactgaaatggaattrccaaagtccaaatatgtgtatctgttgcatttaaagtagcacagtttctc

SEQ ID NO: 283 - DNA, 101 bp, Homo sapiens
ttcacctcccaaaatgttgggattacaggcgtgggccactacacctggccrtaagtacagtacacgtcacccctgcttgaaaaatcatcaaagcctttcac

SEQ ID NO: 284 - DNA, 101 bp, Homo sapiens
tcgaaagatttacatagttttagaaaggaggaaaggcaaagagggagttgrgaaatgaaagaaacagggagaagacatggcttctaaattcagggttggga

SEQ ID NO: 285 - DNA, 101 bp, Homo sapiens
taaattgcctgagagcttagagacaatcaggtcaccaccgccctcacaagrgaaaagcttcttacttccgagcagaacggttcagctgggaagagaggaag

SEQ ID NO: 286 - DNA, 101 bp, Homo sapiens
atttccaagacaatttttcatcctttcgtataatattccaggtttgttggkgcctcttctctgtatttcccagaaaataattctaccctctggagaactgt

SEQ ID NO: 287 - DNA, 101 bp, Homo sapiens
aaagatgtggccatcaaggagaagtctttcccatcgtaaatatccaagggygtgactgagccatcactgaactggacccagcaactgatggctgcttccta

SEQ ID NO: 288 - DNA, 101 bp, Homo sapiens
ttgtccttgttttaaggatcttcctgcaggatccactccctagcacttctkgatggcctggctcagggaaatcttcaggaaagagacccaggcttgcacta

SEQ ID NO: 289 - DNA, 101 bp, Homo sapiens
gtttttgctttgaggaaacttgatatgatgttaaatttctaaaagggcaargaaagtagaattgatcaggtagcagaaattttacacagttttggacatca

SEQ ID NO: 290 - DNA, 101 bp, Homo sapiens
tgcccctaccctgagtgctgagagtagaactattgagagacctctttatgmgaaattttcagaaatccaacatggttcttggtctagaaagtgggatcaag

SEQ ID NO: 291 - DNA, 101 bp, Homo sapiens
gtggtcacatttatctgcttctttgtatttctactaatcgttctattagakgctggacattatggatatcctgttgttgcgtgtctggattttgggttttt

SEQ ID NO: 292 - DNA, 101 bp, Homo sapiens
caaataaaatattttttcttttacatagtacatgaaagtaaatctaatctkggagctcatttaggatgctgagcagagtaactggagttagactataagat

SEQ ID NO: 293 - DNA, 101 bp, Homo sapiens
aacaggctgaggttcagtaagctgtcatagctgagctgagacttgaatgcmggtcagatttcagaatctgggctcctcgcacttctcaccacactgcctgt

SEQ ID NO: 294 - DNA, 101 bp, Homo sapiens
ggactctccaacagcataaattggctccagcccgcaagcccaactttccckcagctgagcccctttcagacttctgcccctgcctctgatctatactttat

SEQ ID NO: 295 - DNA, 101 bp, Homo sapiens
cttaatctatttagactgactacagggatctttgattgcctaaaacaacartatagcaatttctctatctgctctcgtcttcctcccgtcatactcataca

SEQ ID NO: 296 - DNA, 101 bp, Homo sapiens
ctcttttgatatccccttcaaaatgtctgctccacacacagagcatcacayatgtggtttatatgtagctggctgaatttctttcctttctctctttcttt

SEQ ID NO: 297 - DNA, 101 bp, Homo sapiens
gatagcgctattaactgtttacacagtaagcacaattttctattctctctytctctctcactggtttcaaagcagccaaaagctttgagccccccagcaac

SEQ ID NO: 298 - DNA, 101 bp, Homo sapiens
ataagctgaaccgagacctgcttcgcctggtggatgtcggcgagttctccraggaggcccagttccgagacccctgccgctcctacgtgcttcctgaggtc

SEQ ID NO: 299 - DNA, 101 bp, Homo sapiens
ggaactttcaagcttgtgttggggacatggatctctataagtaaccacatrtaagtgtaacaagttttgatatgaaagaaaagaacagagtgccctacaag

SEQ ID NO: 300 - DNA, 101 bp, Homo sapiens
tgccacctcattagcaaagttcctgggagccactgacatggaagaccccckgtttccgcctctcggtttccgagcctcagaaagatggactgtgaggcctc

SEQ ID NO: 301 - DNA, 101 bp, Homo sapiens
acatttctatggggctagacttttccttgtcaagattataatttttcttaygagtttttacctgaaacccctattttctaagaccccatggttaatgagtc

SEQ ID NO: 302 - DNA, 101 bp, Homo sapiens
ataagccgtgggtgtaaccatgtcccccacggagtgagaaggggagggtcytctggtttgttactttctgctcatgaggcggggcgatggggagatgcctt

SEQ ID NO: 303 - DNA, 101 bp, Homo sapiens
ctcaaataaagagaaatttaaatcaaaatgacttggctttgtagagtactmctaattttgatttttgtaatcatttcatcttcctatatatgtccttttac

SEQ ID NO: 304 - DNA, 101 bp, Homo sapiens
gaagtgataggtggaaatgataattgttctgtaagagatattctaaggggyaatttaaaacatgtcaatataggcttcttctaaggtggtaaactcagctt

SEQ ID NO: 305 - DNA, 101 bp, Homo sapiens
actcactaacttattctttgtaaaaaggagagcaggtgcacaggtgtagaracaagaaacaacttggagagtgttggcgttgctggagcaccaagtagaaa

SEQ ID NO: 306 - DNA, 101 bp, Homo sapiens
ttcagaacttacgttagtagagtttgaatagttaagacttgaaattaagayccttgctttagtacataatctcacaaatgactttcagaaaatggtgcatc

SEQ ID NO: 307 - DNA, 101 bp, Homo sapiens
gaaattgctgggccatacatagcgatgcgtttgtaaaccagctcactgaayaagaaagccttgattagcatttgctaacatctgtgatgttaatactccta

SEQ ID NO: 308 - DNA, 101 bp, Homo sapiens
ctgacaaccagaactcaagtctctaaccttctctgctgtcccagtaatccrtgcctgccttttctctgccttcagccctttttgctccatcagtactttta

SEQ ID NO: 309 - DNA, 101 bp, Homo sapiens
gtcatgcggcttgctaatgggtttcaaggagcaagctgcaaagagcccctrgacttgctctgatgggtttcaagggacaagatattagtaacgcactcaca

SEQ ID NO: 310 - DNA, 101 bp, Homo sapiens
acactgtgctccgcttttcctcttagcctcttcccctcaacgaaatggtargagttcagctgacaacagggtaaacagattattgtgttattgctggctga

SEQ ID NO: 311 - DNA, 101 bp, Homo sapiens
caaataactataaaataaactcaaaatctttttttcctgcattagttcackgaaaataaaaagggttagcaattagaatcaatagattctttgaaaacact

SEQ ID NO: 312 - DNA, 101 bp, Homo sapiens
attatcatactgctaaacaccatgaaacactgtgtaagtttgcgctattayagttattttaaactgtttttatatttagttgcttacttttaaatttatat

SEQ ID NO: 313 - DNA, 101 bp, Homo sapiens
aaataagcttggacatgaccttttttagcataatgactactgtcatttcartgtcaacctttgaaagcatccattcttgttaaaaacatttgccactgctg

SEQ ID NO: 314 - DNA, 101 bp, Homo sapiens
gggtttacactgctcccctctgctagagcatggactaccagctgacctgcmgagtcactcaccttaaatgttagcagtagctatggggtgtgtgtgtgtgt

SEQ ID NO: 315 - DNA, 101 bp, Homo sapiens
attagttccacaacaaactagatgtagtattttgcatatatttcccctgcyaacgcacctgtggtagtttctagtacatggtttcacttctatgatctttt

SEQ ID NO: 316 - DNA, 101 bp, Homo sapiens
tccagcatattcccagctgtagtggctacggtaaaagactcattctgtatyagagcagacggaatctagaaagacagccatcatctacaagttgggtttaa

SEQ ID NO: 317 - DNA, 101 bp, Homo sapiens
ctgaacagactgtgctttagagcctctggaagacacccaacagaatgttcygaaaaatgcgattatttttacacaaaattgccaatgtaaattcaacttct

SEQ ID NO: 318 - DNA, 101 bp, Homo sapiens
tgctgtgtgatgaggaagccaagaactgaactgtaacccaaacacaaacaygttgcattgccaggaaatggctaatgcggcctcccattacacagagcttt

SEQ ID NO: 319 - DNA, 101 bp, Homo sapiens
ttctaaagtcatccatccccttgacttaagctccaggatggatgcagacayggacggacgcctgtgcacagacaggagtctggaagagcacctgagccctg

SEQ ID NO: 320 - DNA, 101 bp, Homo sapiens
tttaatggaaagttaattgttatgcaaatatgcattcacatgttattttgyttgtttgtttgtttgagacagggtcttcctctgtcgcccaggctggagtg

SEQ ID NO: 321 - DNA, 101 bp, Homo sapiens
actccaagtgctataagcctgcaatggactgtatgtttgtccccctccacygcaaatgtgtatgttgaaatcctaacccccaatgtgatggggtctttggg

SEQ ID NO: 322 - DNA, 101 bp, Homo sapiens
tgaacttaaacccgagtatactagaaatataaattattatatacaaatggrtgtcttttacagcaatagactccagcctaaattgatggtaggggttttat

SEQ ID NO: 323 - DNA, 101 bp, Homo sapiens
ctttactatttagtctagcctgggattctgtatgtgctggctaactgcaamcccgaacaggcaggccttggtgtgggattctctagttgagctgggtcact

SEQ ID NO: 324 - DNA, 101 bp, Homo sapiens
tcttataataaagattattgttattattataaccacctttcagtgtttctrtcttaccctcacatcttcacttttcccctaatctcaagatagagtggagg

SEQ ID NO: 325 - DNA, 101 bp, Homo sapiens
aagtggtaaggttgtttgtctgaggtaggtgattaatagacagccttcctyagcacgtgcaaattaaaatagaagaaggaattatgattggagctctcctt

SEQ ID NO: 326 - DNA, 101 bp, Homo sapiens
cctgatcaaccttcaaaggaatcctcctgagtttacatgagttggaaaatrtgttttcctggctcgttaaagtggaaccaatctcctccgtgtggtagaga

SEQ ID NO: 327 - DNA, 101 bp, Homo sapiens
cgggatatagtagccatgaggaaaacaatgagggctacccttacagcaccrgactccagatggtcttcagtgcattctttgggtagcagctccccaggagc

SEQ ID NO: 328 - DNA, 101 bp, Homo sapiens
gacttgttcaaggtcatataagcagcagtggagtccagaagccaggtttcygtatgccctcttccacatcacattgcaagacaccctctgaaaacactcct

SEQ ID NO: 329 - DNA, 101 bp, Homo sapiens
ggtccaccctctcagttaggcagtagtaaaagatctaaacataatcaatcrggcacattgtatgtagctgtgagggttagaagtacaaaatgtagttgtga

SEQ ID NO: 330 - DNA, 101 bp, Homo sapiens
ggaaataagctcatagctggacagacagcaacgacatagatccggtggagrtgaatctgcagatagaggataattggtcttggcttcaaggatggaaagaa

SEQ ID NO: 331 - DNA, 101 bp, Homo sapiens
acatatgcataatgatcctcaattacgtgccaagcattatggaagtcatcrctaactcctctgtcacctttactttcctgatagcacctgttgatgctgtc

SEQ ID NO: 332 - DNA, 101 bp, Homo sapiens
aaaaggcccccagggaggaattgatcaaaccaaaatgtggatgagtagatrttaggcgaacaccaggcaaatggtggtgagagaagggagcaaagtgtatt

SEQ ID NO: 333 - DNA, 101 bp, Homo sapiens
aaaataatctaaatcttattgagcatgataggattaagtgggaattggacmgatagtggagttggggatggattgtaattatactacactgcgaaaaagca

SEQ ID NO: 334 - DNA, 101 bp, Homo sapiens
ctactttagccactctcaaaactttgtgataaatctgcaatagaggtattrtatatacatgcagaaagctgtgggaagcccagaggagtaagtgactaacc

SEQ ID NO: 335 - DNA, 101 bp, Homo sapiens
acaaaataattccttcttaaaaattatgtattagaaaacttttcaaaattyatcccatcctccagaaaccaataaaataacacacactagaggtccttcag

SEQ ID NO: 336 - DNA, 101 bp, Homo sapiens
cagagctctaccaatcataacagagaaggcatggaaagctggtgaaaatgytggaacgagtttctttttacatgttgttcaatttttatttttgcaattag

SEQ ID NO: 337 - DNA, 101 bp, Homo sapiens
cttcccccaaaggccctggaaactatcattctactttccatctctatgaakgttatactctaagtacctcatgtaagtggagtcatgcagtgcttgtcttt

SEQ ID NO: 338 - DNA, 101 bp, Homo sapiens
gtaaatttattgcttgctcaatccttccttgtatttcattagcatattgcyactctacacttgtcctgtatttagatatttccttcctctatggtttgttc

SEQ ID NO: 339 - DNA, 101 bp, Homo sapiens
aaaccatggggttgagtgcaggtgggataacaatgtagagattggcaaacrtgatgtggaaggtgcgagagacattgtgtccaaagcgatgggcgaggatg

SEQ ID NO: 340 - DNA, 101 bp, Homo sapiens
cttaacatatgcaaaatgaataagtgacaaccccaaccctcaccattggcyccttagaactgaaaataatggcagttgcagtgtttaagggcaacatgaat

SEQ ID NO: 341 - DNA, 101 bp, Homo sapiens
gataatgactgggaattttctagaattggaaatcctcctgtttgggaccaygaagaatcccaggtaggatatgtaaaactaaatgcacatctggcaatatt

SEQ ID NO: 342 - DNA, 101 bp, Homo sapiens
aacaaaacaaaacaaaacaaaacaaaacaaaacacctcttattctagaatrttatgcttcaggagagtgtagctctcctagttttagtttggttcagaaga

SEQ ID NO: 343 - DNA, 101 bp, Homo sapiens
ggcgttcagccctgggctgtgctgtattcagggctctaaaaacgctggccracttgaatgtgtgaatacagttatggcagggagggaggggaggtgctttg

SEQ ID NO: 344 - DNA, 101 bp, Homo sapiens
tttgtgcatactgtgatgattttagaaggtaagaatgtcaagctgtttgarctgaaagtaaagatagccccttatcaggaaagtgccagccacccttgctg

SEQ ID NO: 345 - DNA, 101 bp, Homo sapiens
aatgttgatgcatttaacagcttagattaaatggacaaaatttatgaaagrcacaaactttcaaagcttactcaagaagaaaaagataaccagagtagccc

SEQ ID NO: 346 - DNA, 101 bp, Homo sapiens
gatctcgactcggagcttcttgcccctcttctgtggaatgaaaggggagckaaggaggagggtgtctgaggggcgagagatgagcctggaagagaagcaag

SEQ ID NO: 347 - DNA, 101 bp, Homo sapiens
cgttgttgcataggactagactaaaccaagcgagctgcattccatgcgaaytattctatcgtggggatcaagatctccagctgagaaaagatgccaccaga

SEQ ID NO: 348 - DNA, 101 bp, Homo sapiens
tgatattactaactggaagtcctctatagaatgcttttaccatgatgtacrtagtctgtctaggattccttatgggaaacatacctaaaattgatggattt

SEQ ID NO: 349 - DNA, 101 bp, Homo sapiens
atcttattctgaaagcagatggggcatcagaaacatcaaacaagttaaaaycacaggaattaaattataaattttaaactcccttttattgaaatataagt

SEQ ID NO: 350 - DNA, 101 bp, Homo sapiens
gctgtagatggctataaagcggtccaaagacatggccagcagcacagctgrctccatcatggataaagaatggatggagaacatctggaaaaagacaagct

SEQ ID NO: 351 - DNA, 101 bp, Homo sapiens
gccttagtggggtttcaggagggagcagagataaaaacacatgtcttcaakccatcatcttgaactggaaatcctaaatatcttttgattccttcttttga

SEQ ID NO: 352 - DNA, 101 bp, Homo sapiens
cagggaatgtttcagaatgaagggagggtacatggataaatcagtcagttmaaatattggtgagccccctgcagcacgcgcagatctttgcttaggtgtaa

SEQ ID NO: 353 - DNA, 101 bp, Homo sapiens
aggaagtacggcatagcagttaggcactcaggcatggattcagaaatacgyggaattcagtagggctctggcacctactaacaatttggttactctccctg

SEQ ID NO: 354 - DNA, 101 bp, Homo sapiens
gcactcaataccctgaaaattcgctcgtctctcatgggcctgcctctgaarctgctatgaaagccggcaaccacacagaatttgcctccggtaagaattat

SEQ ID NO: 355 - DNA, 101 bp, Homo sapiens
ctaagtatgatgtagccctctgtaatgataatagtaatagcaatagccagmactccagcaatagtaatagccaccactgacttcattgttaactacaggcc

SEQ ID NO: 356 - DNA, 101 bp, Homo sapiens
gtgagacacacacagagtctgcacagcatctggctgcggggtggattatgrttagccaagggttcctttttatggatgactgcggtagtgaagttgcagac

SEQ ID NO: 357 - DNA, 101 bp, Homo sapiens
acgataatagctcctgtgccaaagaccctgggcagtgtcaggatagctgtrtagctcagtgggctgtagatggctataaagcggtccaaagacatggccag

SEQ ID NO: 358 - DNA, 101 bp, Homo sapiens
aaaactataaaaagagacaaaaattgtgattatgtattgaatgccaaaggrgtcaattctgcaagaaaaataataattgaaaatatatgcaccccacattg

SEQ ID NO: 359 - DNA, 101 bp, Homo sapiens
ttgggcagagttctgtgcgaggggcagcagaggatgcaaaggcctataatytccctgtcctctttggcgcttactgtccactgacagggaggcagaatgac

SEQ ID NO: 360 - DNA, 101 bp, Homo sapiens
ccaaaaaacggttgggagcaactgctctagaaatttgttgtcttcataaaygtttctgactcttagtttctgtttttatcccttctctaagtaccaacttc

SEQ ID NO: 361 - DNA, 101 bp, Homo sapiens
tattctttctcatcttccaaagctatttcatcctccaaagtgtttgttatrtacttttgaatgaatcacaatataccaataccaacacatattttcattat

SEQ ID NO: 362 - DNA, 101 bp, Homo sapiens
ttggtttccattgataatttggaggcattgtcctctgtggagttgtgtcayctatcagcgggctattaatttagggtatggttatagacaactgcagatcc

SEQ ID NO: 363 - DNA, 101 bp, Homo sapiens
gtggatttacttgcttggtttccattgataatttggaggcattgtcctctrtggagttgtgtcatctatcagcgggctattaatttagggtatggttatag

SEQ ID NO: 364 - DNA, 101 bp, Homo sapiens
aaaagctttatatccttacatgaaggacagaacaggcagctatatggtgargaaatgtacagacacaaatatccatatattgaataattggctggctgggg

SEQ ID NO: 365 - DNA, 101 bp, Homo sapiens
atctccgcgtcttcttcttctgtgtgccccagatataaataagcctctatygtatcgctggaaaaacaaactcaccaagttctatattaggcctattgcac

SEQ ID NO: 366 - DNA, 101 bp, Homo sapiens
ttgtgcacacctattacaggaatggaggactcctgtaatgtgtctattagycttaattcgggctccattatacattcctattctgttccctcccctttccc

SEQ ID NO: 367 - DNA, 101 bp, Homo sapiens
acaggctgtcaaatgagagcacgtacttaagaggctaacacagtatgaccrtatgtggcaataaatgagtgctgagtacatgtctatttcttttccagtct

SEQ ID NO: 368 - DNA, 101 bp, Homo sapiens
accctcacagctgctcccactggagccaggctcttgcctggaagaactgcrggttccctgggagactccccagagcccctccttagtggacccaggcccac

SEQ ID NO: 369 - DNA, 101 bp, Homo sapiens
cagtgattacctgcactttctttctctgacttctttggttagctcttctgyttattgaaacaggtaagcagagaaaagtatttaaaaataatctctctctc

SEQ ID NO: 370 - DNA, 101 bp, Homo sapiens
gtaacacaactacataatatccaaagacaaagtagaatggcaagaacttcrcagagcggaataagccttgatggtaaagggaaacatccaaataagcaagc

SEQ ID NO: 371 - DNA, 101 bp, Homo sapiens
tcatcatcttcttgctgcccaagcctctgttcagtcccccaccagatgcgkcattcaagttgtaaagcaaatgtactatttcttgacatttctagaaaact

SEQ ID NO: 372 - DNA, 101 bp, Homo sapiens
gtttgagtcatggttttggaaaatcacatgatccataccagaggagagctktgtcttcaaattatcttctagaaaggttcaccagaaagtacaaaaatgtt

SEQ ID NO: 373 - DNA, 101 bp, Homo sapiens
taagtcttgaatttgggtagtgtgaatcctccatatttgtttttcctcttmagtattgttttggctattcttggtctcttgtctttacatttaaactttag

SEQ ID NO: 374 - DNA, 101 bp, Homo sapiens
cagtggtaaccaggcagtaagtaccatggattttggatgagactcagtacmttgctggcatcatgtgcaacccagcacattcccagctctggtggccacag

SEQ ID NO: 375 - DNA, 101 bp, Homo sapiens
tgtgtgtgtgtgtgtgtgtacacatgtgtgtgcgtgcatgctttttcatgrggcacacttattttcagatgttcacatggactctttttgagattccccag

SEQ ID NO: 376 - DNA, 101 bp, Homo sapiens
caatgcaagggatttgtaaagaaacagggaaatgaatgatctgacaggccrtttgttaccaccaacatttttcttaatttaacctgaacttacttgctctt

SEQ ID NO: 377 - DNA, 101 bp, Homo sapiens
atccatgcaatgcaataaacagccatagacagaagcgaagcgctgatccaygctacagtgtggagaaatcttgaaaacactagggaagtgaaagaaaccag

SEQ ID NO: 378 - DNA, 101 bp, Homo sapiens
tataccaaggatagtttgtgcagttacaccggaaataagatatttcctgcrtttacagacatctacatgcttgcctttttttccatttcccactgaaccag

SEQ ID NO: 379 - DNA, 101 bp, Homo sapiens
atgggggatgagacaaagaacttcatgggtgcagcaggtctcttggtgtcrtgtgggaaacacaagcagaatcagaagttcccctggcctctccctgggtc

SEQ ID NO: 380 - DNA, 101 bp, Homo sapiens
aaagggagaatggggtggagggccagaaagcaggagtgccatagagtcagkaagtgaaaaattgcaaatgtgggcaatgtgattaggcaactgggtgtgta

SEQ ID NO: 381 - DNA, 101 bp, Homo sapiens
caccctagaaatcctggagggaggaccgaaaggtagcatggagtcaataaygagcctctttttatttaactatgattacatgtcaatcaatgtctgattct

SEQ ID NO: 382 - DNA, 101 bp, Homo sapiens
cttggcatgctagttaacccaagggatggctctacaatgccttacagtttrtaaagtacttccttctgtattatttcatctgaccttcgcaataaggctat

SEQ ID NO: 383 - DNA, 101 bp, Homo sapiens
aaatccacagccattcaggtggcttatgttactggcacttagcattccgcraccatggtccccagaggctctgtggacagaggtgccctgcagttcctttg

SEQ ID NO: 384 - DNA, 101 bp, Homo sapiens
aacagccttattctttcttatttccagtaagtattccaaagaaaaacatgytgactggcccagctcacttttgcacatctctgggtcatgaatctatgtct

SEQ ID NO: 385 - DNA, 101 bp, Homo sapiens
taatgcatctaaagttcaggatgtataatgaaatctaggaatgtgaactaytcaggagaaaaacagacatgatctaagagttcaaaagaaaaacattagca

SEQ ID NO: 386 - DNA, 101 bp, Homo sapiens
gtgagatcatggacttgggccccctaggccagcccagtctctttgcagccraggaaagtgaggcttagctgtcgggggctgtggggggatgcagcttgcca

SEQ ID NO: 387 - DNA, 101 bp, Homo sapiens
ctacactaacaccatgagataggtattcttattagcatcagtttttcgaaygagtacttcaagtttcaggaaagtaaagaaacttccctgaagacagtatc

SEQ ID NO: 388 - DNA, 101 bp, Homo sapiens
ttctttatcattgaatttcaaaatctttactaggacaaatcttggtggtargctttctatatcgaatttccctaggcacattttgcttttgcgatttgcag

SEQ ID NO: 389 - DNA, 101 bp, Homo sapiens
cagggtgtgtccacactctgctcacaggtggatccacggctttccagtgcrgagagtcgagatgctccctgcagcccaggccccgggcacctcctgcaacc

SEQ ID NO: 390 - DNA, 101 bp, Homo sapiens
tacagaaaattgccaacctctggaagcctcagcaggaccaatgtcctccaygcagagcccttcttatcccctaggaccgcaggcccaggctcctctggggg

SEQ ID NO: 391 - DNA, 101 bp, Homo sapiens
actgaaactctctgcccacattccacattctccctctccccaacccttgakaaccttttctttccttctctccttcctttcctctttccctccttccttcc

SEQ ID NO: 392 - DNA, 101 bp, Homo sapiens
ttctgaaccaggcaaaggatgatggggaatgcagtcttacgacgtgatgtygcgtttagagggttttcatcagttttaatgaaatacaaatgcacccaaag

SEQ ID NO: 393 - DNA, 101 bp, Homo sapiens
ctgtccccgtcgtccttcctatgctcacggcagtcacgtgagcctaaagargtcatgaaaggaacatagcgaccactccatgatgtggattaactcatcct

SEQ ID NO: 394 - DNA, 101 bp, Homo sapiens
actggacccagcccagcccagctctttccactgctcacctgctgcccctgygtttccagggactccacgctcaccagggacacctcgctctcccttagggc

SEQ ID NO: 395 - DNA, 101 bp, Homo sapiens
cataaataacaaaaagtctactaaaacagataccttgggatagatttattrtgccattttaggatttcactttcaagttgcttaatagaaaatcagtgact

SEQ ID NO: 396 - DNA, 101 bp, Homo sapiens
aagaaagattttgatacagaggcacacgcagagggaaaacagccatgtgamgacagtgacagaaactaaagtgatgtagctccaagacaaaaaatgccaag

SEQ ID NO: 397 - DNA, 101 bp, Homo sapiens
actagttacaaggcagaattatctttctgattgcatgaaacccatagatcrttttctctccaacagaaatcttttcagtaacctcaatccacgttttggct

SEQ ID NO: 398 - DNA, 101 bp, Homo sapiens
acagtgtctgcccaggtcagacactgtgtttagaattgctggtgattttgkagttcagaattactggtgattctgtgtctccatccttcttcattccaaat

SEQ ID NO: 399 - DNA, 101 bp, Homo sapiens
ctctattacaaagataaaatggcaagctacagagtggtagaaagtatttayaaaccacacgtctggcaaagcacgagtatctagaatacacaaagaatttt

SEQ ID NO: 400 - DNA, 101 bp, Homo sapiens
atcctaacagaagtcacatggctttatttcatggccagaaccaccaggctrttacaggaaagccaaaaagaccagacagagaagaatgtttccttacagta

SEQ ID NO: 401 - DNA, 101 bp, Homo sapiens
ggtgacagccatatgctcctgatcacaagaagaaattatatcgggtccagyggcggctgtcacaaagccatatggggtggcatggcagccttctgcaggtg

SEQ ID NO: 402 - DNA, 101 bp, Homo sapiens
ctagtaaccctttgtgaggctacaaaaaaaaaaggcatatttgcttgcccrgggggcttctcttccagttcacctgggtagaattctgggtgtagtcccca

SEQ ID NO: 403 - DNA, 101 bp, Homo sapiens
gtaggacttactttgtgcctgagttcagtgaccttgtgctcactctcttamttctccttcctccctggctggccattccttctcagtttgctttgtaactc

SEQ ID NO: 404 - DNA, 101 bp, Homo sapiens
tttgcttcctctctcacaatgtgatctctgcacatgttggtcccttgtcamcttctgccataaggagaagcagcctgtggctcgcaccagaagcagatgct

SEQ ID NO: 405 - DNA, 101 bp, Homo sapiens
agtgttgttctgtgttattattctctaatgtagaatcgcaccatcctgggrgtcaggcatcttccgcctcctctttgacctagtttgtggcacacagcagg

SEQ ID NO: 406 - DNA, 101 bp, Homo sapiens
aagtgaaacttaaatcttgaatcatgagtaaaacgtaccaagcaaaaaacrgacaatttgatctttgacgaacctgacacaagcaatggggaaaggattct

SEQ ID NO: 407 - DNA, 101 bp, Homo sapiens
atctgccttctagtatgtgaggcaaccttcatcagcatgtagtagcatgtyggtgctggctagttactttccaagagggagataaacacctcaaaataagc

SEQ ID NO: 408 - DNA, 101 bp, Homo sapiens
tagtgaggagtgagaattatatcacaggatttttgcaaaagctgtaataakataactaatactactgcattttgttcccaacattcacaattgaagaaaat

SEQ ID NO: 409 - DNA, 101 bp, Homo sapiens
aaataaaaagtcataaaaagaggaaagaataaaaatttccattcaataggrattgatcttaaacatagatggagggatcagacaagggaagtcatgtgatt

SEQ ID NO: 410 - DNA, 101 bp, Homo sapiens
acaagtggttaggtagacagaagctatcgggaacattctggactgctggarattgctatagtctcaacattttctaagacagtcgggtatagagctttgta

SEQ ID NO: 411 - DNA, 101 bp, Homo sapiens
gttgcagcccccctgagcccccattcacaggaggtctcctgctacattgamtataacatctccatgcccgcccagaacctctggagactggtgagtaaggc

SEQ ID NO: 412 - DNA, 101 bp, Homo sapiens
ggagtaaggtaagtatgcatggctgacttgaaaagatactttctatatacrttgcttaataaactatcaaattgctgcagaatgatatatgtggatgagat

SEQ ID NO: 413 - DNA, 101 bp, Homo sapiens
atataaggcaaagctcataaccatcctccagtgttcaggctcagcataagycctctaggaaacctttgtacctttctttgggcctcccccaccatagccct

SEQ ID NO: 414 - DNA, 101 bp, Homo sapiens
gtccttaaaaggaagggagctcccgtattcccctcttcttccttcctctgkgctggcatatgaacacaatgactggaagctgaggagtcatcctggatcat

SEQ ID NO: 415 - DNA, 101 bp, Homo sapiens
ctcggttgtcctcaagcaaaaggaatgctatcaataagccttcctaccacrtattgaaaattaaagtccttcctttttacactttaagaccttctaataag

SEQ ID NO: 416 - DNA, 101 bp, Homo sapiens
ccctaattgagaataaatctgtctgaggcagatgtttggcaaaagtagtgygagtgggttttcgttaggtcttttaccgttcttagaaatgctgtcagcat

SEQ ID NO: 417 - DNA, 101 bp, Homo sapiens
ctgcctcagcctggagaccaggatggcacccccaagtcctttcaaagtcayctgcaatggaaactctcttgcttttagtttttcccaggacagtcagccaa

SEQ ID NO: 418 - DNA, 101 bp, Homo sapiens
caaataacccacactttccttacaaatatgaattgacatatttatcacccrtcggtctggttttaggttttctattctgcgttgttctctgcctgactatt

SEQ ID NO: 419 - DNA, 101 bp, Homo sapiens
gaagtatggagacaaaaagttaaggagggtgagaggatagaggagtctcaytgaagatcccctggttaaaaccactgcctcatttctgtgaacagcctact

SEQ ID NO: 420 - DNA, 101 bp, Homo sapiens
ccatgtccctgtgtcatttttactcttggtgcttgtcgcctttcaacataytatatatctcatttgttttccttgtgtattaaccatttcccacattaaaa

SEQ ID NO: 421 - DNA, 101 bp, Homo sapiens
cacacagctgcaattgagtcctccactgatgctaccaggagctctagaackgggatggggccttcagggtgttctgaatttgggcaaggaggctgggcttt

SEQ ID NO: 422 - DNA, 101 bp, Homo sapiens
aactcagagtggatttggccatgaaagataaagtaaaagcaagtataacaygaaagaacaaaaaagcatgactcatatctgtgcaggctttttaatatgtt

SEQ ID NO: 423 - DNA, 101 bp, Homo sapiens
gccctataagagaggacagcagaaacaacagaggaaaaagtgacagggtckgctgttgaaatgcttatcaaagagtgggcatttgaactaagttatgaaag

SEQ ID NO: 424 - DNA, 101 bp, Homo sapiens
gctatcataaaacaaatattaagcacagcccctaaataatctttggcagtrtatgtcttggcaattttgatgtaattatgtttcatcattttctactttcc

SEQ ID NO: 425 - DNA, 101 bp, Homo sapiens
acttacactgaatgcaatacatagtaatttgaacaggagtttaatctagtyaatggggaccctatggagggtcagaggactccaatagccagtgtgagttg

SEQ ID NO: 426 - DNA, 101 bp, Homo sapiens
tagaaaaagaaagtaaaaaaggaaaattcatgaactgaaaaaagagtgacrttttcataaaatgagagaaaataaggtctatttataggtggaagggctga

SEQ ID NO: 427 - DNA, 101 bp, Homo sapiens
atgaataatattcccttctgtatatgcaccacatcttaaaaaattcatttrtctgtagttagacaagtaggttgattccaaatcttgactattgtgaacag

SEQ ID NO: 428 - DNA, 101 bp, Homo sapiens
aaggagataatagtgggtgggtgattacttgaaactgatttttggagaagktcattaattaaatattcattcattaattaaagaaacaatgtatgtcaata

SEQ ID NO: 429 - DNA, 101 bp, Homo sapiens
ttctaacccagaagctttctatttttttgttttcagaagatccccagatarcatctatccaaactaaaatgagaacacagtctgacggacatgaggggatt

SEQ ID NO: 430 - DNA, 101 bp, Homo sapiens
actcgtggagagtgcttctgcattttgatactctgaagtgattcctgcaarcaacagttgtttcacattctagactagaacttcagagtcatgtacaactg

SEQ ID NO: 431 - DNA, 101 bp, Homo sapiens
gcttggtgatactctttcaagccttgaaggggcctgttgatctttccctaytccactgccaacttcagttctccagttctctaaagtggggctttattcta

SEQ ID NO: 432 - DNA, 101 bp, Homo sapiens
gttcaagagttgggcatcttaactactttatcctctgctgtcaaagttctyaaaggtctcttggtctctgatctgctgccagcctctgcctggctggtaaa

SEQ ID NO: 433 - DNA, 101 bp, Homo sapiens
aggactggacatatctgcactcctgccctctgacttcagccgctacttccratatgaggggtctctgactacaccgccctgtgcccagggtgtcatctgga

SEQ ID NO: 434 - DNA, 101 bp, Homo sapiens
gggtctggaaggacctctgcctgggtgtttgacttggaaggggacagtggytctgggcttgggttggaattcagaacccatccccgggcagctgcgtgggc

SEQ ID NO: 435 - DNA, 101 bp, Homo sapiens
gtctttacagaactagagttcagggggaatatcagaggtaaaaaagctgaraaaagcattgacttcaaatgccagataccattttgatttttggcagagca

SEQ ID NO: 436 - DNA, 101 bp, Homo sapiens
tggaggtgtgggatagccagtattacaaccaagagtttacatctgtgttcyccaggcccacttaaatagaaccacagctaccaatcactgccatttatcat

SEQ ID NO: 437 - DNA, 101 bp, Homo sapiens
atccagtgtctgggggtgggaacgagagttatcatatggccaaataacttmaagctgagcgatgggcatgtggcatttattgtacaattctctgtgctttc

SEQ ID NO: 438 - DNA, 101 bp, Homo sapiens
ctttctaaatggaccctaagcttctctaggtcaagaaccatgatttagggktcttcgatgtgcctatcacttgagtcaaaaaccttaaaatagtaatgggc

SEQ ID NO: 439 - DNA, 101 bp, Homo sapiens
tgagattacaacctagtagaagcctgtaagtcagtgtctacatgacagcaytttgcatgccaagtccaggccatgactgctcattgtagacgttgcttgtg

SEQ ID NO: 440 - DNA, 101 bp, Homo sapiens
ctgtatagtttgtgagttattgcaaagggaggattgcccaggaaccatacraggctgctgtggagcagactcagccagtgctctcatatccatggtctccc

SEQ ID NO: 441 - DNA, 101 bp, Homo sapiens
tagttatgaagttttaggggaaatatgtccccctttttcacttggtaccamgttttgagataggcaattttctttgtagtcccctgaggaaggatttgggg

SEQ ID NO: 442 - DNA, 101 bp, Homo sapiens
gttacaagtcagccgtctgggtgttaaatctacacgtaccaaataaccaaytgtacttttttcactgaaatgttagtattatgtagagacagccacgactc

SEQ ID NO: 443 - DNA, 101 bp, Homo sapiens
ttctctcttacatgaacaattgaacatttgttagacatagtgatgctcctyagtattacccattcacttttttgggaggcacaagaaaggattgcacttca

SEQ ID NO: 444 - DNA, 101 bp, Homo sapiens
ttgaatccagaagctggccagctgttccaaatcagctattgttatcaatckcctctgaaaatcaacttatcaagcagttcacagctatcagatgttaaaaa

SEQ ID NO: 445 - DNA, 101 bp, Homo sapiens
cctgctaattctttctccatctgaggggtgagaaagacttctttttagctrtctctttcactgccaacctgctttgataatgttctgggggctttaccaga

SEQ ID NO: 446 - DNA, 101 bp, Homo sapiens
aaggcccttgagactgaggtctcaacagattgggacaaagaaggcaacagrataagggcataggtgttaccctgggaccccagagacctgaattctggctc

SEQ ID NO: 447 - DNA, 101 bp, Homo sapiens
ccagggtttcagacaagtctagagcaagtcaggatatcaataagacccaayaggatgtagggctgcctgtctagggagacatttagcttatcttccccggc

SEQ ID NO: 448 - DNA, 101 bp, Homo sapiens
ctcagctggagagcaaccctttcggtttaaaataaactaatgaaatccctraggacaaatatcactatgatatgcacaaaaacagcacattaatgcaacaa

SEQ ID NO: 449 - DNA, 101 bp, Homo sapiens
ttttctcttaaaagactcagtacattattagaaatgcctttcactaacatytaacaaataaaacagttctatagggacaatgaagttgacatttccattgt

SEQ ID NO: 450 - DNA, 101 bp, Homo sapiens
tctactggtcccatgtcccagagatcacaatgccttcctatctatcactgycggccattgctggtatttaagggtatatctctcttctgcctccaccctag

SEQ ID NO: 451 - DNA, 101 bp, Homo sapiens
acagtcttcagtttatttctcactgaactgatcctttgtttccctcccccyaccacctacagaatctaaattagagtgatttcctcccgcagaaaagtcag

SEQ ID NO: 452 - DNA, 101 bp, Homo sapiens
gcatctttaggacttctcccttgggattatcttcactattagcttttctcrttttgttttattttttcacatcccctcaatggaaggcaatacacttagca

SEQ ID NO: 453 - DNA, 101 bp, Homo sapiens
ttctaatcattcagataaaggtttaatttgtaccaagattatcctcaaaayatcactgaatacagtaaacactggcaattgccattaaaaacaaattatat

SEQ ID NO: 454 - DNA, 101 bp, Homo sapiens
tcatgttcctaaaaggacaacatgaagtataaacccaaacaatagatgtamactaatcatccctaacaatatccatagtgaatggttccaacagagtgcac

SEQ ID NO: 455 - DNA, 101 bp, Homo sapiens
catgtactagcatcaagaaacatctgactcccattctgtcattctgtaccyacgtcatcttgactagacatcaattaagagtttcctggaaaactcggaac

SEQ ID NO: 456 - DNA, 101 bp, Homo sapiens
gaccagactaaccctttttccttcttttggaggttatgattaggattgtcmgagggcaaagggtttaattttttcattaaactaacaacatgttttgagca

SEQ ID NO: 457 - DNA, 101 bp, Homo sapiens
atctcctagcctacaaaattattctttagagaatccattttcccacaagayatgcaaaaactaaaacaaaccacaacacgtgggccagatgtttcttcaat

SEQ ID NO: 458 - DNA, 101 bp, Homo sapiens
gaagaacgagccgtttaaatcacacatcagaccataccattcctctgctcraaaccctgcaatggtttcctgtttcactcagggtaaaagctaaaggtcct

SEQ ID NO: 459 - DNA, 101 bp, Homo sapiens
ggaatttttagagaaactacatgttctaacatgttctcttagggtgcttcrtacagatcgtcaaggaagtatcccaaaaaaaatcaatgaacacccggaat

SEQ ID NO: 460 - DNA, 101 bp, Homo sapiens
ttttgtccccattttttctcccatgtaagacatttttaatctaccttgcartgaagaggctgttaaacacttgtaccagcaccacccagcttttccatgtc

SEQ ID NO: 461 - DNA, 101 bp, Homo sapiens
ctggaagttaggatttgtacaaaagattgaattagttctcagtgacccctygacctaacccttggtccctcactgagtgggctccttggagcgctgtgatc

SEQ ID NO: 462 - DNA, 101 bp, Homo sapiens
ttgaaacattgtttttgtagaatttgcaagtggattttacagcgctttgargattttagagcaaatgaataaatgtaatccaccatataaagagaaccaaa

SEQ ID NO: 463 - DNA, 101 bp, Homo sapiens
ttttaagcaataagcatgctgtgcttaggctgtctcagcactattgttaartgctttaattatgtaacttttgatacattcatgttatcatatgttgtaat

SEQ ID NO: 464 - DNA, 101 bp, Homo sapiens
ctgaggcagtgcatacccaagactgtcacttctgctctgcatacctttaakattcttccttaggattctctagtacacagtggtctcatccaccagctgcc

SEQ ID NO: 465 - DNA, 101 bp, Homo sapiens
caaggagaggagataagcatcctcactacaacctgaccaattcttaaccayagaatctgtaaataaaacaaaatggttgtttgcctctgagtctggggatg

SEQ ID NO: 466 - DNA, 101 bp, Homo sapiens
atgtcaaaatattgcaaagctcctactgcaaatggctcatgtaaccaacaytattagagaatatttcctgtttagaaatttattttaaaaattgaaattaa

SEQ ID NO: 467 - DNA, 101 bp, Homo sapiens
cagaggtgtcacttgttttaaaagtgagaaactaaccagtgcttagaactrtaacccccagagcattgcctatgaataccaaggacctagaaatctcctca

SEQ ID NO: 468 - DNA, 101 bp, Homo sapiens
ggctgaacagatgaaattgctttagctaaaggaagtggcacgaatttactyatttattagatgtgcaggatacatccatcacaccgacctctggatcaact

SEQ ID NO: 469 - DNA, 101 bp, Homo sapiens
ttcctcataaacatcaagtaatgtgctggtaactgggaaatactgcagttkgttagtagaattttatcagaagtcaacaaaatattccgttttgcatgcct

SEQ ID NO: 470 - DNA, 101 bp, Homo sapiens
cacatcatctggaaataaagaacattttgcttcttcctttcaaagctacaygctgatctatcttgaagtttatgggtgtgggttcttctgccatctcaaat

SEQ ID NO: 471 - DNA, 101 bp, Homo sapiens
gcagtatctcctgggtatgtccatctggttatgtaaagtgaattattggtrgctttccccagctctttcaatttttaaaaaataagtaatacatccaatgc

SEQ ID NO: 472 - DNA, 101 bp, Homo sapiens
caggtgatagattaaaaactatggttacttaaaaaatgaccattgaacttyataaaactattctgcctgatttccaactggtatcaaaattttaagtgatc

SEQ ID NO: 473 - DNA, 101 bp, Homo sapiens
caaggataattatggctatcttttgtgtcttaattttgtttgtagtttcaygtgaaagtcttcattctggggggcttagaattaaagccctctttatttag

SEQ ID NO: 474 - DNA, 101 bp, Homo sapiens
tgccaagcataatcttaccatagggcctttgaacgggctatgcctccaccmgaaccacttttcccgtttatctgatcactccttcaccttcaagtcttgat

SEQ ID NO: 475 - DNA, 101 bp, Homo sapiens
ttgtatatactggaatagagtaaaccatacaacaaaacagaactctgtctrtatcaggaaaccttgtttaattttagggaaaatgatatacatttgaatac

SEQ ID NO: 476 - DNA, 101 bp, Homo sapiens
tatcaaaattttaagtgatcaagagtaaaagaactttatcaagaattatarcacttaacaggtcgacacagatgcagcccttttattatataggtataatg

SEQ ID NO: 477 - DNA, 101 bp, Homo sapiens
taaaatgttgggtggagatggtgccttttccagtggaagctactcatggcrtcagaacaaacccaccccacggacaaattcacaaagggtgtaaaactgga

SEQ ID NO: 478 - DNA, 101 bp, Homo sapiens
tgtcatacattggcccagcacatatgtgtgattgtgactctaatatacacrctcaactaaaagttaaaggtgtcaccctcaaagatcaggagattgtgtca

SEQ ID NO: 479 - DNA, 101 bp, Homo sapiens
cgaagaacagagggccaggaagctaattaataaatgacttgctcaagacarcacagctagcaaaggcagcctgatgtggagcacagcccagcctcttccct

SEQ ID NO: 480 - DNA, 101 bp, Homo sapiens
tggttaatttctactattacagtggtccatagactcatttgaagcaaattyatgaaaggaatattgccgtaaattcgatgggatttcatcaatatcttaaa

SEQ ID NO: 481 - DNA, 101 bp, Homo sapiens
agtttaaatgcctacagcaatcttccaagacacaggtgctatttttgatarcactatggaactgtacaaaactatacaaacaacattatgactctgcactt

SEQ ID NO: 482 - DNA, 101 bp, Homo sapiens
gccagtactgatggccctgtgccttcagtctagcgtcctggagtctgaaaygggagatggaagacagtagcttgaatacagagggtgaaagattttcctcc

SEQ ID NO: 483 - DNA, 101 bp, Homo sapiens
acaagcccagagaaaacatccatacaacaggcttgaaagactccaagaatmtctcgcctaaaaaattggtatcatatttccccagacaaaagccaacttaa

SEQ ID NO: 484 - DNA, 101 bp, Homo sapiens
aaagatacagggagtggactgggctttggaacaactcagttttacttccayggtattctgatgctcaagcagccacagaactcagatttcagggcagatga

SEQ ID NO: 485 - DNA, 101 bp, Homo sapiens
ttgagttcagtgtgaggaggtttatgcctagaaaaggtgctcaccaataaygtgcctcagttcccataatagcaagatcgagaaggttctttagtctcccg

SEQ ID NO: 486 - DNA, 101 bp, Homo sapiens
aaaacttcatacctctccagggagacagttcccagaaacctccctcccctrcaaagcactcctataacaaataaataaactacatttcccaaagttctctt

SEQ ID NO: 487 - DNA, 101 bp, Homo sapiens
ttctccttcaggaattcttatcgtgcataagttagttctctagatagggtyccataatcccataggccttctccattttttttcactcctctgactagaaa

SEQ ID NO: 488 - DNA, 101 bp, Homo sapiens
atccctaactggagatcatctcctcagtgctggacttgagattcaaattcrggaccttacttctgagtctgctcaaaagcactctgaaacagcatccagag

SEQ ID NO: 489 - DNA, 101 bp, Homo sapiens
tttattctgtaatgtgattataagccattagcaggatttatgcaagggagygatatggtagatttacacgcttaagagattattttgcctgttgggtagag

SEQ ID NO: 490 - DNA, 101 bp, Homo sapiens
gatactgatctataaaatataagccaaatactgttaagaaaagttaaccaygaataagccaggtatggtggctcatgcctgtaatcccagcactttgggag

SEQ ID NO: 491 - DNA, 101 bp, Homo sapiens
atctggaagacccaccctcaagtggtacataccagtgccattcacattctrctgcctaaattactcactttgcctcacccaactttcacaaagcatggcaa

SEQ ID NO: 492 - DNA, 101 bp, Homo sapiens
tgtgtcatttaaccttgcagaagtttaaattctaccagtatttcctgttayagtttctgcctttggtgtcatgtgaaaaaaaaagaccattactatagcaa

SEQ ID NO: 493 - DNA, 101 bp, Homo sapiens
tattccattaactaaacagcaacctcgaaagaaatcaatactcggaaggtyctgtagtagcagccattccatggatgggacaccagaggtggggcaggagc

SEQ ID NO: 494 - DNA, 101 bp, Homo sapiens
gctcccagcagctcacccctccagtggctgttctttctacctgtcaaagcytgtgctgacacatatactgggaggtgacccccagctgcggctgccccacc

SEQ ID NO: 495 - DNA, 101 bp, Homo sapiens
tcaatatggaagaacttgtccaggcttgtgcagaccaccatgtctctgccktacaggctgacatttaacaatggtgaaggcaatctcttcttggaaaaaat

SEQ ID NO: 496 - DNA, 101 bp, Homo sapiens
agactgtgcagtgtccagttcttttattaagtacatggggtctgtagtcayacttcctggggcaaaatcctgcctcttatgtttttgaccttcggcaagtt

SEQ ID NO: 497 - DNA, 101 bp, Homo sapiens
gtttagcatctgtggtaagtgtgttcgaaggccgtgtaagcacattttatyatgagcatgtcttacttccaagttaagataaagatttggaaattaatgta

SEQ ID NO: 498 - DNA, 101 bp, Homo sapiens
atccagaatctacctacattcattgttattaatttgtacccctggtgttcrgccagtatcaccttctcccaatctatttcagccagtgacaatgaggacat

SEQ ID NO: 499 - DNA, 101 bp, Homo sapiens
agagatgcccccgccctccagggaaactgcacagacattacaaacaagcaygctcttatcaagcaggagaggtctgggtcggggggctggggggaaggatt

SEQ ID NO: 500 - DNA, 101 bp, Homo sapiens
ttattgctgaattggtataaagatgaatatatgcctggctgcattctactyattcttcttatttcaagagaaattaaatcatttcatgggcccctaaaatt

SEQ ID NO: 501 - DNA, 101 bp, Homo sapiens
ggctaatcaatttgatgtcttttaaaactaatattcttcaaattttttttyagtgtctatttaggggaatggctgatggctgcatgaagtgggggactcag

SEQ ID NO: 502 - DNA, 101 bp, Homo sapiens
tcttgcttccaggggaagctgccaggtagaagtagtgaggaatctggtatygcactgtcccaaggggcgggacacctgcctttgaagacccctgggttctg

SEQ ID NO: 503 - DNA, 101 bp, Homo sapiens
ctactgatctttcagactgcactgttcattctaattcttataatacaaagkcagagcagcagatactctagggaaagaatgcttgcaccgtgaaatccaca

SEQ ID NO: 504 - DNA, 101 bp, Homo sapiens
aacctccctccctgctgctatcttatgtacactcttaatgtgcctaacctyccacgagtgtgcagagatgctgctagagcagtccctgcttagatcactgg

SEQ ID NO: 505 - DNA, 101 bp, Homo sapiens
ttgttcaaaatgtatattttctcgtttttaaattatgtaattttggctggrcgtggtggtttacgcctgtaattccagcattttgggaggccgaggcgggt

SEQ ID NO: 506 - DNA, 101 bp, Homo sapiens
attcacacctcaggtcttcactttggggagcgaagccttttagcagaaatrccagaagtaccatcttgccaaatggtcaggaactgtctgatagagatgga

SEQ ID NO: 507 - DNA, 101 bp, Homo sapiens
aggcactgttttatcatggctcatctagattccaaagtccacaataacccrgatgatccatgtggtcatatcatgctcttcacaagtacatgcctctgctt

SEQ ID NO: 508 - DNA, 101 bp, Homo sapiens
tcaactacaggtgtgttcctgatggcctttagctggagcgtactgacacartaacaggctttgaaattcaagtgattcagtttggcatcttagctccacca

SEQ ID NO: 509 - DNA, 101 bp, Homo sapiens
catgaatatgtacaatgattatttgccaatcaaaattctgcatcctccagmagcatgctatccaaacttctttcatcatccctctccctctggaggaggac

SEQ ID NO: 510 - DNA, 101 bp, Homo sapiens
ttaacaaaaaacgaatattataaattgattatgtttccttgcagctggatrgcttagcctgaagtatggattgctagtaattcctccagtcactcaacatt

SEQ ID NO: 511 - DNA, 101 bp, Homo sapiens
ttcatccttaataaaaagaaaattgcatagctttttatattgttgcaaatkcatctcccaatatcattgtcagcttagtgatattctccatattttaaaat

SEQ ID NO: 512 - DNA, 101 bp, Homo sapiens
aatgaagtaaagcaagtttcagctgtttctttccccaatgcacaaccttartttccttttatcttaaacaccaggaatcaaacaatctcaaccatctgaaa

SEQ ID NO: 513 - DNA, 101 bp, Homo sapiens
tagtgtagccatccaatggactactatctagcaatgaaaaagagtgcaccrttggccacatggcaacagggataaatctcaggagtgttagagcaggtgaa

SEQ ID NO: 514 - DNA, 101 bp, Homo sapiens
cctttcctcttccccgcaccaacaccagctccatgtgcatttattgttggrttttaacacccgtgtcctccctccctctccccagtgttctttcacagctt

SEQ ID NO: 515 - DNA, 101 bp, Homo sapiens
gaagagaccaattgccctttttacagatattatgattgccaacacttaacrtgtaaacaaattattagaacaacattgttcagcaagattaccgagtgcaa

SEQ ID NO: 516 - DNA, 101 bp, Homo sapiens
ttgtatttttgacgtcactagtgtcattttttgagtcctctaccaattttycaagggtatatcatcttcagttccaattgaacatacagccctttttgaat

SEQ ID NO: 517 - DNA, 101 bp, Homo sapiens
aaaaaaaagaatacattttgtttagatgtggaaaatgagtagcttgaaagyaaagccaaacaacaacaaaaacaatgacaaaaaatctgtatgtcgtaatc

SEQ ID NO: 518 - DNA, 101 bp, Homo sapiens
cattttaatacgtgtcacactgaataaatttatgcacatttattcatgtcraaaggaaaaattaatgttgtgatgttgactccttgatgaagtttttgaag

SEQ ID NO: 519 - DNA, 101 bp, Homo sapiens
ttacagatttttaccatagatatactcatagagaaggtagcacccactcarccctagcaatagtgctagtgtttacaaaattgcaaaagaagtatgacaca

SEQ ID NO: 520 - DNA, 101 bp, Homo sapiens
tcaggcggcccagagagcagcgctccacattcagcttcacggagccagacrcaaggtctcgaatggtgaagctctggtctgcagccaccgtggttacctgg

SEQ ID NO: 521 - DNA, 101 bp, Homo sapiens
cccaaagaatccttcccttacagcaggccagaaagctattgtcctagcctrtggaaacacctaaaacacactggggagatgtggacactcagcccattgca

SEQ ID NO: 522 - DNA, 101 bp, Homo sapiens
caccactgacactatttacagccaaagaaatcatatgaaaccgtactagcrcatgcaccagaaccaaatccaaagtgccccaccaaaacaacaccataaat

SEQ ID NO: 523 - DNA, 101 bp, Homo sapiens
aattctcattctcctaatccaaggctctgtgtgatacatcacactgtgttyattactttattacagagcaagtaaacagatgcttagtgtagatcacgcag

SEQ ID NO: 524 - DNA, 101 bp, Homo sapiens
ctggattttgtggtcttatgctatttccactcattctccaaatgtaaccgkaaagaccatcccaaaatgtaatacaaacctttttaaatgcccatttaaaa

SEQ ID NO: 525 - DNA, 101 bp, Homo sapiens
caggagcagggtggacgtcaaaaaataatcctgatgctatttggctcatgkatgattcagagcaggtgctgtcagagccctaatttcccttgtttttgaac

SEQ ID NO: 526 - DNA, 101 bp, Homo sapiens
agcaaactgataagtcaaagatgcatatgtaattcccagatcaaatactaraaacagcaaaaagaggataaaatagccttttcagcaaatggttctagaaa

SEQ ID NO: 527 - DNA, 101 bp, Homo sapiens
tctattcaattttgtttcttttttcaaagtaaacactgttttgtaaataayacagaactgaaccccaaaatacataactgggcattggaggattagaacat

SEQ ID NO: 528 - DNA, 101 bp, Homo sapiens
agaggaaatgtcacaaaactcttcatagttacaaagacattgtgacactcrgtagaggtaaaggttccagtattttaaaaacatgagaaatatgggttaaa

SEQ ID NO: 529 - DNA, 101 bp, Homo sapiens
cttgctgctttctgcagaaacccctggaagcagtccaaatgcaaagttagrgcttcagagaatgcaccctgtaaatggtagttgtgtatacccttaccatt

SEQ ID NO: 530 - DNA, 101 bp, Homo sapiens
ttcaaggaaacaggtgaacatataaacgatgtaacagtttatatgtaggartgccctttggctctgtctattgctgtcagtacattatttacctgctccag

SEQ ID NO: 531 - DNA, 101 bp, Homo sapiens
ttttattagcaggtctagattgagagagatttacctcggcagtaccatagygtggataatattcagttaggtttgttcagaggaacttccccatcattctg

SEQ ID NO: 532 - DNA, 101 bp, Homo sapiens
acatgaatagatgggacctgtatttgcttaattccagtagactaaatactytggcctaaatagagttgtcaatctcataaacccaagaaatactcagaaac

SEQ ID NO: 533 - DNA, 101 bp, Homo sapiens
agagctgacttttacccaaggggctgtgggtggaaaccagatgaaatgggrtatgtagttgatggtatgtgaggacctaatactgtcttataaacatttat

SEQ ID NO: 534 - DNA, 101 bp, Homo sapiens
atcgccgttcccgaggtcgtcccctttgcacctgtccgcgggtcctcgggygtgtggcttccgggcacacagaaaaccgtgtggttctaggatacatgggg

SEQ ID NO: 535 - DNA, 101 bp, Homo sapiens
attagtcatggaaaaggaataaaaggcatccaagttggaaaagaagaaatraaattatctctgcttaaagatggtatgatctatatgtagaaaatcctaaa

SEQ ID NO: 536 - DNA, 101 bp, Homo sapiens
tctttctaatacacatattgcatctattccatgccttcaatgaatttcccrttgtttaaactataggtcaagaaactgtccaattgctatacttgtttggg

SEQ ID NO: 537 - DNA, 101 bp, Homo sapiens
taaagaaaaaaatctgtacatgttttggacagacacaatttttttcccagrctatttttgacctatagttatttgaatccacagatgcagaacccacggat

SEQ ID NO: 538 - DNA, 101 bp, Homo sapiens
gccttgtgtgagcatttttatctctggcaagctcccttcctctcttagatratagagattatctcccgggattacaaggacagcttctccacaacgaatcc

SEQ ID NO: 539 - DNA, 101 bp, Homo sapiens
cctagaccagtgtggcgtataggctatagaagattggtcctgaatgctaayggcagggatgagtgtaagatcatcaaaacttatttgtgggaaggagagct

SEQ ID NO: 540 - DNA, 101 bp, Homo sapiens
gggaaggtcgttgttttcctcctatttcaaggtgttgcacctttggccaamgggcccacagcactgcttggaggaaccacagggcttcaggacgtgaccgt

SEQ ID NO: 541 - DNA, 101 bp, Homo sapiens
ccaaaagagaaaaaattctgacgggggcataactggagaataaagtgatcytaaaatactgctgaaacaaaaagtcatctgccccctggaccgttgtctta

SEQ ID NO: 542 - DNA, 101 bp, Homo sapiens
aaatcttggctaatcatttaatctttgggcatcaatttcttcactgttaamatgacagttgtagtatttctccttaaaatacttcagggcagaattaaatc

SEQ ID NO: 543 - DNA, 101 bp, Homo sapiens
tacatgatataagaaaataataagaatgtggtttcgtttaggaagattctyaatacacaaagatatatctgcaaatatattttcctagctttggttttctt

SEQ ID NO: 544 - DNA, 101 bp, Homo sapiens
ccaagtaactataagattcatgtattagagaaaatcatattaaatttgctrttatgtgatcctttagacatataaaaatggtatatgttatggttcaacct

SEQ ID NO: 545 - DNA, 101 bp, Homo sapiens
acaattatatgccaacaaattggataccctagaaaaatgaaaaaaatcctrgatacaacctaccaagaatgaatagtaaaaaaaaaattcttactcaacat

SEQ ID NO: 546 - DNA, 101 bp, Homo sapiens
cagcagatacccttaattcctatttcccagtgagaacaaagggcagaaaaygtgaccgtgcccacattctctgctccctaaccccctaaacaatcagcacc

SEQ ID NO: 547 - DNA, 101 bp, Homo sapiens
atagcagcccttagcccagcgacctccagaagcctcgcccacccccggatrgtatacccaccctagagagtacgagtcctggcatttgaggaagtaccact

SEQ ID NO: 548 - DNA, 101 bp, Homo sapiens
taaactgttcagtaataacattgatttgattttaagaaataatagaaaaayagagtttatactacagcagtgatttccagtagaaatatactgggagccac

SEQ ID NO: 549 - DNA, 101 bp, Homo sapiens
agctagtgtccagtagtcctcccaggattataggtgaaagatggaggagamggttcggtatgcagggaatcacgcgacacagtgtccaattaatttttgtt

SEQ ID NO: 550 - DNA, 101 bp, Homo sapiens
tatgtagcagcaatcttaaaaaatttttatttactaaaaatctcatcatcyaataattatttaaataccttttcatactatctgtataagttagctaatgt

SEQ ID NO: 551 - DNA, 101 bp, Homo sapiens
ttatcccttatagatgcctaagagcttatttataaaatggtaatactaatrtatttaatgtcatcttacagttaccatgtacttttcagtttacaaaatac

SEQ ID NO: 552 - DNA, 101 bp, Homo sapiens
attttttacctgcaacccctgatgtggacattctcagaaaaagccagccaraggaagtctttcattaatcccaggcatgtcacataacctcagaccttttt

SEQ ID NO: 553 - DNA, 101 bp, Homo sapiens
tgagctccaagcaggcaaggaattcacctgaaagcatgaatgaaagacagrtctggaatgcaccaaatgactaggatcaggagtgtctgtaagtgtcagaa

SEQ ID NO: 554 - DNA, 101 bp, Homo sapiens
catgcctggacttcacttgtagcacatcatttgtggaaggctgcagtaagyactcaatactttgctgttgattgatttcagaacggattgatcagattgca

SEQ ID NO: 555 - DNA, 101 bp, Homo sapiens
tcatggaaatataaatggaattttagattcatgttaaacctctcttgtaamgttctcaatgtctatgtgtatacttcaaactgtaactttttttaaaaaaa

SEQ ID NO: 556 - DNA, 101 bp, Homo sapiens
ttcatcacaccactctgacttgctacaatgactgcctggacatgctgactmcagtgagttccaggcatcagtagggtctgaaaatataagcaaaggaaaac

SEQ ID NO: 557 - DNA, 101 bp, Homo sapiens
atctgtgccctcttagaatgtaacactggaaagtggtctccctcttatggyttttaaaattgtgaatgtgctggtttgagcaataaactctgaaggttgag

SEQ ID NO: 558 - DNA, 101 bp, Homo sapiens
atggatcactgcccagcagatagctggtcctccaaacgtatttgctgaatraatcaactgccttagaggcagagatattcttactgcattccttagtctat

SEQ ID NO: 559 - DNA, 101 bp, Homo sapiens
ttctgttacctaggagatgttacttacatatgtaatactgtatcctgcacrtggaaatattcagaattgtagatagcataactctccctgctcctattctt

SEQ ID NO: 560 - DNA, 101 bp, Homo sapiens
aaggcagcttgaccacaggcaatagcttgctgattcctgcataaagtttarcatactcttgaaatttcatttgtctaatattttaacctcaaactgtgcct

SEQ ID NO: 561 - DNA, 101 bp, Homo sapiens
tacagaaagccctctgtccttgtaacaaggtagacgctctaattgagttgrttaacacaaggtgcccgtaggcaaactaagagaacaccctgtaacacacg

SEQ ID NO: 562 - DNA, 101 bp, Homo sapiens
ttgtgcaaatcttctgatttgtgcaaagtcccagaagaaatgacgatagamtgctgctctcctcctaagtaaaatgaagaagtatctaagagaaacagatg

SEQ ID NO: 563 - DNA, 101 bp, Homo sapiens
cagataacccttaaagtgaagaactaggtgtctcaggtagttttaggtacytcacctgcttcctgtaatctctacagacatttgcttaaatatatactaat

SEQ ID NO: 564 - DNA, 101 bp, Homo sapiens
gacctcaggtgatgtttagacttacttcttggcctagacttatgttaacaraaccccaaaaggtctaaagcactaaagaggtttgccaactacacttagat

SEQ ID NO: 565 - DNA, 101 bp, Homo sapiens
tattttagtaccaaatgaaatttccattcagatataatttgcgaacccctygggtgacacttccatgcaatgaaataatactataatgacacaatgacaga

SEQ ID NO: 566 - DNA, 101 bp, Homo sapiens
tcactcagctaatagacagagaatgatgtataaaatcataatgccaacttrtaaatttataaatagaaatatggttgtcatacctccttaaacactgacat

SEQ ID NO: 567 - DNA, 101 bp, Homo sapiens
aagctggctgaatttttacaaggcaggaatgaaatactgaagagagacatmttcttgaaccaaaacaagctgaagaagagtattgtcccaaatattgcaca

SEQ ID NO: 568 - DNA, 101 bp, Homo sapiens
ggaatatactgtctctcagtaagtgatactgggacatctggatatgcatayagggggggaaaaaaagaaacgactcctacattacatcgtacacaaaaatc

SEQ ID NO: 569 - DNA, 101 bp, Homo sapiens
tcaatttctgttcctttaggccagtcagtctgtgttaccttcttacagcgrccccaggaaacgaacaagaaaccagtccaaactgcttagcatgatactta

SEQ ID NO: 570 - DNA, 101 bp, Homo sapiens
tgtggatgcagaacccatagatagagagggctgactgtactaaagattacmtttccttctccacgagtctcaacatattcatctactcagcagtaaataaa

SEQ ID NO: 571 - DNA, 101 bp, Homo sapiens
ggaaaagaaaagaaatggcaacctgaggtcagctgtgtgtgacccacatgyaagactgaagtagaacttgcctccttgtgaacgaaacagggcaacaagag

SEQ ID NO: 572 - DNA, 101 bp, Homo sapiens
catcactctgctccatctcttacctagattccagaactcttctttctccayctacccaaacttttacttctgctagtctctattacccatgcctttctaca

SEQ ID NO: 573 - DNA, 101 bp, Homo sapiens
atcctcaccactgcaagcattaaggagaaacccctaaaattattctgagtrtaaacacagcaaaaggcgcatggaccttaaccaacatgtatgacaccaaa

SEQ ID NO: 574 - DNA, 101 bp, Homo sapiens
tggcacaataactaactgtatttttagagtttatcaataaatatgatgttrccataaacacacatgaacacactgatctctttaaaagatttacaatggaa

SEQ ID NO: 575 - DNA, 101 bp, Homo sapiens
cccaaaggttgttatgaggtgcatgactttacttatttgtgtggattgatygttaatcagtatgccatattcctaaaaatgagcacttgctccaggtcttt

SEQ ID NO: 576 - DNA, 101 bp, Homo sapiens
cagtcagaacatctttggtacacccatggaatgaatagtaatgactggcaygggcagaggtgaagccatatccatatgtttattttttattaaaaaatgta

SEQ ID NO: 577 - DNA, 101 bp, Homo sapiens
tttcacaggctcctgggcccaagatacagtgagtacaatgggtcccacgcrgttctcccctttgagtttgaaagcttagagttcttgagctgaagcaagca

SEQ ID NO: 578 - DNA, 101 bp, Homo sapiens
gggaaggcacttgtttcgtggaggagtaggatttgtgtctctggcagttgycctgcacattcaagatgcaagagctttctgtgcaacacaagcaaagcaga

SEQ ID NO: 579 - DNA, 101 bp, Homo sapiens
caggtcatgttttcacaaaatgtgacatttcatgtcgttgttatgaaaacmgtggcaccaaattcaatctgcaccaatcatatttttattttaatatttta

SEQ ID NO: 580 - DNA, 101 bp, Homo sapiens
gatgggaactggcctccttttaatagcacattaacaacattattctacccraaggaagacagcttccctttggccttagctgccttgtgagtttggtgaac

SEQ ID NO: 581 - DNA, 101 bp, Homo sapiens
aaaattctgtcaatagacacataggtagggagactattcctgagtggtgcmtgcctctagaaaaacaaacctataagtgagataaagtttagatttcataa

SEQ ID NO: 582 - DNA, 101 bp, Homo sapiens
tacatatgcttcagaagaaggctaagggttcgttatcttaaagggggaaargagtgtcttggacaccagccttagctgtcagacaggtctcatcttaattc

SEQ ID NO: 583 - DNA, 101 bp, Homo sapiens
actattcccctcagtctcctcactatgcatcaaaactagcaggtaaatccytggctcatgatgcatccataagcttttctctcacttttctaaaatattag

SEQ ID NO: 584 - DNA, 101 bp, Homo sapiens
tctcaaacttctgctctaaactggcaacatttaaagagtctatttgggaaytttggggaacccagtactctcctattggtgaaaatgagagaggatgcagc

SEQ ID NO: 585 - DNA, 101 bp, Homo sapiens
aagtgtaatttacaagacagaaaggccaagatactcgaattgatttaacamgtacaggcaaagtatttttgaagaagttatttaacccatttgaaactgat

SEQ ID NO: 586 - DNA, 101 bp, Homo sapiens
tcccatgtttacacatatattcattatacattttatgtacctattatgatrtgccagtcaccttgttaggctttgggtataaaaagaatacaaagatgaaa

SEQ ID NO: 587 - DNA, 101 bp, Homo sapiens
gaattgcaaaaggcatttcaaagcaccttcccacattcccagaaagatgtyttcccctctttccaaacagctgagacagaagtacaacgtgtggtccctgc

SEQ ID NO: 588 - DNA, 101 bp, Homo sapiens
gtcatatatcaattatacttcaattaagttgtaaaaatagttataaaagcmaaaggtatgtctgcactgttttatatatattcattttaatttaaaatgtg

SEQ ID NO: 589 - DNA, 101 bp, Homo sapiens
cgtcatcccttaacagaactgctgcaacagcagtaactgatgttccatgcycccaccccttatagtgggttaccaacccagatgccagagttacgcttttc

SEQ ID NO: 590 - DNA, 101 bp, Homo sapiens
gaggatatggactgaagagtagtatttacacagtaaatgctaccagccagrggaagaagaggaagatgtgtgtgaacctgagcagtcccacagtcctgtcg

SEQ ID NO: 591 - DNA, 101 bp, Homo sapiens
gagagctgttaaagggtttggagcagaggagggacatgacccaaccagccyattaacaagagcacaggctgatgtgttaggactgaactggagaagacagg

SEQ ID NO: 592 - DNA, 101 bp, Homo sapiens
ttatttaatgttgctcttgtatccagcaaccttgctgaatttttttatttktaatagtttggagtagatactccagttttacaggtaaatcgtcattttca

SEQ ID NO: 593 - DNA, 101 bp, Homo sapiens
ggaaaagctgttaggaggtgctgaataataatcacagttgagtcactttcygacactgctgtcttgcatgatttactgaatataatccttcaaatgatctt

SEQ ID NO: 594 - DNA, 101 bp, Homo sapiens
tggccacatgtgcctgttgagcacttgaaatgtggctagtccaaattgagragttgtgctataagtgtataatacacactggacttcaaagacttatcttt

SEQ ID NO: 595 - DNA, 101 bp, Homo sapiens
tgcttcaatgctttctgatttcatacctgcataataaaattcctgattcgyccatcacattttggcaaacaaccaccgccacatctctctggatactggct

SEQ ID NO: 596 - DNA, 101 bp, Homo sapiens
gtagcttttggcaaatcttctactgcatctcaccactgtgggaaattgcarcttccaaggaaaaggagtagaaactacaggctcaaaaaaatgagatcagt

SEQ ID NO: 597 - DNA, 101 bp, Homo sapiens
aaaatagaaatgtattttatattctaaatcttaagagtcattaggttgatrtttgcaattttttatagttaatgcaaggcatgttaaaatataatttgtct

SEQ ID NO: 598 - DNA, 101 bp, Homo sapiens
ccggaatagaactcaggctaaatgctggtggtatggaattgggaacatgtrccaagtaaagacagaggcttgtttggaaggaatagcagaggaagatgaaa

SEQ ID NO: 599 - DNA, 101 bp, Homo sapiens
atgacgtccccatgacacagagaagccagaacccagcacgcaccccatggycattgcacttcttcccacagccttcagtttcaaagaaggaggtgttcctg

SEQ ID NO: 600 - DNA, 101 bp, Homo sapiens
cattaccagatattctgtagttctttatttctgaaattccttaattggaaracaaaacaatagtaatagccaaaataaaagttacatggatatagtttcat

SEQ ID NO: 601 - DNA, 101 bp, Homo sapiens
ggttaagaagcttaattgcaatccctatgaataacaaaagttgttagaacyacaacatatcattttcctttctctttagtagcagattgacaaaaactggg

SEQ ID NO: 602 - DNA, 101 bp, Homo sapiens
atgtatcctgtaggcagtaggtcgtgtggatggtttttaatgtaaaagtgyggcacgatgacagcattgctttataatgattattctggtggcattattca

SEQ ID NO: 603 - DNA, 101 bp, Homo sapiens
catggcaatgtggagaatgaattggaaaggaggtgtggaggtcacctagcrgttcaactgaggtaatataaaggtttgaaatcaagcagtgatgagcaaga

SEQ ID NO: 604 - DNA, 101 bp, Homo sapiens
cttgtatcaacttgttgtttatgctctctactaaatacatcctgtatgttycaatccttgtgtcttttcttctctcctttaatttaaatattacttcttgc

SEQ ID NO: 605 - DNA, 101 bp, Homo sapiens
tggcccacctgggatcttctaggtctttctatcacaatactgctttagaaragtctgtgtgaaggaggggactctggtatttaactccatccatcaatgtc

SEQ ID NO: 606 - DNA, 101 bp, Homo sapiens
attttgaattgtacaatacatcataattattggagatagtcactccactaygcaatagactccaaaggtattccatctgtttacctgaaactcttgggcca

SEQ ID NO: 607 - DNA, 101 bp, Homo sapiens
ggagaattttcccttgctccggcttcccactgacggacgtttcacttaacygtattaattcctctgcactattagttacgcatgatgcatgacaagcagat

SEQ ID NO: 608 - DNA, 101 bp, Homo sapiens
taaatccttcctactgaccagtgatgaagacagtgtccatttctagggtamattgtctgcgattgctgcactctgatacatgagaaatacatgggagggag

SEQ ID NO: 609 - DNA, 101 bp, Homo sapiens
aacagttttcttaagttactttttctgtccttttagtggcttcatttaaaktacagtaaaatctcagacacaaaattatcaaggatttaggaataaaggga

SEQ ID NO: 610 - DNA, 101 bp, Homo sapiens
attccagaaatggtaaaaggtagattcaaagtgtagcaggataaaaggaaragctatttcagggtctctgttaatgaggacatcaaccaaagttttcccag

SEQ ID NO: 611 - DNA, 101 bp, Homo sapiens
gcattccaggtagaaggcaagggtcagagtgccccttcctagtttctctcyatccatcattgggacaaaatcttccccagacgcctcagtatacttcccct

SEQ ID NO: 612 - DNA, 101 bp, Homo sapiens
ctcctttctttggctatttttgatatgcctcattttgtatcatataaaacygtggctcttcttctcttactgcatataactttaccttctactttatagaa

SEQ ID NO: 613 - DNA, 101 bp, Homo sapiens
gtcttcaggaggtaagaaatagtaggagcttcttgaattttggaaatcagracacaaaatagaggatacccctctgcagcagaattttaattcaacatcat

SEQ ID NO: 614 - DNA, 101 bp, Homo sapiens
agttatcactgacccattttctatgttatcctaagcatcctttgaacgatrtcctctaaactcttctcacatattgacttcaagctcaatagcctgtgatt

SEQ ID NO: 615 - DNA, 101 bp, Homo sapiens
ctctttgtacttttctctcccaaaggagcattccttgagaagccggaggarttctactgattacatctccagcacagccacattccagcgggtaggagggt

SEQ ID NO: 616 - DNA, 101 bp, Homo sapiens
gaagcagagataatgacagagagtgggatactagagaaacgcccaagaccrtctttagctgcagagttctatcctggatttcatgtgtgaccttagacaaa

SEQ ID NO: 617 - DNA, 101 bp, Homo sapiens
taagagggggcactgctggatttggtccatgttataggatttgctgcacakcccgttactcagaaaatggggctgtggtatcagacccggctttgaaactg

SEQ ID NO: 618 - DNA, 101 bp, Homo sapiens
agcatggttataatagaataagttaagttccaaataggattacttatttcrtgttgtagccctaattttgcctcaaccactcaccctctggtaaattcctc

SEQ ID NO: 619 - DNA, 101 bp, Homo sapiens
accatgagtaattcagtattcattcaacttgaataactacagggttaggakagtcattttgaaaatggttaggattattagttagtgttaagaaaatattt

SEQ ID NO: 620 - DNA, 101 bp, Homo sapiens
tgaaaagagaaatgcatatagattttttagatgaaagaggggagcacacarcatcccaaattgtgatatcgtttttgcctaagcaccaggggttttaggga

SEQ ID NO: 621 - DNA, 101 bp, Homo sapiens
gaaacccgagcgaaagacatttcaaagagggtttagatttaaagcaaatayctattcactctaatctgctttaaaatctgttgttttcctggagagactta

SEQ ID NO: 622 - DNA, 101 bp, Homo sapiens
gtgagaatgttgatttttgaaaaaatgatccctcaaatgcttacagccccrtgcatgtacaaagatgaaaaatcagtgcaattggagaaaaaaacaatggt

SEQ ID NO: 623 - DNA, 101 bp, Homo sapiens
taaaggattctaagtcacctttttccctcattcaaaatgaaaacctctctrtttttatttattttttgagacaaggtgtctatcacccatgctgcagtaca

SEQ ID NO: 624 - DNA, 101 bp, Homo sapiens
gacaatttccctgatataaaggaaagatgaatttgccaaatgagcagcaartaattttccagggtaaggtgatggagaatgagccacactgatacaaatcc

SEQ ID NO: 625 - DNA, 101 bp, Homo sapiens
caggctttaccacattaattcccagggtattttcctaaattaacatcaacmttacacttaccattgtttctttagtttctcaaaactttatcataatgtga

SEQ ID NO: 626 - DNA, 101 bp, Homo sapiens
taccattttgtgtctttacatcttttactcctggcaaaatgaaataatttmttgatgaatgtattagtttttgtcttttaataaatatgctgtaagtgttg

SEQ ID NO: 627 - DNA, 101 bp, Homo sapiens
tactcaccattatctctctatggaaataatctgcctattattgcctccctrtggaatctgcctctttatggaaataatccccaacataaagcagcaactcc

SEQ ID NO: 628 - DNA, 101 bp, Homo sapiens
ctcagtaagtggcactctcatgttttaaagttattcaggccgaaacttcaytctttctatgtctctcactgtgtaaccagtacattagataatcctactga

SEQ ID NO: 629 - DNA, 101 bp, Homo sapiens
ctaattgactgctgctgaagcaattaactgattatgttttcccctcatttraaagtttctgtgatatagacaagtaactttgtgttacaaaagtaatctag

SEQ ID NO: 630 - DNA, 101 bp, Homo sapiens
aagctggcttcctcagccatcttgattttgaatactttgccacttctgaayagtttagtgtttttctgttctatccatatggtgacatcagctcttagttc

SEQ ID NO: 631 - DNA, 101 bp, Homo sapiens
caagacttgctagacacaaggtccaagctgacatagatacctgggaggccraaagcagcaacactctcctgcttgggagaggatggtacttattaaatgga

SEQ ID NO: 632 - DNA, 101 bp, Homo sapiens
gctgatttaactatgtttctttttggagcaattatttttatgataagtaamagaaaagtttactcatacagagaaaaattcaagaaggtatgaggcactga

SEQ ID NO: 633 - DNA, 101 bp, Homo sapiens
atgattctgaatgtgattggatgagttcctaggaagatgggtcatacagayaaaatgatcattgtagagaagatgctatttcctcctgtgggaaagaaaca

SEQ ID NO: 634 - DNA, 101 bp, Homo sapiens
ggatgcatagagttattctatgtaaattacccaccagagagaattcaggcrtgtttcaaaatctataaaaccgtgtgctgggggaccatgaaaagttgtag

SEQ ID NO: 635 - DNA, 101 bp, Homo sapiens
aatttgataattaaaatttcattgatgtgtttgcacttattctcttaaaaytgtaacatttaataagtaaaaagttatgctcattaactcaaacagatttt

SEQ ID NO: 636 - DNA, 101 bp, Homo sapiens
ctcaggtaaattcacctatgtgtgtatggtaagacactgcttctactctgytcatcagcaaaacacttattatcattttcataactttcctagaattttag

SEQ ID NO: 637 - DNA, 101 bp, Homo sapiens
tctcagggtgaaattcagtacaacttcattttacagtaaggatcttggggyccgcaggagattttctgtgagaaaattgtaagagagggcccctgagaagg

SEQ ID NO: 638 - DNA, 101 bp, Homo sapiens
tcataatgggactgcagaaccagaagcaaaagagtaaaatgcttattttcrtacaacattgagttttggggtccttggtttgtaacattattgcagtaaaa

SEQ ID NO: 639 - DNA, 101 bp, Homo sapiens
aaaaccatatgccattgtatctgaaatgttggcccccttcaagactctcamccaagaaattgcaccataatttacctcattgttgaagccaagaaaatgga

SEQ ID NO: 640 - DNA, 101 bp, Homo sapiens
gcatcatcttccataggcacagtgatcattgccagccagtggcacttctargtgaggaggctcttaggcgaggcccccaggatttgccctgtaggaaccgc

SEQ ID NO: 641 - DNA, 101 bp, Homo sapiens
acacattaaatcaccacttctagggaaaggttgagctcactcatagctctrttgatagtgacactgagagggtattaaatgttgaaaggtctaaaagggag

SEQ ID NO: 642 - DNA, 101 bp, Homo sapiens
gcatctggatgaatagatctacgatgaccatattgccttcactgtacatgrcctaaactcatctctctggaaagttaatctttcataacattaacatcagt

SEQ ID NO: 643 - DNA, 101 bp, Homo sapiens
ttctcttctgttgtttctacccgtgttcttctccgggatattatcagaaartaaacacaccaaaggaaataaacaaaatatgcatttccaatatattttcc

SEQ ID NO: 644 - DNA, 101 bp, Homo sapiens
atggaatttgcacattatatatgttatttatggaatacagatcattcattkaggcatttttctagattgtctttgagcttccctgaccaacttgcagttta

SEQ ID NO: 645 - DNA, 101 bp, Homo sapiens
ctgaacttaaacattatagacacacgctatgtctataatttttgacattayagacatgaaggtccttaatgggctagtgggcaaaagccatctaggaatca

SEQ ID NO: 646 - DNA, 101 bp, Homo sapiens
gttacctgatcggctgatccgggagttgaactgtaatcaggggcttgtagkagttagagctgtgtgggcctctgaggagctcccagcctcccaggagcggc

SEQ ID NO: 647 - DNA, 101 bp, Homo sapiens
tcactgccgttaagttgtagagttgctctaggtccctgcattcggctgtcrtatttcactgaacttactttgaagttgcttatgtcactctcaccattgcc

SEQ ID NO: 648 - DNA, 101 bp, Homo sapiens
gtatttttgttttttttttaagttttcagaactttaagatggtgtgtagayagatgcttttatgggccaagaaagcatgttgatatccattattttatttg

SEQ ID NO: 649 - DNA, 101 bp, Homo sapiens
ggtgctactgcttccagagacagcaaggtaaaagatgagacccttacagaygcaaatagttgacctgcatgtcaaattttacttattttttaagaaaataa

SEQ ID NO: 650 - DNA, 101 bp, Homo sapiens
attcatgtctaagcatttcgtagaaggatgcacgtgagaaaaagcacctgygctgtcatagcgatcctttggtgttttaagatgaaaaagttcaaagcatt

SEQ ID NO: 651 - DNA, 101 bp, Homo sapiens
aaacttccattaggaagtatgtgaaagaaactttcctttaaataaaaatgygtaagtgtttagaattgcccttgcaaagctctaaatcaatcacccagggc

SEQ ID NO: 652 - DNA, 101 bp, Homo sapiens
tagtcacctcctttgaacagctttctagtaacaggtccctggatccatggygcttatttttagaagagacagtagtatattattttgaggtcatggaatta

SEQ ID NO: 653 - DNA, 101 bp, Homo sapiens
tatgcttgttcccaatctccttgggagaaagcagtgtcaatcttttaccaycaagtataattttagctatagattttgtacaaataacttttatgagtcta

SEQ ID NO: 654 - DNA, 101 bp, Homo sapiens
gcatgtagatgcaagacatagcatttaagaatatcaatgtgtgtgcctacyatgccttactagctaaatattctactgttgtataacaggatgatttggtt

SEQ ID NO: 655 - DNA, 101 bp, Homo sapiens
tgtcccccaaccatctgtagacattcccaaaagcctccatcgcatatgctygtgcacccacttgtcagaagcatacccatgctgcaccgccccggatttgc

SEQ ID NO: 656 - DNA, 101 bp, Homo sapiens
aataccaaggagagcagagctgtgctgtcaagcccctgacaattcgtgaayttctgctgctgaaattattagtgctgccttggatcaagttccatttgtat

SEQ ID NO: 657 - DNA, 101 bp, Homo sapiens
tttttaatatcaattggaattgccgcaacacccaacactgacacacagttyccagagcaaagctccgtggtcagactcccaagctccttagtagtggtggc

SEQ ID NO: 658 - DNA, 101 bp, Homo sapiens
gttcaatacatctcaatgagaagcatgcaaccttaatccatgacgcttgtragtggagctatttttcaatctacgttaattttgaatttaactgtgtcaag

SEQ ID NO: 659 - DNA, 101 bp, Homo sapiens
acaaaattcttgaaggtcaatatgggatagcctcaagcctcggacacaaargagtttgtattcacactcaagcttttctttagggcccctaactgggtgct

SEQ ID NO: 660 - DNA, 101 bp, Homo sapiens
ctggattcaattctttctttgtttccatatccaatcctccatggatcattmtttttccttagcacttctgatgatgtttcccaggatacatccttagcctc

SEQ ID NO: 661 - DNA, 101 bp, Homo sapiens
taaacaagaatcacttttcccgtaatcttactacgaaaaatggtattaatygatatttgtacactaagatatggctaaaaagccaggtacctaagcccatg

SEQ ID NO: 662 - DNA, 101 bp, Homo sapiens
ttatgcttctttacaacttgtgcaactattacctaagataaagccctgaargaaaagaaactgtagtctgagtgactgtgagaaatcataaatgacagtcc

SEQ ID NO: 663 - DNA, 101 bp, Homo sapiens
cccacacttctcccatatctgtaacctctccatctcttttgttctgtctaytggcatataaacagattaaaatttctcccaccctaaaaattaagaataag

SEQ ID NO: 664 - DNA, 101 bp, Homo sapiens
gcatataaacagattaaaatttctcccaccctaaaaattaagaataagaaytctgtcaaatcaataaccaccctgactttctcctcttcacaacccaaaat

SEQ ID NO: 665 - DNA, 101 bp, Homo sapiens
aatacatcacatccattttatccatatcacttttcctgggtttggctaccrgcgcagattaatagttgtctttgcattatgcagtggaacttaatttctat

SEQ ID NO: 666 - DNA, 101 bp, Homo sapiens
atcagaacaagattctgaatgaaaacgtgttcccccaggtgagccatatgyagacgaatgcttgggatgctgggtagatgttgaaaaaaagttttgcccga

SEQ ID NO: 667 - DNA, 101 bp, Homo sapiens
gctcaggtttgcttcttaaacacagatttgaatacattactgtaaatctcygttttgcttttaggtcaaatagaaatggtcatggaatgacagcccagatg

SEQ ID NO: 668 - DNA, 101 bp, Homo sapiens
gaatcaatcacatccttgttgcctcccttttcttcaaccccatgttcaatyagtcgctgagctgctggtaaatccctaggagaaggagagtgatgtgtctc

SEQ ID NO: 669 - DNA, 101 bp, Homo sapiens
caaccctttcaaaaaatctctgggagttgaaccaggattgatcttgtggcraagaatcttcatcggctgctaggacagccattcagtctcactttcccatt

SEQ ID NO: 670 - DNA, 101 bp, Homo sapiens
ttctaccaagctcctaggtgatgatgttggggattcatggaccacgctttragaggcaaggataaagaaaactactgtatacgaattagggccacgatgtg

SEQ ID NO: 671 - DNA, 101 bp, Homo sapiens
aataaggaagcccatttattttatcattattacttttatcactaataacargctctttacacctacacatgagaatgacaatagcaaaggaaacaatcatt

SEQ ID NO: 672 - DNA, 101 bp, Homo sapiens
gaaaaagtattaatacttcctcagggtaacctccttcagcactatcagcarttacaatgagattgaatactaattaacctttaaatataggctttggggct

SEQ ID NO: 673 - DNA, 101 bp, Homo sapiens
tgtatcattctatggtaagactacgtttagctttgcaagaaactgtcaaaytgtcattcaacgtggctgtgtcatgttacattccctacaatgattgggag

SEQ ID NO: 674 - DNA, 101 bp, Homo sapiens
ccatcttgctgatttccaggttgcttcggggaccccaagagaattcatatkctggtggattggtgtgaggcacccgcctgtaactgagatatcgctgctgc

SEQ ID NO: 675 - DNA, 101 bp, Homo sapiens
accgcaaaatgtaccttgttgggtatttagcagaaggaaatgtgttgactrttacacatcccttatctacagtgcttgagactgttttgaatttcttattc

SEQ ID NO: 676 - DNA, 101 bp, Homo sapiens
gttgaatgatttcattttacatagattgccttttatgatttttatgatttyttcaactttcattttaggttcagggttacgtgtgtaggtttgttatatag

SEQ ID NO: 677 - DNA, 101 bp, Homo sapiens
cctaggcgaataaacaaaggaatgatttctccacttggatggacataccarttgtagcctgttggtctgtttctcaccctacttatcagagtaacctctcc

SEQ ID NO: 678 - DNA, 101 bp, Homo sapiens
ttggcttagattattttttaagtttcatattgtgccaccacgggcgggtcytctccatacagcagtgactgtaaaatcaaaccccactttcagtgagtgag

SEQ ID NO: 679 - DNA, 101 bp, Homo sapiens
cctgaaaatcagtttcttcccttcgattgacaaccaaggaggaagtcagtkggaagacctggggcattcataaagggacaagaatctttttctcattaagt

SEQ ID NO: 680 - DNA, 101 bp, Homo sapiens
acctttgtgatgctttatctcccaactgacactgaactacatactaaataygtattgctactatgttctcctaagcttttttatacatgctactttcttta

SEQ ID NO: 681 - DNA, 101 bp, Homo sapiens
actggccctgcagcactgagacactcaggagcccatgatcctccaccagcygtgaagcagcagagaaactcatggtccgaaaccgcaaccaaagcctccag

SEQ ID NO: 682 - DNA, 101 bp, Homo sapiens
aatacttttattaatatacaggaatccccccttacctgcagggcatccaaractcccgagtgaatgcctaaaaccacagatagtaccaagccctacacata

SEQ ID NO: 683 - DNA, 101 bp, Homo sapiens
agtgttggcagatgtcaaataactgcatttattcaaccagaactgatcatyatttagagtgaaatgatcaattattggagtaaaatgcattttgtttgcaa

SEQ ID NO: 684 - DNA, 101 bp, Homo sapiens
gcctgggttcaaatttggactctgccatttccttatctgtgacttggagarctcatttaaacttctcaattcttccattccctcatctataatggaaatgt

SEQ ID NO: 685 - DNA, 101 bp, Homo sapiens
tggtttctctctagttaaaaaggaatgttcaaaataactcaagaggttcgytttctggcaatttgcctctctagcaattcagaatttccttgtagtttttt

SEQ ID NO: 686 - DNA, 101 bp, Homo sapiens
gtttttccttaagaatggtgaagttgttttttttttttaaaaaaaggaaaygcatatgagttctggatagtttgaatacttggaaaaattattgtcctgga

SEQ ID NO: 687 - DNA, 101 bp, Homo sapiens
aaaccatcagaaaaaaaaaactatattcccctttccactctttatcataartataacttcaattaaaggaaataactttgatttatagttagaccacaaca

SEQ ID NO: 688 - DNA, 101 bp, Homo sapiens
cagttcacaacccatacccacagagaaacatacacatataccttatattayattggttcttttttttcctgaaacaaaaggtctcacatatttattactga

SEQ ID NO: 689 - DNA, 101 bp, Homo sapiens
ggaagtcaaaagttataagccaagtttcaaccgcttgcaaatgtacccctraaccccatgttgtacaagggtcaactgtactgttactgtcccctgttaca

SEQ ID NO: 690 - DNA, 101 bp, Homo sapiens
caaacctaggaggcaatattgcccagctgtaaggagcatgggctttagaayctctggttgctcttgttaatggtgcgactttaggcatgttatttaacctc

SEQ ID NO: 691 - DNA, 101 bp, Homo sapiens
ttggagttagtgtcagtagtgttgaatcattcaggactggatattaagtaygtaagggcaatagaagagcctggagcatatttcatatccctctatccctc

SEQ ID NO: 692 - DNA, 101 bp, Homo sapiens
cagcataatgcttggtatttgacatgttatcaagtatgaataggggagtakcaagggatatgaaaggggtcagaccaaaaagggattcattttatacctag

SEQ ID NO: 693 - DNA, 101 bp, Homo sapiens
ttacacccttcacagaattgcttgagggcacaagtacaaagaattaatatrttaattatcataagtgaatcattaaacagcaacagtaattaacagcttaa

SEQ ID NO: 694 - DNA, 101 bp, Homo sapiens
aagcactttaggtttttcagataacataatcagagaggcaagagtatattrtatttgcttttctgcctcttgtctgggcttaaaatatttcacttggagtg

SEQ ID NO: 695 - DNA, 101 bp, Homo sapiens
tctgatcgtctagttccaatatattctctgcctcttccttgatagcttaartcctgaattctgttcttaaatactgttgcagcttaagctgtcctgcctga

SEQ ID NO: 696 - DNA, 101 bp, Homo sapiens
gtggaaagtatagggactaagccaaaccaggagaaagtgtcaactccagtyaagatccagcagaaccctctggattggataagggacccagaataatccat

SEQ ID NO: 697 - DNA, 101 bp, Homo sapiens
ccaaagcagtttatctgtgtaccccaagactgcaaataaatttatagaacrgtgttgcctggtagaattttctataatgatagaaatgttttatgatctgt

SEQ ID NO: 698 - DNA, 101 bp, Homo sapiens
aaagcacagcttaacaagtactctgacacccagaaaaggcctacataaacycagtaggaaagaaacctaaaatagcagaagtgctggatgagagtaaggaa

SEQ ID NO: 699 - DNA, 101 bp, Homo sapiens
caatctcaacaaacattggaagaaaactgttcaaagccactggctcatagmctgctatctctatgaggatgtttaggatgatgtcattatgggttgaatcc

SEQ ID NO: 700 - DNA, 101 bp, Homo sapiens
tatttaatttggggctcagaagggctgaaaactgcattccatgaataagaraactggaaataatcaaagaactatatggactgcagcatctctctgccatc

SEQ ID NO: 701 - DNA, 101 bp, Homo sapiens
acagatgcaagtaaaaaaattaaaaagtattacggaaccacaatatttatragggacagtcctaagaatcccatgatttcccagattgataagggaacagt

SEQ ID NO: 702 - DNA, 101 bp, Homo sapiens
ggataagggagaatgtatatacaccaccaaaaaggagagagtcacaccgaraagtcagttttgagatcagtttagagaaaatgcaggccaaggcagtgtca

SEQ ID NO: 703 - DNA, 101 bp, Homo sapiens
cccttcccttcaagcaaaactcttgtgattcccctacactattttatggckccatgtgcttgtatattctgatccctctccccaaatgccctatcctgact

SEQ ID NO: 704 - DNA, 75 bp, Homo sapiens
accaataatttgattttgttgatayatccagatttgaccatttcaaggaagtaattcgtgtttatttaaattctc

SEQ ID NO: 705 - DNA, 101 bp, Homo sapiens
taagtatttctatatgctactattttttcttagattaaggtcctgaggatmtccaacttttgggttttagagaggtaacgtgttgcctttaacctctatta

SEQ ID NO: 706 - DNA, 101 bp, Homo sapiens
ccagccccaccttcctcttctttgaatcctgcccctcccttgctccagacytcaccaagtctctgcattacagttcacatcaaccctaagttgctctttcc

SEQ ID NO: 707 - DNA, 101 bp, Homo sapiens
gataggaacaaaaatggaatggtattcatctacatattatttgggcctctktactttttatgttgtaaatgaaggagataatttattcttaccacatactg

SEQ ID NO: 708 - DNA, 101 bp, Homo sapiens
agctacaacaggaaaaatgtgtggacatgaagggaacttgtgagtaggtgytgttgagtacatgcctgtgtgtgtatatgtgctagggacacctaccaggg

SEQ ID NO: 709 - DNA, 101 bp, Homo sapiens
tacattttactcttgtaccagtatcacaggttttgaatccaagaaatgtgrgtctatctacattgttctttttctaattattctgacgattttgtgtcctt

SEQ ID NO: 710 - DNA, 101 bp, Homo sapiens
ccaaggatgttcccatcaaatccttccctcatttgattttcacaacctgcraggaaggcaaggcaactggcatccatatggacatggaaaccgagggccag

SEQ ID NO: 711 - DNA, 101 bp, Homo sapiens
atctgattaattcagattagtttatggattagttcctctggggttggatarcttctcttggctcaatcagccatgtcaggggaatgacattgctaatgaag

SEQ ID NO: 712 - DNA, 101 bp, Homo sapiens
aagtagggtctgtatggcaaggacattacctatcttgtttaccatgaaatygccagtgcctagtggatcaccacctagtacacgctcaataaacactaggt

SEQ ID NO: 713 - DNA, 101 bp, Homo sapiens
acacgaaactgttacccatgccttttcattttccccttcattatcctctgyaccttacatttctaaatggaaacccttcaatgactacctacttaactctc

SEQ ID NO: 714 - DNA, 101 bp, Homo sapiens
gatgatgtgcttacatttttctgcaaccgatcttctgacattttctcgttyccccagccacgagattgtaatttaacctcaactttttgtgtgtgtgcaag

SEQ ID NO: 715 - DNA, 101 bp, Homo sapiens
cctggctgagctctgcccgcctggaggctcccacaggatggccctggggaytgctgctgcactcggtaggtgcccttggccagggtcttcctgatgggctc

SEQ ID NO: 716 - DNA, 101 bp, Homo sapiens
tggcacacacaggaagcttgcatctgacaacaggaaggctggaacgccacktggatttgctcaaggagggtacaagcatctcctgctcattgtctcctttg

SEQ ID NO: 717 - DNA, 101 bp, Homo sapiens
aaaggattttccccacatttatagctctgaagttgagctttttatcacctygctttttggctcccaagtcttgctgctgggtagaattacctggaaagctg

SEQ ID NO: 718 - DNA, 101 bp, Homo sapiens
tgagtatttagattctcaagatgactatttcaaaggacagtagttccttgyatgcactaaaaataccccgaaacatgaatacttcttttttaaaatgaatc

SEQ ID NO: 719 - DNA, 101 bp, Homo sapiens
tgagtgtctttgacagtaactccttcatagatgctttcttatgatgtaccmtttaattttgatgaaggtcctgtgaaataagcagagcagattttatgatc

SEQ ID NO: 720 - DNA, 101 bp, Homo sapiens
gttttggaaatgttgttgcattgtcactttctgcagtagaaactgaaaaaygagaaacacactgtgtttgactggaagcccaaaggagacaaaatgttttc

SEQ ID NO: 721 - DNA, 101 bp, Homo sapiens
caaaatagcatataatctagtttggttgaccctttgctttccacaggcacrgaatgggaaataaggatggaaatgagaattggggatgtattgcagaggaa

SEQ ID NO: 722 - DNA, 101 bp, Homo sapiens
cagagcaggaaagtgagctcctcagcagagaccaggctgggatgaggacamcgcggtgcagaagaaaatctgcctggccgtggtgcctaaagctgccatgc

SEQ ID NO: 723 - DNA, 101 bp, Homo sapiens
cacgatataggaagaccaaccaattcttgaaaagcttttttcttttcccarttgcttcagtgatagccacacatttcaataaacccaattttcctccatct

SEQ ID NO: 724 - DNA, 101 bp, Homo sapiens
tctgggccataagatataccttaacagatttaaacaagtagaaatgatacraagtgtgctctaataatgccataatggagctaaatgagaaatgtaaaaaa

SEQ ID NO: 725 - DNA, 101 bp, Homo sapiens
cctggtccctggaggaacagtagcctctgtctgagtcctaaactggggcarcaggccgggcacaatgtctcaagcttgtaatcctagcactttgaggcacc

SEQ ID NO: 726 - DNA, 101 bp, Homo sapiens
gaaataggatttcctcaataaggacaaaatggctcagggccaaaatgaaarcatcactcagcactttttttttttttttacttttatagtcaatgcaaaga

SEQ ID NO: 727 - DNA, 101 bp, Homo sapiens
gtctggtgtccgagcagcgtgtggtcctgggaacatcttacatgaagtgargtgtccatccttgggtgggtccctctgactcaaggcgagtcttgtggagg

SEQ ID NO: 728 - DNA, 101 bp, Homo sapiens
gtaaaaaaaactgaaggtagtaaatgtggtcgttcagagaaattcagagtraaatgaaggagaatgagggacaggatggcaatactaatagataagggagc

SEQ ID NO: 729 - DNA, 101 bp, Homo sapiens
tacttctaggtatacttctaggtaaaactccccaagaaacactcatatatrtgcacaaggaaacaaacataagtatgttccatgaagtactatttgcgaca

SEQ ID NO: 730 - DNA, 101 bp, Homo sapiens
actgaagactccaagctatatggactgaatccacccccaattcccccgccyaattcatacactgaagccctagacccagtgtgactgtactggagacagag

SEQ ID NO: 731 - DNA, 101 bp, Homo sapiens
tacttacccccttcagataaacagaaaatgcaactctatgtaaatattccytaagaatattttgcagcacactggaattaaattagtgctaaagatgatga

SEQ ID NO: 732 - DNA, 101 bp, Homo sapiens
gttccacttacacaaacgtccacaacacataaatctagaaacagaaactaygttagtggctgcctagggtttaggatgaggagggtagatgtgaagaatga

SEQ ID NO: 733 - DNA, 101 bp, Homo sapiens
atgtggtgatgattaaccttgtcaacttattttttaaataatcctcatcgyttataccattgtagtaaagggttcccctctcccatgcagcaagtccagaa

SEQ ID NO: 734 - DNA, 101 bp, Homo sapiens
gaggaaccacccctctccctctctctgccaatctgtattggggcaaggttkggaagtactggcgagggtattacatttcaagaaacatgaccagggaagcc

SEQ ID NO: 735 - DNA, 101 bp, Homo sapiens
aagtcaaaagactagatagagaaatgatgtccagggagctcataatctgcytgtgcaagaattctagtttctagaaagtcactgattaataaattcatgtg

SEQ ID NO: 736 - DNA, 101 bp, Homo sapiens
ctacacaaagccctcttcaacagatagcataaacgctaccctgtaaaatcrccagcaagcctttgtctccttgcagtcagtttctctctgctgcctgccta

SEQ ID NO: 737 - DNA, 101 bp, Homo sapiens
tattgttttctctttaatggtgaaacttgatagggaacctaaaaagaattktaagactgcattcacttaatttgaagcttaactagaaatttgtttgctgt

SEQ ID NO: 738 - DNA, 101 bp, Homo sapiens
ccactctactgcttgggagtaagcggccaccaaaaccccgcttccagcagrtgctaggagcaacatgacaggaaaaacacaacctaattaaaatggtagag

SEQ ID NO: 739 - DNA, 101 bp, Homo sapiens
ctccatcctcatctgtctggtcgctgtctccacttctctcttcagatatcrgttcaggcccagctgcaatagatacctgcatgactccacccaaggacaaa

SEQ ID NO: 740 - DNA, 101 bp, Homo sapiens
gatgacttactttgctgccaaagggctgggcctgggcctgggcctctgagycaggttctccatcctcatctgtctggtcgctgtctccacttctctcttca

SEQ ID NO: 741 - DNA, 101 bp, Homo sapiens
acttctaaattaccaccatccaggttgcatctatttatggttccattcccygaactgatccaataaagcttgttttccacatagtctatcgatagacctgt

SEQ ID NO: 742 - DNA, 101 bp, Homo sapiens
tcacagtaacccccagtcctcaaaacatcaacaataaacacagacctgcaytgattgtggtattctgggtatttctataacatttctaggtttctgtagat

SEQ ID NO: 743 - DNA, 101 bp, Homo sapiens
atcttggtttttctgccttgacctttggctctttctaatgtaattggctcmgactccatttctggccatctgaactctggttccaagaattaatccaggtg

SEQ ID NO: 744 - DNA, 101 bp, Homo sapiens
atctctccttaattattacagaaaaaaatgttattaaagaaacaatcaggkgatccagcaaaagctgacaatgcacagtagtttagaaaccataagatgca

SEQ ID NO: 745 - DNA, 101 bp, Homo sapiens
gaggttattagcatccccttttacagaagaaaaaactgagaaaccaagcayatacagctggtaagtaacgtagtctgggtgcaaaaccacgaagctcatga

SEQ ID NO: 746 - DNA, 101 bp, Homo sapiens
cttctgctttcaaaaggaattgaagaaaccctaagataaaagagacaagayacactcaagccattcaaagaacaaggacggcacagaaagtacaggttata

SEQ ID NO: 747 - DNA, 101 bp, Homo sapiens
tctgggaacagactacttgctgaaacgaacaaattcccaggcagttgaaarcctttgtgtttcctactgggaataacctgcattcacaaattcattagcct

SEQ ID NO: 748 - DNA, 101 bp, Homo sapiens
tctgcccactgtttctctccttctgctgcagatctttgagctgaataagcrtatttcagctgtggaatgcctgctgacctacctggagaacacagttgtgc

SEQ ID NO: 749 - DNA, 101 bp, Homo sapiens
tctcagttttggagaccaaaagttggctgttttggtgggctgaaatagagytgtgggaagggccccactccagatggaggctctgggggagaatccttttt

SEQ ID NO: 750 - DNA, 101 bp, Homo sapiens
tatatatgtcaagcaataccttagtaaggtactcacttattttatccctartggcatattaatcaggcaatgtcatagatctctggttactattccacctc

SEQ ID NO: 751 - DNA, 101 bp, Homo sapiens
cactagttattggcggtggtgaattcagtttacatggctctgaattcatarcaagtttatttctttaggaaaatgcaaatagttattgtggttggcagaat

SEQ ID NO: 752 - DNA, 101 bp, Homo sapiens
tctgaagggctaagcaagggtaagttgtttatgctgttgcaggaaccacartgatgggaaagaaaaatgatatggtatttccatcccgggccttaaaataa

SEQ ID NO: 753 - DNA, 101 bp, Homo sapiens
aaatgttgactatatacctgcttgataataagaaacattcacctctcttcrtttaagttcaacttaaagaagaaacatttttgaaaagtgagaagtgtgtt

SEQ ID NO: 754 - DNA, 101 bp, Homo sapiens
caagatagccttctttagaatatgatttggctagaaagattcttaaatatrtggaatatgattattcttagctggaatattttctctacttcctgtctgca

SEQ ID NO: 755 - DNA, 101 bp, Homo sapiens
gctttataactgagatgtgtacttcaggcttgcatgggaattgtctgtacrgcccacaaactggcccccaggtctttgggactccttcctgtaacttagtg

SEQ ID NO: 756 - DNA, 101 bp, Homo sapiens
ttattatctctgaatcacagatgagtaaactgaggcacagaggttttttgktttttttttcccttaaggacagaaaacagcatattcaaaccgaggcatgt

SEQ ID NO: 757 - DNA, 102 bp, Homo sapiens
aatcacaggtttttatcaataaatgtccagctgggtacattcctccctctmtctaaacacaactcctgccggtcaggcactgtgtcctagaacctttgccat

SEQ ID NO: 758 - DNA, 101 bp, Homo sapiens
cactttgctgctgctctttctgcctctgtgaccactccttataggttcctyttcttcttgtgcctgcccctttaatgctgatattgatgttttctcccaag

SEQ ID NO: 759 - DNA, 101 bp, Homo sapiens
cacaaaagaaatgtttcctctcacagttggtgaagctagatgtctaaaaaycaaggtatcagtagggccatgctcccactgaaggctgtagggaagattcc

SEQ ID NO: 760 - DNA, 101 bp, Homo sapiens
ccttgtacttctccttggtgtcatgaagacaaatagcattaaaaaaagttytcccagtgaagcagctctcattttctcctctctcatccccttccaaacat

SEQ ID NO: 761 - DNA, 79 bp, Homo sapiens
gagcgtagctttctagagtgtgcgagtggtggctagatgtgctttgtttcrtgtgctgtgcatttcagtgctagtgtga

SEQ ID NO: 762 - DNA, 101 bp, Homo sapiens
ttccctcattgccaatcaccccatttagttatgaaaatacttcattggtartagtggccaaacaggcaaatatctattcagtaattagatgaataaatggg

SEQ ID NO: 763 - DNA, 101 bp, Homo sapiens
aaaaacaacaaaaatacaaaattttcatgatgatataataggaagctctcraaggttggattcaggtaaggaaatgggggaaagtttcctgataccctgac

SEQ ID NO: 764 - DNA, 101 bp, Homo sapiens
cagcaggagtggactgaatagcgtgcccctgggaggtttgtcttcctaagyagatccaatcggtcttcttgttctgatgaagtaaaacagagtggatatcc

SEQ ID NO: 765 - DNA, 101 bp, Homo sapiens
aaacaaactgttctaaattcaaggagtctctgccagttatgtgactttgcrtgactgactctgctttacccctccaggcccaagagacaaggctgtccaga

SEQ ID NO: 766 - DNA, 101 bp, Homo sapiens
taatctcccagaggtgtttccttttgttactctccaaaatgaaaagtctayttttttcttatcaaagccatacatgcttcctgtaaaatcaactcagataa

SEQ ID NO: 767 - DNA, 101 bp, Homo sapiens
tcacagggaatggggtttcttttatcactgacgatagcaagacctactttyttgctctggacagctcctatgaaaatatggcattcagaactgcttccctg

SEQ ID NO: 768 - DNA, 101 bp, Homo sapiens
gaaaggatgataaatcttaggaataataccaatggcattaatgtaatcccrcgtaagtttcgaaaaacctttccaagtataaattcagtaagaaaagctgg

SEQ ID NO: 769 - DNA, 101 bp, Homo sapiens
tgccgttcttggcatcatttctatttggctgtgagtcgtccgcttgatgcrtggtccacagctgattttcatgccccaaacaatccccatcgaaggtcaca

SEQ ID NO: 770 - DNA, 101 bp, Homo sapiens
caatggttaagaattaatttctatgtgttttgttatccgttaaacacaggytgtgagctagcaagaaacaagatacttttggaggcttagtgacttttttt

SEQ ID NO: 771 - DNA, 101 bp, Homo sapiens
aacagaggacattctgttttggagccatgttcccctgtccctggaataccycgctacttattagaaaagcagaaatgcaaaaaatcacagacatgtggggg

SEQ ID NO: 772 - DNA, 101 bp, Homo sapiens
tctttctgggctaacaccaagggggtggcagggctgtctgtgttcctgctrgtggttataagggagaaattccttccttgctttttccagatcctagaggc

SEQ ID NO: 773 - DNA, 101 bp, Homo sapiens
gaaaagcttcctagagaaggggcagctggaacctgaagaacaaaaccagarctgacgacgacggatgaggcaggtgtttcaggtggcagagcaacacaggc

SEQ ID NO: 774 - DNA, 101 bp, Homo sapiens
caatttctccatttttaaaattggtaagtcccccagcccaaggatatggtragtgattgtgtgacctccagaaaccacacttctcccatggatctttgcag

SEQ ID NO: 775 - DNA, 101 bp, Homo sapiens
cttcctcttttcctttgttctctattgcctttacctattttaaaaagtttyaaattattagccagtcgggttttagtttaaattgtaaggtctagctccag

SEQ ID NO: 776 - DNA, 101 bp, Homo sapiens
ataggtgagagggatctagattacgaaaggcctctgaagccagggagaaaytgaacttaatatgacaggtagtgaggagtcagtgtgagttcctcctgggc

SEQ ID NO: 777 - DNA, 101 bp, Homo sapiens
cttttccatttccatttttacttcctctcctacagtctcttttaaatccayaaccaattaggttttcattccaccaaagctgctcattaaaatcccttact

SEQ ID NO: 778 - DNA, 101 bp, Homo sapiens
aaggcatttaggtcctgggcatgcaggtctgtctcctctcactagaatgcmagttctggatggtcagcaattttgtttcattcactgtcatgggctgtgac

SEQ ID NO: 779 - DNA, 101 bp, Homo sapiens
tctctctcgctgctatcaggttgtcagtgtttgtccttgctgagccaggtragcaggcttctgatgtatttacgtaggtcaatggtctctaaaattatttg

SEQ ID NO: 780 - DNA, 101 bp, Homo sapiens
ggcatcacattagagactccaaaatcagactacctacttcaaatattaackctgtggccttaagatattaaacccttatgtgtctcagtttcttcatctat

SEQ ID NO: 781 - DNA, 101 bp, Homo sapiens
aagacaagcaaatttttcatcaatgaagttatacaaatgtgaaacatacaraaagatgttcaacactattcattattggagaaatgcaaattaaaaccaca

SEQ ID NO: 782 - DNA, 101 bp, Homo sapiens
tatgtgtgagtgtacatatatgttttaaaaatccctagcaagagtaagtaygttatttggtcagtcagctgttaaaacttccactttctccagttgtctgg

SEQ ID NO: 783 - DNA, 101 bp, Homo sapiens
taactggcaacacatgcactttcttttgagcttttaaaaacattgctccaytgctatcattgtagacccccaaggagaaggtaccccagcctcctggaaac

SEQ ID NO: 784 - DNA, 101 bp, Homo sapiens
ggtccaaaagggccacagtttgctggcagaaaccatacgaagtagattttrttgttacccccattttaaagatgaagaaactgagtcccagagaggttcag

SEQ ID NO: 785 - DNA, 101 bp, Homo sapiens
tctaacctttggtgtgcgctgtccctaagggaggaaggagtgcagctcacmaaagcccccttgaaacaaaggaaatgtgaacgcaacaccaaccactgaag

SEQ ID NO: 786 - DNA, 101 bp, Homo sapiens
gcctttatctctgcttcttctacccaacaggtgactccttttagctagggyatcacttatacctaacaggggactcaatttagccaggatttcactctggc

SEQ ID NO: 787 - DNA, 101 bp, Homo sapiens
atctttccactggagggaaattgggttcatagagtagaaatactttgcccragcctcaacagctgctaagaggtgcaatgaaaactcaacttgaggctgtc

SEQ ID NO: 788 - DNA, 101 bp, Homo sapiens
ccatcttggcatcattaaaaagggccaaccaagatgttacatgtccacgaygtgacacaggaggaatcaaacagcctgcctatgaagtagtcttgacaaca

SEQ ID NO: 789 - DNA, 101 bp, Homo sapiens
tttagctatctgccatttccagacacttcatgctctctgagtcttatcttycactcccagaagattgtcaaagtattttccaaaacaaagatagtttcccc

SEQ ID NO: 790 - DNA, 101 bp, Homo sapiens
aacataactttggggaatagctatagatactaaaggggcaacataaaacakttattgattacaaagtgtatgaagacccagttgcttggcagagtgatatc

SEQ ID NO: 791 - DNA, 101 bp, Homo sapiens
aatggggcaggaggtagaatggcacaggaattcaagtagaggaggatttaycatgaagctaatgaagtctaagtttcagggcttctcacctgtgcaggcca

SEQ ID NO: 792 - DNA, 101 bp, Homo sapiens
tatagcatctatttataagccacacacaccatcttatattaatgcttataytgtcttggctcacttagatacaaataaaggtttgcatctgatagaggaat

SEQ ID NO: 793 - DNA, 101 bp, Homo sapiens
ttaatggatgaagatatgtagacatctatggtgttctgggaagctgagcaygtctgatataaggcatgtgaggtttaaatgcatgcatgtgttagatatgt

SEQ ID NO: 794 - DNA, 101 bp, Homo sapiens
gccaagttcccaaggtcgcagcaaggtaaatgggattccacttgtgttcgraaaatctgtttataggccttctcctgaatcaaaacacacaggggaaaagc

SEQ ID NO: 795 - DNA, 101 bp, Homo sapiens
ataagggtgaggctagatctgctatgtccgaaatggcagccactggatgcrtgactagatttacattaattacaatgattctaataaaaaatgaagttctc

SEQ ID NO: 796 - DNA, 101 bp, Homo sapiens
aaccatgccttgttttgcgtcttctcaaagaaccccgggggcacgtggccracaatgtacacctacaagggaggggttcccagaagaggctcacagatgcc

SEQ ID NO: 797 - DNA, 101 bp, Homo sapiens
aaccccctcctttctcctgtactgatgactctgtagctttaaccagggcgrcggtgtcactctaaatgtcaccttggcattcagccccatagagtggggaa

SEQ ID NO: 798 - DNA, 101 bp, Homo sapiens
caagcaaaagaaccttgaataagccaatatttcactcataatgtgagtgcraaacatgaaacccaactttccgggttcaaatgccaagtacagctagagtc

SEQ ID NO: 799 - DNA, 101 bp, Homo sapiens
gccctggggctaggataagcttcttctctgattcaaagaagcattctccaragttgcttgccagataccaggttctgagctagttggcctcccaaaaaccc

SEQ ID NO: 800 - DNA, 101 bp, Homo sapiens
aaatatttatgatgttgtctaaaaatgagtaggtaacacactccacattaycagtacagagactattctagcatcaatgaatgccaccatagataactatt

SEQ ID NO: 801 - DNA, 101 bp, Homo sapiens
atggcagtaagtcatacccaaatttggttcacttcactcaaatatttgtgrggcacttaacgattaaagggtttgtaggtactttgtttaatgaataaatt

SEQ ID NO: 802 - DNA, 101 bp, Homo sapiens
gggactttctggacactaccacatggagactgaagatgaagctaacacttyccagagcaagctgagtgacagacagaaatcaaagcctgatgataccattt

SEQ ID NO: 803 - DNA, 101 bp, Homo sapiens
taataatggaggaaacccggtgggtgtgaggtatatgggagttttctggaytctttgcagtttttcagcaatctaaaactgttccaaaataaagtttagac

SEQ ID NO: 804 - DNA, 101 bp, Homo sapiens
ttaggtcttcctaagaatgtatttctgcctcagaatgcacaatgttttcayataaatgtcagtatgattagggtttattagcaattgtaaaaaattcaaca

SEQ ID NO: 805 - DNA, 101 bp, Homo sapiens
tctactccaacttgttggaaagtagtagtagaatacaaactagtcaaatamaccacgttgtgtaatgaactgaaactttaacttattttgttggagtcaaa

SEQ ID NO: 806 - DNA, 101 bp, Homo sapiens
tgccctggagacgttttccccattgtcttggtaactaacattcagctccgygtgcagcaccaacttacttatgcaaatttctgtcactggtttgaatttct

SEQ ID NO: 807 - DNA, 101 bp, Homo sapiens
tcaggtctactcatctgtaaaatgagaataataactgccaccaactccctrgattatgttgaggatttgattaggtagtgtttatggagcatggcacgtgt

SEQ ID NO: 808 - DNA, 101 bp, Homo sapiens
actccacctctctctgatgagaagaggtaagtaggatttacagataagcayacgagaagcaggggaagatgctaaggcaaagaaggggcctgaacaccacc

SEQ ID NO: 809 - DNA, 101 bp, Homo sapiens
tgtgaaatttcaagttttattcttccattgttccatttgtgacctaatctrtgaagcctttcttatcttttccaagtaaaggatcactctcttcttacttc

SEQ ID NO: 810 - DNA, 101 bp, Homo sapiens
acagaggctgtccttaaaggagctgagcctcccttctctcaagggcatctrtgtctgcgaatccatccaggctgatgactgtcaacctggggctttttgtt

SEQ ID NO: 811 - DNA, 101 bp, Homo sapiens
tcttctctgaaagctgaattaactagtcaggaatcgcagatctccacttaygaagaagaattggcaaaagctagagaagagctgagccgtctacagcaaga

SEQ ID NO: 812 - DNA, 101 bp, Homo sapiens
tggcaggagtgatgctggcctaatgacaacctcagccaatgaccaacctcrtggagatactttagagctaaaaccgtatcttaatgttgtcatgcattggc

SEQ ID NO: 813 - DNA, 101 bp, Homo sapiens
ggcagaccataatgattcaaccacttggattctacaaacaatactttaacrtggaaatgtgtacttggatgaagaagagagaaggcaatgcctgatttttc

SEQ ID NO: 814 - DNA, 101 bp, Homo sapiens
agcactgaagaggtctttgctgactctggctcaggaattagaagtttctcrgcaagggccatttaaattgggccttgatggatgtataggagctcaataag

SEQ ID NO: 815 - DNA, 101 bp, Homo sapiens
agttacaagagctcactgaccaacacaaactcaggttagcagagcttttcygaaattccacccttttcctctgcgttcagtgctacttactcccttcactt

SEQ ID NO: 816 - DNA, 101 bp, Homo sapiens
gctccgctattcagtttcaggtagggacatagagtcttagagagggtgagrcatttatacaaggctacatagcaagtagaaggcaaaaccaggacaggatc

SEQ ID NO: 817 - DNA, 101 bp, Homo sapiens
ctttactgagctcctattagatgctttgcatgaggtatttcaatttaatgygtaggaactgtggcttatttgacttgttacacccaacagcgcctggcacc

SEQ ID NO: 818 - DNA, 101 bp, Homo sapiens
cagtgctcccagtggtgggtacctaccccagagaactctcacatgtatcaraagtgggcatgtatacagttcagaactgtccatcatggtcagagttgaag

SEQ ID NO: 819 - DNA, 101 bp, Homo sapiens
tgcaaactgggaagctgagggtgcccatgttttctgttatgtggactaggragcagagaatgccacccacaaggaaagagaaaactcaagcatatgttcag

SEQ ID NO: 820 - DNA, 101 bp, Homo sapiens
caccagcctcattggccttgtccctgaatcttacacacctaaatgcaaacrcaccttccaattatctgcttgttcttctttttatccacttctttgtctcc

SEQ ID NO: 821 - DNA, 101 bp, Homo sapiens
cttacatttcatttctttcattcttattccttaatagatatgtttgttctytctgcctgttctttttcttattcctccatctttgcttgtctatcccaccc

SEQ ID NO: 822 - DNA, 101 bp, Homo sapiens
  allele (51)..(51)
tcagagaccccagggaattcacatgtgtattgctttgtaaatgttctgacratgctctcaggtgccagggctgtgtcttcagcatcagtttgggagttcct

SEQ ID NO: 823 - DNA, 101 bp, Homo sapiens
ataaaacactctctgaagaaaatacatttctctatatctatgtcttgttayttacttatacatctataaattttttgattaacctatgttctttgttttct

SEQ ID NO: 824 - DNA, 101 bp, Homo sapiens
tgacttccagaactagtaatttggtgtcggagaactacgtgtatatggcamctttgtacattcattaagttggcacaatttgaccttttgttgcatgacag

SEQ ID NO: 825 - DNA, 101 bp, Homo sapiens
cagatttcactaacataggagattgtactgatttgcactctcactagccayatatgagcatgtcaatttctcctacatctcaccaacacagtatattagca

SEQ ID NO: 826 - DNA, 101 bp, Homo sapiens
ccaagaaagtctctagaagaagggtgacataaattgcaaggacgtgggttrgtgtgaggttttcatgacttcatctgccacccaaattatttattttccat

SEQ ID NO: 827 - DNA, 101 bp, Homo sapiens
tacggttttttgcctttttctatttgatttagaatggatacaagcggcctytaggactgattcatggaaggtcaggcagagtctccctggatgcctggaaa

SEQ ID NO: 828 - DNA, 101 bp, Homo sapiens
tcaagaaagtcatttcctaatgaaaaatggttctggtcatcttccctctgkctgtatgtgttcatcaacagagtttgacttcagattaattgagattaaag

SEQ ID NO: 829 - DNA, 101 bp, Homo sapiens
gtttctggaagagattcacatttgcatcagtaggctgagtaaagaagatcyacctcaccaatgtgggccaacagcatccaatccaaatagaataaaaagga

SEQ ID NO: 830 - DNA, 101 bp, Homo sapiens
cgagttttgcgtttagcagatggacctgacgaagttcatctttcagcaatygcaacaatggagctgcgggaccaagccaaaagactgacagccaagatata

SEQ ID NO: 831 - DNA, 101 bp, Homo sapiens
ataaaatctggcttacagaaaaacaggcaaaatacctttggcccagtgaaygaattgtccttggtttccatgttaatacccagaggccagtactgggcgtt

SEQ ID NO: 832 - DNA, 101 bp, Homo sapiens
gttaatgaaaagtaaggctaccaaatatatcaagctatatctgcttggacrtagctttgtttactttgtgacaggaatttcttagagtcagcctccagtaa

SEQ ID NO: 833 - DNA, 101 bp, Homo sapiens
aaaaggcccccccaaaagtagatacaaggcaaagtctgtctcatgattctkctgtagaaaagaaacattaaaaagtatgagcaacattaatagcagatggc

SEQ ID NO: 834 - DNA, 101 bp, Homo sapiens
tgggagcaaagggaagcactgaagcaaaccacatgaagtaagagcaggaartagctctcacccccaggactgggaaagaaagggaagataatagagtaacc

SEQ ID NO: 835 - DNA, 101 bp, Homo sapiens
tgcaaatagcaggggaacatcctctagttattttaataattcaagggaggkacaaggttttccactttttctcccctgcaaagcactctctaaattgcagc

SEQ ID NO: 836 - DNA, 101 bp, Homo sapiens
cttttgaataactggtcgaactcatagcaaggggagctgagcccacttaayccagaatgacagaaagaagctaagcctgtttatttgattgcagatctcgc

SEQ ID NO: 837 - DNA, 101 bp, Homo sapiens
ggtttcctttctgggagaatggggctggtgacaattaatgcagaaaacttytattggagatagacatgcaaaaacactcagagatagagttgaggaatttg

SEQ ID NO: 838 - DNA, 101 bp, Homo sapiens
ctgtgtgcccatggggttcttcgtgaagagtttcatggttgctgcttcagractactgagatttgggaattctcctttcttgttcttttaaaataagcaat

SEQ ID NO: 839 - DNA, 101 bp, Homo sapiens
tttgttaatctcagaaaagaatgtatcattttaagaaatacactaaaaggrccagtcgtaagcaaagagctctgttatttagtaagtagaaagtgtagata

SEQ ID NO: 840 - DNA, 101 bp, Homo sapiens
gaaagcagaggtagagtgggaaggactttttattgtgctttgggtaccagytcagctgcagtagaatagagcaccaggtagctttctaaggttcctgactc

SEQ ID NO: 841 - DNA, 101 bp, Homo sapiens
tgtggtgaataatgacgaaataacaaagcaataaaagcagcagcagcattrtatgtattttgttttcgatatgcaaatgaagggcagacttatttttgtca

SEQ ID NO: 842 - DNA, 101 bp, Homo sapiens
agtgatttgtcctcaatctcacccagaagagagtaagaactgacctcatamatttaaggaacaaagtgggacgaaggaagcctgatgtgactggggttggg

SEQ ID NO: 843 - DNA, 101 bp, Homo sapiens
gtgggctgtaatctttgaatctagactttatctttgcagttccagtctttmggcaaccgtccaggaggacaagactcattcagtgaaagtggcttcaattt

SEQ ID NO: 844 - DNA, 101 bp, Homo sapiens
acatagtccaagtcaccttcgtctctaccctggactacactataacctccyaattagtcttcctacagccccttatccccctcagtattttcttttgctgc

SEQ ID NO: 845 - DNA, 101 bp, Homo sapiens
ctgttggatcatggcccatacctcagcagtgccatatccaattaggatgcrtcattacattatggttcgcaacagaaaaactgtctccaccaattggaagg

SEQ ID NO: 846 - DNA, 101 bp, Homo sapiens
atgtggaacagagtactcctttatttgtgccatgattaggccaactctgargtgaagaagaggaatagaaaaaagcaaaagttagaagcatttaatgaaat

SEQ ID NO: 847 - DNA, 101 bp, Homo sapiens
ttggaagagtacaaatttaaaggagccaatgaggtgcatacatgagaaggragccttgcaaaaatatactagtttattactgtgggtgaagacaaatgtta

SEQ ID NO: 848 - DNA, 101 bp, Homo sapiens
atagaagaagcagcggagactactgctgctaatgactgtagacatgttgarcattagaatttgtagcaggagttgccctgccatagcaagaaatgacaatg

SEQ ID NO: 849 - DNA, 101 bp, Homo sapiens
ttttgttatttaaatcccctataattatatttttaattttctcgatttgaytatttctgagaacagagttggggtaggcagaataatgatccaccaaagac

SEQ ID NO: 850 - DNA, 52 bp, Homo sapiens
gagagcatcatctgcggcatcacgtcygtggccttctccctcagtggccgcc

SEQ ID NO: 851 - DNA, 52 bp, Homo sapiens
cgccgccaggcgcacggcgtaggggarcctcgcaggcggcggcggcggcggc

SEQ ID NO: 852 - DNA, 52 bp, Homo sapiens
gctctcgccgccaggcgcacggcgtargggagcctcgcaggcggcggcggcg

SEQ ID NO: 853 - DNA, 52 bp, Homo sapiens
gagaaccagttcagagtggactacatyctgagtgtgatgaacgtgcctgact

SEQ ID NO: 854 - DNA, 52 bp, Homo sapiens
atctgcagcttaagccagtgacacaayattttgcatttttaaatggtgattc

SEQ ID NO: 855 - DNA, 52 bp, Homo sapiens
ctggtcttctcggtgcgcagcccctcrtgggtgctcaacttcctgctgcaga

SEQ ID NO: 856 - DNA, 52 bp, Homo sapiens
ttagcacccagggtcacatcccagttyaaaaatatcccatggagtgcagtca

SEQ ID NO: 857 - DNA, 52 bp, Homo sapiens
tgttcatctattcaaaatgtagtatarttttatttgagattgtcttttttta

SEQ ID NO: 858 - DNA, 52 bp, Homo sapiens
gcgtccgcagagcccgcgggggccgkyccagcccgggagccgcgcgggcgag

(19) United States
(12) Patent Application Publication
(10) Pub. No.: US 2011/0143956 A1
(43) Pub. Date: Jun. 16, 2011

(54) Diagnostic Kits and Methods for SCD or SCA Therapy Selection

(75) Inventors: Orhan Soykan, Shoreview, MN (US); Tara Nahey, Minneapolis, MN (US); Jeffrey Lande, Minneapolis, MN (US)

(73) Assignee: MEDTRONIC, INC., Minneapolis, MN (US)

(21) Appl. No.: 12/961,694

(22) Filed: Dec. 7, 2010

Related U.S. Application Data
(63) Continuation-in-part of application No. 12/271,385, filed on Nov. 14, 2008.
(60) Provisional application No. 60/987,968, filed on Nov. 14, 2007.

(51) Int. Cl. C40B 30/04; C07H 21/00; C12Q 1/68; C07H 21/04; C40B 40/08

(52) U.S. Cl. 506/9; 536/24.31; 435/6.11; 506/17

(57) ABSTRACT

Variations in certain genomic sequences useful as genetic markers of Sudden Cardiac Death (“SCD”) or Sudden Cardiac Arrest (“SCA”) risk are described. Novel diagnostic kits, DNA microarrays, and methods employing these genetic markers are used in assessing the risk of SCD or SCA. Methods of distinguishing patients having an increased susceptibility to SCD or SCA, through use of these markers, alone or in combination with other markers, are also provided. Further, methods of detecting a polymorphism associated with SCD or SCA are taught.

### CROSS-REFERENCE TO RELATED APPLICATIONS

This application is a continuation-in-part of U.S. patent application Ser. No. 12/271,385, filed on Nov. 14, 2008, and claims priority to U.S. Provisional Application Ser. No. 60/987,968, filed Nov. 14, 2007.

### REFERENCE TO SEQUENCE LISTING

This application contains a Sequence Listing submitted as an electronic text file named ST25.txt. The information contained in the Sequence Listing is hereby incorporated by reference.

### BACKGROUND

Implantable cardioverter-defibrillator (ICD) therapy is effective in primary and secondary prevention for patients at high risk of Sudden Cardiac Arrest (SCA). ICDs can effectively terminate life threatening ventricular tachy-arrhythmias, such as ventricular tachycardia (“VT”) and ventricular fibrillation (“VF”). For many patients, ICDs are indicated for various cardiac related ailments including myocardial infarction, ischemic heart disease, coronary artery disease, and heart failure. The use of these devices, however, remains low due in part to lack of reliable markers to select patients who are in need of these devices. Left ventricular function, clinical comorbidity, QRS duration, and various electrophysiological testing methods have been proposed as criteria for the screening of patients potentially at high risk for arrhythmic death. But risk stratification remains unsatisfactory because it is mainly performed using a single clinical marker, namely left ventricular ejection fraction. Hence, despite the effectiveness of ICDs in Sudden Cardiac Death (“SCD”) or Sudden Cardiac Arrest (“SCA”) prevention, many susceptible patients who might benefit from an ICD do not receive one due to a lack of reliable methods for the identification of SCD or SCA. The financial burden and potential risks associated with this therapy make better identification of patients with a propensity towards SCA a desirable goal.

One possible type of genetic maker for improved risk stratification is a Single Nucleotide Polymorphism (SNP). Human beings share 99.9% of their gene sequences. Given the approximate size of the human genome, which is approximately 3 billion base pairs, it is believed that there can be as many as 3 million sequence differences between any two individuals. These base pair differences predominantly show up as polymorphisms, defined as variants that occur at a frequency >1% in the population. If these polymorphisms result from the substitution of one nucleotide for another in the DNA sequence, it is called a single nucleotide polymorphism (SNP). Polymorphisms affecting the coding region of a gene may influence the structure of the protein product, whereas others located within the regulatory sequences (also referred to as the promoter region) of a gene can influence the regulation of expression levels of the protein product. In some cases, these genetic variations may alter phenotypic expression following a change in physiological conditions, such as an ischemic event or the administration of a medication. Diagnostic data from a medical device such as an ICD can be used to obtain information of various diagnostic markers, including information about tachyarrhythmia episodes for the identification of possible genetic markers for SCA.

### SUMMARY OF THE INVENTION

Novel diagnostic kits and methods for assessing the risk of Sudden Cardiac Death (“SCD”) and Sudden Cardiac Arrest (“SCA”) and useful genetic markers thereof are provided. Methods of distinguishing patients having an increased susceptibility to SCD and SCA using the diagnostic kits and methods, including various DNA microarrays, through use of the genetic markers, alone or in combination with other markers, are also provided. The DNA microarrays can be in situ synthesized oligonucleotides, randomly or non-randomly assembled bead-based arrays, and mechanically assembled arrays of spotted material where the materials can be an oligonucleotide, a cDNA clone, or a Polymerase Chain Reaction (PCR) amplicon.

Specifically, a diagnostic kit for detecting one or more Sudden Cardiac Arrest (SCA)-associated polymorphisms in a genetic sample having at least one probe for assessing the presence of a Single Nucleotide Polymorphism (SNP) in any one of SEQ ID Nos. 1-858 is provided. Preferably, the SNP is selected from the group of SEQ ID Nos. 850-855 and 858. Also provided is a DNA microarray for detecting one or more Sudden Cardiac Arrest (SCA)-associated polymorphisms in a genetic sample made up of at least one probe for assessing the presence of a Single Nucleotide Polymorphism (SNP) in any one of SEQ ID Nos. 1-858, more preferably SEQ ID Nos. 850-855 and 858.

The SNPs in the kits, compositions, and methods of the invention include any one or more selected from the group of SEQ ID Nos. 1-858. The SNPs are preferably selected from the group of SEQ ID Nos. 850-855 and 858. It is also understood that the group of SNPs may further include any of the following groups of SEQ ID Nos.: 850-851, 850-852, 850-853, 850-854, 850-855, 851-852, 851-853, 851-854, 851-855, 851-855 and 858, 852-853, 852-854, 852-855, 852-855 and 858, 853-854, 853-855, 853-855 and 858, 854-855, 854-855 and 858, 855 and 858. It is also understood that the group of SNPs may further include any of the following groups of SEQ ID Nos.: 850 and 852; 850 and 853; 850 and 854; 850 and 855; 850 and 858; 851 and 853; 851 and 854; 851 and 855; 851 and 858; 852 and 854; 852 and 855; 852 and 858; 853 and 855; 853 and 858; 854 and 858. It is also understood that the group of SNPs may further include any of the following groups of SEQ ID Nos.: 850 and 852-853; 850 and 853-854; 850 and 854-855; 850, 855 and 858; 851 and 853-854; 851 and 854-855; 851, 855 and 858; 852 and 854-855; 852, 855 and 858; 853, 855 and 858. It is also understood that the group of SNPs may further include any of the following groups of SEQ ID Nos.: 850 and 852-854; 850 and 853-855; 850, 854-855 and 858; 851 and 853-855; 851, 854-855 and 858; 852, 854-855 and 858; 850 and 852-855; 850, 853-855 and 858; 851, 853-855 and 858; 850, 852-855 and 858.

A system for detecting one or more Single Nucleotide Polymorphisms (SNPs) associated with SCA is also provided. The system comprises a computer system having a computer processor programmed with an algorithm and one or more genetic databases in communication the programmed processor. The system imputes p-values for one or more known SNPs that are detected from one or more genetic samples obtained from a patient. Additionally or alternatively, the system imputes p-values for one or more known SNPs obtained from the one or more genetic databases. A p-value of less than a specified range indicates association with SCA.

Novel genetic markers useful in assessing the risk of Sudden Cardiac Death (“SCD”) and Sudden Cardiac Arrest (“SCA”) are provided. Methods of distinguishing patients having an increased susceptibility to SCD, or SCA, through use of these markers, alone or in combination with other markers, are also provided. Further, methods of assessing the need for an ICD in a patient are taught. Specifically, an isolated nucleic acid molecule is contemplated that is useful to predict SCD, or SCA risk, and Single Nucleotide Polymorphisms (“SNPs”) selected from the group of SEQ ID Nos. 1-858 that can be used in the diagnosis, distinguishing, and detection thereof.

Provided are isolated nucleotides, to be used in the diagnostic kits and methods that are useful to predict SCD, or SCA risk, which are complementary to any one of SEQ ID Nos. 1-849 where the complement is between 3 to 101 nucleotides in length and overlaps a position 51 in any of the SEQ ID Nos. 1-849, which represents a SNP. The invention also contemplates isolated nucleotides useful to predict SCD or SCA risk, complementary to any one of SEQ ID Nos. 850-858, where the complement is between 3 to 101 nucleotides in length and overlaps at position 26 or 27 in any of SEQ ID Nos. 850-858, each of which represent a SNP. An amplified nucleotide is further contemplated for use in the diagnostic kits containing a SNP embodied in any one of SEQ ID Nos. 1-849, or a complement thereof, overlapping position 51, wherein the amplified nucleotide is between 3 and 101 base pairs in length. An amplified nucleotide is contemplated containing a SNP embodied in any one of SEQ ID Nos. 850-858, or a complement thereof, overlapping position 26 or 27, wherein the amplified nucleotide is between 3 and 101 base pairs in length. The lower limit of the number of nucleotides in the isolated nucleotides, and complements thereof, can range from about 3 base pairs from position 50 to 52 in any one of SEQ ID Nos. 1-849 such that the SNP at position 51 is flanked on either the 5′ and 3′ side by a single base pair, to any number of base pairs flanking the 5′ and 3′ side of the SNP sufficient to adequately identify, or result in hybridization. The lower limit of the number of nucleotides in the isolated nucleotides, and complements thereof, can range from about 3 base pairs from position 26 to 27 in any one of SEQ ID Nos. 850-858 such that the SNP at position 26 or 27 is flanked on either the 5′ and 3′ side by a single base pair, to any number of base pairs flanking the 5′ and 3′ side of the SNP sufficient to adequately identify, or result in hybridization. This lower limit of nucleotides can be from about 3 to 99 base pairs, the optimal length being determinable by a person of ordinary skill in the art. For example, the isolated nucleotides or complements thereof, can be from about 5 to 101 nucleotides in length, or from about 7 to 101, or from about 9 to 101, or from about 15 to 101, or from about 20 to 101, or from about 25 to 101, or from about 30 to 101, or from about 40 to 101, or from about 50 to 101, or from about 60 to 101, or from about 70 to 101, or from about 80 to 101, or from about 90 to 101, or from about 99 to 101 nucleotides, so long as position 51 in any of SEQ ID Nos. 1-849 and position 26 or 27 of SEQ ID Nos. 850-858 are overlapped. Preferred primer lengths can be from 25 to 35, 18 to 30, and 17 to 24 nucleotides.

The nucleotide lengths can be described by n for the lower bound, and (n+i) for the upper bound for n={xε [symbol] |3≦x≦101} and i={yε [symbol] |0≦y≦(101−n)}. For example, the isolated nucleotides or complements thereof, can be for n=3, for every i={yε [symbol] |0≦y≦(98)} from about 3 to 4 nucleotides in length, or from about 3 to 5, 3 to 6, 3 to 7, 3 to 8, . . . , 3 to 99, 3 to 100, 3 to 101, where position 51 in any of SEQ ID Nos. 1-849 is overlapped, or where positions 26 or 27 in any of SEQ ID Nos. 850-858 is overlapped. Some preferred primer and nucleotide lengths can be from 25 to 35, 18 to 30, and 17 to 24 nucleotides. Preferred primer lengths can be from 25 to 35, 18 to 30, and 17 to 24 nucleotides. A preferred length is 52 nucleotides with the polymorphism at position 27 for SEQ ID Nos. 850-858. An amplified nucleotide is further contemplated containing a SNP embodied in any one of SEQ ID Nos. 1-4, or a complement thereof, overlapping position 27, wherein the amplified nucleotide is between 3 and 101 base pairs in length described by n for the lower bound, and (n+i) for the upper bound for n={xε [symbol] |3≦x≦101} and i={yε [symbol] |0≦y≦(101−n)}.

The isolated nucleic acid molecules of the invention may also consist of nucleotide sequences having a SNP that is selected as being associated with SCA using the system of the invention.

A method of distinguishing patients having an increased or decreased susceptibility to SCD or SCA from patients who do not is provided, and a diagnostic kit or method thereof, where at least one SNP is detected at position 51 in any of SEQ ID Nos. 1-858 in a nucleic acid sample from the patients. The presence or absence of the SNP can be used to assess increased susceptibility to SCD or SCA.

A method of determining SCA or SCD risk in a patient, and a diagnostic thereof, is contemplated which requires identifying one or more SNP at position 51 in any of SEQ ID Nos. 1-858 in a nucleic acid sample from the patient.

A method for determining whether a patient needs an Implantable Cardio Defibrillator (“ICD”), and a diagnostic thereof, is contemplated by identifying one or more SNPs at position 51 in any of SEQ ID Nos. 1-858 in a nucleic acid sample from the patient.

A method of detecting SCA or SCD-associated polymorphisms, and a diagnostic kit or method thereof, is further contemplated by extracting genetic material from a biological sample and screening the genetic material for at least one SNP in any of SEQ ID Nos. 1-858, which is at position 51.

Those skilled in the art will recognize that the analysis of the nucleotides present in one or several of the SNP markers in an individual's nucleic acid can be done by any method or technique capable of determining nucleotides present at a polymorphic site. One of skill in the art would also know that the nucleotides present in SNP markers can be determined from either nucleic acid strand or from both strands.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

The foregoing and other features and aspects of the present disclosure will be best understood with reference to the following detailed description of a specific embodiment of the disclosure, when read in conjunction with the accompanying drawings, wherein:

FIG. 1 depicts increase in the Number Needed to Treat (“NNT”) observed for the ICD therapy as devices are implanted in patients with lower risks.

FIG. 2 is a flow chart of a MAPP sub-study design. MAPP was a preliminary genetic association study conducted to search for markers of SCA. The study involved collection of blood samples from 240 ICD patients who were then followed for more than 2 years for their arrhythmic outcomes. Resulting data was used for the search of statistical associations between life threatening events and SNPs.

FIG. 3 is a statistical plot of Single Nucleotide Polymorphisms (“SNPs”).

FIG. 4 is a decision tree based on a recursive partitioning algorithm.

FIGS. 5A and 5B are genomic groupings of MAPP based on the recursive partitioning algorithm.

FIG. 6 is a chromosomal plot of 849 SNPs with p=0.1 for both MAPP and an IDEA-VF study. IDEA-VF was a pilot study to demonstrate the feasibility of collecting blood samples from post Myocardial Infarction (“MI”) patients to search for genetic markers that indicate the patient risk for SCA. Approximately 100 post-MI patients participated in the study and roughly half of them were ICD patients with life threatening arrhythmias and the rest were patients without ICDs.

FIG. 7A represents a listing of SNPs potentially useful as genetic markers based on logical criteria (CART tree).

FIG. 7B represents a listing of SNPs potentially useful as genetic markers based on biological criteria (clustering in genome).

FIG. 7C represents a listing of SNPs potentially useful as genetic markers based on statistical criteria (min radius).

FIG. 8 shows graphically the operation of a genetic screen in conjunction with existing medical tests.

FIG. 9 shows 25 SNPs identified as SCD or SCA-associated SNPs having p-values less than 0.0001 from the analysis of the MAPP data.

FIG. 10 shows the SNPs identified by the MAPP and IDEA-VF studies associated with risk at SCD.

FIG. 11 is a list of rs numbers and corresponding SEQ ID Nos.

FIG. 12 is a schematic of a two-color analysis of SNPs using microarray technology.

FIG. 13 is a Cox proportional hazards model adjusted for age, sex, and race/ethnicity for GPC5. Individuals homozygous for the protective allele (GG) are shown in green, heterozygotes (AG) in blue, and homozygous for the risk allele (AA) are in red.

FIG. 14 shows individuals classified by counting their number of QT-prolonging alleles in all ten identified markers (max score 20). Dosages for the QT-prolonging allele as calculated by MACH1 were added and then rounded to the nearest integer.

FIG. 15 depicts schematics showing the National Center for Biotechnology Information (NCBI) SNP database model.

FIG. 16 is mosaic plot illustrating the probability of experiencing life threatening arrhythmia (LTA) as a function of allele specific inheritance of the SNP rs1439098. The horizontal width corresponds to the three genotypes and is proportional to their percentage distribution within the study. The vertical axis divides the case and control groups.

FIG. 17 is a mosaic plot illustrating the probability of experiencing LTA as a function of allele specific inheritance of the SNP rs4878412. The horizontal width corresponds to the three genotypes and is proportional to their percentage distribution within the study. The vertical axis divides the case and control groups.

FIG. 18 is a mosaic plot illustrating the probability of experiencing LTA as a function of allele specific inheritance of the SNP rs2839372. The horizontal width corresponds to the three genotypes and is proportional to their percentage distribution within the study. The vertical axis divides the case and control groups.

FIG. 19 is a mosaic plot illustrating the probability of experiencing LTA as a function of allele specific inheritance of the SNP rs10505726. The horizontal width corresponds to the three genotypes and is proportional to their percentage distribution within the study. The vertical axis divides the case and control groups.

FIG. 20 is a mosaic plot illustrating the probability of experiencing LTA as a function of allele specific inheritance of the SNP rs10919336. The horizontal width corresponds to the three genotypes and is proportional to their percentage distribution within the study. The vertical axis divides the case and control groups.

FIG. 21 is a mosaic plot illustrating the probability of experiencing LTA as a function of allele specific inheritance of the SNP rs6828580. The horizontal width corresponds to the three genotypes and is proportional to their percentage distribution within the study. The vertical axis divides the case and control groups.

FIG. 22 is a mosaic plot illustrating the probability of experiencing LTA as a function of allele specific inheritance of the SNP rs 16952330. The horizontal width corresponds to the three genotypes and is proportional to their percentage distribution within the study. The vertical axis divides the case and control groups.

FIG. 23 is a mosaic plot illustrating the probability of experiencing LTA as a function of allele specific inheritance of the SNP rs2060117. The horizontal width corresponds to the three genotypes and is proportional to their percentage distribution within the study. The vertical axis divides the case and control groups.

FIG. 24 is a mosaic plot illustrating the probability of experiencing LTA as a function of allele specific inheritance of the SNP rs9983892. The horizontal width corresponds to the three genotypes and is proportional to their percentage distribution within the study. The vertical axis divides the case and control groups.

FIG. 25 is a mosaic plot illustrating the probability of experiencing LTA as a function of allele specific inheritance of the SNP rs1500325. The horizontal width corresponds to the three genotypes and is proportional to their percentage distribution within the study. The vertical axis divides the case and control groups.

FIG. 26 is a mosaic plot illustrating the probability of experiencing LTA as a function of allele specific inheritance of the SNP rs1679414. The horizontal width corresponds to the three genotypes and is proportional to their percentage distribution within the study. The vertical axis divides the case and control groups.

FIG. 27 is a mosaic plot illustrating the probability of experiencing LTA as a function of allele specific inheritance of the SNP rs486427. The horizontal width corresponds to the three genotypes and is proportional to their percentage distribution within the study. The vertical axis divides the case and control groups.

FIG. 28 is a mosaic plot illustrating the probability of experiencing LTA as a function of allele specific inheritance of the SNP rs6480311. The horizontal width corresponds to the three genotypes and is proportional to their percentage distribution within the study. The vertical axis divides the case and control groups.

FIG. 29 is a mosaic plot illustrating the probability of experiencing LTA as a function of allele specific inheritance of the SNP rs11610690. The horizontal width corresponds to the three genotypes and is proportional to their percentage distribution within the study. The vertical axis divides the case and control groups.

FIG. 30 is a mosaic plot illustrating the probability of experiencing LTA as a function of allele specific inheritance of the SNP rs 10823151. The horizontal width corresponds to the three genotypes and is proportional to their percentage distribution within the study. The vertical axis divides the case and control groups.

FIG. 31 is a mosaic plot illustrating the probability of experiencing LTA as a function of allele specific inheritance of the SNP rs1346964. The horizontal width corresponds to the three genotypes and is proportional to their percentage distribution within the study. The vertical axis divides the case and control groups.

FIG. 32 is a mosaic plot illustrating the probability of experiencing LTA as a function of allele specific inheritance of the SNP rs6790359. The horizontal width corresponds to the three genotypes and is proportional to their percentage distribution within the study. The vertical axis divides the case and control groups.

FIG. 33 is a mosaic plot illustrating the probability of experiencing LTA as a function of allele specific inheritance of the SNP rs7591633. The horizontal width corresponds to the three genotypes and is proportional to their percentage distribution within the study. The vertical axis divides the case and control groups.

FIG. 34 is a mosaic plot illustrating the probability of experiencing LTA as a function of allele specific inheritance of the SNP rs10487115. The horizontal width corresponds to the three genotypes and is proportional to their percentage distribution within the study. The vertical axis divides the case and control groups.

FIG. 35 is a mosaic plot illustrating the probability of experiencing LTA as a function of allele specific inheritance of the SNP rs2240887. The horizontal width corresponds to the three genotypes and is proportional to their percentage distribution within the study. The vertical axis divides the case and control groups.

FIG. 36 is a mosaic plot illustrating the probability of experiencing LTA as a function of allele specific inheritance of the SNP rs248670. The horizontal width corresponds to the three genotypes and is proportional to their percentage distribution within the study. The vertical axis divides the case and control groups.

FIG. 37 is a mosaic plot illustrating the probability of experiencing LTA as a function of allele specific inheritance of the SNP rs4691391. The horizontal width corresponds to the three genotypes and is proportional to their percentage distribution within the study. The vertical axis divides the case and control groups.

FIG. 38 is a mosaic plot illustrating the probability of experiencing LTA as a function of allele specific inheritance of the SNP rs2270801. The horizontal width corresponds to the three genotypes and is proportional to their percentage distribution within the study. The vertical axis divides the case and control groups.

FIG. 39 is a mosaic plot illustrating the probability of experiencing LTA as a function of allele specific inheritance of the SNP rs12891099. The horizontal width corresponds to the three genotypes and is proportional to their percentage distribution within the study. The vertical axis divides the case and control groups.

FIG. 40 is a mosaic plot illustrating the probability of experiencing LTA as a function of allele specific inheritance of the SNP rs17694397. The horizontal width corresponds to the three genotypes and is proportional to their percentage distribution within the study. The vertical axis divides the case and control groups.

FIG. 41 is a list of rs numbers and corresponding risk alleles.

### DETAILED DESCRIPTION OF THE INVENTION

The invention relates to diagnostic kits and methods using a nucleic acid molecule that can predict Sudden Cardiac Death (“SCA”) or Sudden Cardiac Arrest (“SCA”) risk having a single nucleotide polymorphisms (“SNPs”) selected from the group of SEQ ID Nos. 1-858 that can be used in the diagnosis, distinguishing, and detection thereof.

### DEFINITIONS

Unless defined otherwise, all technical and scientific terms used herein have the same meaning as commonly understood by one of ordinary skill in the art to which this invention belongs. For purposes of the present invention, the following terms are defined below.

The terms “a,” “an,” and “the” include plural referents unless the context clearly dictates otherwise.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

The term “plurality” as described herein means more than one, and also defines a multiple of items.

The term “isolated” refers to nucleic acid, or a fragment thereof, that has been removed from its natural cellular environment.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The term “nucleic acid” encompasses the terms “oligonucleotide” and “polynucleotide.”

The term “amplified polynucleotide” or “amplified nucleotide” as used herein refers to polynucleotides or nucleotides that are copies of a portion of a particular polynucleotide sequence and/or its complementary sequence, which correspond to a template polynucleotide sequence and its complementary sequence. An “amplified polynucleotide” or “amplified nucleotide” according to the present invention, may be DNA or RNA, and it may be double-stranded or single-stranded.

“Synthesis” and “amplification” as used herein are used interchangeably to refer to a reaction for generating a copy of a particular polynucleotide sequence or increasing in copy number or amount of a particular polynucleotide sequence. It may be accomplished, without limitation, by the in vitro methods of polymerase chain reaction (PCR), ligase chain reaction (LCR), polynucleotide-specific based amplification (NSBA), or any other method known in the art. For example, polynucleotide amplification may be a process using a polymerase and a pair of oligonucleotide primers for producing any particular polynucleotide sequence, i.e., the target polynucleotide sequence or target polynucleotide, in an amount which is greater than that initially present.

As used herein, the term “primer pair” means two oligonucleotides designed to flank a region of a polynucleotide to be amplified.

As used herein, an implantable cardioverter-defibrillator (ICD) is a small battery-powered electrical impulse generator implanted in patients who are at risk of sudden cardiac death due to ventricular fibrillation and/or ventricular tachycardia. The device is programmed to detect cardiac arrhythmia and correct it by delivering a jolt of electricity. In known variants, the ability to revert ventricular fibrillation has been extended to include both atrial and ventricular arrhythmias as well as the ability to perform biventricular pacing in patients with congestive heart failure or bradycardia.

“Single nucleotide polymorphisms” (SNPs) refers to a variation in the sequence of a gene in the genome of a population that arises as the result of a single base change, such as an insertion, deletion or, a change in a single base. A locus is the site at which divergence occurs.

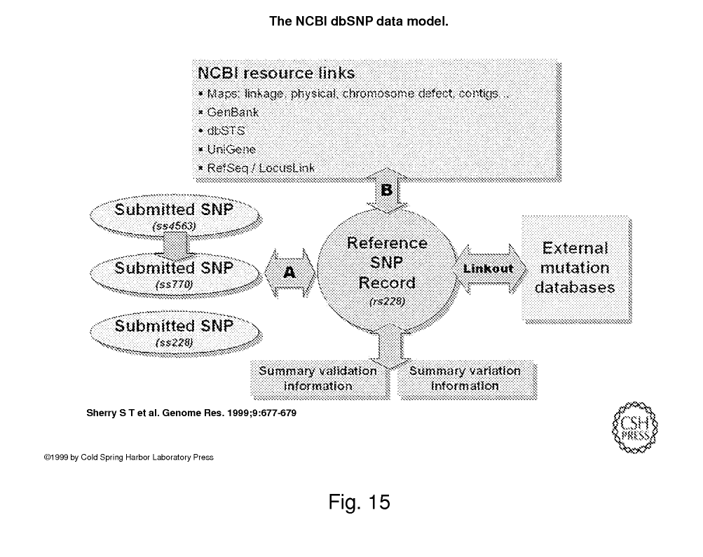

An “rs number” refers to a SNP database record archived and curated on dbSNP, which is a database for Single Polymorphism Polynucleotides and Other Classes of Minor Genetic Variations. The dbSNP database maintains two types of records: ss records of each original submission and rs records. The ss records may represent variations in submissions for the same genome location. The rs numbers represent a unique record for a SNP and are constructed and periodically reconstructed based on subsequent submissions and Builds. In each new build cycle, the set of new data entering each build typically includes all submissions received since the close of data in the previous build. Some refSNP (rs) numbers might have been merged if they are found to map the same location at a later build, however, it is understood that a particular rs number with a Build number provides the requisite detail so that one of ordinary skill in the art will be able to make and use the invention as contemplated herein. Hence, one of ordinary skill will generally be able to determine a particular SNP by reviewing the entries for an rs number and related ss numbers. Data submitted to the NCBI database are clustered and provide a non-redundant set of variations for each organism in the database. The clusters are maintained as rs numbers in the database in parallel to the underlying submitted data. Reference Sequences, or RefSeqs, are a curated, non-redundant set of records for mRNAs, proteins, contigs, and gene regions constructed from a GenBank exemplar for that protein or sequence. The accession numbers under “Submitter-Referenced Accessions” is annotation that is included with a submitted SNP (ss) when it is submitted to dbSNP as shown in FIG. 15 (Sherry et al., “dbSNP—Database for Single Polymorphism Polynucleotides and Other Classes of Minor Genetic Variation,” Genome Res. 1999; 9: 677-679). However, other alternate forms of the rs number as provided in refseqs, ss numbers, etc. are contemplated by the invention such that one of ordinary skill in the art would understand that the scope and nature of the invention is not departed by using follow-on builds of dbSNP.

The term “MACH” or “MACH 1.0” refers to a haplotyper program using a Hidden Markov Model (HMM) that can resolve long haplotypes or infer missing genotypes in samples of unrelated individuals as known within the art.

The term “Hidden Markov Model (HMM)” describes a statistical method for determining a state, which has not been observed or “hidden.” The HMM is generally based on a Markov chain, which describes a series of observations in which the probability of an observation depends on a number of previous observations. For a HMM, the Markov process itself cannot be observed, but only the steps in the sequence.

“Probes” or “primers” refer to single-stranded nucleic acid sequences that are complementary to a desired target nucleic acid. The 5′ and 3′ regions flanking the target complement sequence reversibly interact by means of either complementary nucleic acid sequences or by attached members of another affinity pair. Hybridization can occur in a base-specific manner where the primer or probe sequence is not required to be perfectly complementary to all of the sequences of a template. Hence, non-complementary bases or modified bases can be interspersed into the primer or probe, provided that base substitutions do not inhibit hybridization. The nucleic acid template may also include “nonspecific priming sequences” or “nonspecific sequences” to which the primers or probes have varying degrees of complementarity. As used in the phrase “priming polynucleotide synthesis,” a probe is described that is of sufficient length to initiate synthesis during PCR. In certain embodiments, a probe or primer comprises from about 3 to 101 nucleotides.

The following formula is provided in support of every possible range within 3 to 101 nucleotides. The formula is intended to provide express support for ranges such as 3 to 4 nucleotides in length, or from about 3 to 5, 3 to 6, 3 to 7, 3 to 8, . . . , 3 to 99, 3 to 100, 3 to 101, 4 to 5, 4 to 6, etc., with no limitation on the permutations of various ranges that can be selected from the range of about 3 to 101 nucleotides. Thus, in certain embodiments, a probe or primer comprises from about 3 to 101 nucleotides, wherein the length of the complement is described by a length n for the lower bound, and (n+i) for the upper bound for n={xε [symbol] |3≦x≦101} and i={yε [symbol] |0≦y≦(101−n)}, or from about any number of base pairs flanking the 5′ and 3′ side of a region of interest to sufficiently identify, or result in hybridization. Hence, where x is the integer 3, the lower bound (n) is 3, and the upper bound (n+i) ranges from 3 to 101 where i ranges from 0 to 98, so that the following ranges of nucleotides are provided: 3 to 3, 3 to 4, 3 to 5, 3 to 6, . . . 3 to 101.

Similarly, where x is the integer 4, the lower bound (n) is 4, and the upper bound (n+i) ranges from 4 to 101 for i equals 0 to 97, so that the following ranges of nucleotides are provided: 4 to 4, 4 to 5, 4 to 6, 4 to 7, . . . 4 to 101.

Similarly, where x is the integer 5, the lower bound (n) is 5, and the upper bound (n+i) ranges from 5 to 101 for i equals 0 to 96, so that the following ranges of nucleotides are provided: 5 to 5, 5 to 6, 5 to 7, 5 to 8, . . . 5 to 101, and so forth for each x.

Hence, where x is the integer 100, the lower bound (n) is 100, and the upper bound (n+i) ranges from 100 to 101 for i equals 0 to 1, so that the following ranges of nucleotides are provided: 100 to 100 and 100 to 101.

Finally, where x is the integer 101, the lower bound (n) is 101 and the upper bound (n+i) is 101 because i equals 0.

Further, the ranges can be chosen from group A and B where for A: the probe or primer is greater than 5, greater than 10, greater than 15, greater than 20, greater than 25, greater than 30, greater than 40, greater than 50, greater than 60, greater than 70, greater than 80, greater than 90 and greater than 100 base pairs in length. For B, the probe or primer is less than 102, less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10 base pairs in length. In other embodiments, the probe or primer is at least 70% identical to the contiguous nucleic acid sequence or to the complement of the contiguous nucleotide sequence, for example, at least 80% identical, at least 90% identical, at least 95% identical, and is capable of selectively hybridizing to the contiguous nucleic acid sequence or to the complement of the contiguous nucleotide sequence. Preferred primer lengths include 25 to 35, 18 to 30, and 17 to 24 nucleotides. Often, the probe or primer further comprises a label, e.g., radioisotope, fluorescent compound, enzyme, or enzyme co-factor. One primer is complementary to nucleotides present on the sense strand at one end of a polynucleotide to be amplified and another primer is complementary to nucleotides present on the antisense strand at the other end of the polynucleotide to be amplified. The polynucleotide to be amplified can be referred to as the template polynucleotide. The nucleotide of a polynucleotide to which a primer is complementary is referred to as a target sequence. A primer can have at least about 15 nucleotides, preferably, at least about 20 nucleotides, most preferably, at least about 25 nucleotides. Typically, a primer has at least about 95% sequence identity, preferably at least about 97% sequence identity, most preferably, about 100% sequence identity with the target sequence to which the primer hybridizes. The conditions for amplifying a polynucleotide by PCR vary depending on the nucleotide sequence of primers used, and methods for determining such conditions are routine in the art.

To obtain high quality primers, primer length, melting temperature (Tₘ), GC content, specificity, and intra- or inter-primer homology are taken into account in the present invention. You et al., BatchPrimer3: A high throughput web application for PCR and sequencing primer design, BMC Bioinformatics, 2008, 9:253; Yang X, Scheffler B E, Weston L A, Recent developments in primer design for DNA polymorphism and mRNA profiling in higher plants, Plant Methods, 2006, 2(1):4. Primer specificity is related to primer length and the final 8 to 10 bases of the 3″ end sequence where a primer length of 18 to 30 bases is one possible embodiment. Abd-Elsalam K A, Bioinformatics tools and guideline for PCR primer design, Africa Journal of Biotechnology, 2003, 2(5):91-95. Tₘ is closely correlated to primer length, GC content and primer base composition. One preferred primer Tₘ is in the range of 50 to 65° C. with GC content in the range of 40 to 60% for standard primer pairs. Dieffenbatch C W, Lowe T M J, Dveksler G S, General concepts for PCR primer design, PCR Primer, A Laboratory Manual, Edited by: Dieffenbatch C W, Dveksler G S. New York, Cold Spring Harbor Laboratory Press; 1995:133-155. However, an optimal primer length varies depending on different types of primers. For example, SNP genotyping primers may require a longer primer length of 25 to 35 bases to enhance their specificity, and thus the corresponding Tₘ might be higher than 65° C. Also, a suitable Tₘ can be obtained by setting a broader GC content range (20 to 80%).

The probes or primers can also be variously referred to as “antisense nucleic acid molecules,” “polynucleotides,” or “oligonucleotides” and can be constructed using chemical synthesis and enzymatic ligation reactions known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids. The primers or probes can further be used in “Polymerase Chain Reaction” (PCR), a well known amplification and analytical technique that generally uses two “primers” of short, single-stranded DNA synthesized to correspond to the beginning of a DNA stretch to be copied, and a polymerase enzyme that moves along the segment of DNA to be copied that assembles the DNA copy.

The term “genetic material” refers to a nucleic acid sequence that is sought to be obtained from any number of sources, including without limitation, whole blood, a tissue biopsy, lymph, bone marrow, hair, skin, saliva, buccal swabs, purified samples generally, cultured cells, and lysed cells, and can comprise any number of different compositional components (e.g., DNA, RNA, tRNA, siRNA, mRNA, or various non-coding RNAs). The nucleic acid can be isolated from samples using any of a variety of procedures known in the art. In general, the target nucleic acid will be single stranded, though in some embodiments the nucleic acid can be double stranded, and a single strand can result from denaturation. It will be appreciated that either strand of a double-stranded molecule can serve as a target nucleic acid to be obtained. The nucleic acid sequence can be methylated, non-methylated, or both, and can contain any number of modifications. Further, the nucleic acid sequence can refer to amplification products as well as to the native sequences.

The term “screening” within the phrase “screening for a genetic sample” means any testing procedure known to those of ordinary skill in the art to determine the genetic make-up of a genetic sample.

As used herein, “hybridization” is defined as the ability of two nucleotide sequences to bind with each other based on a degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Stringent conditions are conditions under which a probe can hybridize to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of S×SSPE (750 mM NaCl, 50 mM Na Phosphate, 5 mM EDTA, pH 7.4) and a temperature of 25-30° C. are suitable for allele-specific probe hybridizations. Sambrook et al., Molecular Cloning, 1989.

Allele Specific Oligomer (“ASO”) refers to a primary oligonucleotide having a target specific portion and a target-identifying portion, which can query the identity of an allele at a SNP locus. The target specific portion of the ASO of a primary group can hybridize adjacent to the target specific portion and can be made by methods well known to those of ordinary skill. The ordinary meaning of the term “allele” is one of two or more alternate forms of a gene occupying the same locus in a particular chromosome or linkage structure and differing from other alleles of the locus at one or more mutational sites. (Rieger et al., Glossary of Genetics, 5th Ed., Springer-Verlag, Berlin 1991; 16).

The ordinary meaning of the term “allele” is one of two or more alternate forms of a gene occupying the same locus in a particular chromosome or linkage structure and differing from other alleles of the locus at one or more mutational sites. (Rieger et al., Glossary of Genetics, 5th Ed., Springer-Verlag, Berlin 1991; 16).

Bi-allelic and multi-allelic refers to two, or more than two alternate forms of a SNP, respectively, occupying the same locus in a particular chromosome or linkage structure and differing from other alleles of the locus at a polymorphic site.

The phrase “assessing the presence of said one or more SNPs in a genetic sample” encompasses any known process that can be implemented to determine if a polymorphism is present in a genetic sample. For example, amplified DNA obtained from a genetic sample can be labeled before it is hybridized to a probe on a solid support. The amplified DNA is hybridized to probes which are immobilized to known locations on a solid support, e.g., in an array, microarray, high density array, beads or microtiter dish. The presence of labeled amplified DNA products hybridized to the solid support indicates that the nucleic acid sample contains at the polymorphic locus a nucleotide which is indicative of the polymorphism. The quantities of the label at distinct locations on the solid support can be compared, and the genotype can be determined for the sample from which the DNA was obtained. Two or more pairs of primers can be used for determining the genotype of a sample. Each pair of primers specifically amplifies a different allele possible at a given SNP. The hybridized nucleic acids can be detected, e.g., by detecting one or more labels attached to the target nucleic acids. The labels can be incorporated by any convenient means. For example, a label can be incorporated by labeling the amplified DNA product using a terminal transferase and a fluorescently labeled nucleotide. Useful detectable labels include labels that can be detected by spectroscopic, photochemical, biochemical, immunochemical, and electrical, optical, or chemical means. Radioactive labels can be detected using photographic film or scintillation counters. Fluorescent labels can be detected using a photodetector.

The term “detecting” as used in the phrase “detecting one or more Single Nucleotide Polymorphisms (SNPs)” refers to any suitable method for determining the identity of a nucleotide at a position including, but not limited to, sequencing, allele specific hybridization, primer specific extension, oligonucleotide ligation assay, restriction enzyme site analysis and single-stranded conformation polymorphism analysis.

In double-stranded DNA, only one strand codes for the RNA that is translated into protein. This DNA strand is referred to as the “antisense” strand. The strand that does not code for RNA is called the “sense” strand. Another way of defining antisense DNA is that it is the strand of DNA that carries the information necessary to make proteins by binding to a corresponding messenger RNA (mRNA). Although these strands are exact mirror images of one another, only the antisense strand contains the information for making proteins. “Antisense compounds” are oligomeric compounds that are at least partially complementary to a target nucleic acid molecule to which they hybridize. In certain embodiments, an antisense compound modulates (increases or decreases) expression of a target nucleic acid. Antisense compounds include, but are not limited to, compounds that are oligonucleotides, oligonucleosides, oligonucleotide analogs, oligonucleotide mimetics, and chimeric combinations of these. Consequently, while all antisense compounds are oligomeric compounds, not all oligomeric compounds are antisense compounds.

Mutations are changes in a genomic sequence. As used herein, “naturally occurring mutants” refers to any preexisting, not artificially induced change in a genomic sequence. Mutations, mutant sequences, or, simply, “mutants” include additions, deletions and substitutions or one or more alleles.

The optimal probe length, position, and number of probes for detection of a single nucleotide polymorphism or for hybridization may vary depending on various hybridization conditions. Thus, the phrase “sufficient to identify the SNP or result in a hybridization” is understood to encompass design and use of probes such that there is sufficient specificity and sensitivity to detect and identify a SNP sequence or result in a hybridization. Hybridization is described in further detail below.

The phrases “increased susceptibility,” “decreased susceptibility,” or the term “risk,” generally, relates to the possibility or probability of a particular event occurring either presently or at some point in the future. Determining an increase or decrease in susceptibility to a medical disease, disorder or condition involves “risk stratification” or “assessing susceptibility,” which refers to an arraying of known clinical risk factors that allow physicians and others of skill in the relevant art to classify patients from a low to high range of risk of developing a particular disease, disorder, or condition.

“cDNA” refers to DNA that is synthesized to be complementary to a mRNA molecule, and that represents a portion of the DNA that specifies a protein (is translated). If the sequence of the cDNA is known, by complementarity, the sequence of the DNA is known.

The phrase “selectively hybridizing” refers to the ability of a probe used in the invention to hybridize, with a target nucleotide sequence with specificity.

The term “treatable” means that a patient is potentially or would be expected to be responsive to a particular form of treatment.

In statistical significance testing, the “p-value” is the probability of obtaining a test statistic at least as extreme as the one that was actually observed, assuming that the null hypothesis is true. The lower the p-value, the less likely the result is if the null hypothesis is true, and consequently the more “significant” the result is, in the sense of statistical significance.

As used herein, to impute a p-value to one or more SNPs outside of a test sample means to mathematically attribute a p-value to one or more known and documented SNPs, using the methods described herein, that are not present on the test microchips used in a specific experiment or study. Using the p-values obtained from the tested microchips, p-values may be mathematically imputed to other known SNPs using algorithms such as those described herein.

By the phrase “indicate association,” it is meant that the statistical analysis suggests, by, for example, a p-value, that a SNP may be linked to or associated with a particular medical disease, condition, or disorder.

The term “isolated” as used herein with reference to a nucleic acid molecule refers to a nucleic acid that is not immediately contiguous with both of the sequences with which it is immediately contiguous in the naturally occurring genome of the organism from which it is derived. The term “isolated” also includes any non-naturally occurring nucleic acid because such engineered or artificial nucleic acid molecules do not have immediately contiguous sequences in a naturally occurring genome.

### DNA Microarrays

Numerous forms of diagnostic kits employing arrays of nucleotides are known in the art. They can be fabricated by any number of known methods including photolithography, pipette, drop-touch, piezoelectric, spotting and electric procedures. The DNA microarrays generally have probes that are supported by a substrate so that a target sample is bound or hybridized with the probes. In use, the microarray surface is contacted with one or more target samples under conditions that promote specific, high-affinity binding of the target to one or more of the probes as shown in FIG. 12. A sample solution containing the target sample typically contains radioactively, chemoluminescently or fluorescently labeled molecules that are detectable. The hybridized targets and probes can also be detected by voltage, current, or electronic means known in the art.

Optionally, a plurality of microarrays may be formed on a larger array substrate. The substrate can be diced into a plurality of individual microarray dies in order to optimize use of the substrate. Possible substrate materials include siliceous compositions where a siliceous substrate is generally defined as any material largely comprised of silicon dioxide. Natural or synthetic assemblies can also be employed. The substrate can be hydrophobic or hydrophilic or capable of being rendered hydrophobic or hydrophilic and includes inorganic powders such as silica, magnesium sulfate, and alumina; natural polymeric materials, particularly cellulosic materials and materials derived from cellulose, such as fiber-containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. The surface of the substrate is then chemically prepared or derivatized to enable or facilitate the attachment of the molecular species to the surface of the array substrate. Surface derivatizations can differ for immobilization of prepared biological material, such as cDNA, and in situ synthesis of the biological material on the microarray substrate. Surface treatment or derivatization techniques are well known in the art. The surface of the substrate can have any number of shapes, such as strip, plate, disk, rod, particle, including bead, and the like. In modifying siliceous or metal oxide surfaces, one technique that has been used is derivatization with bifunctional silanes, i.e., silanes having a first functional group enabling covalent binding to the surface and a second functional group that can impart the desired chemical and/or physical modifications to the surface to covalently or non-covalently attach ligands and/or the polymers or monomers for the biological probe array. Adsorbed polymer surfaces are used on siliceous substrates for attaching nucleic acids, for example cDNA, to the substrate surface. Since a microarray die may be quite small and difficult to handle for processing, an individual microarray die can also be packaged for further handling and processing. For example, the microarray may be processed by subjecting the microarray to a hybridization assay while retained in a package.

Various techniques can be employed for preparing an oligonucleotide for use in a microarray. In situ synthesis of oligonucleotide or polynucleotide probes on a substrate is performed in accordance with well-known chemical processes, such as sequential addition of nucleotide phosphoramidites to surface-linked hydroxyl groups. Indirect synthesis may also be performed in accordance with biosynthetic techniques such as Polymerase Chain Reaction (“PCR”). Other methods of oligonucleotide synthesis include phosphotriester and phosphodiester methods and synthesis on a support, as well as phosphoramidate techniques. Chemical synthesis via a photolithographic method of spatially addressable arrays of oligonucleotides bound to a substrate made of glass can also be employed. The probes or oligonucleotides, themselves, can be obtained by biological synthesis or by chemical synthesis. Chemical synthesis provides a convenient way of incorporating low molecular weight compounds and/or modified bases during specific synthesis steps. Furthermore, chemical synthesis is very flexible in the choice of length and region of target polynucleotides binding sequence. The oligonucleotide can be synthesized by standard methods such as those used in commercial automated nucleic acid synthesizers.

Immobilization of probes or oligonucleotides on a substrate or surface may be accomplished by well-known techniques. One type of technology makes use of a bead-array of randomly or non-randomly arranged beads. A specific oligonucleotide or probe sequence is assigned to each bead type, which is replicated any number of times on an array. A series of decoding hybridizations is then used to identify each bead on the array. The concept of these assays is very similar to that of DNA chip based assays. However, oligonucleotides are attached to small microspheres rather than to a fixed surface of DNA chips. Bead-based systems can be combined with most of the allele-discrimination chemistry used in DNA chip based array assays, such as single-base extension and oligonucleotide ligation assays. The bead-based format has flexibility for multiplexing and SNP combination. In bead-based assays, the identity of each bead needs is determined where that information is combined with the genotype signal from the bead to assign a “genotype call” to each SNP and individual.

One bead-based genotyping technology uses fluorescently coded microspheres developed by Luminex. Fulton R, McDade R, Smith P, Kienker L, Kettman J. J. Advanced multiplexed analysis with the FlowMetrix system, Clin. Chem. 1997; 43: 1749-1756. These beads are coated with two different dyes (red and orange), and can be identified and separated using flow cytometry, based on the amount of these two dyes on the surface. By having a hundred types of microspheres with a different red:orange signal ratio, a hundred-plex detection reaction can be performed in a single tube. After the reaction, these microspheres are distinguished using a flow fluorimeter where a genotyping signal (green) from each group of microspheres is measured separately. This bead-based platform is useful in allele-specific hybridization, single-base extension, allele-specific primer extension, and oligonucleotide ligation assay. In a different bead-based platform commercialized by Illumina, microspheres are captured in solid wells created from optical fibers. Michael K., Taylor L., Schultz S, Walt D. Randomly ordered addressable high-density optical sensor arrays, Anal. Chem., 1998; 70: 1242-1248; Steemers F., Ferguson J, Walt D., Screening unlabeled DNA targets with randomly ordered fiber-optic gene arrays, Nat. Biotechnol., 2000; 18: 91-94. The diameter of each well is similar to that of the spheres, allowing only a single sphere to fit in one well. Once the microspheres are set in these wells, all of the spheres can be treated like a high-density microarray. The high degree of replication in DNA microarray technology makes robust measurements for each bead type possible. Bead-array technology is particularly useful in SNP genotyping. Software used to process raw data from a DNA microarray or chip is well known in the art and employs various known methods for image processing, background correction and normalization. Many available public and proprietary software packages are available for such processing whereby a quality assessment of the raw data can be carried out, and the data then summarized and stored in a format which can be used by other software to perform additional analyses.

### Single Nucleotide Polymorphism (“SNP”)

Generally, genetic variations are associated with human phenotypic diversity and sometimes disease susceptibility. As a result, variations in genes may prove useful as markers for disease or other disorder or condition. Variation at a particular genomic location is due to a mutation event in the conserved human genome sequence, leading to two possible nucleotide variants at that genetic locus. If both nucleotide variants are found in at least 1% of the population, that location is defined as a Single Nucleotide Polymorphism (“SNP”). Moreover, SNPs in close proximity to one another are often inherited together in blocks called haplotypes. These single base nucleotide exchanges result in modified amino acid sequences, altering the structure and function of the coded protein. They also influence the splicing process when present at exon-intron transitions and modify gene transcription when part of promoters. This leads to an altered level of protein expression.

One phenomenon of SNPs is that they can undergo linkage disequilibrium, which refers to the tendency of specific alleles at different genomic locations to occur together more frequently than would be expected by random change. Alleles at given loci are said to be in complete equilibrium if the frequency of any particular set of alleles (or haplotype) is the product of their individual population frequencies. Several statistical measures can be used to quantify this relationship. (Devlin and Risch, A comparison of linkage disequilibrium measures for fine-scale mapping, Genomics, 1995 Sep. 20; 29(2):311-22).

With respect to alleles, a more common nucleotide is known as the major allele and the less common nucleotide is known as the minor allele. An allele found to have a higher than expected prevalence among individuals positive for a given outcome is considered a risk allele for that outcome. An allele found to have a lower than expected prevalence among individuals positive for an outcome is considered a protective allele for that outcome. But while the human genome harbors 10 million “common” SNPs, minor alleles indicative of heart disease are often only shared by as little as one percent of a population.

Hence, as provided herein, certain SNPs found by one or a combination of these methods have been found useful as genetic markers for risk-stratification of SCD or SCA in individuals. Genome-wide association studies are used to identify disease susceptibility genes for common diseases and involve scanning thousands of samples, either as case-control cohorts or in family trios, utilizing hundreds of thousands of SNP markers located throughout the human genome. Algorithms can then be applied that compare the frequencies of single SNP alleles, genotypes, or multi-marker haplotypes between disease and control cohorts. Regions (loci) with statistically significant differences in allele or genotype frequencies between cases and controls, pointing to their role in disease, are then analyzed. For example, following the completion of a whole genome analysis of patient samples, SNPs for use as clinical markers can be identified by any, or combination, of the following three methods:

(1) Statistical SNP Selection Method: Univariate or multivariate analysis of the data is carried out to determine the correlation between the SNPs and the study outcome, life threatening arrhythmias for the present invention. SNPs that yield low p-values are considered as markers. These techniques can be expanded by the use of other statistical methods such as linear regression.

(2) Logical SNP Selection Method: Clustering algorithms are used to segregate the SNP markers into categories which would ultimately correlate with the patient outcomes. Classification and Regression Tree (“CART”) is one of the clustering algorithms that can be used. In that case, SNPs forming the branching nodes of the tree will be the markers of interest.

(3) Biological SNP Selection Method: SNP markers are chosen based on the biological effect of the SNP, as it might affect the function of various proteins. For example, a SNP located on a transcribed or a regulatory portion of a gene that is involved in ion channel formation would be good candidates. Similarly, a group of SNPs that are shown to be located closely on the genome would also hint the importance of the region and would constitute a set of markers.

Genetic markers are non-invasive, cost-effective and conducive to mass screening of individuals. The SNPs identified herein can be effectively used alone or in combination with other SNPs as well as with other clinical markers for risk-stratification/assessment and diagnosis of SCD, or SCA. Further, these genetic markers in combination with other clinical markers for SCA are effectively used for identification and implantation of ICDs in individuals at risk for SCA. The genetic markers taught herein provide greater specificity and sensitivity in identification of individuals at risk.

An explanation of an rs number and the National Center for Biotechnology Information (NCBI) SNP database is provided herein. In collaboration with the National Human Genome Research Institute, The National Center for Biotechnology Information has established the dbSNP database to serve as a central repository for both single base nucleotide substitutions, single nucleotide polymorphisms (SNPs) and short deletion and insertion polymorphisms. Reference Sequences, or RefSeqs (rs), are a curated, non-redundant set of records for mRNAs, proteins, contigs, and gene regions constructed from a GenBank exemplar for that protein or sequence. The rs numbers represent a unique record for a SNP. Submitted SNPs (ss) are records that are independently submitted to NCBI, are used to construct the rs record, and are cross-referenced with the rs record for the corresponding genome location. Submitter-Referenced Accession numbers are annotations that are included with a SS number. For rs records relevant to the present invention, these accession numbers may be associated with a GenBank accession record, which will start with one or two letters, such as “AL” or “AC,” followed by five or six numbers. The NCBI RefSeq database accession numbers have different formatting: “NT_{—}123456.” The RefSeq accession numbers are unique identifiers for a sequence, and when minor changes are made to a sequence, a new version number is assigned, such as “NT_{—}123456.1,” where the version is represented by the number after the decimal. The rs number represents a specific range of bases at a certain contig position. Although the contig location of the rs sequence may move relative to the length of the larger sequence encompassed by the accession number, that sequence of bases represented by the rs number, i.e., the SNP, will remain constant. Hence, it is understood that rs numbers can be used to uniquely identify a SNP and fully enables one of ordinary skill in the art to make and use the invention using rs numbers. The sequences provided in the Sequence Listing each correspond to a unique sequence represented by an rs number known at the time of invention. Thus, the SEQ ID Nos. and the rs numbers claimed disclosed herein are understood to represent uniquely identified sequences for identified SNPs and may be used interchangeably.

### Sudden Cardiac Arrest (“SCA”)

SCA, also known as Sudden Cardiac Death (“SCD”), results from an abrupt loss of heart function. It is commonly brought on by an abnormal heart rhythm. Sudden cardiac death occurs within a short time period, generally less than an hour from the onset of symptoms. Despite recent progress in the management of cardiovascular disorders generally, and cardiac arrhythmias in particular, SCA remains both a problem for the practicing clinician and a major public health issue.

In the United States, SCA accounts for approximately 325,000 deaths per year. More deaths are attributable to SCA than to lung cancer, breast cancer, or AIDS. This represents an incidence of 0.1-0.2% per year in the adult population. Myerburg, R J et al., Cardiac arrest and sudden cardiac death, Braunwald E, ed., A Textbook of Cardiovascular Medicine. 6^{th} ed. Philadelphia, Saunders, W B., 2001: 890-931; American Cancer Society, Cancer FACTS and Figures 2003: 4, Center for Disease Control 2004.

In 60% to 80% of cases, SCA occurs in the setting of Coronary Artery Disease (“CAD”). Most instances involve Ventricular Tachycardias (“VT”) degenerating to Ventricular Fibrillation (“VF”) and subsequent asystole. Fibrillation occurs when transient neural triggers impinge upon an unstable heart causing normally organized electrical activity in the heart to become disorganized and chaotic. Complete cardiac dysfunction results. Non-ischemic cardiomyopathy and infiltrative, inflammatory, and acquired valvular diseases account for most other SCA, or SCD, events. A small percentage of SCAs occur in the setting of ion channel mutations responsible for inherited abnormalities such as the long/short QT syndromes, Brugada syndrome, and catecholaminergic ventricular tachycardia. These conditions account for a small number of SCAs. In addition, other genetic abnormalities such as hypertrophic cardiomyopathy and congenital heart defects such as anomalous coronary arteries are responsible for SCA.

Currently, five arrhythmia markers are often used for risk assessment in Myocardial Infarction (“MI”) patients: (1) Heart Rate (“HR”) Variability, (2) severe ventricular arrhythmia, (3) signal averaged Electro Cardio Gram (“ECG”), (4) left ventricular Ejection Fraction (“EF”) and (5) electrophysiology (“EP”) (studies). Table 1 illustrates the mean sensitivity and specificity values for each of these five arrhythmia markers. As shown, these markers have relatively high specificity values, but low sensitivity values.

TABLE 1

| | | Severe | | | |
|---|---|---|---|---|---|
| | HR | Ventricular | Signal | Left Ventricular | |
| | Variability | Arrhythmia | Averaged | Ejection | Electrophysiology |
| Test | on AECG | on AECG | ECG | Fraction (EF) | (EP) Studies |
| Sensitivity | 49.8% | 42.8% | 62.4% | 59.1% | 61.8% |
| Specificity | 85.8% | 81.2% | 77.4% | 77.8% | 84.1% |

The most commonly used marker, EF, has a sensitivity of 59%, meaning that 41% of the patients would be missed if EF were the only marker used. Although EP studies provide slightly better indications, they are not performed very frequently due to their rather invasive nature. Hence, the identification of patients who have a propensity toward SCA remains as an unmet medical need.

ECG parameters indicative of SCA, or SCD, are QRS duration, late potentials, QT dispersion, T-wave morphology, Heart rate variability and T-wave alternans. Electrical alternans is a pattern of variation in the shape of the ECG waveform that appears on an every-other-beat basis. In humans, alternation in ventricular repolarization, namely, repolarization alternans, has been associated with increased vulnerability to ventricular tachycardia/ventricular fibrillation and sudden cardiac death. Pham, Q., et al., T-wave alternans: marker, mechanism, and methodology for predicting sudden cardiac death. Journal of Electrocardiology, 36: 75-81. Analysis of the morphology of an ECG (i.e., T-wave alternans and QT interval dispersion) has been recognized as means for assessing cardiac vulnerability.

Certain biological factors are predictive of risk for SCA such as a previous clinical event, ambient arrhythmias, cardiac response to direct stimulations, and patient demographics. Similarly, analysis of heart rate variability has been proposed as a means for assessing autonomic nervous system activity, the neural basis for cardiac vulnerability. Heart rate variability, a measure of beat-to-beat variations of sinus-initiated RR intervals, with its Fourier transform-derived parameters, is blunted in patients at risk for SCD. Bigger, J T. Heart rate variability and sudden cardiac death, Zipes D P, Jalife J, Eds. Cardiac Electrophysiology: From Cell to Bedside, Philadelphia, Pa.: WB Saunders; 1999.

Patient history is helpful to analyze the risk of SCA, or SCD. For example, in patients with ventricular tachycardia after myocardial infarction, on the basis of clinical history, the following four variables identify patients at increased risk of sudden cardiac death: (1) syncope at the time of the first documented episode of arrhythmia, (2) New York Heart Association (“NYHA”) Classification class III or IV, (3) ventricular tachycardia/fibrillation occurring early after myocardial infarction (3 days to 2 months), and (4) history of previous myocardial infarctions. Unfortunately, most of these clinical indicators lack sufficient sensitivity, specificity, and predictive accuracy to pinpoint the patient at risk for SCA, with a degree of accuracy that would permit using a specific therapeutic intervention before an actual event.

For example, the disadvantage of focusing solely on ejection fraction is that many patients whose ejection fractions exceed commonly used cut offs still experience sudden death or cardiac arrest. Since EF is not specific in predicting mode of death, decision making for the implantation of an ICD solely on ejection fraction will not be optimal. Buxton, A E et al., Risk stratification for sudden death: do we need anything more than ejection fraction? Card. Electrophysiology Rev. 2003; 7: 434-7. Although electrophysiological (“EP”) studies provide slightly better indication, they are not performed very frequently due to their invasive nature and high cost.

Conventional methods for assessing vulnerability to SCA, or SCD, often rely on power spectral analysis (Fourier analysis) of the cardiac electrogram. However, the power spectrum lacks the ability to track many of the rapid arrhythmogenic changes which characterize T-wave alternans, dispersions and heart rate variability. As a result, a non-invasive diagnostic method of predicting vulnerability to SCA, or SCD, by the analysis of ECG has not achieved wide spread clinical acceptance.

Similarly, both, baroflex sensitivity and heart rate variability, judge autonomic modulation at the sinus node, which is taken as a surrogate for autonomic actions at the ventricular level. Autonomic effects at the sinus node and ventricle can easily be dissociated experimentally and may possibly be a cause of false-positive or false-negative test results. Zipes, D P et al., Sudden Cardiac Death, Circulation, 1998; 98:2334-2351.

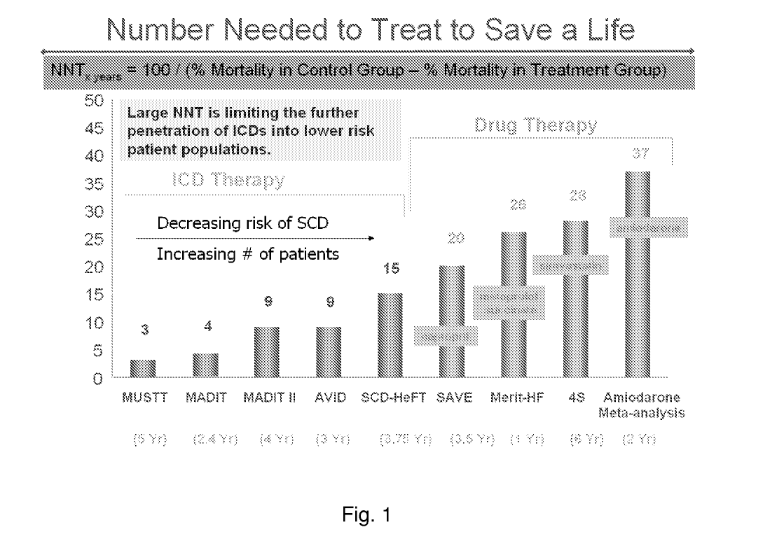

Moreover, as shown in FIG. 1, an increase in the Number Needed to Treat (“NNT”) has been observed for the ICD therapy as the devices are implanted in patients with lower risks. NNT is an epidemiological measure used in assessing the effectiveness of a health-care intervention. The NNT is the number of patients who need to be treated in order to prevent a single negative outcome. In the case of ICDs, currently, devices must be implanted in approximately 17 patients to prevent one death. The other 16 patients may not experience a life threatening arrhythmia and may not receive a treatment. Reduction of the NNT for ICDs would yield to better patient identification methods and allow delivery of therapies to individuals who need them. As a result, it is believed that the need for risk stratification of patients might increase over time as the ICDs are implanted in patients who are generally considered to be at lower risk categories. The net result of the lack of more specific markers for life threatening arrhythmias is the presence of a population of patients who would benefit from ICD therapy, but are not currently indicated, and a subgroup of patients who receive ICD implants, but may not benefit from them.

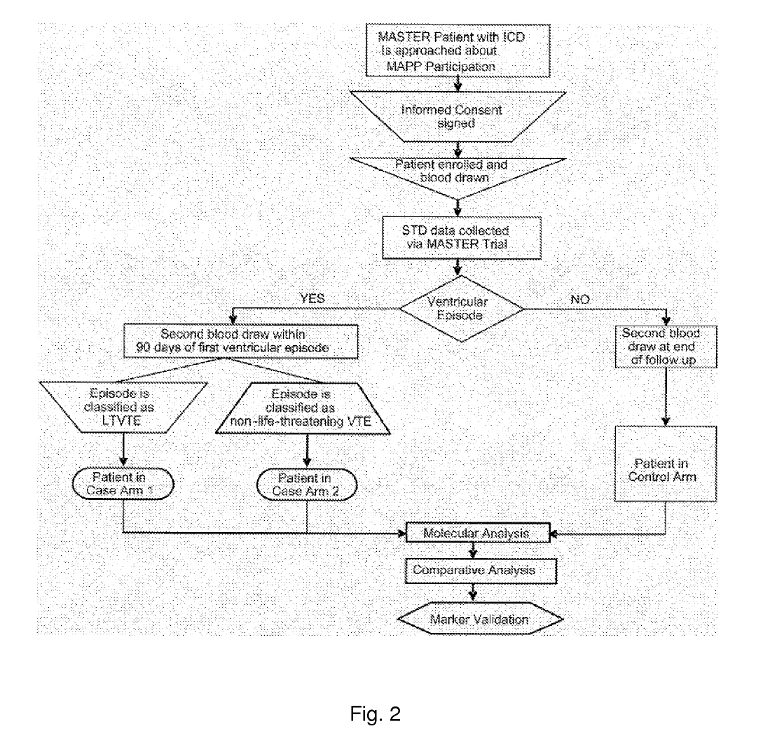

Therefore, in order to identify genetic markers associated with SCA, or SCD, a sub-study (also referred to herein as “MAPP”) to an ongoing clinical trial (also referred to herein as “MASTER”) was designed and implemented. The MASTER study was undertaken to determine the utility of T-wave-alternans test for the prediction of SCA in patients who have had a heart attack and are in heart failure. The overall aim of the study was to assist in identification of patients most likely to benefit from receiving an ICD. Resulting data was used for the search of statistical associations between life threatening events and SNPs. FIG. 2′ is a graphical representation of the study design. All patients participating in the MAPP study had defibrillators (ICD) implanted at enrollment and they were followed up for an average of 2.6 years following the ICD implantation. Based on the arrhythmic events that the patients had during this follow-up, they were categorized in three groups as shown in Table 2.

TABLE 2
Outcome of MAPP Patients

| Patient Category | Number |
|---|---|
| CASE 1 - Life Threatening Left Ventricular Event | 33 |
| CASE 2 - Non-life Threatening Left Ventricular Events | 2 |
| CONTROL - No Events | 205 |
| Total | 240 |

Table 3 provides a brief summary of the demographic and physiologic variables that were recorded at the time of enrollment. Except for the Ejection Fraction (“EF”), none of the variables were found to be predictive of the patient outcome, as shown by the large p-values in Table 3. Although the EF gave a p-value less than 0.05, indicating a correlation with the presence of arrhythmic events, it did not provide a sufficient separation of the two groups to act as a prognostic predictor for individual patients, which in turn further confirmed the initial assessment that there is no strong predictor for SCA.

Table 3 provides a brief summary of the demographic and physiologic variables that were recorded at the time of enrollment. Except for the Ejection Fraction (“EF”), none of the variables were found to be predictive of the patient outcome, as shown by the large p-values in Table 3. Although the EF gave a p-value less than 0.05, indicating a correlation with the presence of arrhythmic events, it did not provide a sufficient separation of the two groups to act as a prognostic predictor for individual patients, which in turn further confirmed the initial assessment that there is no strong predictor for SCA.

TABLE 3
Demographic and Physiologic Variable Summary For
the MAPP Patient Population

| Variable | Entire MAPP | Case 1 | Control | |
|---|---|---|---|---|
| Name | N = 240 | N = 33 | N = 205 | p-value |
| | Mean (SD) | | | |
| Age (years) | 63.2 (11.0) | 61.6 (8.5) | 63.5 (11.3) | 0.3694 |
| EF (%) | 27.1 (6.5) | 25.0 (6.3) | 27.5 (6.4) | 0.0449 |
| NYHA Class | 2.7 (1.4) | 2.9 (1.4) | 2.7 (1.4) | 0.4015 |
| QRS Width | 115.4 (29.8) | 115.0 (23.8) | 115.5 (30.7) | 0.9443 |
| (msec) | | | | |
| | N (%) | | | |
| Sex (Male) | 209 (87.1) | 26 (78.8) | 183 (88.4) | 0.1582 |
| MTWA | 77 (32.2) | 13 (39.4) | 64 (31.0) | 0.4223 |
| (Negative) | | | | |
| Race | 224 (93.3) | 31 (93.9) | 193 (93.2) | 1 |
| (Caucasian) | | | | |

(EF: Ejection fraction; NYHC: New York Heart Class; MTWA: Microvolt T-Wave Alternans test)

Association of genetic variation and disease can be a function of many factors, including, but not limited to, the frequency of the risk allele or genotype, the relative risk conferred by the disease-associated allele or genotype, the correlation between the genotyped marker and the risk allele, sample size, disease prevalence, and genetic heterogeneity of the sample population. In order to search for associations between SNPs and patient outcomes, genomic DNA was isolated from the blood samples collected from the 240 patients who participated in this study. Following the DNA isolation, a whole genome scan consisting of 317,503 SNPs was conducted using Illumina 300K HapMap gene chips. For each locus, two nucleic acid reads were done from each patient, representing the nucleotide variants on two chromosomes, except for the loci chromosomes on male patients. Four letter symbols were used to represent the nucleotides that were read: cytosine (C), guanine (G), adenine (A), and thymine (T). The structure of the various alleles is described by any one of the nucleotide symbols of Table 4.

TABLE 4
Allele Key used in Sequence Listings

| Nucleotide symbol | Full Name |
|---|---|
| R | Guanine/Adenine (purine) |
| Y | Cytosine/Thymine (pyrimidine) |
| K | Guanine/Thymine |
| M | Adenine/Cytosine |
| S | Guanine/Cytosine |
| W | Adenine/Thymine |
| B | Guanine/Thymine/Cytosine |
| D | Guanine/Adenine/Thymine |
| H | Adenine/Cytosine/Thymine |
| V | Guanine/Cytosine/Adenine |
| N | Adenine/Guanine/Cytosine/Thymine |

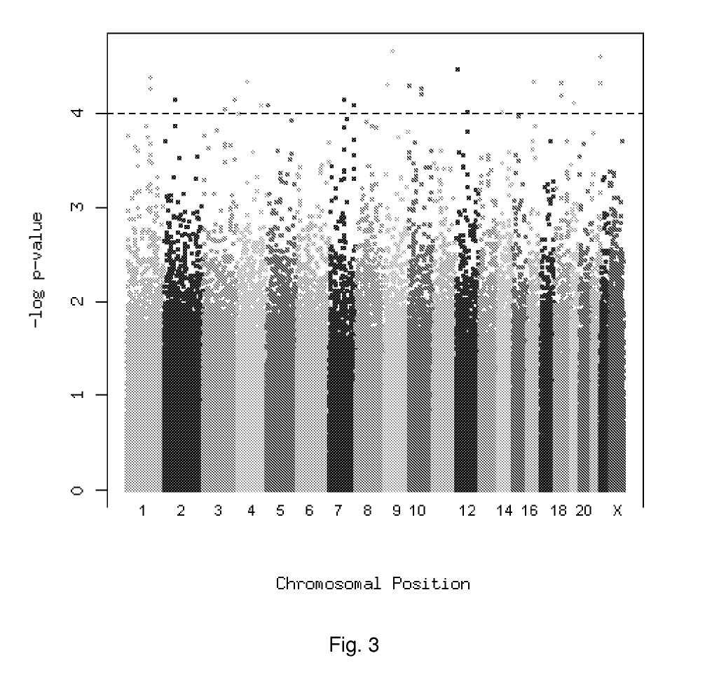

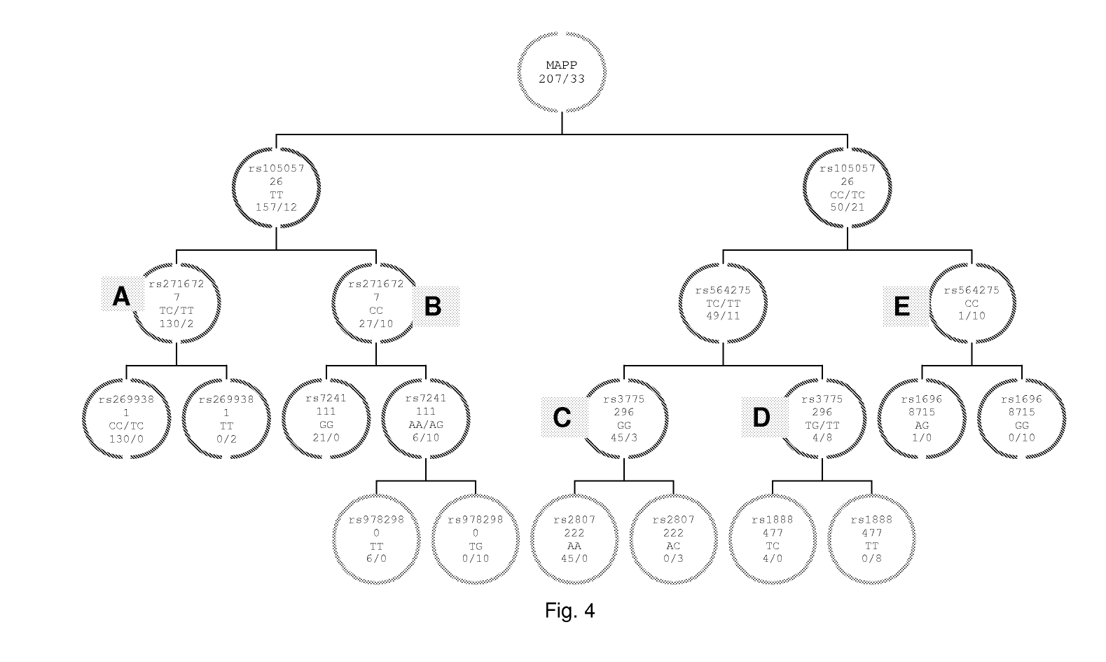

Following the compilation of the genetic data into an electronic database, statistical analysis was carried out. Results from this analysis are provided in FIG. 3. As shown in FIG. 3, a statistical plot of SNPs: p-values graphed as a function of chromosomal position. The dotted line corresponds to a p-value of 0.0001. There were 25 SNPs found in this analysis with a p-value at or less than 0.0001. The y-axis is the negative base 10 logarithm of the p-value. The x-axis is the chromosome and chromosomal position of each SNP on the Illumina gene chip for which a chromosomal location could be determined (N=314,635).

For each SNP, Fisher exact test p-value was calculated. Fisher's exact test is a statistical significance test used in the analysis of categorical data where sample sizes are small. For 2 by 2 tables, the null of conditional independence is equivalent to the hypothesis that the odds ratio equals one. “Exact” inference can be based on observing that in general, given all marginal totals are fixed, the first element of the contingency table has a non-central hypergeometric distribution with non-centrality parameter given by the odds ratio (Fisher, 1935). The alternative for a one-sided test is based on the odds ratio, so alternative=“greater” is a test of the odds ratio being bigger than one.

For a 2×2 contingency table

| | |
|---|---|
| a | C |
| b | D |

the probability of the observed table is calculated by the hypergeometric distribution formula

$$\begin{matrix}p = ( \begin{matrix}a + b \\ a\end{matrix} ) ( \begin{matrix}c + d \\ c\end{matrix} ) / ( \begin{matrix}n \\ a + c\end{matrix} ) \\ = \frac{( a + b ) ! ( c + d ) ! ( a + c ) ! ( b + d ) !}{n ! a ! b ! c ! d !}\end{matrix}$$

Two-sided tests are based on the probabilities of the tables, and take as ‘more extreme’ all tables with probabilities less than or equal to that of the observed table, the p-value being the sum of all such probabilities. Simulation is done conditional on the row and column marginals, and works only if the marginals are strictly positive. Fisher, R. A., The Logic of Inductive Inference, Journal of the Royal Statistical Society Series A, 1935; 98, 39-54.

Statistical analysis of the data continued with the use of a recursive partitioning algorithm. Recursive partitioning is a nonparametric technique that recursively partitions the data up into homogeneous subsets (with regard to the response). A multi-level “tree” is formed by bisecting each subset of patients based on their value of a given predictor variable. This point of bisection is called a “node.” In this analysis, SNPs were the predictors and the three potential genotypes for each SNP (major allele homozygotes, heterozygotes and minor allele homozygotes) were split into two groups, where the heterozygotes were compacted with one of the two homozygote groups. For a prospectively defined response (in this case, whether a patient is a case or control patient) and set of predictors (SNPs), this method recursively splits the data at each node until either the patients at the resulting end nodes are homogeneous with respect to the response or the end nodes contain too few observations. The decision tree is a visual diagram of the results of recursive partitioning, with the topmost nodes indicating the most discriminatory SNP and each node further split into subnodes accordingly. When this algorithm was applied to 317,498 SNPs, at least a subset of the patients in the analysis cohort was successfully genotyped, and the decision tree shown in FIG. 4 resulted. FIG. 4 provides the decision tree resulting from the application of the recursive partitioning algorithm to the SNPs that were found to be correlated with the patient outcomes in the MAPP study. The two numbers shown in each node correspond to the case and the control patients grouped in that node.

Using only the non-shaded decision nodes on the tree shown in FIG. 4, patients can be categorized in five groups as illustrated in Table 5.

TABLE 5
Genomic Grouping of MAPP Patients Based on
the Results of the Recursive Partitioning Algorithm

| Group | Genome | SCD Risk | ICD Recommendation |
|---|---|---|---|
| A | rs10505726 = TT rs2716727 = TC/TT | $\frac{2}{132} = 1.5 \%$ | Do not implant |
| B | rs10505726 = TT rs2716727 = CC | $\frac{10}{37} = 27 \%$ | Implant |
| C | rs10505726 = CC/TC rs564275 = TC/TT rs3775296 = GG | $\frac{3}{48} = 6.3 \%$ | Do not implant |
| D | rs10505726 = CC/TC rs564725 = TC/TT rs3775296 = TG/TT | $\frac{8}{12} = 66.7 \%$ | Implant |
| E | rs10505726 = CC/TC rs564275 = CC | $\frac{10}{11} = 90.1 \%$ | Implant |

The overall specificity and sensitivity of the combined tests described by Groups A through E in Table 5 can be determined by examining the contingency table (Table 6) of the combined test and MAPP patients in Case 1 patients, who experienced a life threatening VTNF event versus Case 2 and Control patients who did not. It is desirable that the given test should have a high sensitivity and specificity value. Furthermore, it is not acceptable to sacrifice either one of these features to enhance the other. Therefore, values that are high enough to improve the clinical patient selection process, but low enough to be achievable with current research capabilities were chosen as indicative of SCA. The goal is to have 80% sensitivity and 80% specificity, which is met by 84.8% and 84.5%, respectively, based on calculations from the data in Table 6.

TABLE 6
Sensitivity and Specificity if the Combined Tests Enumerated in Table 5,
Based on the Results of the Recursive Partitioning Algorithm

| | | Experienced VT/VF | | |
|---|---|---|---|---|
| | | Yes | No | Total |
| Combined Tests | Implant | A = 28 | B = 32 | 60 |
| | Do not Implant | C = 5 | D = 175 | 180 |
| | Total | 33 | 207 | 240 |

$Sensitivity\_of \_combined \_test = \frac{A}{A + C} = \frac{28}{28 + 5} = 84.8 \%$ $Specificity\_of \_combined \_test = \frac{D}{B + D} = \frac{175}{175 + 32} = 84.5 \%$

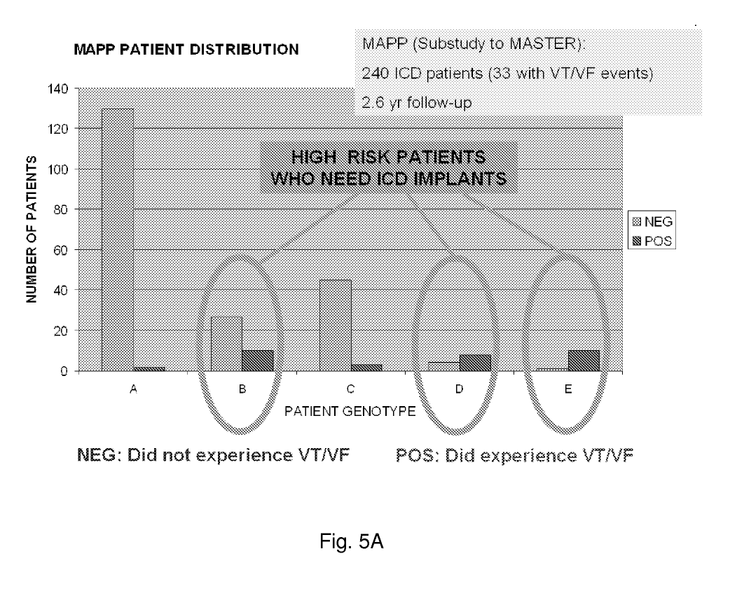

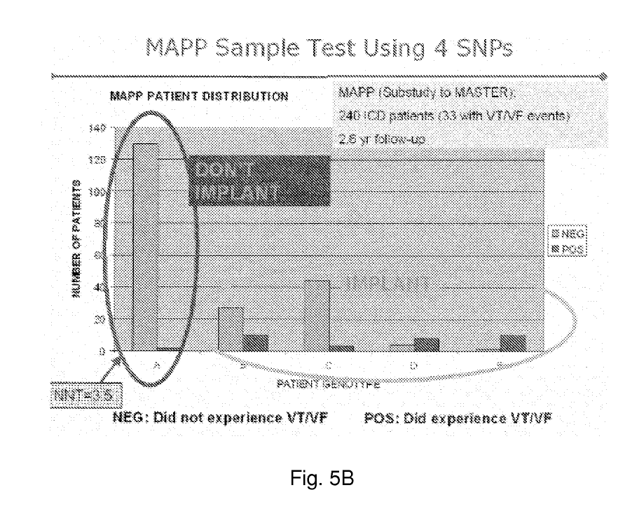

The same results are also shown in the graphical format provided in FIGS. 5A and 5B.

FIGS. 5A and 5B indicates how 4 SNP markers could potentially be used to differentiate patients into high risk and low risk groups. The five SNPs indicated in Table 7 are shown visually among the SNPs in the decision tree in FIG. 4. Group A consists of patients with the TT genotype for rs10505726 and the TC or TT genotype for rs2716727. As indicated by FIG. 5B, these patients would not be considered to be at relatively high risk for a life threatening VT/VF based solely on the genetic diagnostic test. Alternatively, Group B consists of patients with the TT genotype for rs10505726, but with the CC genotype for rs2716727. As indicated by FIG. 5A, these patients would be considered to be at relatively high risk for a life threatening VT/VF based solely on the genetic test and would be considered to be candidates for ICD implantation. Similar logic dictates that Groups D and E are relatively high risk and Group C is relatively low risk for life threatening VT/VF based on the genotypes of rs10505726, rs564275 and rs3775296. Rs7241111 from Table 7 is not used in FIG. 5A, but could be used to further risk stratify the patients.

Additional investigations were conducted using references to determine the nature of the five polymorphisms that were identified by the recursive partitioning algorithm. Results of this work are summarized in Table 7.

TABLE 7
SNPs That Were Found to Be Statistically Significant
Using the Recursive Partitioning Analysis

| | Fisher | | | | | |
|---|---|---|---|---|---|---|
| | Exact Test | Chromosome | Gene | Entrez | Functional | Chromosome |
| SNP | p-value | number | Name | ID | Class | Position |
| rs10505726 | 3.46 × 10⁻⁵ | 12 | PARP11 | 57097 | Intron | 12:3848218 |
| rs2716727 | 3.67 × 10⁻³ | 2 | — | — | — | 2:39807249 |
| rs564275 | 3.72 × 10⁻³ | 9 | GLIS3 | 169792 | Intron | 9:4084320 |
| rs7241111 | 7.33 × 10⁻³ | 18 | — | — | — | 18:63002332 |
| rs3775296 | 6.01 × 10⁻² | 4 | TLR3 | 7098 | Mrna-utr | 4:187234760 |

Persons skilled in the art of medical diagnosis will appreciate that there are multiple methods for the combination of measurements from a patient contemplated by the present invention. For example, a triple test given during pregnancy utilizes the three factors measured from a female subject, and a medical decision is made by further addition of the age of the subject. Similarly, SNPs described in this invention can be combined with other patient information, such as co-morbidities (e.g., diabetes, obesity, cholesterol, family history), parameters derived from electrophysiological measurements such as T-wave alternans, heart rate variability and heart rate turbulence, hemodynamic variables such as ejection fraction and end diastolic left ventricular volume, to yield a superior diagnostic technique. Furthermore, such a combination of a set markers can be achieved by multiple methods, including logical, linear, or non-linear combination of these markers, or by the use of clustering algorithms known in the art.

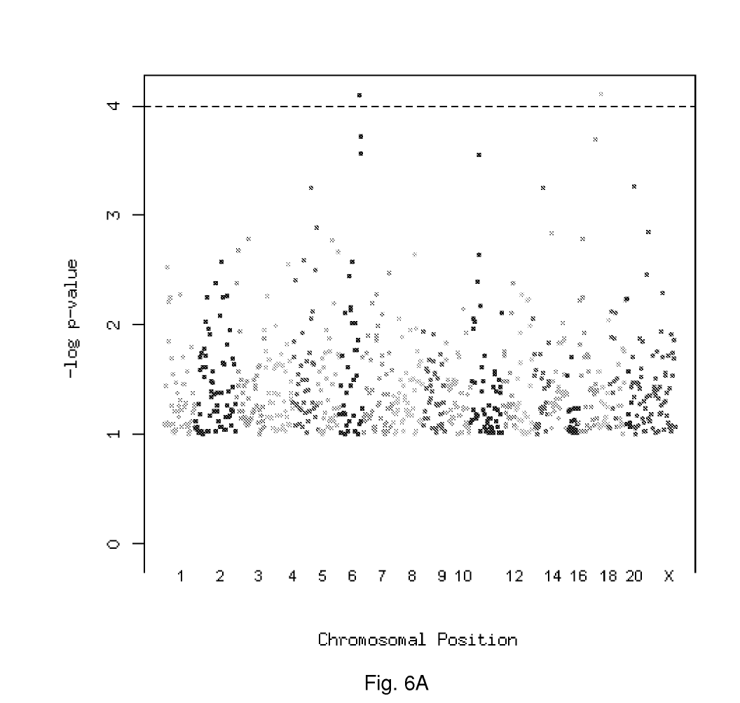

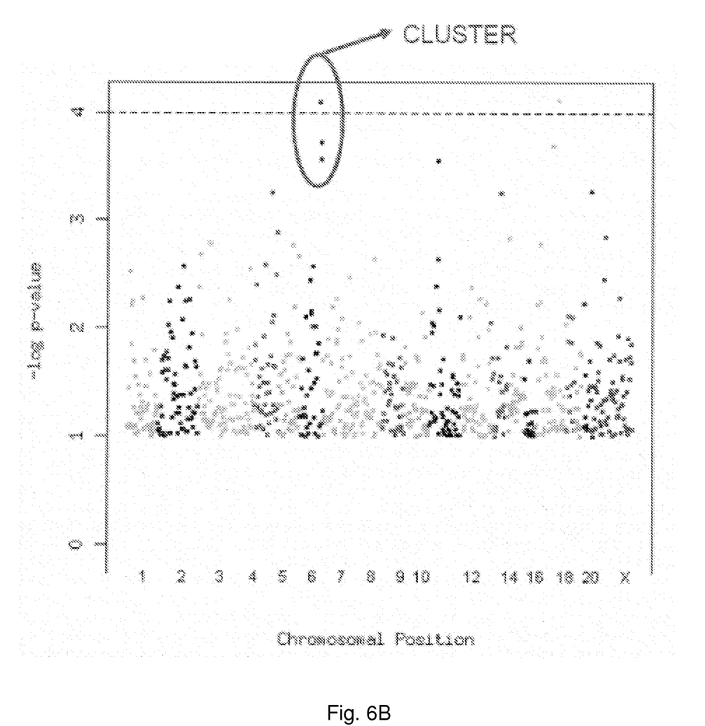

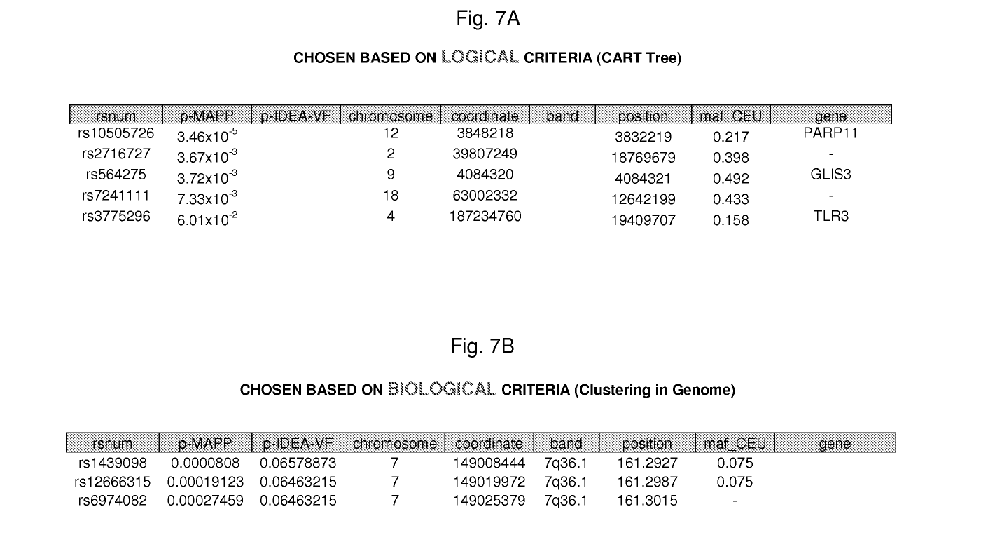

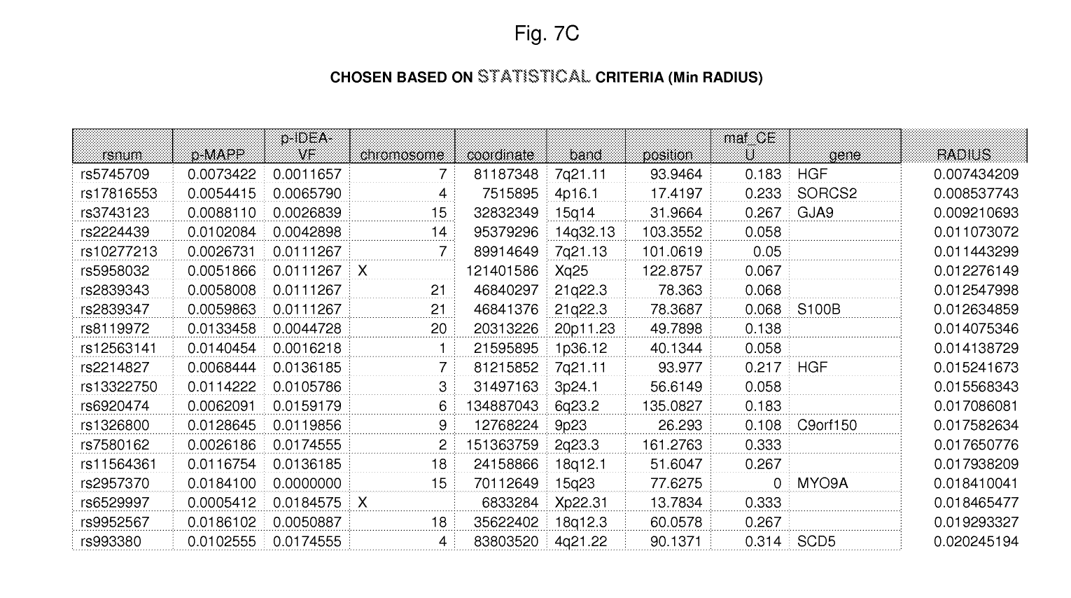

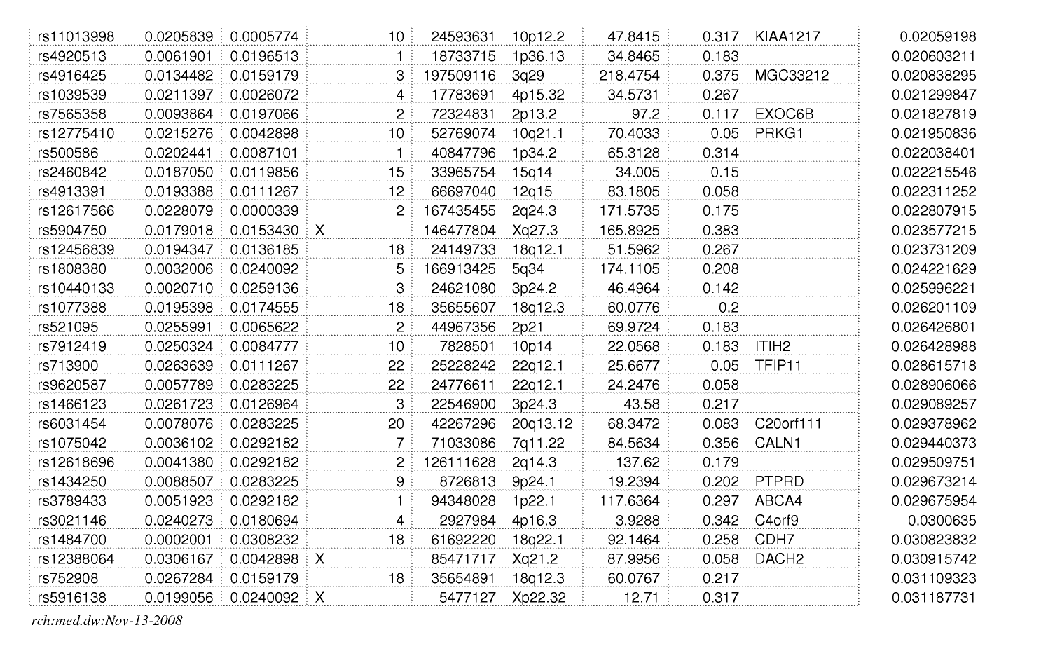

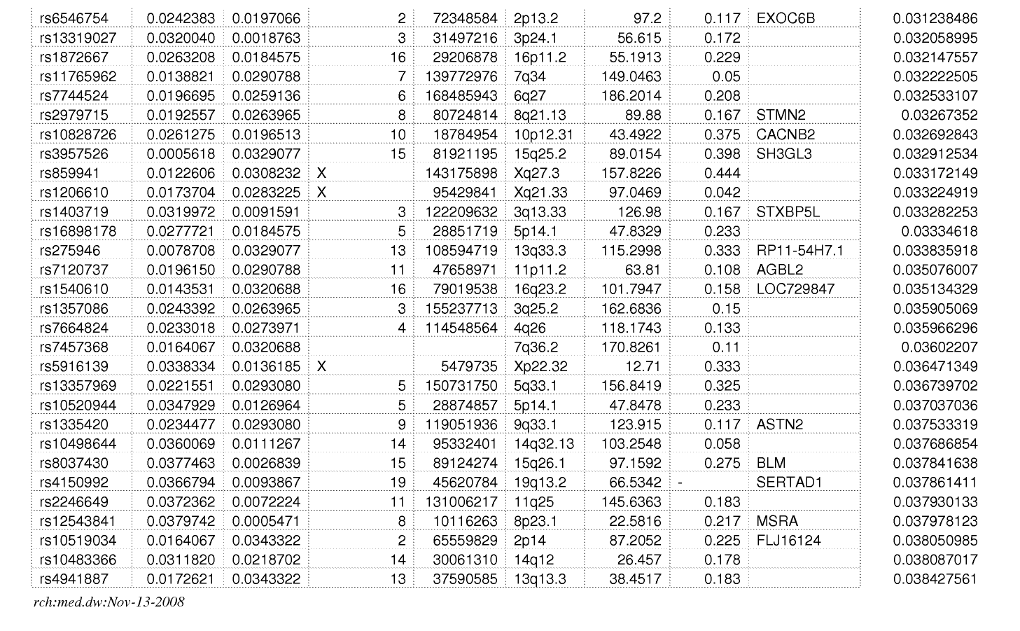

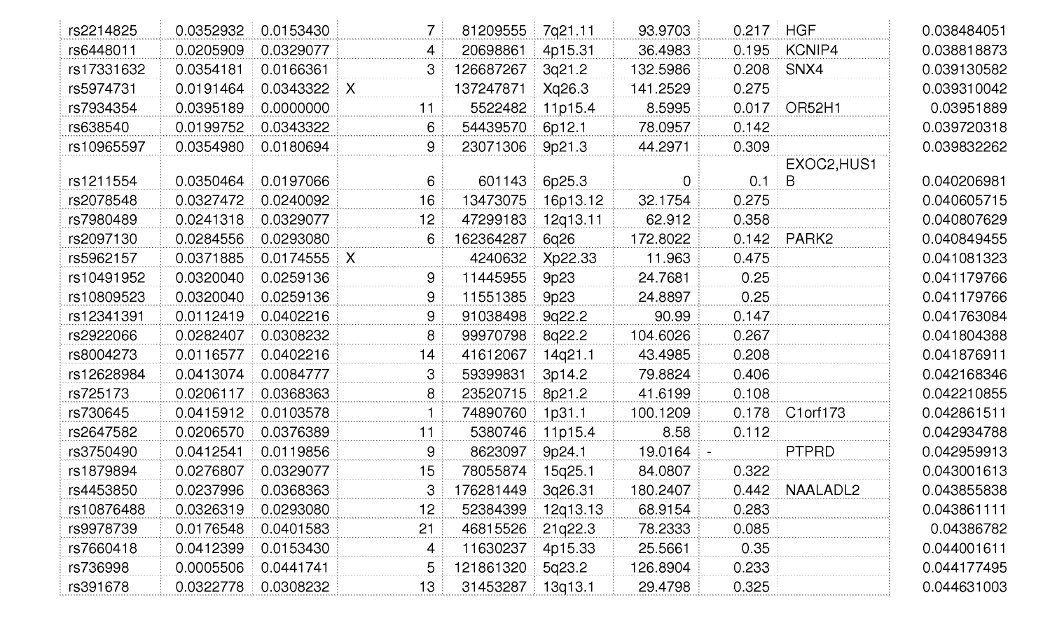

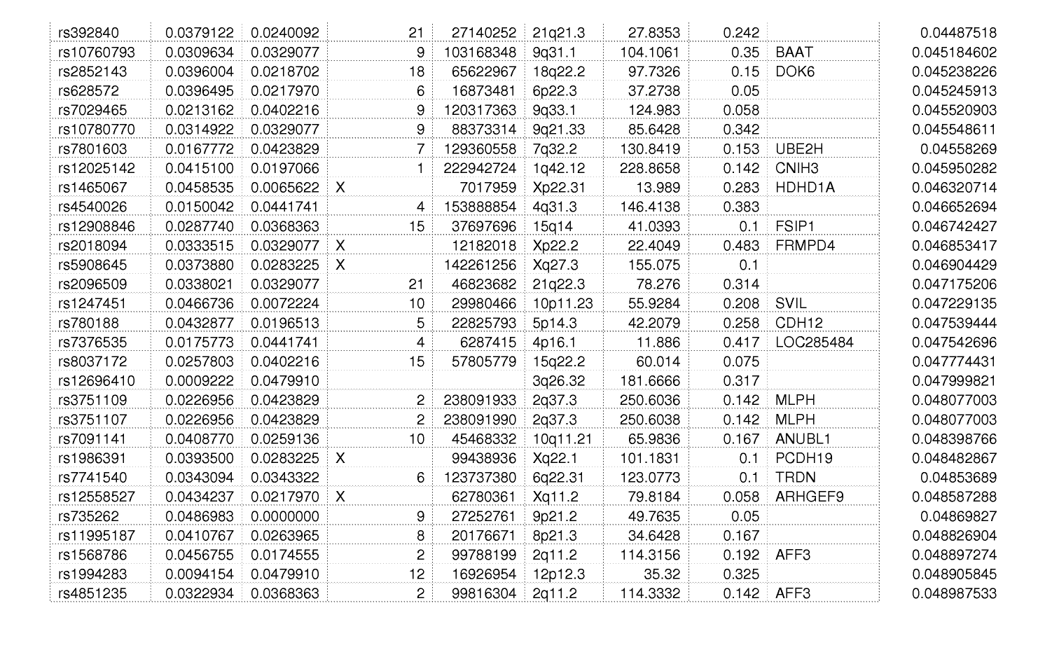

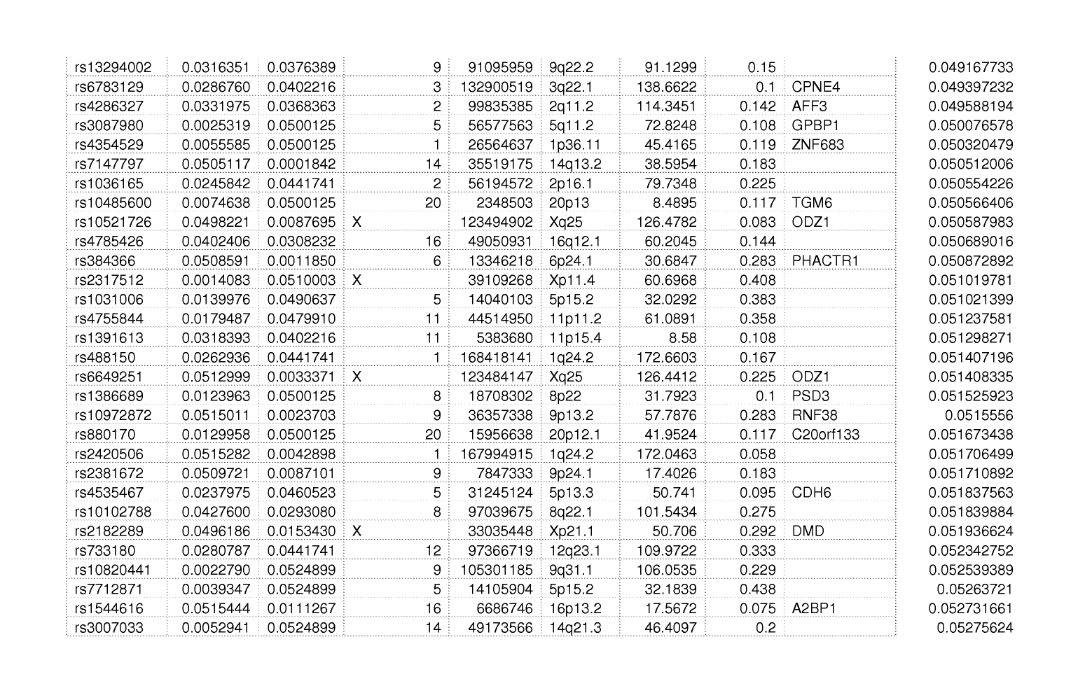

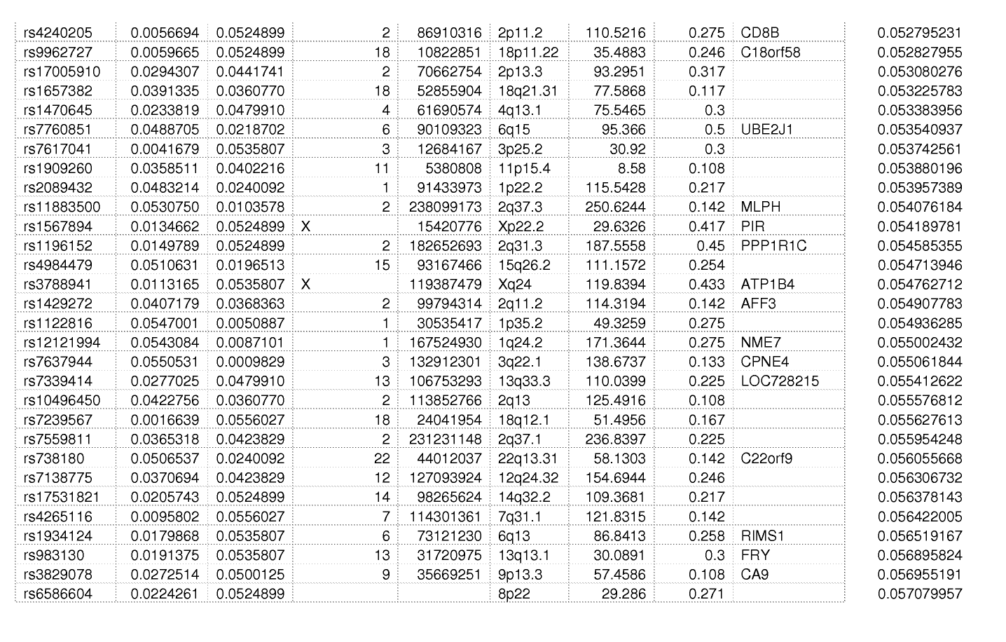

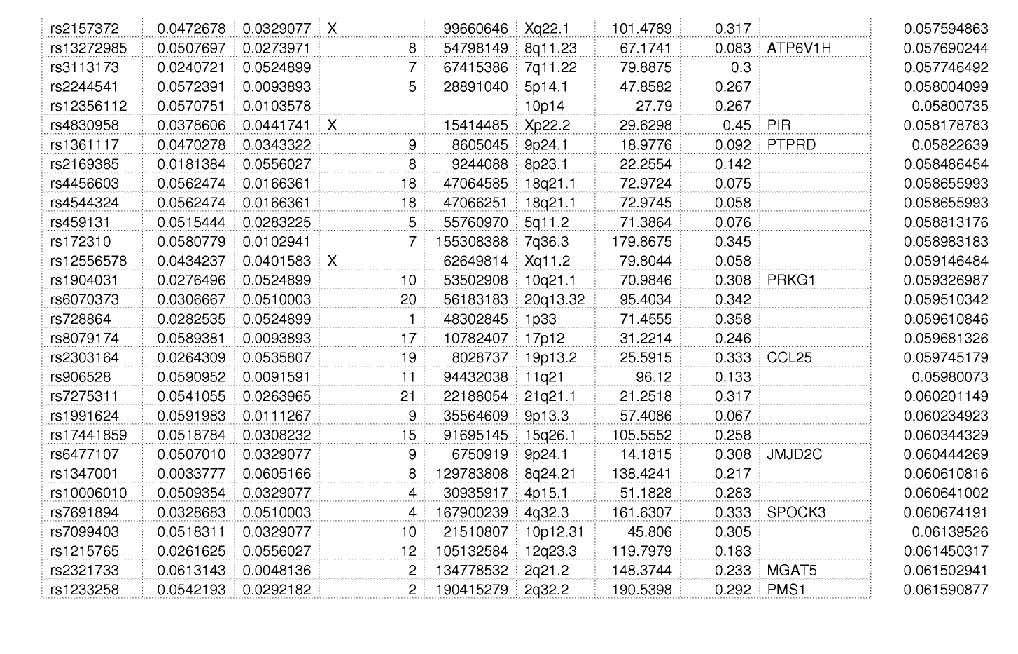

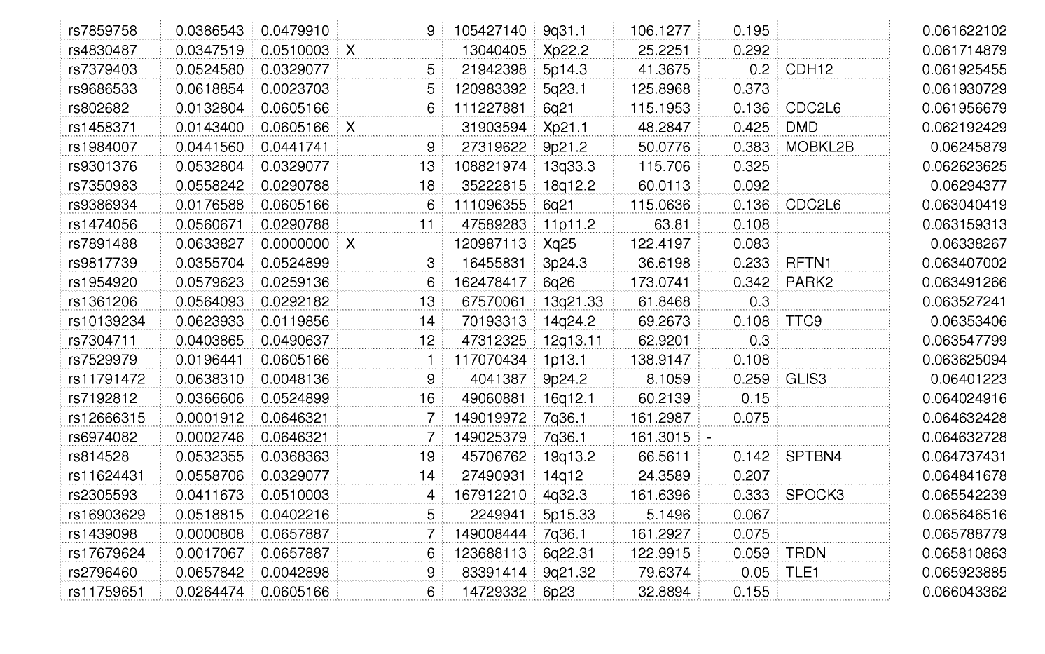

Furthermore, analysis was done using the data obtained from another study, namely the IDEA-VF, where SNP data from 37 ICD and 51 control patients was available. Again, the 317,503 SNPs in the MAPP study were scanned to identify the SNPs with p≦0.1, and 31,008 SNPs were found. These SNPs were tested in the IDEA-VF set, and only 849 of them were found to have p≦0.1, meaning that all 849 SNPs showed p values that were less than 0.1 in two independent studies. The chromosomal plot for these 849 SNPs with p≦0.1 for both MAPP and IDEA-VF are shown in FIG. 6. FIGS. 7A, 7B and 7C contain a detailed table of all the 849 SNPs (SEQ ID Nos. 1 to 849) chosen based on logical, biological and statistical criteria. For SEQ ID Nos. 1-849 of the Sequence Listing of the invention, the SNP is located at position 51.

To determine the presence or absence of an SNP in an individual or patient, an array having nucleotide probes from each of the sequences listed in SEQ ID Nos. 1 to 849 can be constructed where each probe is a different nucleotide sequence from 3 to 101 base pairs overlapping the SNP at position 51. In a further embodiment, the nucleotide probes are from each of the sequences listed in SEQ ID Nos. 850-858 and can be constructed where each probe is a different nucleotide sequence from 3 to 101 base pairs overlapping the SNP at position 26 or 27. In a further embodiment, the sequences of SEQ ID Nos. 1 to 858 can be individually used to monitor loss of heterozygosity, identify imprinted genes; genotype polymorphisms, determine allele frequencies in a population, characterize bi-allelic or multi-allelic markers, produce genetic maps, detect linkage disequilibrium, determine allele frequencies, do association studies, analyze genetic variation, or to identify markers linked to a phenotype or, compare genotypes between different individuals or populations.

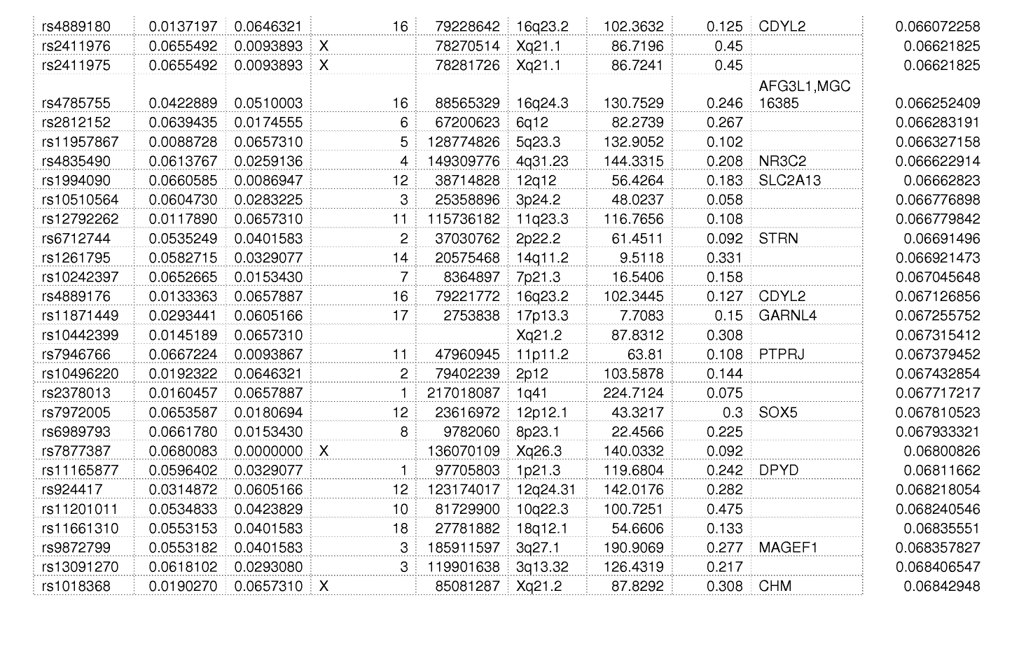

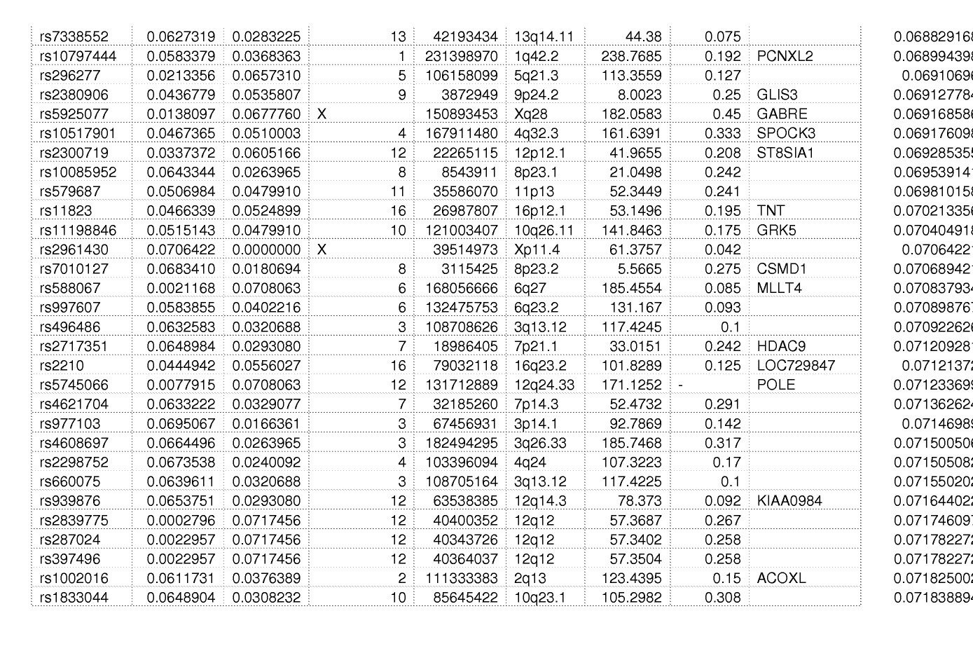

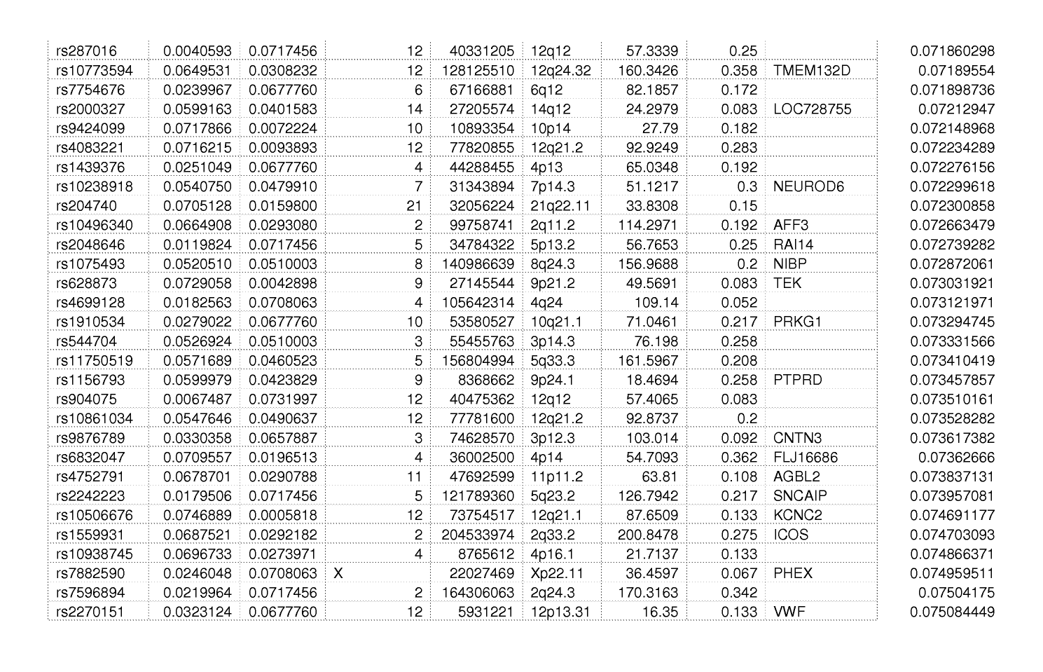

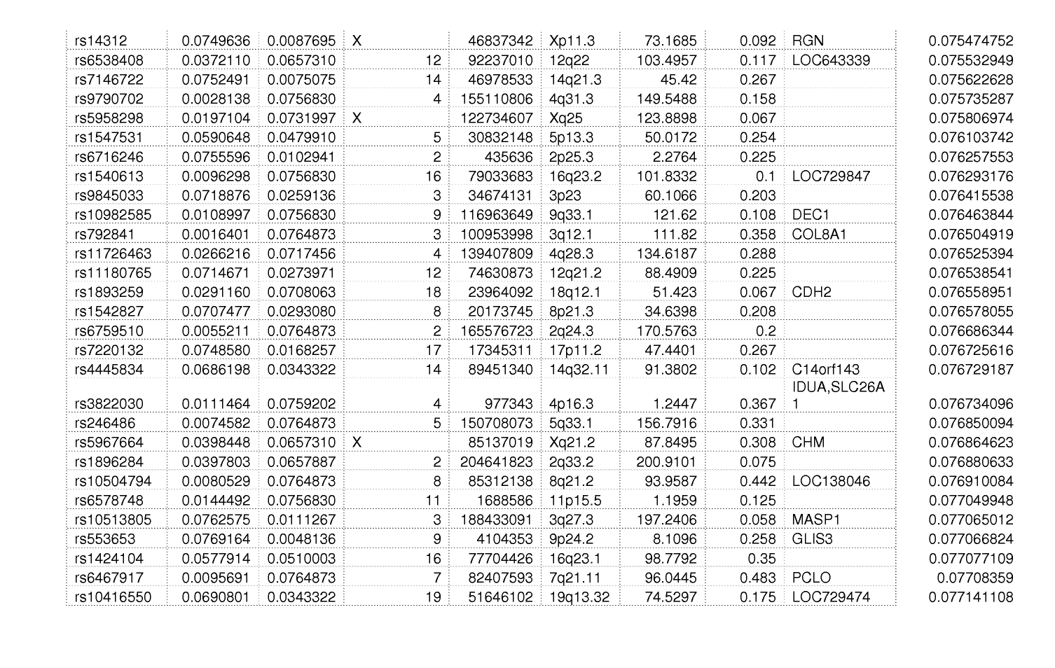

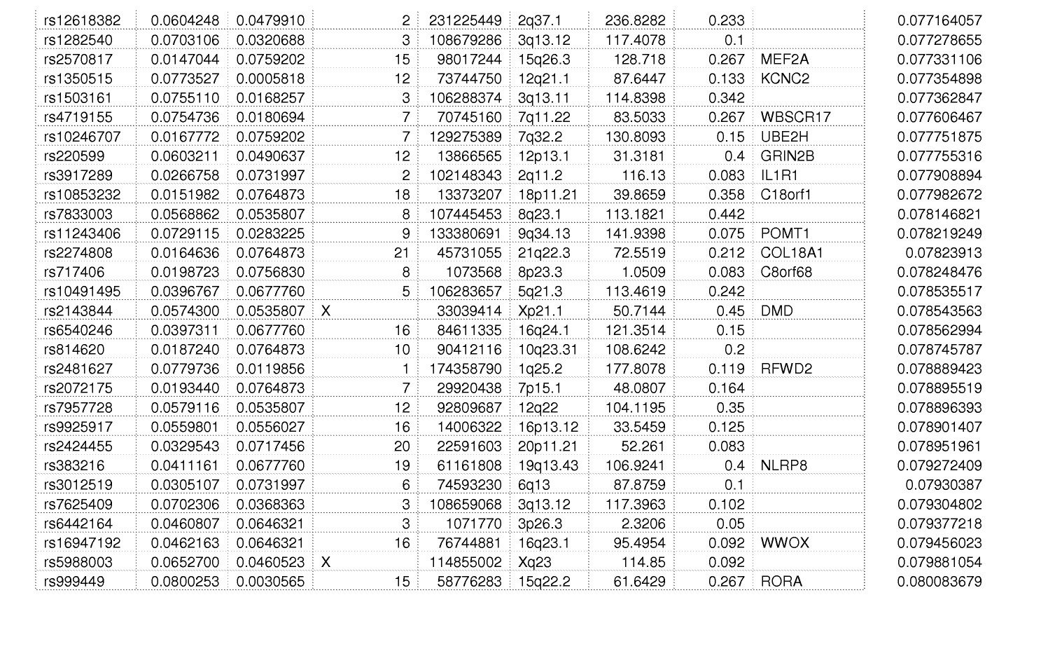

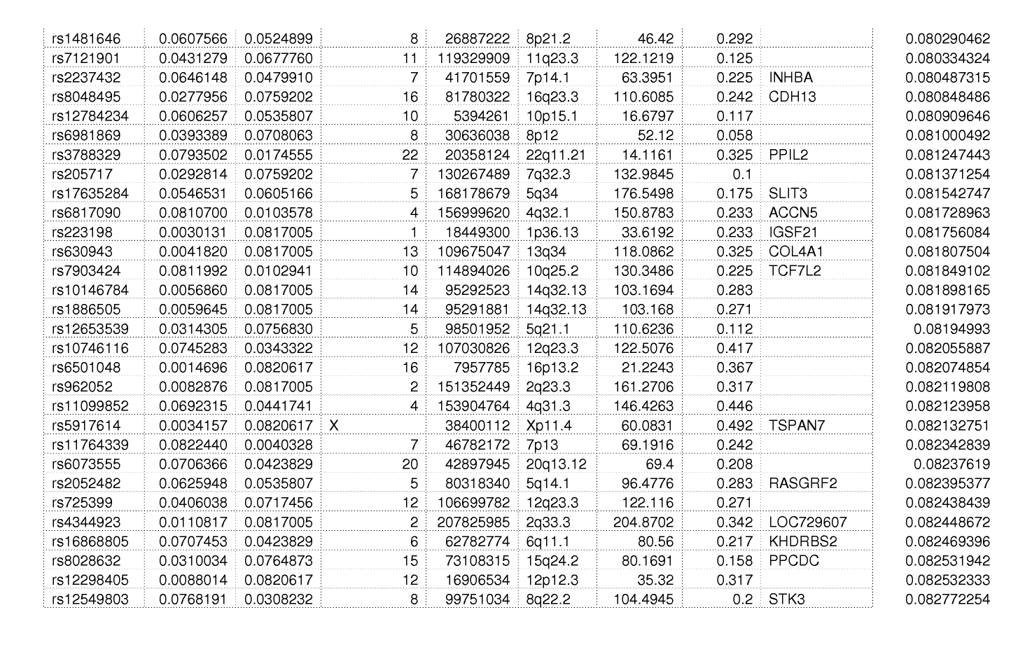

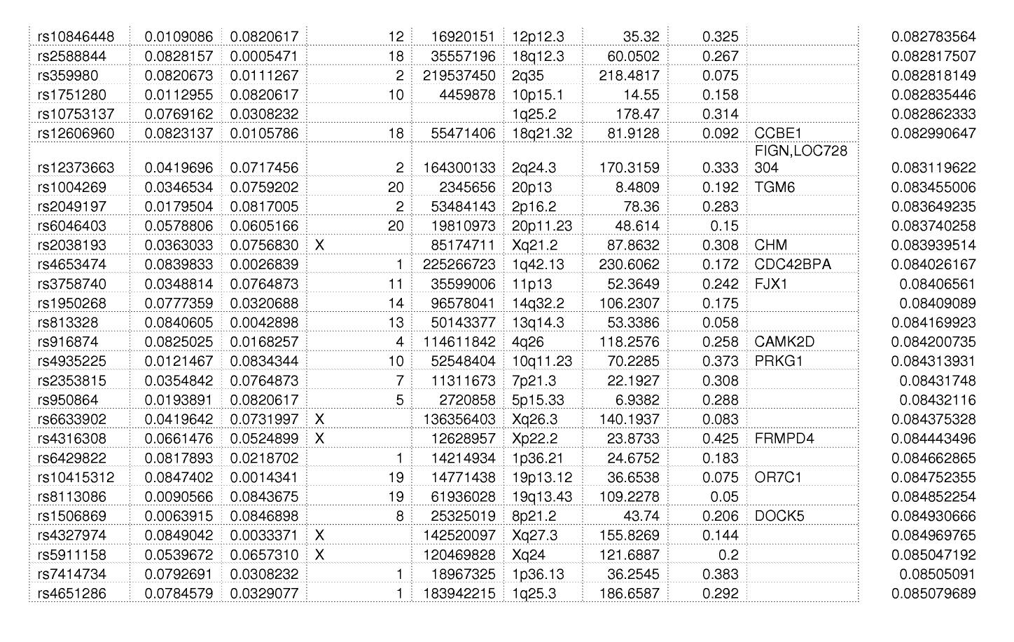

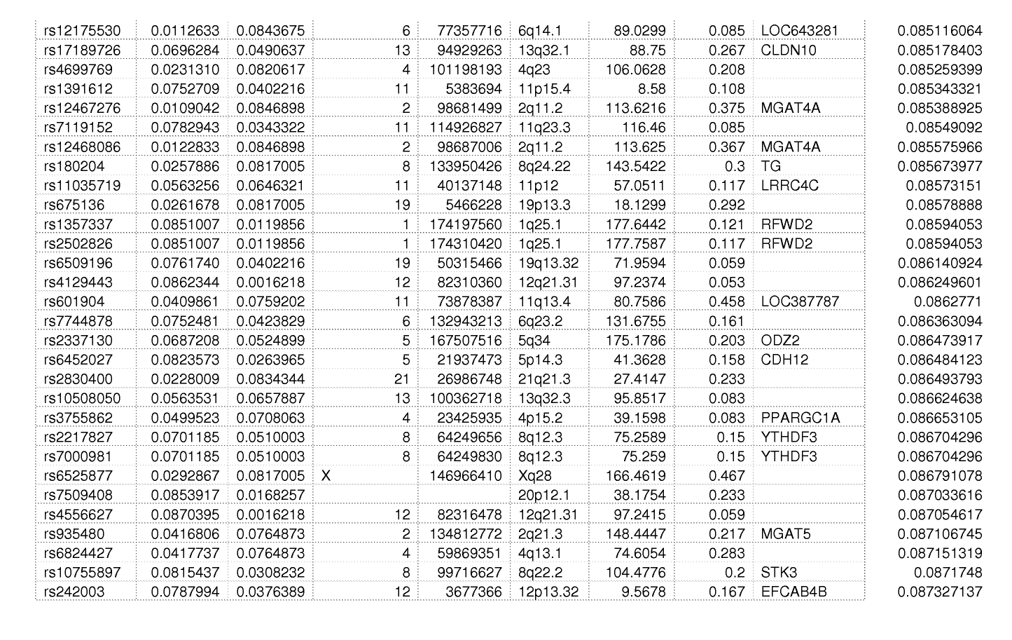

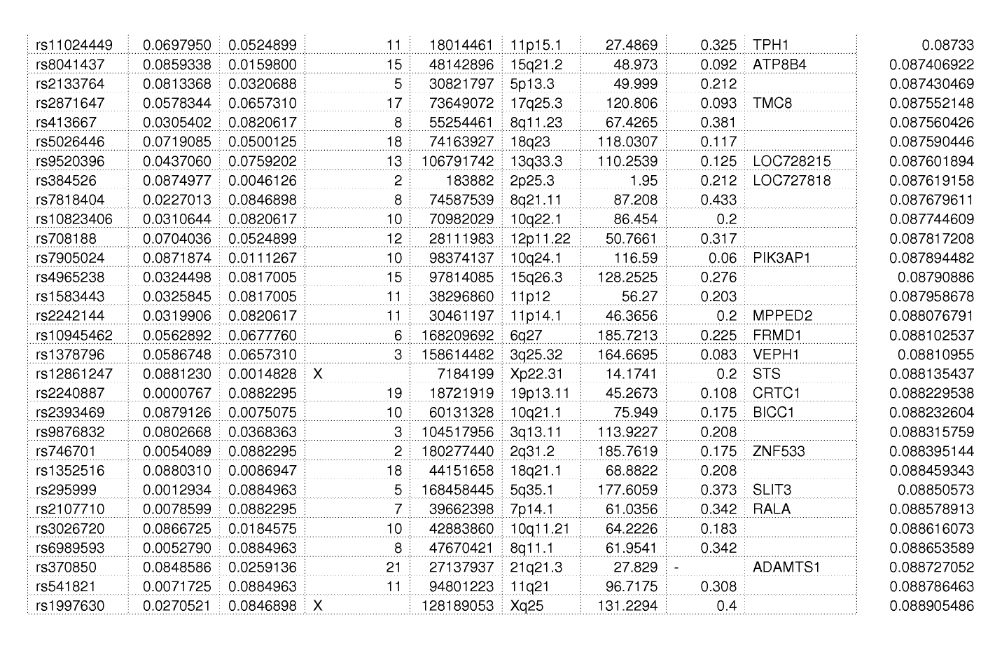

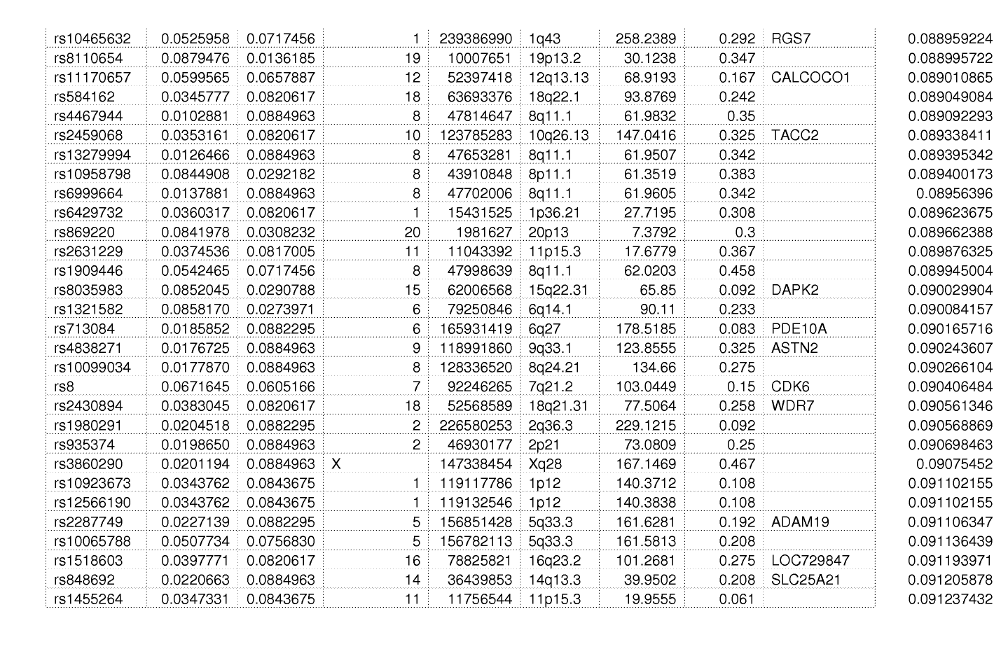

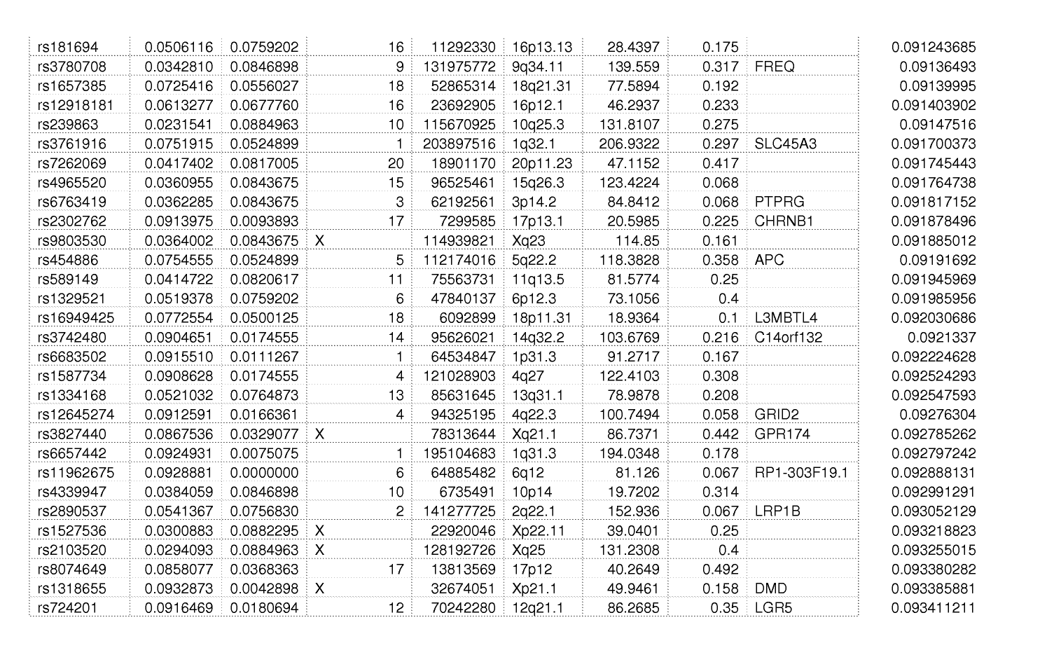

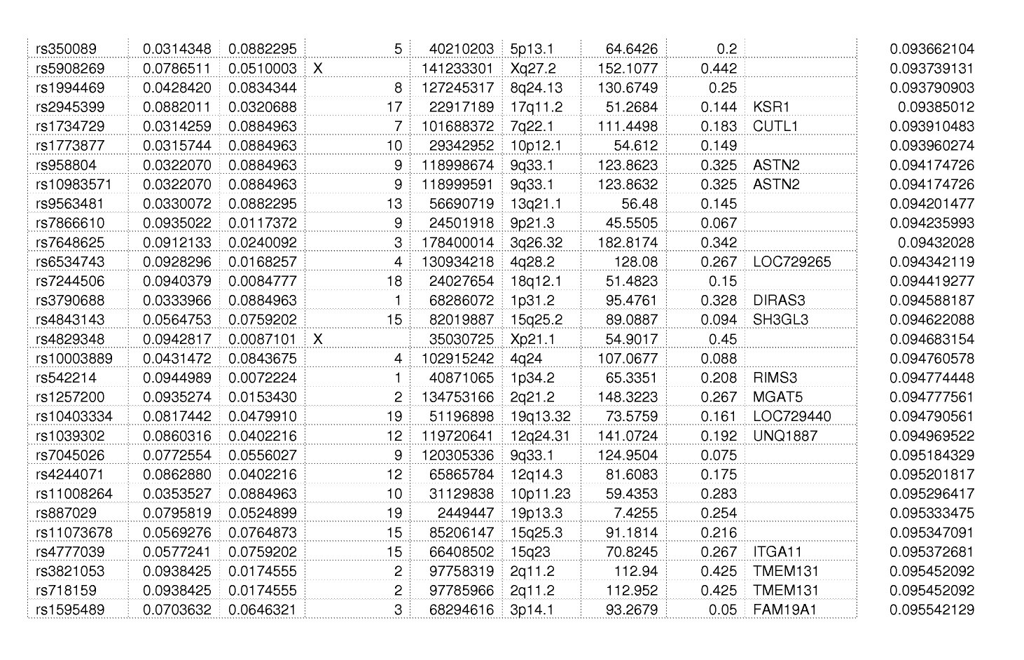

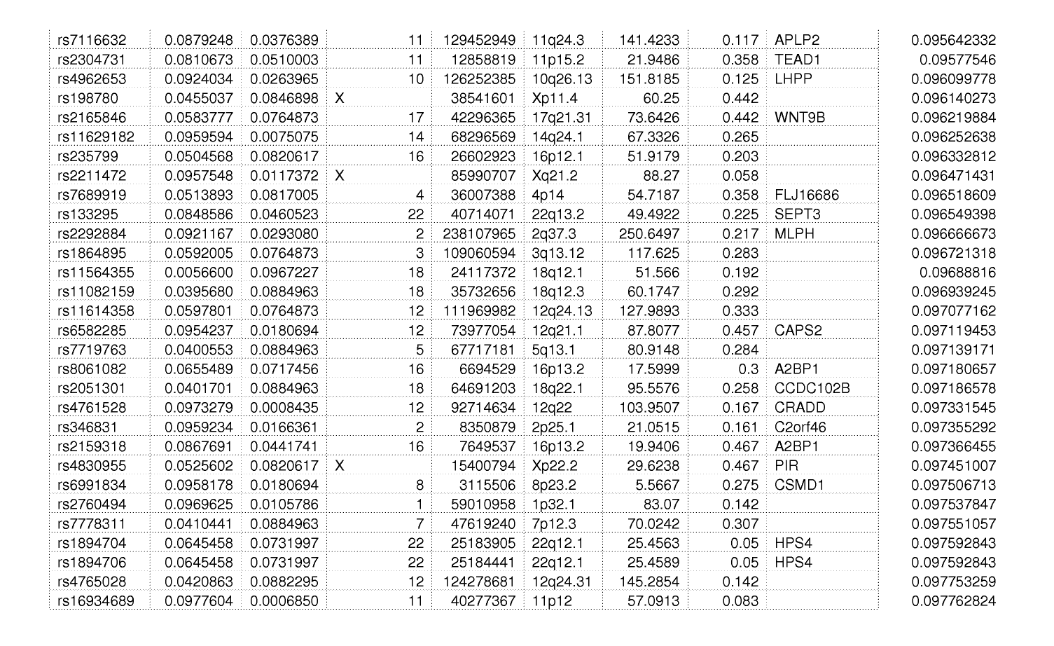

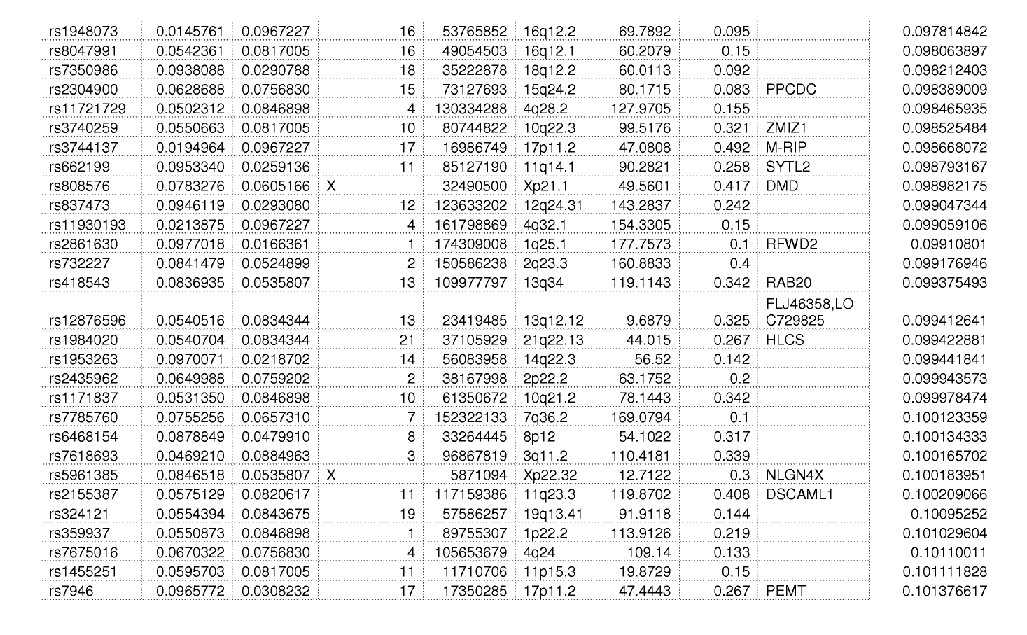

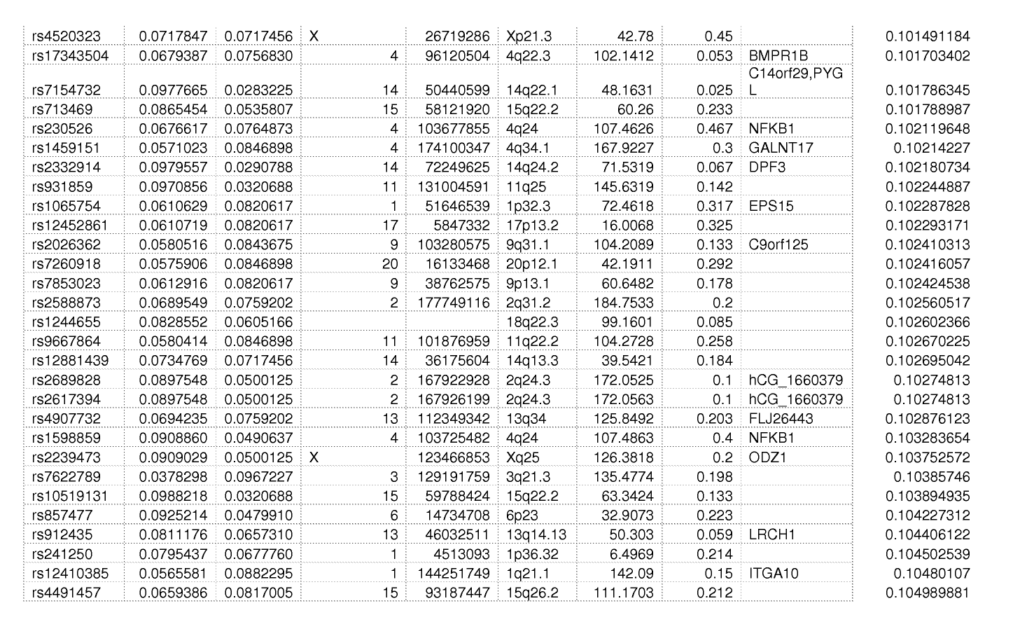

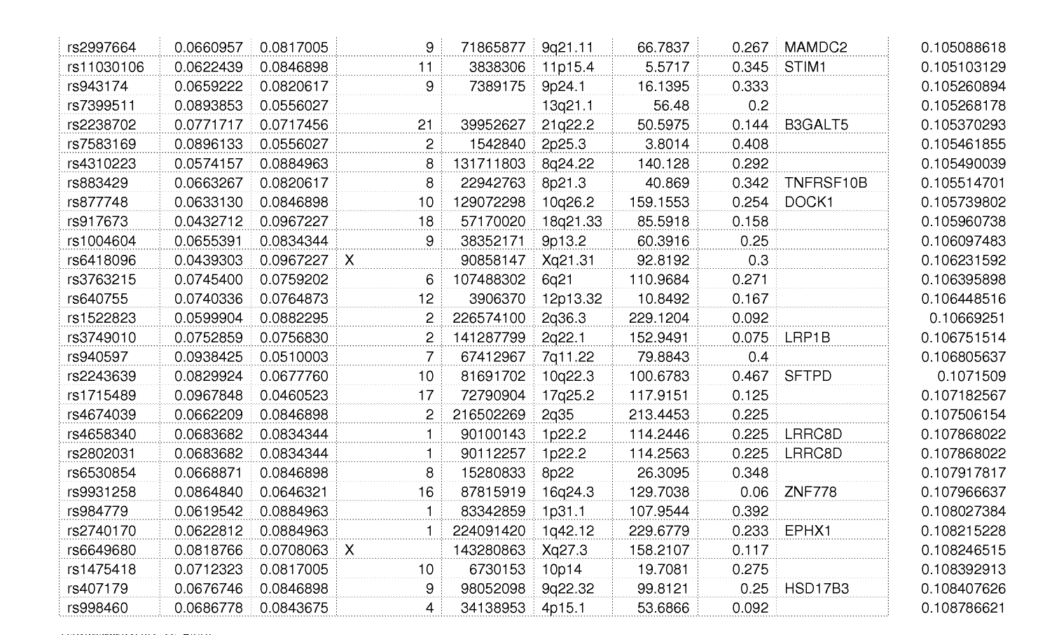

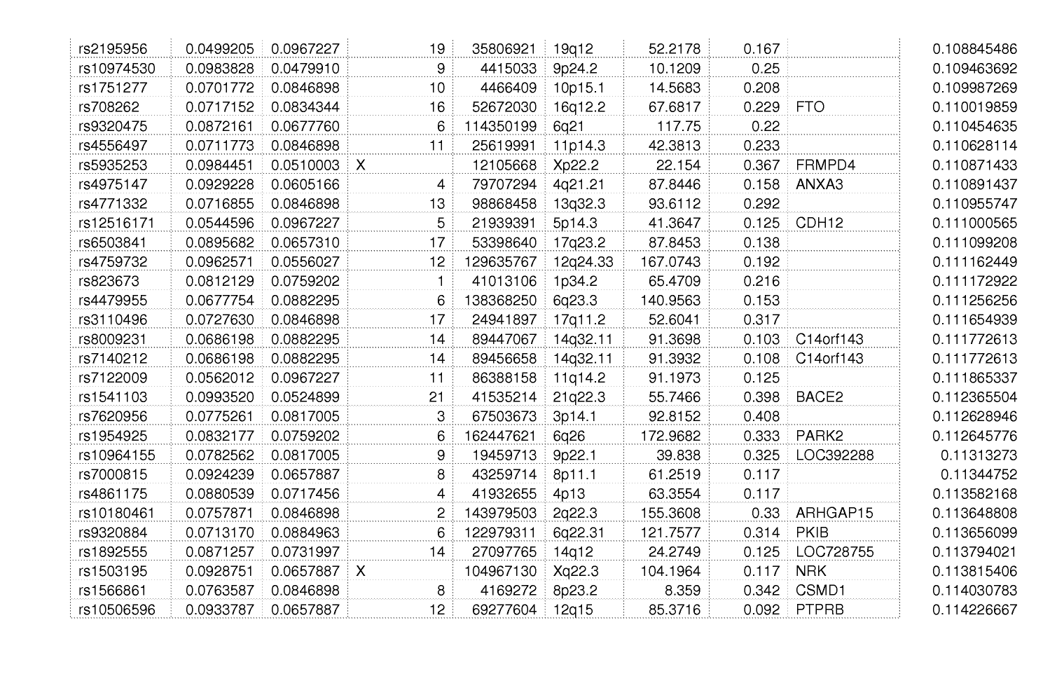

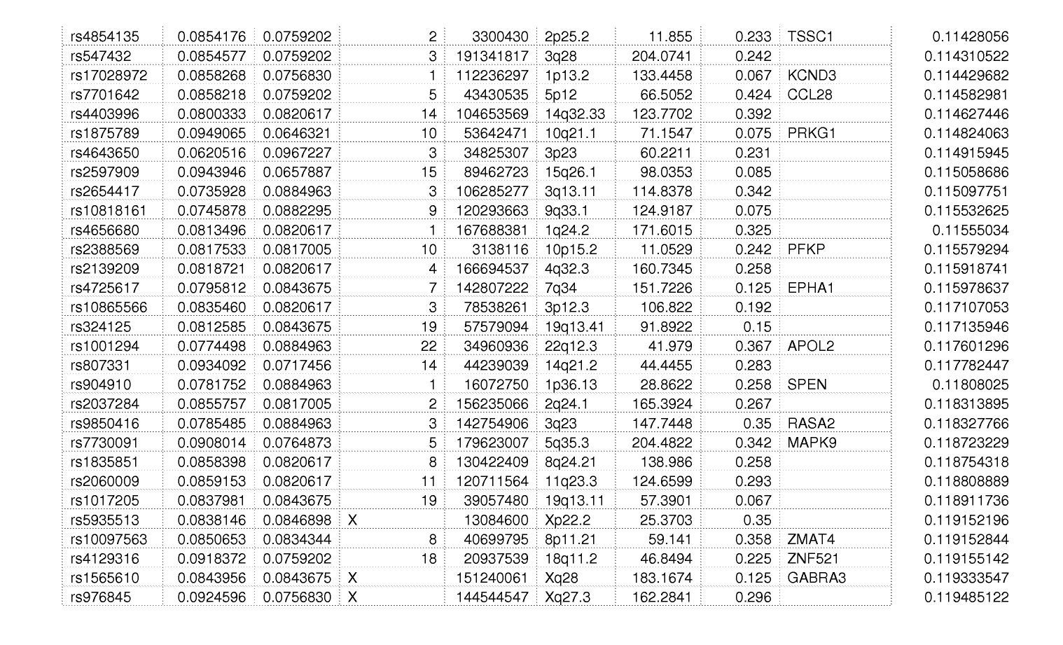

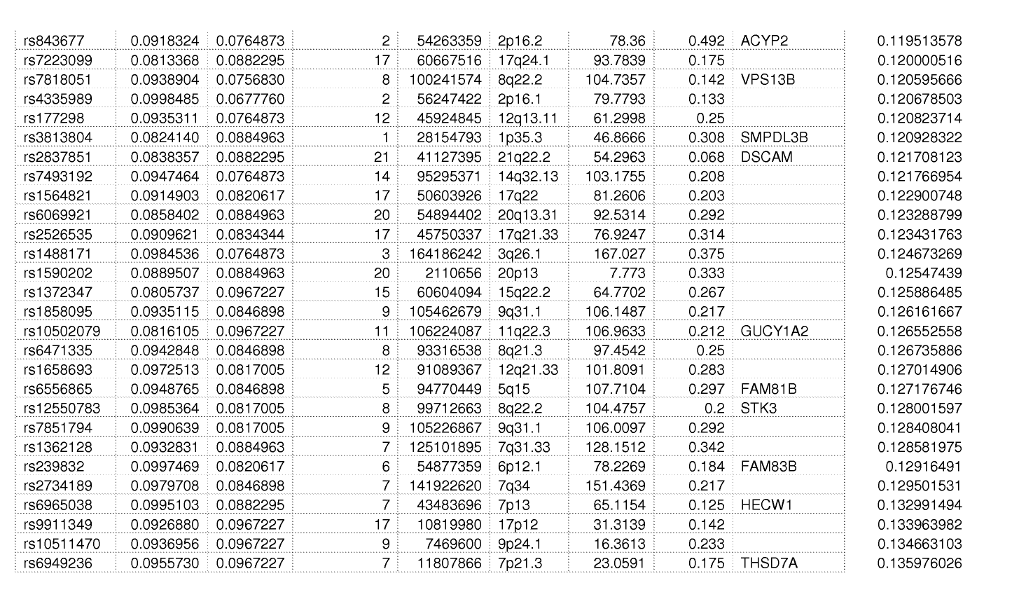

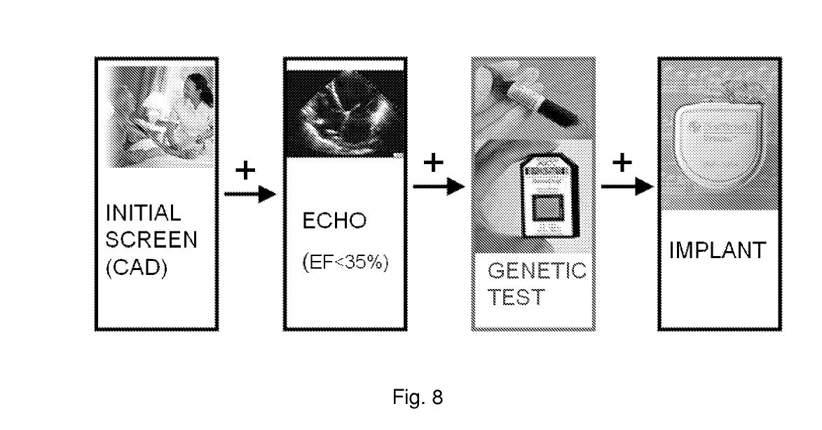

FIG. 8 depicts one embodiment of a clinical utilization of the genetic test created for screening of patients for susceptibility to life threatening arrhythmias. In this embodiment, patients already testing positively for CAD and a low EF would undergo the test for genetic susceptibility using any of the methods described herein. Positive genetic test results would then be used in conjunction with the other test, such as the ones based on the analysis of ECG, and be used to make the ultimate decision of whether or not to implant an ICD.

Patients who are presenting a cardiac condition such as MI are usually subjected to echocardiographic examination to determine the need for an ICD. Based on the present invention, blood samples could also be taken from the patients who have low left ventricular EF. If the genetic tests in combination with the hemodynamic and demographic parameters indicate a high risk for sudden cardiac arrest, then a recommendation is made for an ICD implant. A schematic of this overall process is shown in FIG. 8. A similar recommendation can be made for individuals with no previous history of cardiovascular disease based on a positive genetic screen for one or more of the SNPs taught herein in combination with one or more biological factors including markers, clinical parameters and the like.

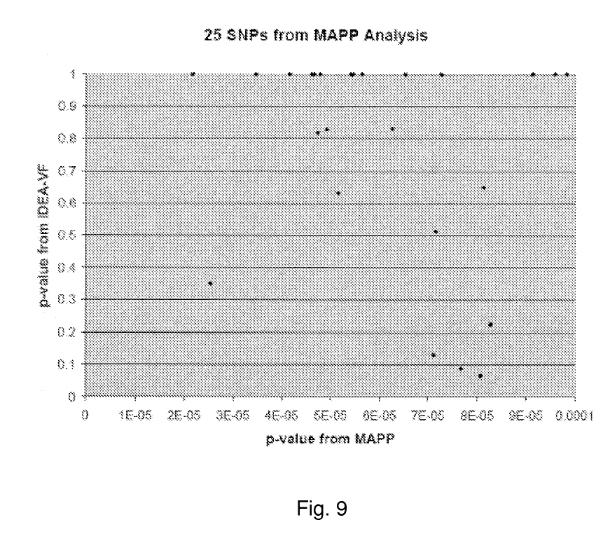

FIG. 9 shows the performance of the genetic markers obtained from the MAPP Study when they were applied to the IDEA-VF patient population. Only the markers with MAPP p-values that are less than 0.0001 were tested. As it can be seen from this graph, not all the markers identified as highly significant in MAPP did not give low p-values when they are applied to the IDEA-VF population. A total of 25 SNPs are represented in FIG. 9: rs4878412, rs2839372, rs10505726, rs10919336, rs6828580, rs16952330, rs2060117, rs9983892, rs1500325, rs1679414, rs486427, rs6480311, rs7305353, rs10823151, rs1346964, rs6790359, rs7591633, rs10487115, rs2240887, rs1439098, rs248670, rs4691391, rs2270801, rs12891099, and rs17694397.

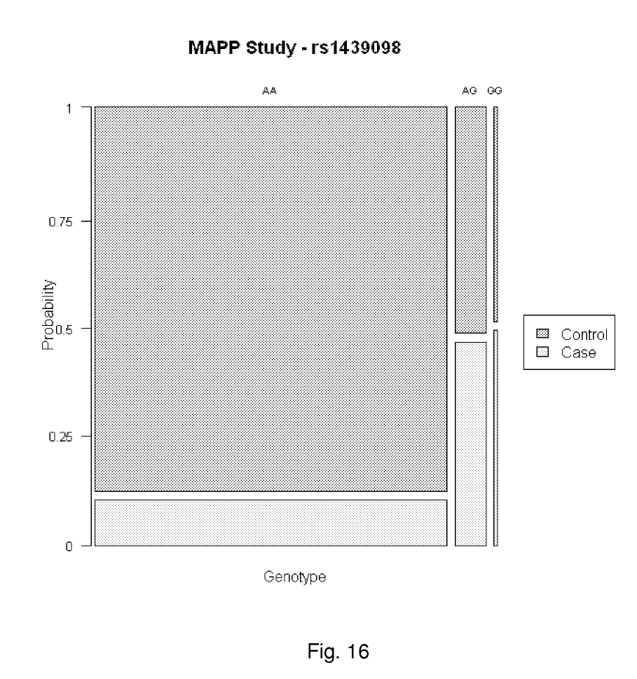

FIGS. 16-40 contain mosaic plots illustrating the probability of experiencing LTA as a function of allele specific inheritance of the 25 SNPs represented in FIG. 9. FIG. 16 illustrates the resulting risk stratification of rs1439098. As shown in the plot, the presence of a at the SNP position indicates decreased susceptibility to SCA or SCD as compared to the presence of g at the SNP position. Patients with genotype a/a have about an 11% probability of experiencing SCA or SCD, while the a/g genotype indicates about a 47% probability, and the g/g genotype indicates about a 50% probability of experiencing SCA or SCD. Table 8 shows the statistical breakdown of the genotypes for this SNP.

The first (top) value in each cell in each of the statistical tables is the number, or count, of patients placed in that set. The second value is the percentage of the total number of patients placed in the set. The third value is the percentage of control or case patients (depending on the column) having a specific genotype from the total number of patients having that specific genotype. The fourth value is the percentage of patients from either the control or case patients (depending on the column) placed in the set. The bottom right cell is the total number of patients (100%) utilized for the SNP analysis.

TABLE 8
Table of rs1439098 by arm

| rs1439098 | arm | | |
|---|---|---|---|
| Count | | | |
| Frequency % | | | |
| Row % | | | |
| Col % | Control | Case | Total |
| AA | 195 | 23 | 218 |
| | 81.59 | 9.62 | 91.21 |
| | 89.45 | 10.55 | |
| | 94.66 | 69.70 | |
| AG | 10 | 9 | 19 |
| | 4.18 | 3.77 | 7.95 |
| | 52.63 | 47.37 | |
| | 4.85 | 27.27 | |
| GG | 1 | 1 | 2 |
| | 0.42 | 0.42 | 0.84 |
| | 50.00 | 50.00 | |
| | 0.49 | 3.03 | |
| Total | 206 | 33 | 239 |
| | 86.19 | 13.81 | 100.00 |

Frequency Missing = 1

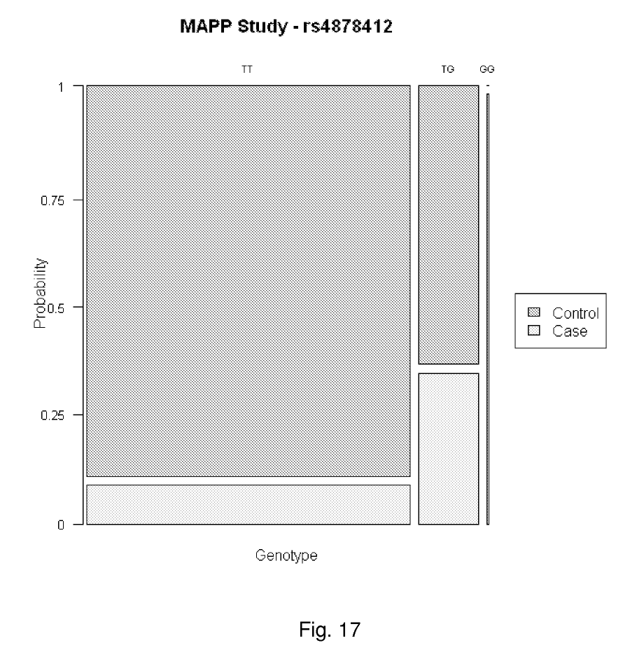

FIG. 17 illustrates the resulting risk stratification of rs4878412. As shown in the plot, the presence of t at the SNP position indicates decreased susceptibility to SCA or SCD as compared to the presence of g at the SNP position. Patients with genotype t/t have about a 9% probability of experiencing SCA or SCD, while the t/g genotype indicates about a 35% probability, and the g/g genotype indicates greater than 99% probability of experiencing SCA or SCD. Table 9 shows the statistical breakdown of the genotypes for this SNP.

TABLE 9
Table of rs4878412 by arm

| rs4878412 | arm | | |
|---|---|---|---|
| Count | | | |
| Frequency % | | | |
| Row % | | | |
| Col % | Control | Case | Total |
| GG | 0 | 1 | 1 |
| | 0.00 | 0.42 | 0.42 |
| | 0.00 | 100.00 | |
| | 0.00 | 3.13 | |
| GT | 24 | 13 | 37 |
| | 10.13 | 5.49 | 15.61 |
| | 64.86 | 35.14 | |
| | 11.71 | 40.63 | |
| TT | 181 | 18 | 199 |
| | 76.37 | 7.59 | 83.97 |
| | 90.95 | 9.05 | |
| | 88.29 | 56.25 | |
| Total | 205 | 32 | 237 |
| | 86.50 | 13.50 | 100.00 |

Frequency Missing = 3

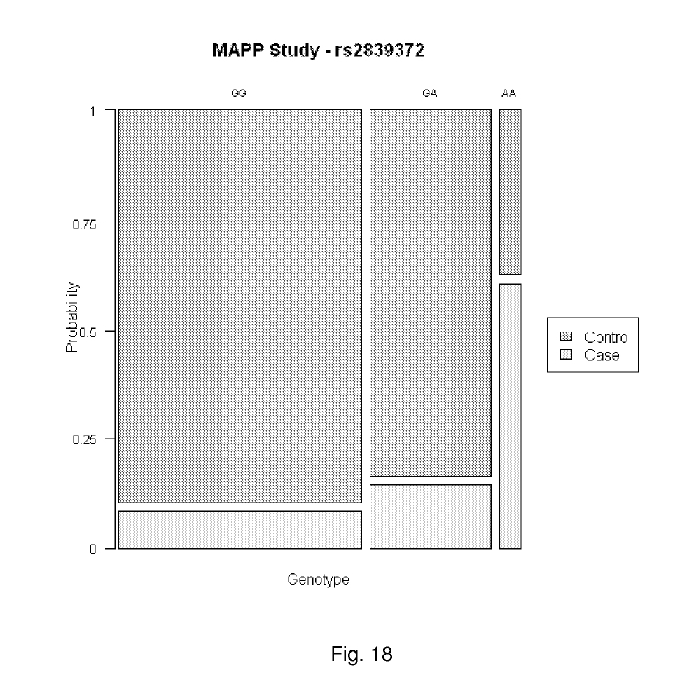

FIG. 18 illustrates the resulting risk stratification of rs2839372. As shown in the plot, the presence of g at the SNP position indicates &creased susceptibility to SCA or SCD as compared to the presence of a at the SNP position. Patients with genotype g/g have about a 9% probability of experiencing SCA or SCD, while the g/a genotype indicates about a 15% probability, and the a/a genotype indicates about a 62% probability of experiencing SCA or SCD. Table 10 shows the statistical breakdown of the genotypes for this SNP.

TABLE 10
Table of rs2839372 by arm

| rs2839372 | arm | | |
|---|---|---|---|
| Count | | | |
| Frequency % | | | |
| Row % | | | |
| Col % | Control | Case | Total |
| AA | 5 | 8 | 13 |
| | 2.10 | 3.36 | 5.46 |
| | 38.46 | 61.54 | |
| | 2.43 | 25.00 | |
| AG | 64 | 11 | 75 |
| | 26.89 | 4.62 | 31.51 |
| | 85.33 | 14.67 | |
| | 31.07 | 34.38 | |
| GG | 137 | 13 | 150 |
| | 57.56 | 5.46 | 63.03 |
| | 91.33 | 8.67 | |
| | 66.50 | 40.63 | |
| Total | 206 | 32 | 238 |
| | 86.55 | 13.45 | 100.00 |

Frequency Missing = 2

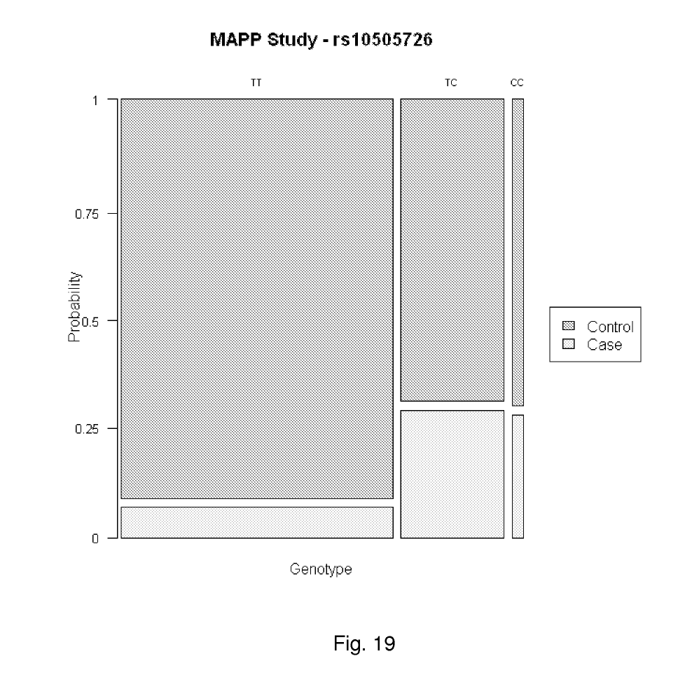

FIG. 19 illustrates the resulting risk stratification of rs10505726. As shown in the plot, the presence of t at the SNP position indicates decreased susceptibility to SCA or SCD as compared to the presence of c at the SNP position. Patients with genotype t/t have about a 7% probability of experiencing SCA or SCD, while the t/c genotype indicates about a 30% probability, and the c/c genotype indicates about a 29% probability of experiencing SCA or SCD. Table 11 shows the statistical breakdown of the genotypes for this SNP.

TABLE 11
Table of rs10505726 by arm

| rs10505726 | arm | | |
|---|---|---|---|
| Count | | | |
| Frequency % | | | |
| Row % | | | |
| Col % | Control | Case | Total |
| CC | 5 | 2 | 7 |
| | 2.08 | 0.83 | 2.92 |
| | 71.43 | 28.57 | |
| | 2.42 | 6.06 | |
| CT | 45 | 19 | 64 |
| | 18.75 | 7.92 | 26.67 |
| | 70.31 | 29.69 | |
| | 21.74 | 57.58 | |
| TT | 157 | 12 | 169 |
| | 65.42 | 5.00 | 70.42 |
| | 92.90 | 7.10 | |
| | 75.85 | 36.36 | |
| Total | 207 | 33 | 240 |
| | 86.25 | 13.75 | 100.00 |

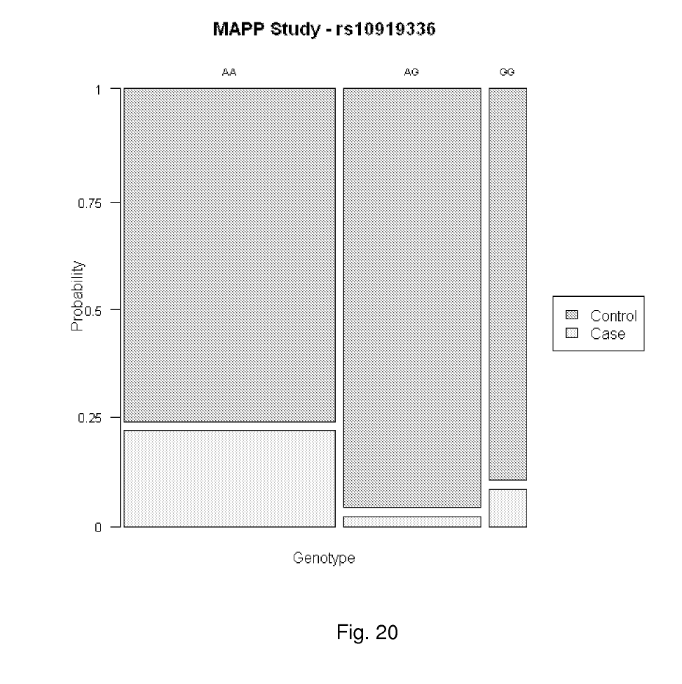

FIG. 20 illustrates the resulting risk stratification of rs10919336. As shown in the plot, the presence of a at the SNP position indicates increased susceptibility to SCA or SCD as compared to the presence of g at the SNP position. Patients with genotype a/a have about a 22% probability of experiencing SCA or SCD, while the a/g genotype indicates less than 5% probability, and the g/g genotype indicates about a 9% probability of experiencing SCA or SCD. Table 12 shows the statistical breakdown of the genotypes for this SNP.

TABLE 12
Table of rs10919336 by arm

| rs10919336 | arm | | |
|---|---|---|---|
| Count | | | |
| Frequency % | | | |
| Row % | | | |
| Col % | Control | Case | Total |
| AA | 101 | 29 | 130 |
| | 42.62 | 12.24 | 54.85 |
| | 77.69 | 22.31 | |
| | 49.51 | 87.88 | |
| AG | 82 | 2 | 84 |
| | 34.60 | 0.84 | 35.44 |
| | 97.62 | 2.38 | |
| | 40.20 | 6.06 | |
| GG | 21 | 2 | 23 |
| | 8.86 | 0.84 | 9.70 |
| | 91.30 | 8.70 | |
| | 10.29 | 6.06 | |
| Total | 204 | 33 | 237 |
| | 86.08 | 13.92 | 100.00 |

Frequency Missing = 3

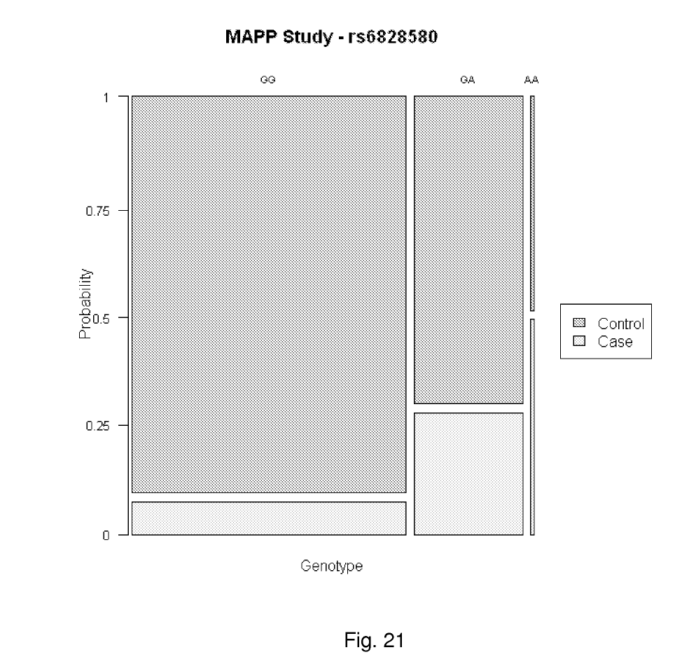

FIG. 21 illustrates the resulting risk stratification of rs6828580. As shown in the plot, the presence of g at the SNP position indicates decreased susceptibility to SCA or SCD as compared to the presence of a at the SNP position. Patients with genotype g/g have about an 8% probability of experiencing SCA or SCD, while the g/a genotype indicates about a 28% probability, and the a/a genotype indicates about a 50% probability of experiencing SCA or SCD. Table 13 shows the statistical breakdown for the genotypes of this SNP.

TABLE 13
Table of rs6828580 by arm

| rs6828580 | arm | | |
|---|---|---|---|
| Count | | | |
| Frequency % | | | |
| Row % | | | |
| Col % | Control | Case | Total |
| AA | 1 | 1 | 2 |
| | 0.42 | 0.42 | 0.84 |
| | 50.00 | 50.00 | |
| | 0.49 | 3.03 | |
| AG | 48 | 19 | 67 |
| | 20.08 | 7.95 | 28.03 |
| | 71.64 | 28.36 | |
| | 23.30 | 57.58 | |
| GG | 157 | 13 | 170 |
| | 65.69 | 5.44 | 71.13 |
| | 92.35 | 7.65 | |
| | 76.21 | 39.39 | |
| Total | 206 | 33 | 239 |
| | 86.19 | 13.81 | 100.00 |

Frequency Missing = 1

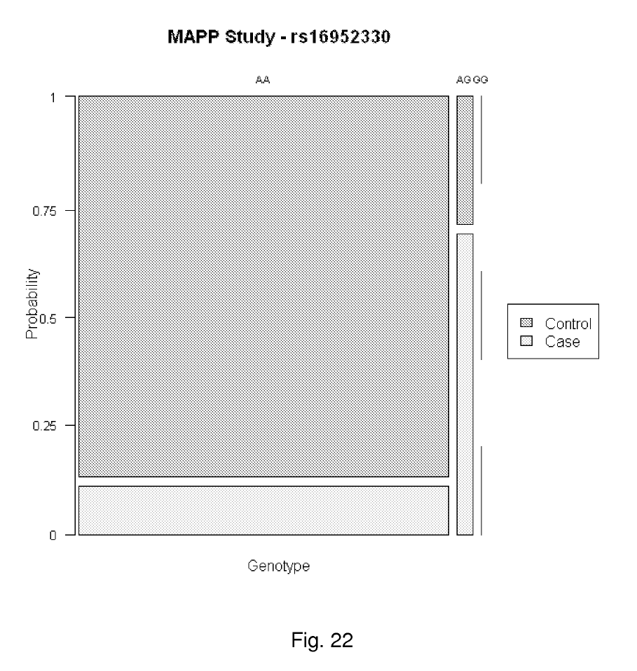

FIG. 22 illustrates the resulting risk stratification of rs16952330. As shown in the plot, the presence of a at the SNP position indicates increased susceptibility to SCA or SCD as compared to the presence of g at the SNP position. Patients with genotype a/a have about an 11% probability of experiencing SCA or SCD, while the a/g genotype indicates about a 70% probability, and no patients in the case or control populations had the genotype g/g. Table 14 shows the statistical breakdown of the genotypes for this SNP.

TABLE 14
Table of rs16952330 by arm

| rs16952330 | arm | | |
|---|---|---|---|
| Count | | | |
| Frequency % | | | |
| Row Pct | | | |
| Col Pct | Control | Case | Total |
| AA | 203 | 26 | 229 |
| | 84.94 | 10.88 | 95.82 |
| | 88.65 | 11.35 | |
| | 98.54 | 78.79 | |
| AG | 3 | 7 | 10 |
| | 1.26 | 2.93 | 4.18 |
| | 30.00 | 70.00 | |
| | 1.46 | 21.21 | |
| Total | 206 | 33 | 239 |
| | 86.19 | 13.81 | 100.00 |

Frequency Missing = 1

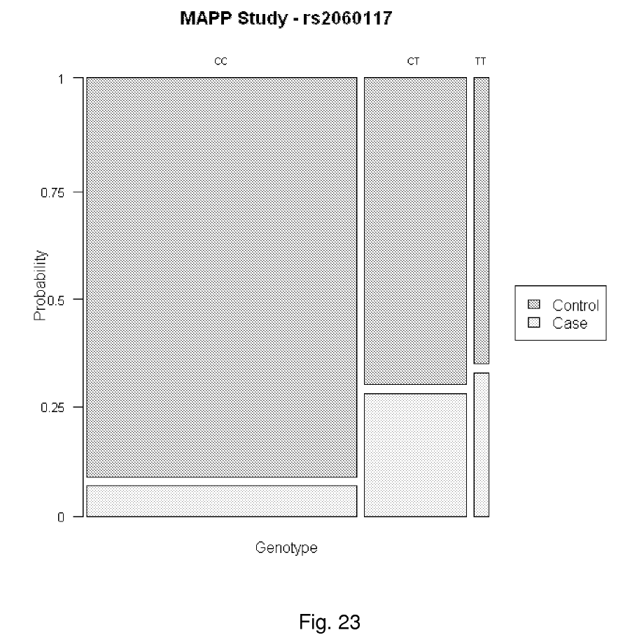

FIG. 23 illustrates the resulting risk stratification of rs2060117. As shown in the plot, the presence of c at the SNP position indicates decreased susceptibility to SCA or SCD as compared to the presence of t at the SNP position. Patients with genotype c/c have about a 7% probability of experiencing SCA or SCD, while the c/t genotype indicates about a 29% probability, and the tit genotype indicates about a 33% probability of experiencing SCA or SCD. Table 15 shows the statistical breakdown of the genotypes for this SNP.

TABLE 15
Table of rs2060117 by arm

| rs2060117 | arm | | |
|---|---|---|---|
| Count | | | |
| Frequency % | | | |
| Row Pct | | | |
| Col Pct | Control | Case | Total |
| CC | 156 | 12 | 168 |
| | 65.00 | 5.00 | 70.00 |
| | 92.86 | 7.14 | |
| | 75.36 | 36.36 | |
| CT | 45 | 18 | 63 |
| | 18.75 | 7.50 | 26.25 |
| | 71.43 | 28.57 | |
| | 21.74 | 54.55 | |
| TT | 6 | 3 | 9 |
| | 2.50 | 1.25 | 3.75 |
| | 66.67 | 33.33 | |
| | 2.90 | 9.09 | |
| Total | 207 | 33 | 240 |
| | 86.25 | 13.75 | 100.00 |

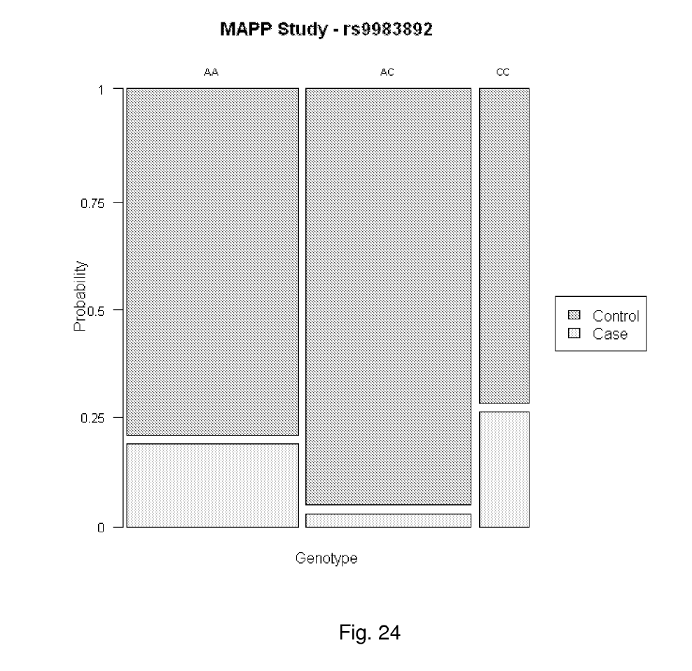

FIG. 24 illustrates the resulting risk stratification of rs9983892. As shown in the plot, the presence of a at the SNP position indicates decreased susceptibility to SCA or SCD as compared to the presence of c at the SNP position. Patients with genotype a/a have about a 19% probability of experiencing SCA or SCD, while the a/c genotype indicates less than 5% probability, and the c/c genotype indicates about a 27% probability of experiencing SCD or SCA. Table 16 shows the statistical breakdown of the genotypes for this SNP.

TABLE 16
Table of rs9983892 by arm

| rs9983892 | arm | | |
|---|---|---|---|
| Count | | | |
| Frequency % | | | |
| Row % | | | |
| Col % | Control | Case | Total |
| AA | 84 | 20 | 104 |
| | 35.90 | 8.55 | 44.44 |
| | 80.77 | 19.23 | |
| | 41.38 | 64.52 | |
| AC | 97 | 3 | 100 |
| | 41.45 | 1.28 | 42.74 |
| | 97.00 | 3.00 | |
| | 47.78 | 9.68 | |
| CC | 22 | 8 | 30 |
| | 9.40 | 3.42 | 12.82 |
| | 73.33 | 26.67 | |
| | 10.84 | 25.81 | |
| Total | 203 | 31 | 234 |
| | 86.75 | 13.25 | 100.00 |

Frequency Missing = 6

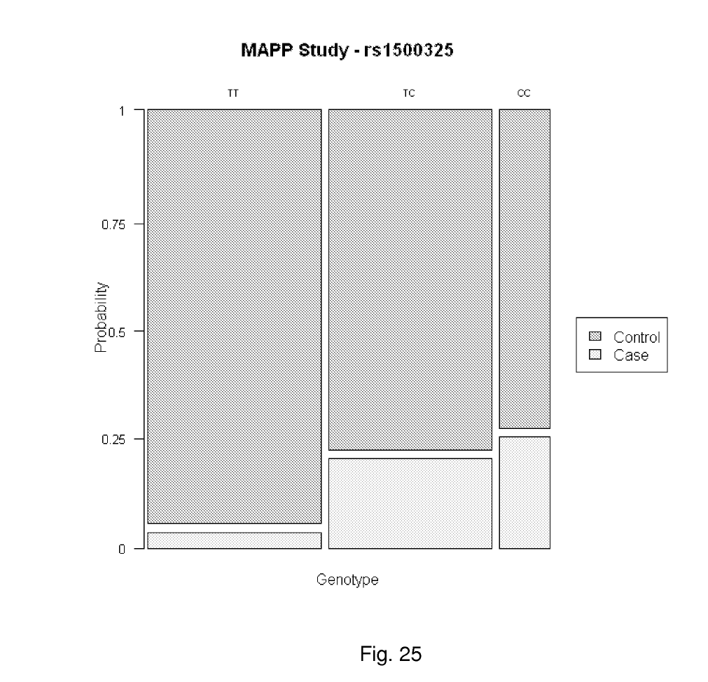

FIG. 25 illustrates the resulting risk stratification of rs1500325. As shown in the plot, the presence of t at the SNP position indicates decreased susceptibility to SCA or SCD as compared to the presence of c at the SNP position. Patients with genotype t/t have less than 5% probability of experiencing SCA or SCD, while the t/c genotype indicates about a 21% probability, and the c/c genotype indicates about a 26% probability of experiencing SCA or SCD. Table 17 shows the statistical breakdown of the genotypes for this SNP.

TABLE 17
Table of rs1500325 by arm

| rs1500325 | arm | | |
|---|---|---|---|
| Count | | | |
| Frequency % | | | |
| Row % | | | |
| Col % | Control | Case | Total |
| CC | 23 | 8 | 31 |
| | 9.58 | 3.33 | 12.92 |
| | 74.19 | 25.81 | |
| | 11.11 | 24.24 | |
| CT | 80 | 21 | 101 |
| | 33.33 | 8.75 | 42.08 |
| | 79.21 | 20.79 | |
| | 38.65 | 63.64 | |
| TT | 104 | 4 | 108 |
| | 43.33 | 1.67 | 45.00 |
| | 96.30 | 3.70 | |
| | 50.24 | 12.12 | |
| Total | 207 | 33 | 240 |
| | 86.25 | 13.75 | 100.00 |

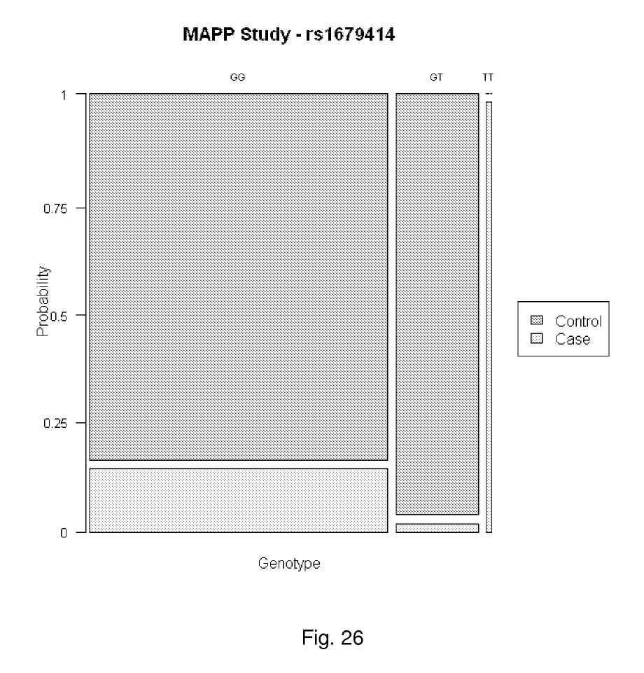

FIG. 26 illustrates the resulting risk stratification of rs1679414. As shown in the plot, the presence of g at the SNP position indicates decreased susceptibility to SCA or SCD as compared to the presence of t at the SNP position. Patients with genotype g/g have about a 15% probability of experiencing SCA or SCD, while the g/t genotype indicates less than 5% probability, and the t/t genotype indicates greater than 99% probability of experiencing SCA or SCD. Table 18 shows the statistical breakdown of the genotypes for this SNP.

TABLE 18
Table of rs1679414 by arm

| rs1679414 | arm | | |
|---|---|---|---|
| Count | | | |
| Frequency % | | | |
| Row % | | | |
| Col % | Control | Case | Total |
| GG | 157 | 27 | 184 |
| | 65.97 | 11.34 | 77.31 |
| | 85.33 | 14.67 | |
| | 75.85 | 87.10 | |
| GT | 50 | 1 | 51 |
| | 21.01 | 0.42 | 21.43 |
| | 98.04 | 1.96 | |
| | 24.15 | 3.23 | |
| TT | 0 | 3 | 3 |
| | 0.00 | 1.26 | 1.26 |
| | 0.00 | 100.00 | |
| | 0.00 | 9.68 | |
| Total | 207 | 31 | 238 |
| | 86.97 | 13.03 | 100.00 |

Frequency Missing = 2

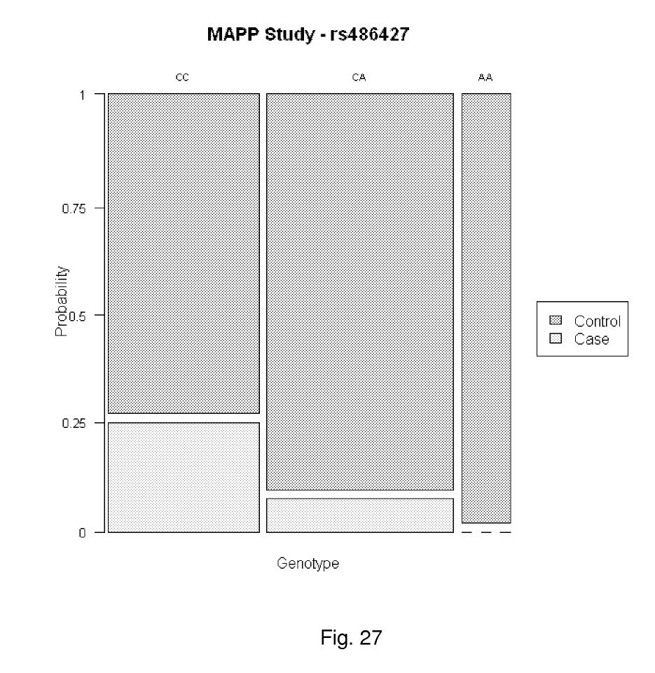

FIG. 27 illustrates the resulting risk stratification of rs486427. As shown in the plot, the presence of c at the SNP position indicates increased susceptibility to SCA or SCD as compared to the presence of a the SNP position. Patients with genotype c/c have about a 26% probability of experiencing SCA or SCD, while the c/a genotype indicates about an 8% probability, and the a/a genotype indicates less than 1% probability of experiencing SCA or SCD. Table 19 shows the statistical breakdown of the genotypes for this SNP.

TABLE 19
Table of rs486427 by arm

| rs486427 | arm | | |
|---|---|---|---|
| Count | | | |
| Frequency % | | | |
| Row % | | | |
| Col % | Control | Case | Total |
| AA | 30 | 0 | 30 |
| | 12.50 | 0.00 | 12.50 |
| | 100.00 | 0.00 | |
| | 14.49 | 0.00 | |
| AC | 107 | 9 | 116 |
| | 44.58 | 3.75 | 48.33 |
| | 92.24 | 7.76 | |
| | 51.69 | 27.27 | |
| CC | 70 | 24 | 94 |
| | 29.17 | 10.00 | 39.17 |
| | 74.47 | 25.53 | |
| | 33.82 | 72.73 | |
| Total | 207 | 33 | 240 |
| | 86.25 | 13.75 | 100.00 |

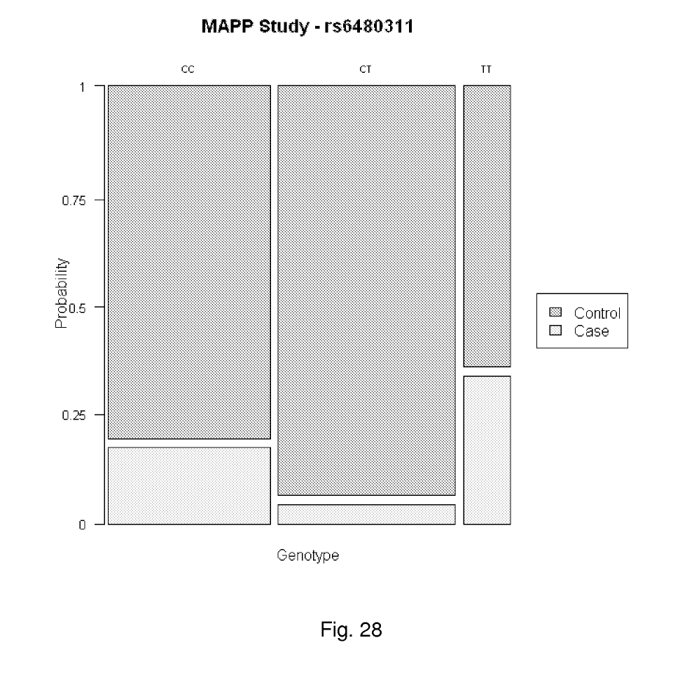

FIG. 28 illustrates the resulting risk stratification of rs6480311. As shown in the plot, the presence of c at the SNP position indicates decreased susceptibility to SCA or SCD as compared to the presence of t at the SNP position. Patients with genotype c/c have about an 18% probability of experiencing SCA or SCD, while the c/t genotype indicates about a 5% probability, and the t/t genotype indicates about a 35% probability of experiencing SCA or SCD. Table 20 shows the statistical breakdown of the genotypes for this SNP.

TABLE 20
Table of rs6480311 by arm

| rs6480311 | arm | | |
|---|---|---|---|
| Count | | | |
| Frequency % | | | |
| Row % | | | |
| Col % | Control | Case | Total |
| CC | 83 | 18 | 101 |
| | 34.58 | 7.50 | 42.08 |
| | 82.18 | 17.82 | |
| | 40.10 | 54.55 | |
| CT | 105 | 5 | 110 |
| | 43.75 | 2.08 | 45.83 |
| | 95.45 | 4.55 | |
| | 50.72 | 15.15 | |
| TT | 19 | 10 | 29 |
| | 7.92 | 4.17 | 12.08 |
| | 65.52 | 34.48 | |
| | 9.18 | 30.30 | |
| Total | 207 | 33 | 240 |
| | 86.25 | 13.75 | 100.00 |

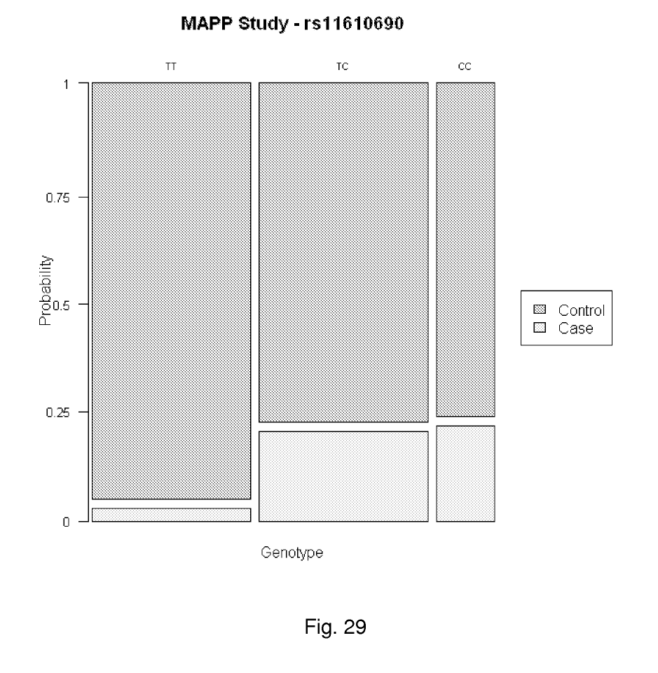

FIG. 29 illustrates the resulting risk stratification of rs11610690. As shown in the plot, the presence of t at the SNP position indicates decreased susceptibility to SCA or SCD as compared to the presence of c at the SNP position. Patients with genotype t/t have less than 5% probability of experiencing SCA or SCD, while the t/c genotype indicates about a 21% probability, and the c/c genotype indicates about a 22% probability of experiencing SCA or SCD. Table 21 shows the statistical breakdown of the genotypes for this SNP.

TABLE 21
Table of rs11610690 by arm

| rs11610690 | arm | | |
|---|---|---|---|
| Count | | | |
| Frequency % | | | |
| Row % | | | |
| Col % | Control | Case | Total |
| CC | 28 | 8 | 36 |
| | 11.67 | 3.33 | 15.00 |
| | 77.78 | 22.22 | |
| | 13.53 | 24.24 | |
| CT | 83 | 22 | 105 |
| | 34.58 | 9.17 | 43.75 |
| | 79.05 | 20.95 | |
| | 40.10 | 66.67 | |
| TT | 96 | 3 | 99 |
| | 40.00 | 1.25 | 41.25 |
| | 96.97 | 3.03 | |
| | 46.38 | 9.09 | |
| Total | 207 | 33 | 240 |
| | 86.25 | 13.75 | 100.00 |

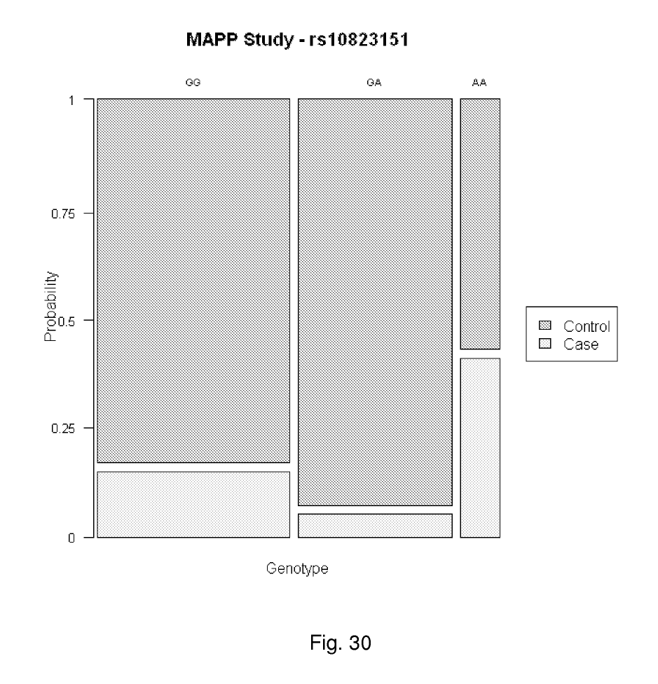

FIG. 30 illustrates the resulting risk stratification of rs10823151. As shown in the plot, the presence of g at the SNP position indicates decreased susceptibility to SCA or SCD as compared to the presence of a at the SNP position. Patients with genotype g/g have about a 15% probability of experiencing SCA or SCD, while the g/a genotype indicates about a 5% probability, and the a/a genotype indicates about a 42% probability of experiencing SCA or SCD. Table 22 shows the statistical breakdown of the genotypes for this SNP.

TABLE 22
Table of rs10823151 by arm

| rs10823151 | arm | | |
|---|---|---|---|
| Count | | | |
| Frequency % | | | |
| Row % | | | |
| Col % | Control | Case | Total |
| AA | 14 | 10 | 24 |
| | 5.93 | 4.24 | 10.17 |
| | 58.33 | 41.67 | |
| | 6.90 | 30.30 | |
| AG | 89 | 5 | 94 |
| | 37.71 | 2.12 | 39.83 |
| | 94.68 | 5.32 | |
| | 43.84 | 15.15 | |
| GG | 100 | 18 | 118 |
| | 42.37 | 7.63 | 50.00 |
| | 84.75 | 15.25 | |
| | 49.26 | 54.55 | |
| Total | 203 | 33 | 236 |
| | 86.02 | 13.98 | 100.00 |

Frequency Missing = 4

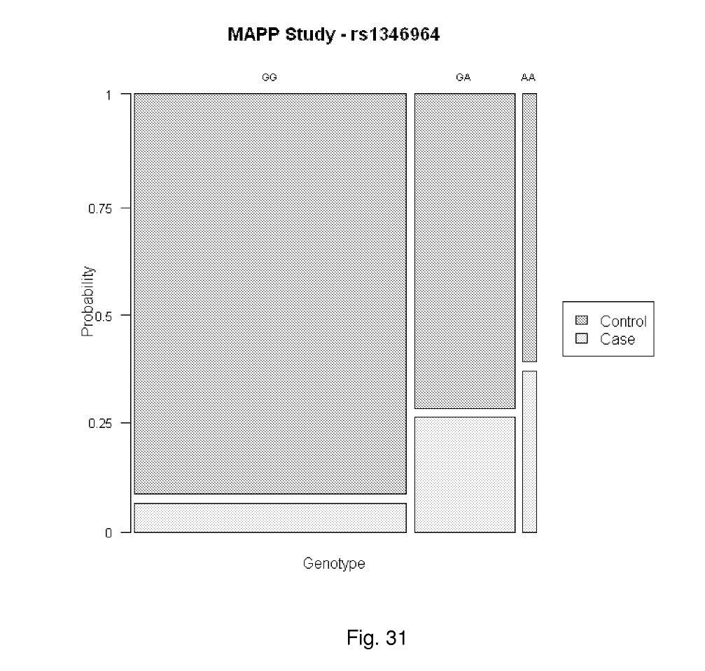

FIG. 31 illustrates the resulting risk stratification of rs1346964. As shown in the plot, the presence of g at the SNP position indicates decreased susceptibility to SCA or SCD as compared to the presence of a at the SNP position. Patients with genotype g/g have about a 7% probability of experiencing SCA or SCD, while the g/a genotype indicates about a 27% probability, and the a/a genotype indicates about a 37% probability of experiencing SCA or SCD. Table 23 shows the statistical breakdown of the genotypes for this SNP.

TABLE 23
Table of rs1346964 by arm

| rs1346964 | arm | | |
|---|---|---|---|
| Count | | | |
| Frequency % | | | |
| Row % | | | |
| Col % | Control | Case | Total |
| AA | 5 | 3 | 8 |
| | 2.16 | 1.30 | 3.46 |
| | 62.50 | 37.50 | |
| | 2.49 | 10.00 | |
| AG | 44 | 16 | 60 |
| | 19.05 | 6.93 | 25.97 |
| | 73.33 | 26.67 | |
| | 21.89 | 53.33 | |
| GG | 152 | 11 | 163 |
| | 65.80 | 4.76 | 70.56 |
| | 93.25 | 6.75 | |
| | 75.62 | 36.67 | |
| Total | 201 | 30 | 231 |
| | 87.01 | 12.99 | 100.00 |

Frequency Missing = 9

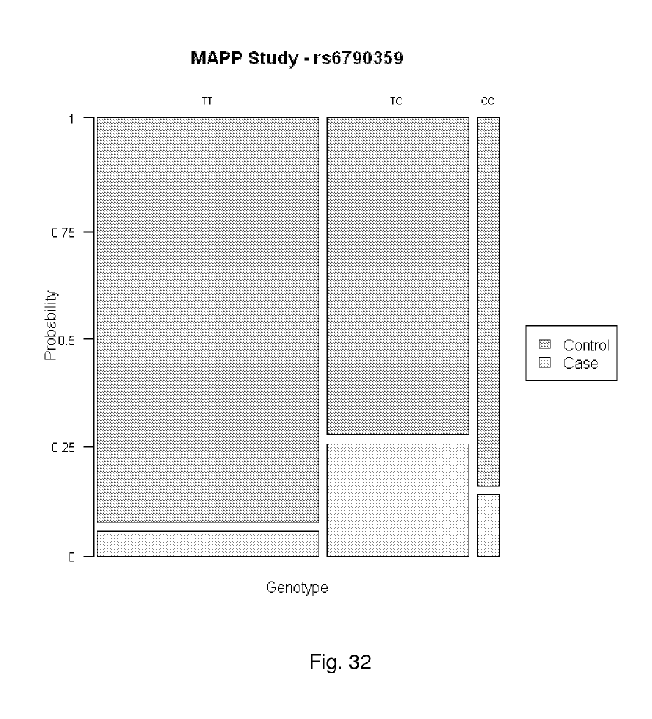

FIG. 32 illustrates the resulting risk stratification of rs6790359. As shown in the plot, the presence of t at the SNP position indicates decreased susceptibility to SCA or SCD as compared to the presence of c at the SNP position. Patients with genotype t/t have about a 65% probability of experiencing SCA or SCD, while the t/c genotype indicates about a 26% probability, and the c/c genotype indicates about a 14% probability of experiencing SCA or SCD Table 24 shows the statistical breakdown of the genotypes for this SNP.

TABLE 24
Table of rs6790359 by arm

| rs6790359 | arm | | |
|---|---|---|---|
| Count | | | |
| Frequency % | | | |
| Row Pct | | | |
| Col Pct | Control | Case | Total |
| CC | 12 | 2 | 14 |
| | 5.00 | 0.83 | 5.83 |
| | 85.71 | 14.29 | |
| | 5.80 | 6.06 | |
| CT | 65 | 23 | 88 |
| | 27.08 | 9.58 | 36.67 |
| | 73.86 | 26.14 | |
| | 31.40 | 69.70 | |
| TT | 130 | 8 | 138 |
| | 54.17 | 3.33 | 57.50 |
| | 94.20 | 5.80 | |
| | 62.80 | 24.24 | |
| Total | 207 | 33 | 240 |
| | 86.25 | 13.75 | 100.00 |

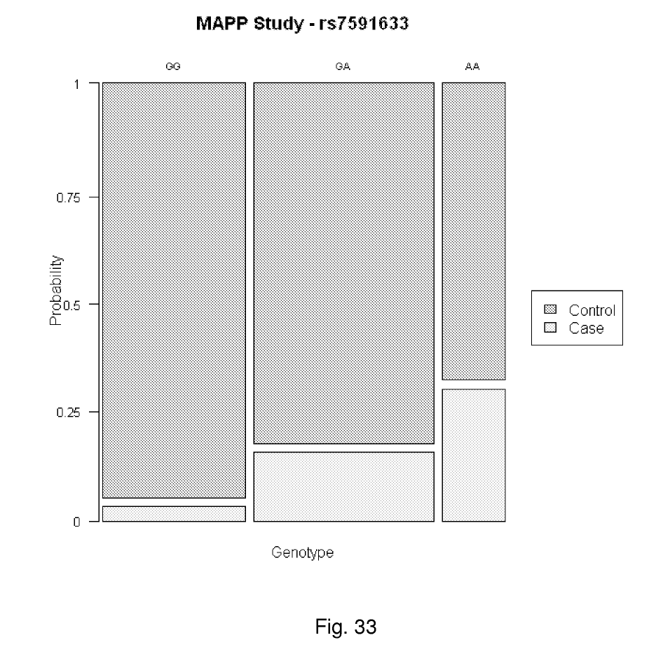

FIG. 33 illustrates the resulting risk stratification of rs7591633. As shown in the plot, the presence of g at the SNP position indicates decreased susceptibility to SCA or SCD as compared to the presence of a at the SNP position. Patients with genotype g/g have less than 5% probability of experiencing SCA or SCD, while the g/a genotype indicates about a 16% probability, and the a/a genotype indicates about a 31% probability of experiencing SCA or SCD. Table 25 shows the statistical breakdown of the genotypes for this SNP.

TABLE 25
Table of rs7591633 by arm

| rs7591633 | arm | | |
|---|---|---|---|
| Count | | | |
| Frequency % | | | |
| Row % | | | |
| Col % | Control | Case | Total |
| AA | 27 | 12 | 39 |
| | 11.25 | 5.00 | 16.25 |
| | 69.23 | 30.77 | |
| | 13.04 | 36.36 | |
| AG | 94 | 18 | 112 |
| | 39.17 | 7.50 | 46.67 |
| | 83.93 | 16.07 | |
| | 45.41 | 54.55 | |
| GG | 86 | 3 | 89 |
| | 35.83 | 1.25 | 37.08 |
| | 96.63 | 3.37 | |
| | 41.55 | 9.09 | |
| Total | 207 | 33 | 240 |
| | 86.25 | 13.75 | 100.00 |

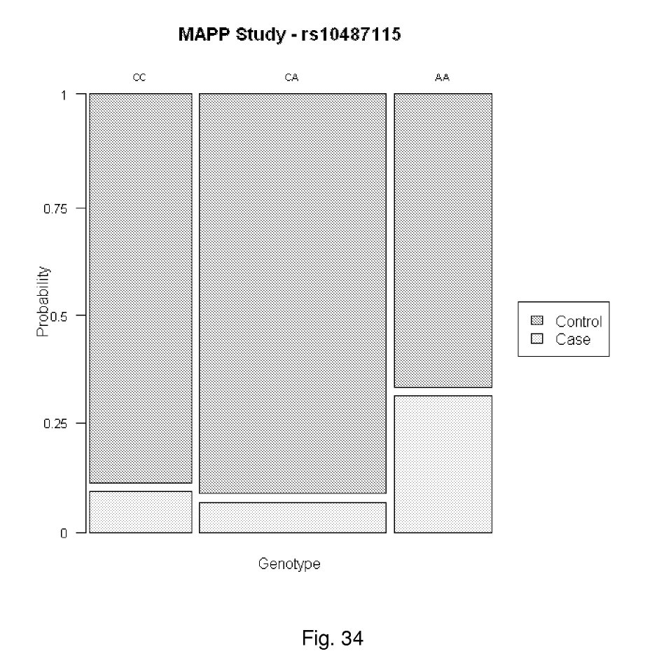

FIG. 34 illustrates the resulting risk stratification of rs10487115. As shown in the plot, the presence of c at the SNP position indicates decreased susceptibility to SCA or SCD as compared to the presence of a at the SNP position. Patients with genotype c/c have about a 10% probability of experiencing SCA or SCD, while the c/a genotype indicates about a 7% probability, and the a/a genotype indicates about a 32% probability of experiencing SCA or SCD. Table 26 shows the statistical breakdown of the genotypes for this SNP.

TABLE 26
Table of rs10487115 by arm

| rs10487115 | arm | | |
|---|---|---|---|
| Count | | | |
| Frequency % | | | |
| Row % | | | |
| Col % | Control | Case | Total |
| AA | 41 | 19 | 60 |
| | 17.23 | 7.98 | 25.21 |
| | 68.33 | 31.67 | |
| | 20.00 | 57.58 | |
| AC | 107 | 8 | 115 |
| | 44.96 | 3.36 | 48.32 |
| | 93.04 | 6.96 | |
| | 52.20 | 24.24 | |
| CC | 57 | 6 | 63 |
| | 23.95 | 2.52 | 26.47 |
| | 90.48 | 9.52 | |
| | 27.80 | 18.18 | |
| Total | 205 | 33 | 238 |
| | 86.13 | 13.87 | 100.00 |

Frequency Missing = 2

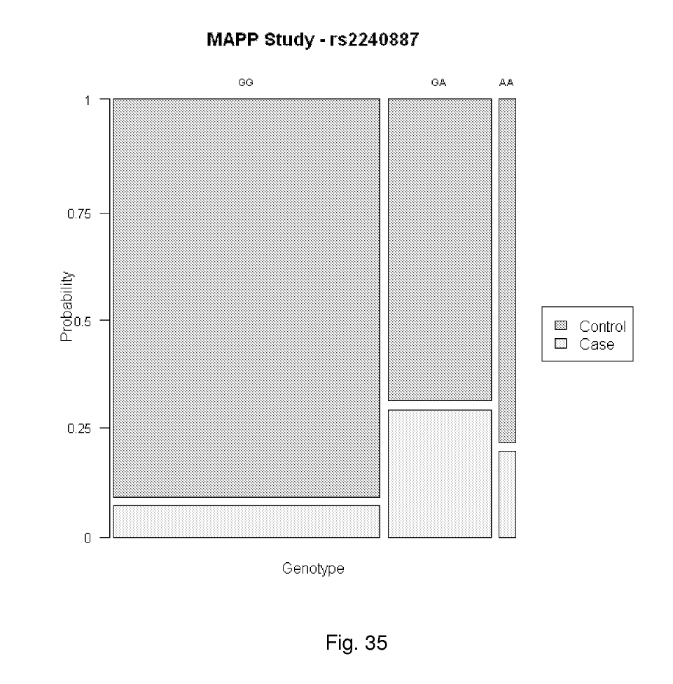

FIG. 35 illustrates the resulting risk stratification of rs2240887. As shown in the plot, the presence of g at the SNP position indicates decreased susceptibility to SCA or SCD as compared to the presence of a at the SNP position. Patients with genotype g/g have about a 7% probability of experiencing SCA or SCD, while the g/a genotype indicates about a 30% probability, and the a/a genotype indicates about a 20% probability of experiencing SCA or SCD. Table 27 shows the statistical breakdown of the genotypes for this SNP.

TABLE 27
Table of rs2240887 by arm

| rs2240887 | arm | | |
|---|---|---|---|
| Count | | | |
| Frequency % | | | |
| Row % | | | |
| Col % | Control | Case | Total |
| AA | 8 | 2 | 10 |
| | 3.33 | 0.83 | 4.17 |
| | 80.00 | 20.00 | |
| | 3.86 | 6.06 | |
| AG | 45 | 19 | 64 |
| | 18.75 | 7.92 | 26.67 |
| | 70.31 | 29.69 | |
| | 21.74 | 57.58 | |
| GG | 154 | 12 | 166 |
| | 64.17 | 5.00 | 69.17 |
| | 92.77 | 7.23 | |
| | 74.40 | 36.36 | |
| Total | 207 | 33 | 240 |
| | 86.25 | 13.75 | 100.00 |

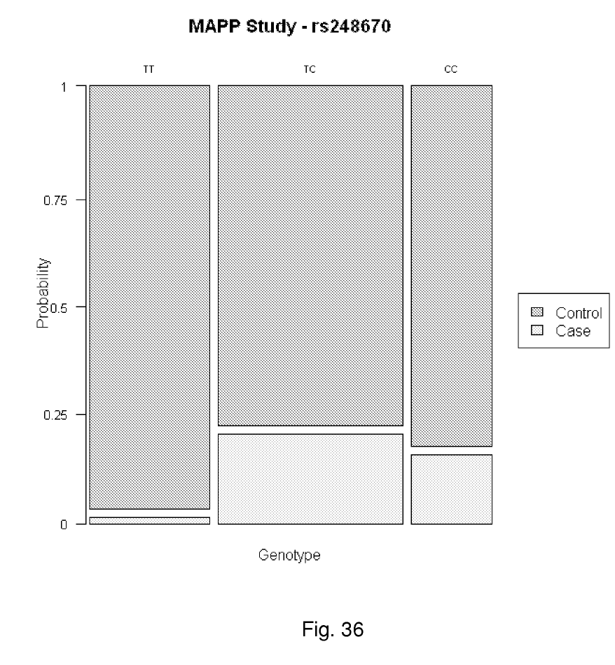

FIG. 36 illustrates the resulting risk stratification of rs248670. As shown in the plot, the presence of t at the SNP position indicates decreased susceptibility to SCA or SCD as compared to the presence of c at the SNP position. Patients with genotype t/t have less than 5% probability of experiencing SCA or SCD, while the t/c genotype indicates about a 21% probability, and the c/c genotype indicates about a 16% probability of experiencing SCA or SCD. Table 28 shows the statistical breakdown of the genotypes for this SNP.

TABLE 28
Table of rs248670 by arm

| rs248670 | arm | | |
|---|---|---|---|
| Count | | | |
| Frequency % | | | |
| Row % | | | |
| Col % | Control | Case | Total |
| CC | 42 | 8 | 50 |
| | 17.50 | 3.33 | 20.83 |
| | 84.00 | 16.00 | |
| | 20.29 | 24.24 | |
| CT | 91 | 24 | 115 |
| | 37.92 | 10.00 | 47.92 |
| | 79.13 | 20.87 | |
| | 43.96 | 72.73 | |
| TT | 74 | 1 | 75 |
| | 30.83 | 0.42 | 31.25 |
| | 98.67 | 1.33 | |
| | 35.75 | 3.03 | |
| Total | 207 | 33 | 240 |
| | 86.25 | 13.75 | 100.00 |

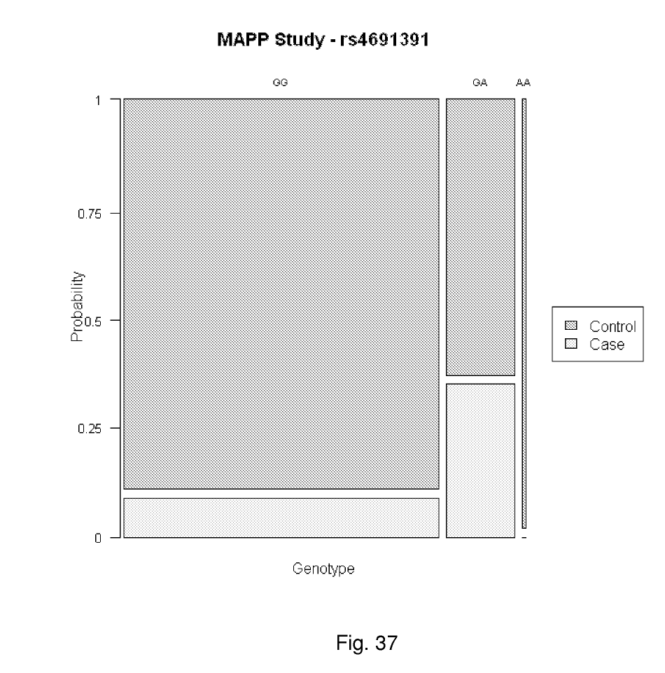

FIG. 37 illustrates the resulting risk stratification of rs4691391. As shown in the plot, the presence of g at the SNP position indicates increased susceptibility to SCA or SCD as compared to the presence of a at the SNP position. Patients with genotype g/g have about a 9% probability of experiencing SCA or SCD, while the g/a genotype indicates about a 36% probability, and the a/a genotype indicates less than 1% probability of experiencing SCA or SCD. Table 29 shows the statistical breakdown of the genotypes for this SNP.

TABLE 29
Table of rs4691391 by arm

| rs4691391 | arm | | |
|---|---|---|---|
| Count | | | |
| Frequency % | | | |
| Row % | | | |
| Col % | Control | Case | Total |
| AA | 2 | 0 | 2 |
| | 0.83 | 0.00 | 0.83 |
| | 100.00 | 0.00 | |
| | 0.97 | 0.00 | |
| AG | 27 | 15 | 42 |
| | 11.25 | 6.25 | 17.50 |
| | 64.29 | 35.71 | |
| | 13.04 | 45.45 | |
| GG | 178 | 18 | 196 |
| | 74.17 | 7.50 | 81.67 |
| | 90.82 | 9.18 | |
| | 85.99 | 54.55 | |
| Total | 207 | 33 | 240 |
| | 86.25 | 13.75 | 100.00 |

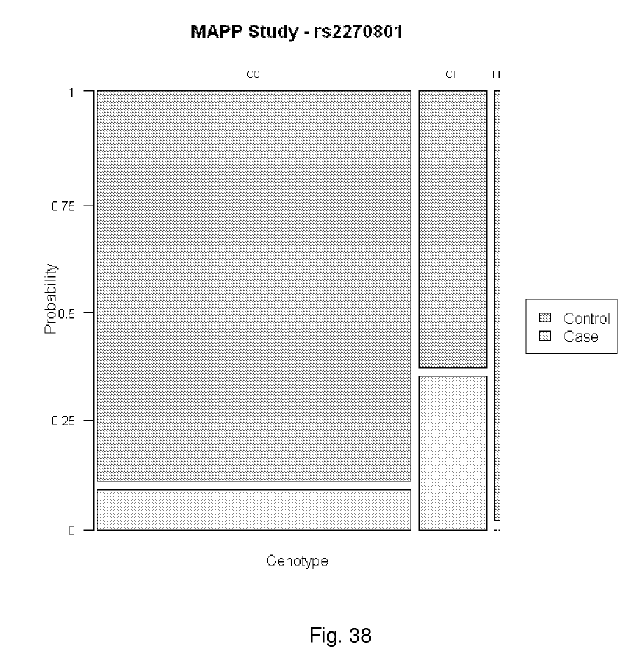

FIG. 38 illustrates the resulting risk stratification of rs2270801. As shown in the plot, the presence of c at the SNP position indicates increased susceptibility to SCA or SCD as compared to the presence of t at the SNP position. Patients with genotype c/c have about a 9% probability of experiencing SCA or SCD, while the c/t genotype indicates about a 36% probability, and the t/t genotype indicates less than 1% probability of experiencing SCA or SCD. Table 30 shows the statistical breakdown of the genotypes for this SNP.

TABLE 30
Table of rs2270801 by arm

| rs2270801 | arm | | |
|---|---|---|---|
| Count | | | |
| Frequency % | | | |
| Row % | | | |
| Col % | Control | Case | Total |
| CC | 177 | 18 | 195 |
| | 73.75 | 7.50 | 81.25 |
| | 90.77 | 9.23 | |
| | 85.51 | 54.55 | |
| CT | 27 | 15 | 42 |
| | 11.25 | 6.25 | 17.50 |
| | 64.29 | 35.71 | |
| | 13.04 | 45.45 | |
| TT | 3 | 0 | 3 |
| | 1.25 | 0.00 | 1.25 |
| | 100.00 | 0.00 | |
| | 1.45 | 0.00 | |
| Total | 207 | 33 | 240 |
| | 86.25 | 13.75 | 100.00 |

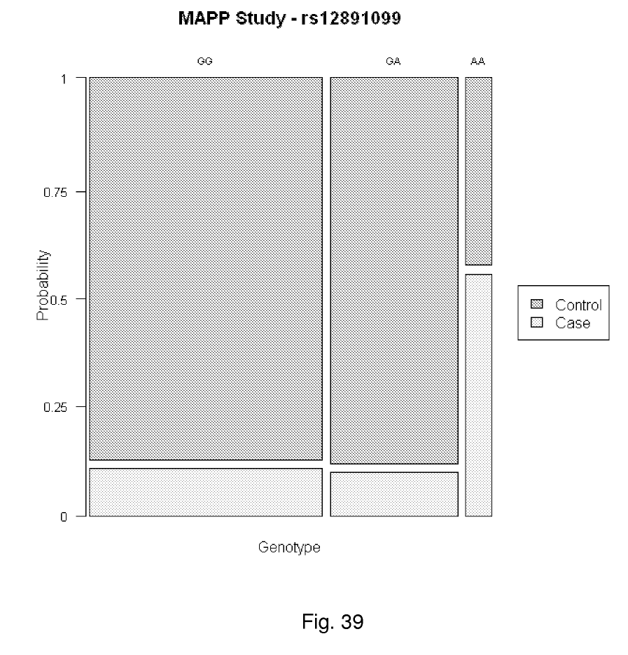

FIG. 39 illustrates the resulting risk stratification of rs12891099. As shown in the plot, the presence of g at the SNP position indicates decreased susceptibility to SCA or SCD as compared to the presence of a at the SNP position. Patients with genotype g/g have about an 11% probability of experiencing SCA or SCD, while the g/a genotype indicates about a 10% probability, and the a/a genotype indicates about a 56% probability of experiencing SCA or SCD. Table 31 shows the statistical breakdown of the genotypes for this SNP.

TABLE 31
Table of rs12891099 by arm

| rs12891099 | arm | | |
|---|---|---|---|
| Count | | | |
| Frequency % | | | |
| Row % | | | |
| Col % | Control | Case | Total |
| AA | 7 | 9 | 16 |
| | 2.92 | 3.75 | 6.67 |
| | 43.75 | 56.25 | |
| | 3.38 | 27.27 | |
| AG | 71 | 8 | 79 |
| | 29.58 | 3.33 | 32.92 |
| | 89.87 | 10.13 | |
| | 34.30 | 24.24 | |
| GG | 129 | 16 | 145 |
| | 53.75 | 6.67 | 60.42 |
| | 88.97 | 11.03 | |
| | 62.32 | 48.48 | |
| Total | 207 | 33 | 240 |
| | 86.25 | 13.75 | 100.00 |

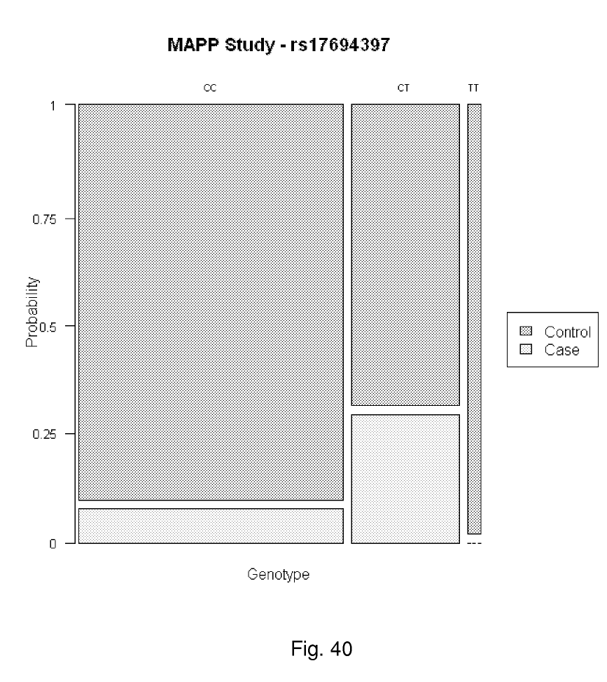

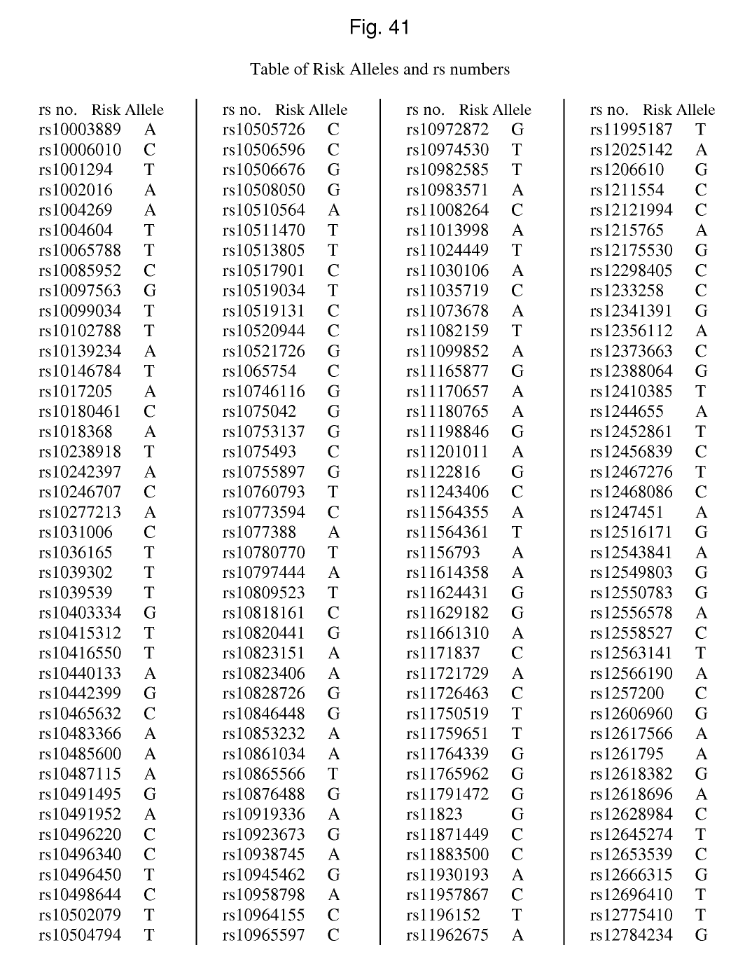

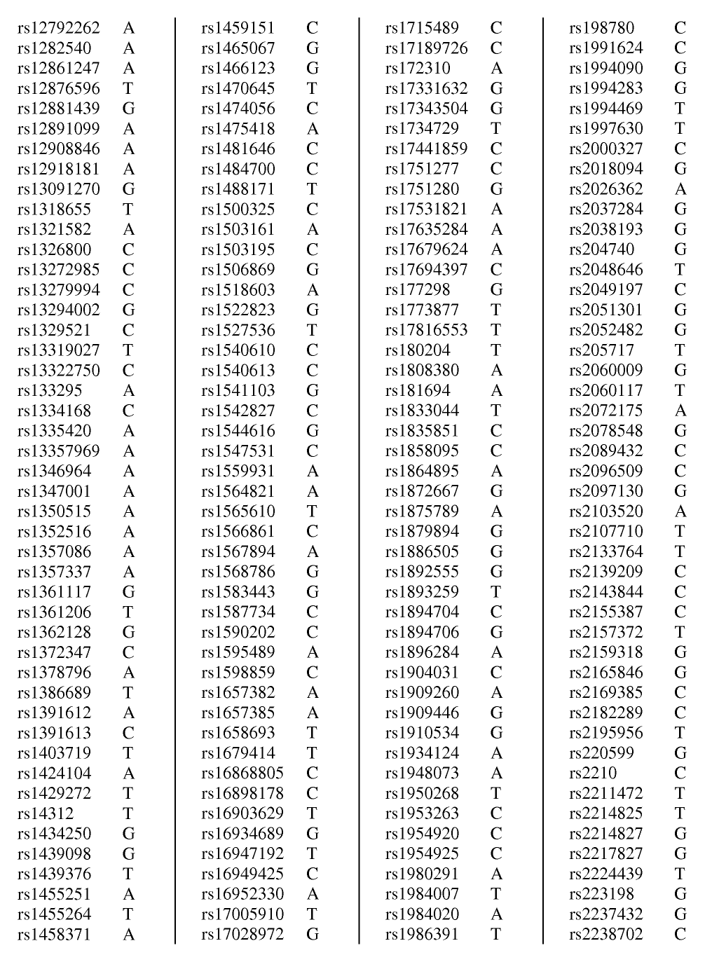

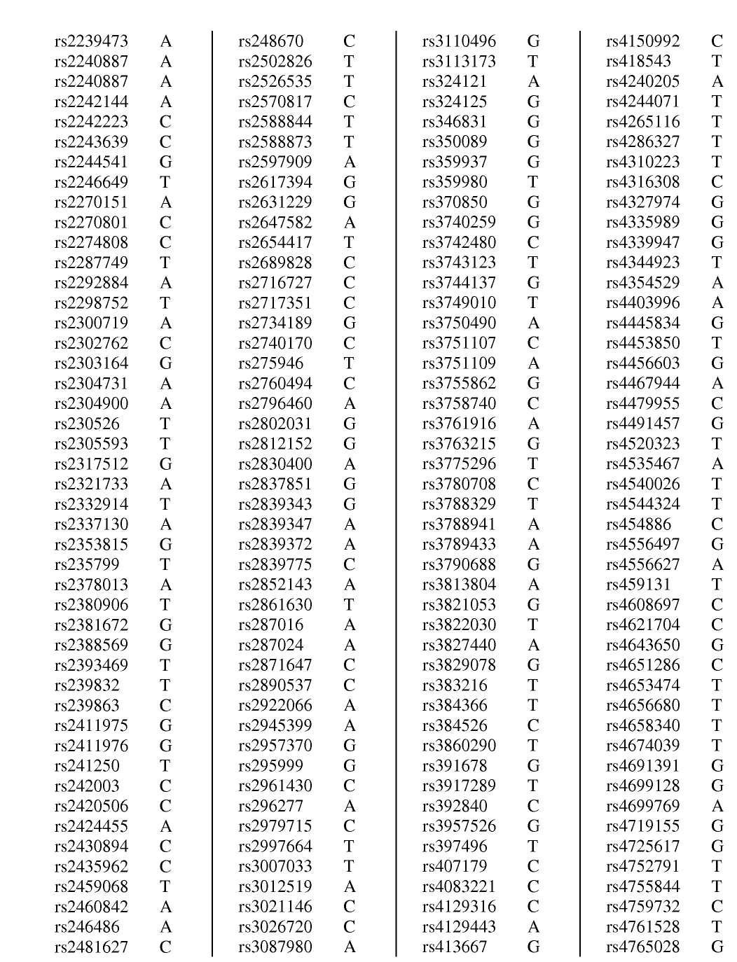

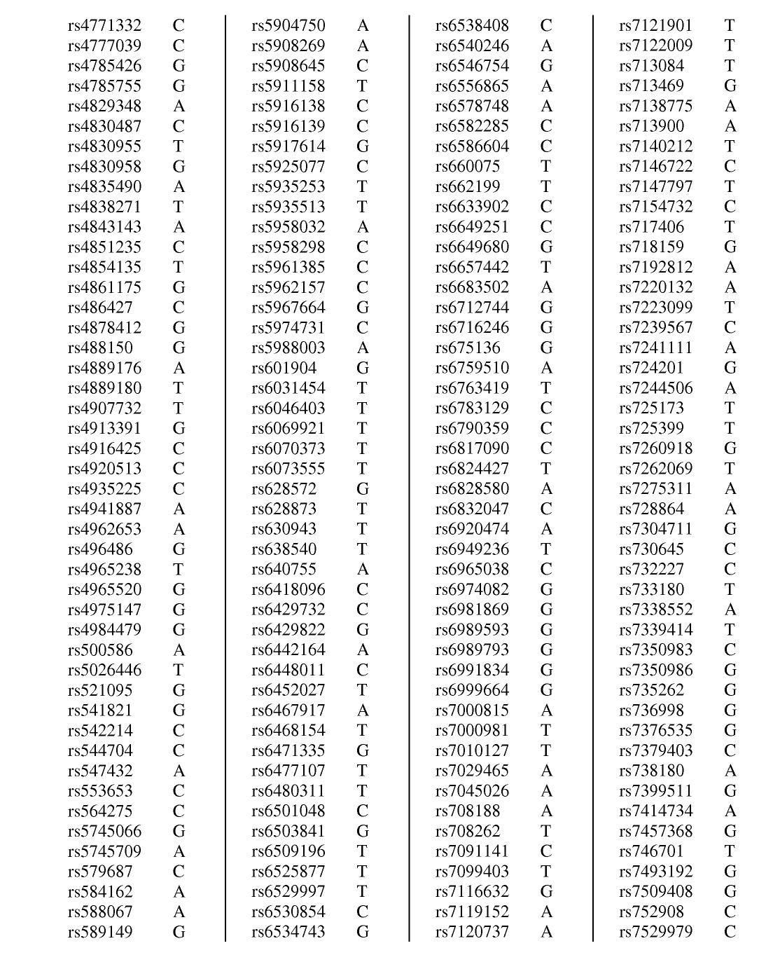

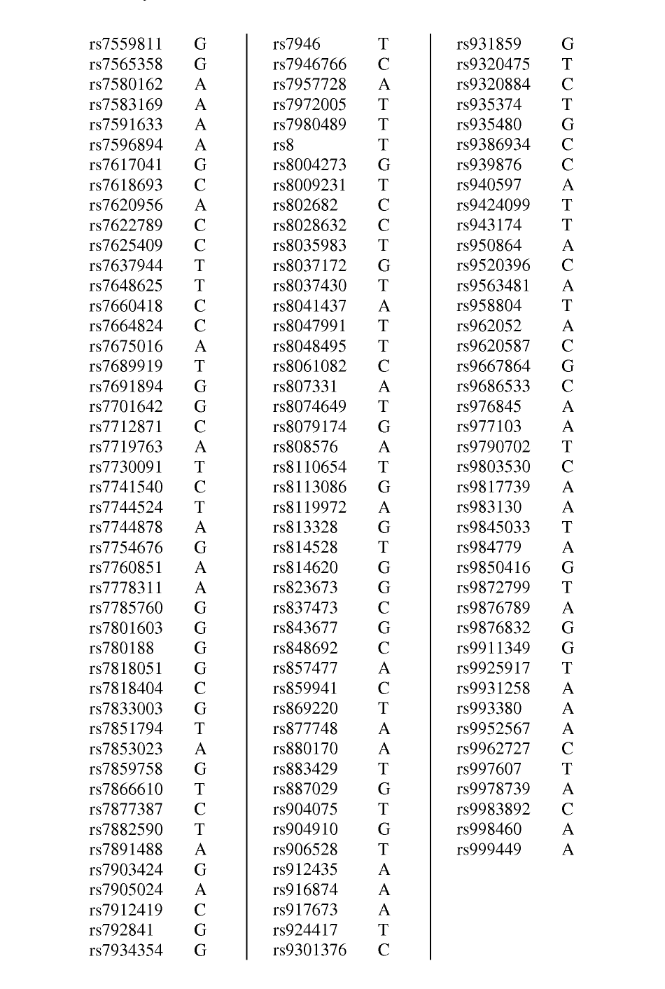

FIG. 40 illustrates the resulting risk stratification of rs17694397. As shown in the plot, the presence of c at the SNP position indicates increased susceptibility to SCA or SCD as compared to the presence of t at the SNP position. Patients with genotype c/c have about an 8% probability of experiencing SCA or SCD, while the c/t genotype indicates about a 30% probability, and the t/t genotype indicates less than 1% probability of experiencing SCA or SCD. Table 32 shows the statistical breakdown of the genotypes for this SNP.

TABLE 32
Table of rs17694397 by arm

| rs17694397 | arm | | |
|---|---|---|---|
| Count | | | |
| Frequency % | | | |
| Row % | | | |
| Col % | Control | Case | Total |
| CC | 151 | 13 | 164 |
| | 63.18 | 5.44 | 68.62 |
| | 92.07 | 7.93 | |
| | 73.30 | 39.39 | |
| CT | 47 | 20 | 67 |
| | 19.67 | 8.37 | 28.03 |
| | 70.15 | 29.85 | |
| | 22.82 | 60.61 | |
| TT | 8 | 0 | 8 |
| | 3.35 | 0.00 | 3.35 |
| | 100.00 | 0.00 | |
| | 3.88 | 0.00 | |
| Total | 206 | 33 | 239 |
| | 86.19 | 13.81 | 100.00 |

Frequency Missing = 1

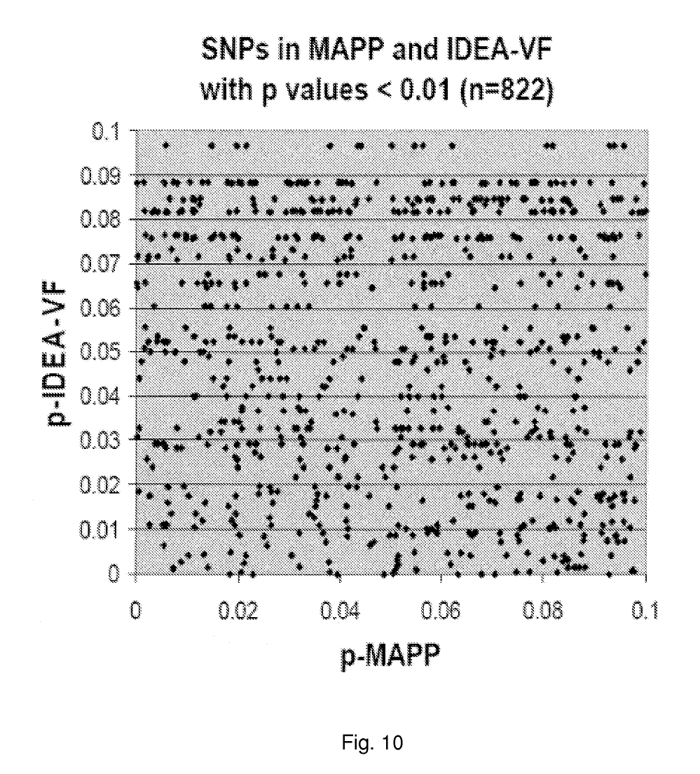

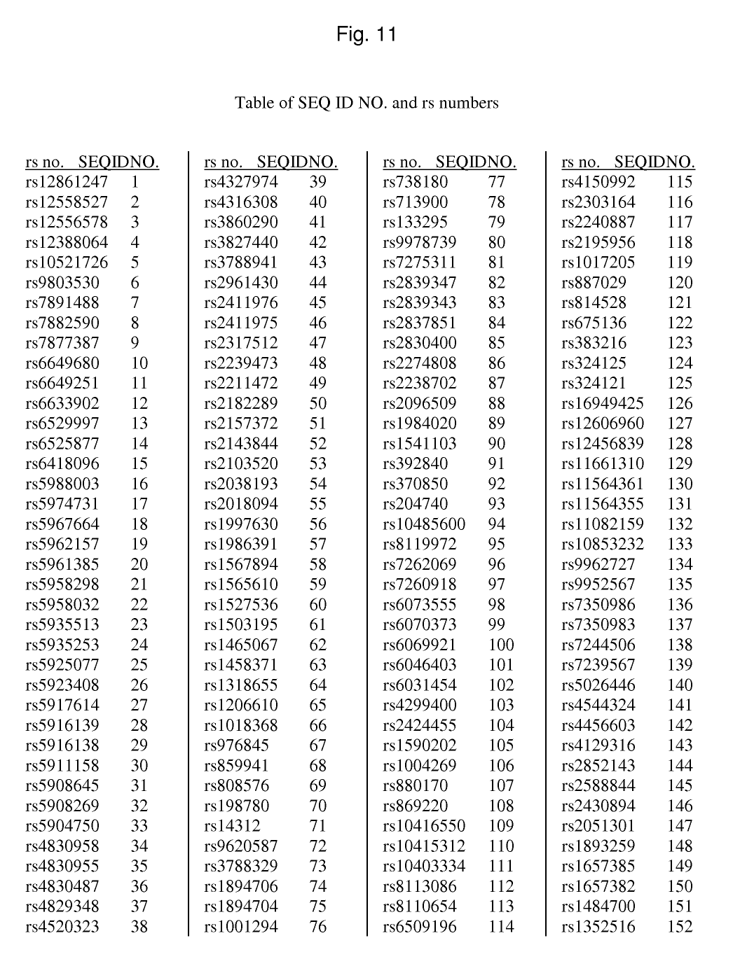

FIG. 10 shows 849 SNPs identified by the MAPP and IDEA-VF studies that are associated with risk of SCA, and is a subset of the total number of 317,503 SNPs scanned from the whole genome using the Illumina 300K HapMap gene chips described herein. FIG. 11 is a list of rs numbers and corresponding SEQ ID Nos. Both the rs numbers and the SEQ ID Nos. can be used interchangeably to identify a particular SNP.

A third study to identify genetic markers associated with SCA or SCD (referred to herein as “DISCOVERY”) has been designed and implemented. The DISCOVERY study is undertaken to determine if certain cardiac ion-channel genetic polymorphisms predispose a patient to ventricular and atrial arrhythmia. In particular, the study aimed to identify which polymorphism combinations, optionally in further combination with other markers, serve as prognostics that identify appropriate candidates for ICD therapy. The DISCOVERY study's primary objectives are to correlate genetic polymorphisms with a diagnostic stratification of patients through a determination of risk of ventricular tachycardia and to evaluate the utility of ICD-based diagnostic information on the long-term treatment and management of primary prevention ICD patients. In particular, the predictive utility of SNPs in specific genes for ventricular arrhythmia of <400 ms was evaluated. In particular, the genes studied were GNB3, GNAS and GNAQ genes, and the positive value was determined for SNPs as predictor for death, sudden cardiac death and atrial fibrillation or flutter in the genes GNB3, GNAS, GNAQ and other SNPs involving signal transduction components that have an impact on the activity of cardiac ion channels. Other genes under consideration include the CAPON and GPC5 genes. These data may be used to determine the optimal combination of all genetic parameters, including the presence or absence of any of the SNPs disclosed herein or otherwise known to be markers for cardiovascular diseases or disorders, patient baseline data, and patient follow-up data as a predictor for use in diagnostic and treatment methods and further methods of classification or stratification of patients based on the likelihood of SCA or SCD.

### Polymorphism in GNB3

The GNB3 gene consists of 12 exons localized on chromosome 12p13. It codes for the β₃-subunit of the hetero-trimeric G-proteins. The widely distributed C825T polymorphism exhibits exchange between Cytosine (C) and Thymine (T) in nucleotide position 825 of the cDNA as shown in Table 33. (Siffert, W. et al., Association of a Human G-Protein beta3 Subunit Variant with Hypertension, Nat. Genet., 1998; 18:45-48). This SNP is localized in exon 10 and associated with changes in cellular signal transduction. Id. This polymorphism is represented by rs5443 (SEQ ID No. 850) and is known by the following sequence: SEQ ID No. 850: 5′-gag agc atc atc tgc ggc atc acg tc [c/t] gtg gcc ttc tcc ctc agt ggc cgc c-3′.

As G-proteins participate in signal transduction in almost all body cells, it was shown that the C825T polymorphism is correlated with arterial hypertension, (Hengstenberg, C. et al., Association Between a Polymorphism in the G protein beta3 Subunit Gene (GNB3) with Arterial Hypertension but not with Myocardial Infarction, Cardiovasc. Res., 2001; 49:820-827) arteriosclerosis and obesity (Gutersohn, A. et al., G Protein beta3 Subunit 825 TT Genotype and Post-Pregnancy Weight Retention, Lancet, 2000; 355:1240-1241) along with changes in the response to hormones and drugs (Mitchell, A. et al., Increased Haemodynamic Response to Clonidine in Subjects Carrying the 825T-allele of the G Protein beta3 Subunit, Abstract, Naunyn-Schmiedeberg's Arch. Pharmacol., 2002; 265: Suppl. 1; Mitchell, A. et al., Insulin-mediated Venodilation Is Impaired in Young, Healthy Carriers of the 825T-allele of the G-protein beta3 Subunit Gene (GNB3), Clin. Pharmacol. Ther., 2005; 77:495-502; Mitchell, A. et al., Effects of Systemic Endothelin A Receptor Antagonism in Various Vascular Beds in Men: In Vivo Interactions of the Major Blood Pressure-regulating Systems and Associations with the GNB3 C825T Polymorphism, Clin. Pharmacol. Ther., 2004; 76:396-408; Sarrazin, C. et al., GNB3 C825T Polymorphism and Response to Interferon-alfa/ribavirin Treatment in Patients with Hepatitis C Virus Genotype 1 (CHV-1) Injection, J. Hepatol., 2005; 43:388-393; Sperling, H. et al., Sildenafil Response is Influenced by the G Protein beta3 Subunit GNB3 C825T Polymorphism: A Pilot Study, J. Urol., 2003; 169:1048-1051). U.S. Pat. No. 6,924,100 describes a method for evaluating responsiveness of an individual to treatment with an in vivo pharmaceutical wherein the in vivo pharmaceutical is one which activates G protein heterodimers containing a G protein subunit wherein the genetic modification is a substitution of cytosine by thymidine at position 825. Similarly, U.S. Pat. No. 6,242,181 describes a method for diagnosing an increased likelihood of hypertension in a human subject comprising determining the presence of a genetic modification in a gene obtained from said subject which encodes a human G protein β₃ subunit wherein a genetic modification is a substitution of cytosine by thymine at position 825. Homozygotes of the 825T-allele exhibit changes in ion current in atrial cells (Dobrev, D. et al., G-Protein beta(3)-Subunit 825T Allele Is Associated with Enhanced Human Atrial Inward Rectifier Potassium Currents, Circulation, 2000; 102:692-697) and results in reduced risk of atrial fibrillation. (Schreieck, J. et al., C825T Polymorphism of the G-protein beta3 Subunit Gene and Atrial Fibrillation: Association of the TT Genotype with a Reduced Risk for Atrial Fibrillation, Am. Heart. J., 2004; 148:545-550). A pilot study has shown that ventricular arrhythmias are more prevalent in CC-homozygotes than in TC-heterozygotes and TT-homozygotes. (Wieneke, H. et al., Better Identification of Patients Who Benefit from Implantable Cardioverter Defibrillators by Genotyping the G Protein beta3 Subunit (GNB3) C825T Polymorphism, Basic Res. Cardiol., 2006).

### Polymorphisms in GNAQ

The GNAQ gene codes for the Gαq subunit of hetero-trimeric G-proteins. The Gαq protein transmits signals over α1-adrenoceptors (nor-adrenaline), endothelin receptors and similar receptors. Gαq directly regulates many ion channels. Hyper-expression of Gαq in the heart leads to cardiac hypertrophy (Adams, J. W. et al., Enhanced Galphaq signaling: A Common Pathway Mediates Cardiac Hypertrophy and Apoptotic Heart Failure, Proc. Natl. Acad. Sci. U.S.A., 1998; 95:10140-10145), whereas the knockout of Gαq (plus Gα11) counteracts pressure-induced hypertrophy. (Wettschureck, N. et al., Absence of Pressure Overload Induced Myocardial Hypertrophy After Conditional Inactivation of Galphaq/Galpha11 in Cardiomyocytes, Nat. Med., 2001; 7:1236-1240). Three polymorphisms have recently been described in the promoter of gene GNAQ that cause alterations in the expression of the Gαq protein: (GC (−909/−908)TT), G (−382)A and G (−387)A as shown in Table 33. GC (−909/−908TT) (SEQ ID No. 858) has the following sequence: 5′-gcg tcc gca gag ccc gcg ggg gcc g [g/t] [c/t] cca gcc cgg gag ccg cgc ggg cga g-3′. The polymorphism G (−382)A is known by rs72466454 (SEQ ID No. 851) and has the following sequence: 5′-cgc cgc cag gcg cac ggc gta ggg ga [a/g] cct cgc agg cgg cgg cgg cgg cgg c-3′. The polymorphism G (−387)A is known by rs72466453 (SEQ ID No. 852) and has the following sequence: 5′-gct ctc gcc gcc agg cgc acg gcg to [a/g] ggg agc ctc gca ggc ggc ggc ggc g-3′.

### Polymorphisms in GNAS

The GNAS gene codes for the Gas-subunit of hetero-trimeric G-proteins. Activation of Gas (formerly the stimulating G-protein), activates adenyl cyclase, leading to increases in cAMP. Gas is activated by many hormone receptors. The activation of β1-adenoceptors is particularly important for the heart, as this leads to positive chronotropy and inotropy. Several somatic mutations in GNAS lead to rare endocrinological diseases (Weinstein, L. S. et al., Genetic Diseases associated with Heterotrimeric G Proteins, Trends Pharmacol. Sci., 2006; 27:260-266). There is also a silent C393T polymorphism thought to influence the response to beta-blocker medications (Jia, H, et al., Association of the G(s)alpha Gene with Essential Hypertension and Response to beta-blockade. Hypertension, 19991; 34:8-14). A series of polymorphisms in the promoter and intron-1 of gene GNAS has recently been described that modify the transcription rate and protein expression (C393T, G-1211A, C2291T) as shown in Table 33. C393T is known by rs7121 (SEQ ID No. 853) and has the following sequence: 5′-gag aac cag ttc aga gtg gac tac at [c/t] ctg agt gtg atg aac gtg cct gac t-3′. The polymorphism G-1211A is known by rs6123837 (SEQ ID No. 855) and has the following sequence: 5′-ctg gtc ttc tcg gtg cgc agc ccc tc [a/g] tgg gtg ctc aac ttc ctg ctg cag a-3′. The polymorphism C2291T is known by rs6026584 (SEQ ID No. 854) and has the following sequence: 5′-atc tgc agc tta agc cag tga cac aa [c/t] att ttg cat at taa atg gtg att c-3′.

TABLE 33
Prevalence of the SNPs analyzed in the
DISCOVERY study

| | Frequency of |
|---|---|
| SNP | minor allele |
| GNB3 c.825C > T | 30% T |
| GNAQ c.−909/−908GC > TT | 50% TT |
| GNAQ c.−382G > A | 5% A |
| GNAQ c.−387G > A | 8% A |
| GNAS c.393C > T | 50% T |
| GNAS c.2291C > T | 30% T |
| GNAS c.−1211G > A | 25% T |

### Polymorphism in GPC5

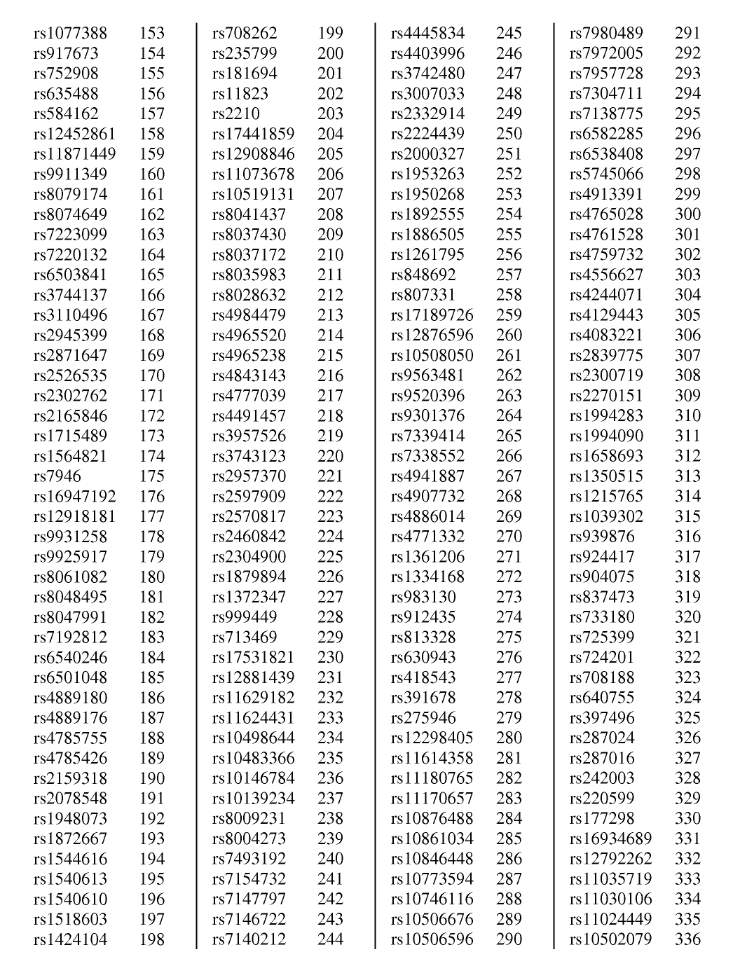

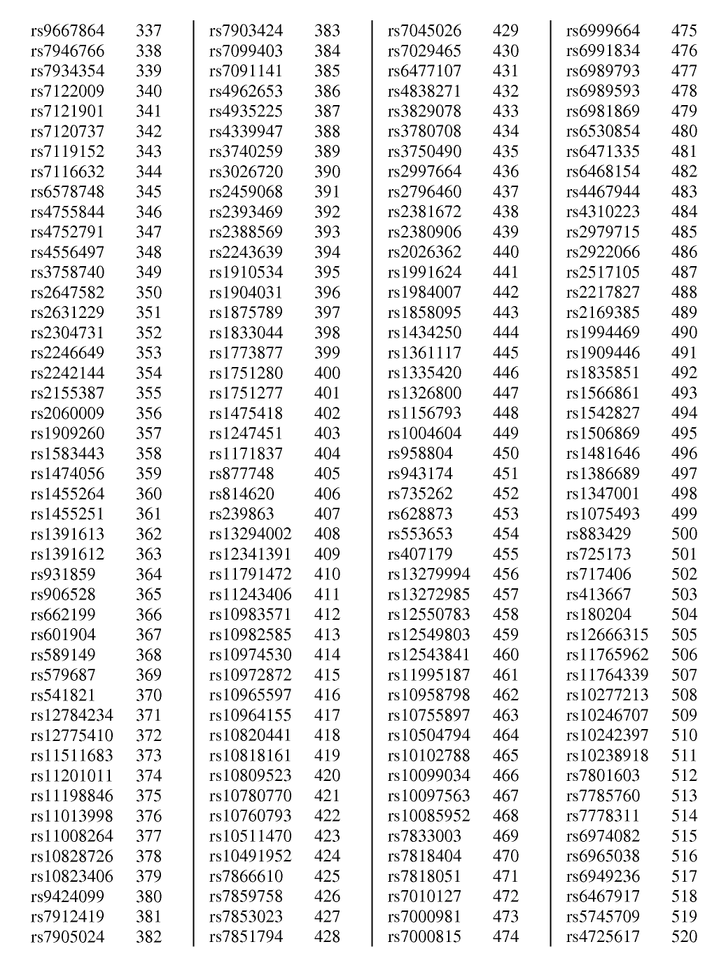

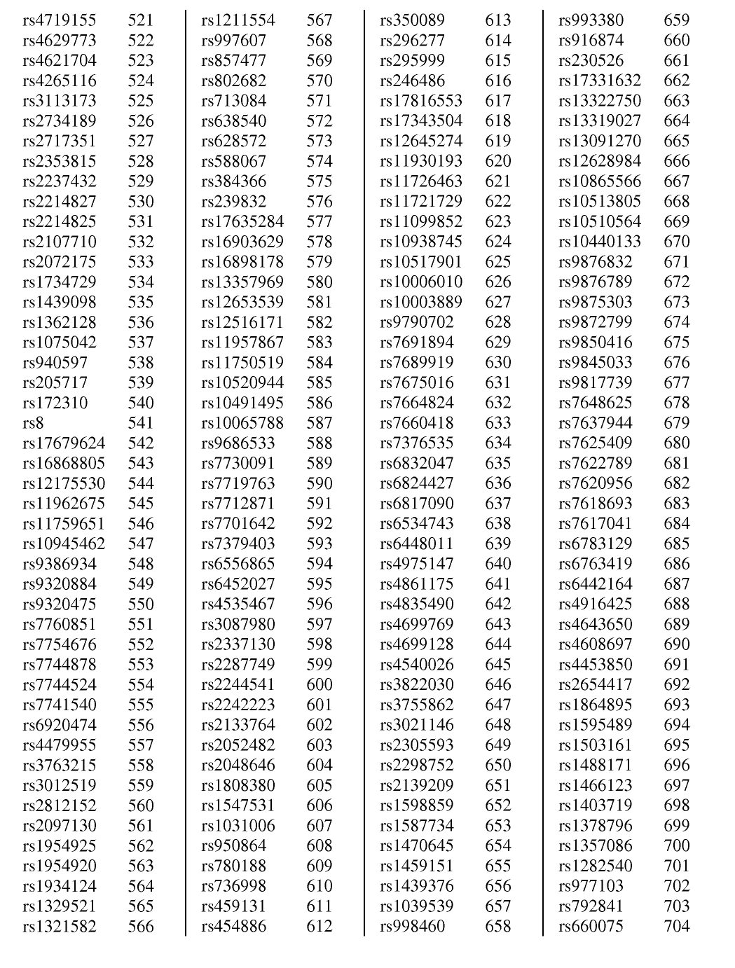

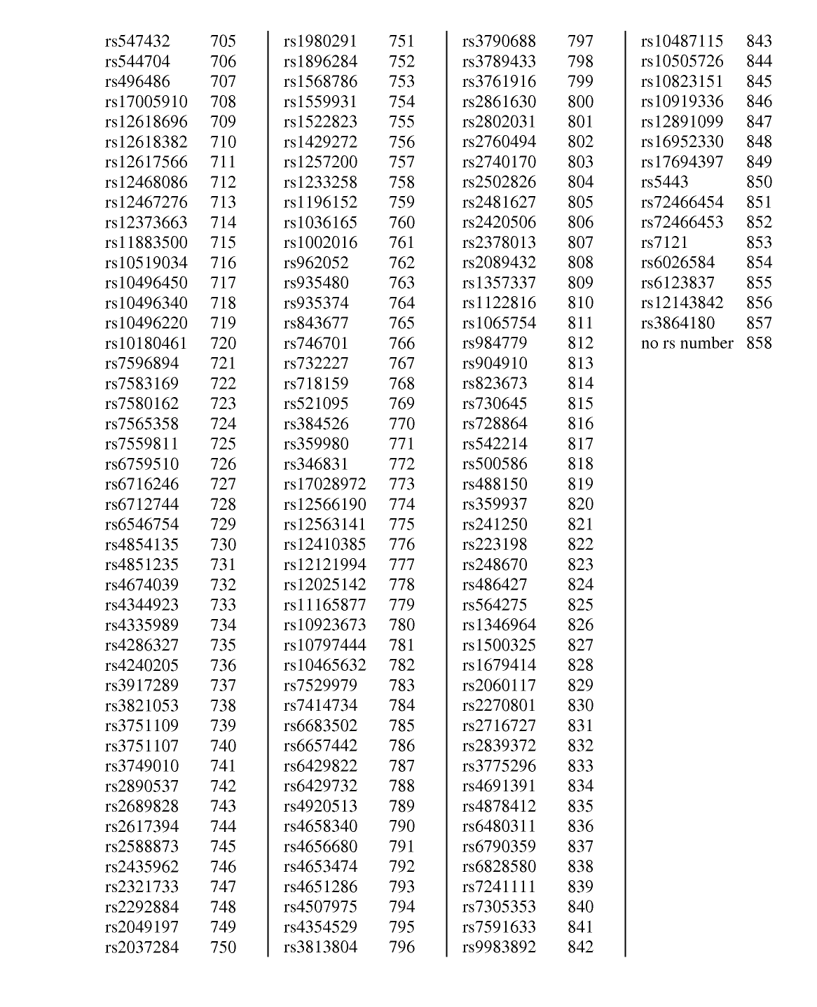

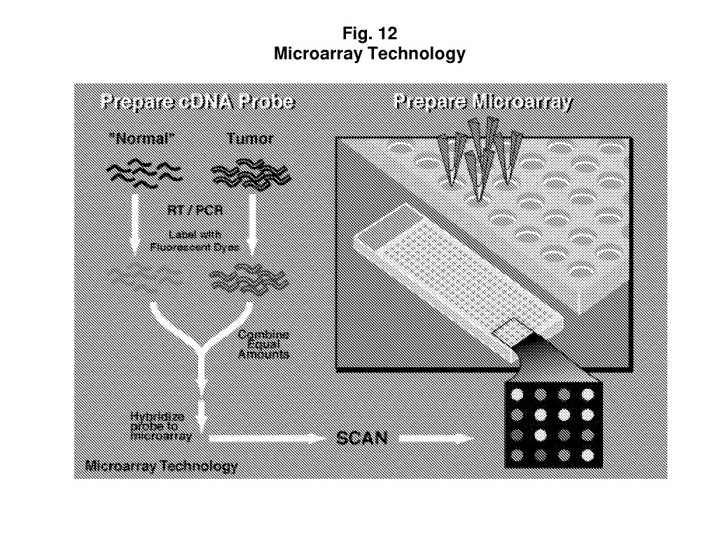

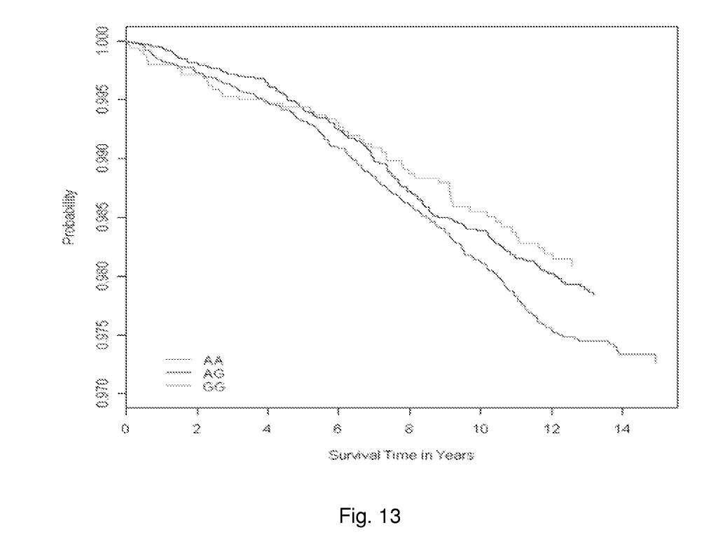

The minor allele of GPC5 (GLYPICAN 5, rs3864180) was associated with a lower risk of SCA in Oregon-SUDS, an effect that was also observed in ARIC/CHS whites (p<0.05) and blacks (p<0.04). Genome-Wide Association Study Identifies GPC5 as a Novel Genetic Locus Protective against Sudden Cardiac Arrest, Arking et al., PLosOne 2010 http://www.plosone.org/article/info:doi %2F10.1371%2Fjournal.pone.0009879. In a combined Cox proportional hazards model analysis that adjusted for race, the minor allele exhibited a hazard ratio of 0.85 (95% CI 0.74 to 0.98; p<0.01). FIG. 13 shows Cox proportional hazards model was adjusted for age, sex, and race/ethnicity. Individuals homozygous for the protective allele (GG) are shown in green, heterozygotes (AG) in blue, and homozygous for the risk allele (AA) are in red. Further, a statistically significant interaction between rs3864180 and sex (P<0.012), with a stronger effect in women, has been reported in the association with SCA. However, the GPC5 association to SCD was shown in a non-ICD population. The polymorphism of GPC5 is known by rs3864180 (SEQ ID No. 856) and has the following sequence: 5′-tgt tca tct att caa aat gta gta to [a/g] ttt tat ttg aga ttg tct ttt ttt a-3′.

### Polymorphisms in the CAPON(NOS1AP) Gene

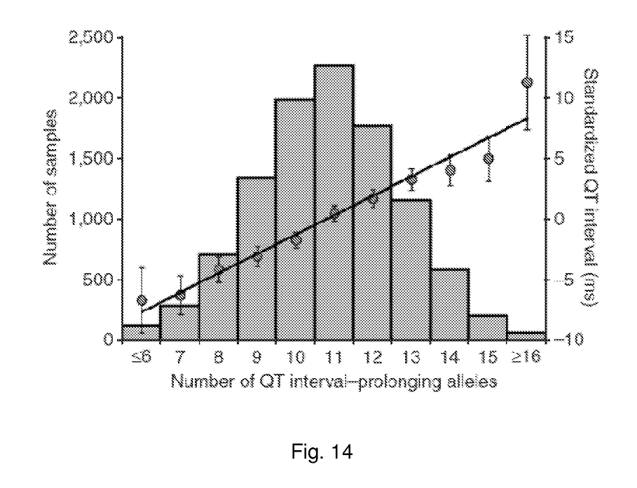

The CAPON(NOS1AP) gene was shown to modulate the QT duration. “Common variants at ten loci modulate the QT interval (A. Pfeufer et al., Nature Genetics 2009). FIG. 14 shows individuals classified by counting their number of QT-prolonging alleles in all ten identified markers (max score 20). Dosages for the QT-prolonging allele as calculated by MACH1 were added and then rounded to the nearest integer. Gray bars indicate the number of individuals in each score class, blue dots indicate the mean QT interval for each class, and the black line is the linear regression though these points. The polymorphism of GPC5 is known by rs12143842 (SEQ ID No. 857) and has the following sequence: 5′-tta gca ccc agg gtc aca tcc cag tt [c/t] aaa aat atc cca tgg agt gca gtc a-3′.

### DISCOVERY Study

The DISCOVERY study determined whether a correlation exists between genotypes and the incidence of atrial and ventricular arrhythmia, as measured by a dual chamber ICD produced by Medtronic, Inc. The differentiated diagnostic data (Saoudi, N. et al., How Smart Should Pacemakers Be? Am. J. Cardiol, 1999; 83:180-186) afforded by the ICDs can produce information on arrhythmia trigger (Marshall, A. J., et al., Pacemaker Diagnostics to Determine Treatment and Outcome in Sick Sinus Syndrome with Paroxysmal Atrial Fibrillation, PACE, 2004; 27: 1130-1135) and IEGMs (Mitrani, R. D., et al., The Use Of Pacemaker Diagnostic Data To Guide Clinical Decision Making, Presented at Cardiostim, 2006), supraventricular tachycardia which are a known independent risk factor for mortality and stroke. (Benjamin, E. J. et al., Impact Of Atrial Fibrillation On The Risk Of Death: The Framingham Heart Study, Circulation, 1998; 98:946-952; Glotzer, T. V. et al., Atrial High Rate Episodes Detected By Pacemaker Diagnostics Predicts Death And Stroke, Circulation 2003; 107(12):1614-1619). These data complement the follow-up data collected during unscheduled cardiology, specialized pacing and electrophysiology examinations.

The second part of the DISCOVERY study evaluated the therapeutic utility of ICD-based diagnostic information on patient treatment or management of symptoms related to cardiovascular disease. Recently developed ICD algorithms target improved patient-specific therapies. However, ICDs can also provide physicians with increasingly differentiated diagnostic information (Saoudi, N. et al., 1999). First, the diagnostic information available in the ICDs is separated into system-related and patient-related diagnoses. (Nowak, B., Taking Advantage of Sophisticated Pacemaker Diagnostics, Am. J. Cardiol., 1999; 83:172-179). This separation provided a systematic approach for the classification of the information generated.

System-related diagnostic data includes device query, battery and lead status, thresholds, sensing, and related long-term trends. This data enables early detection of hardware dysfunction. Patient-related diagnostics include intra-cardiac EGM, sensor data, and channel markers. This data supports the evaluation of device reactions to a patient's intrinsic rhythm and provides information on arrhythmia and heart disease progression. Patient-related diagnostic data may also be used to evaluate device programming and the impact of medication on the treatment or suppression of cardiovascular disease. The study evaluated the use of system-based and patient-based diagnostics and the resulting medical consequences, including medical interventions, prescription of medication and changes in medication, surgery, additional diagnostics, and changes in ICD programming. Similarly, the frequency of programming changes involving AF-prevention or AF-therapy algorithms and programming changes involving changes in pacing parameters were evaluated along with the resulting medical consequences.

The Medtronic, Inc. ICDs used in the study stored long-term trends for numerous diagnostic parameters over a period of up to 14 months. The device long-term diagnostics complement the information collected during patient follow-up examinations, which reflect only a brief exposure to a physician. For example, early identification of lead defects is improved by examining long-term impedance and sensing trends where major fluctuations are visible (Soudi, N. et al., 1999). Arrhythmia therapy also significantly relies on stored ICD information and can be qualified by device-based system diagnostics (Mitriani, R. D. et al., 2006). For example, the stored information related to atrial arrhythmia trends permits differentiated diagnosis of atrial arrhythmias, which comprise an independent risk factor for morality, stroke, and atrial fibrillation (“AF”) in pacemaker patients with sinus node disease (Benjamin, E. J. et al., 1998; Glotzer, T. V. et al., 2003). Understanding the triggers of atrial arrhythmias can be of decisive importance in the treatment or reduction of atrial arrhythmias (Marshall, A. J. et al., 2004). Additionally, assessment of atrial coherence is important for the diagnostic interpretation of atrial arrhythmias provided by ICDs. Atrial leads with long-term trends in sensing values and EGM episodes support the evaluation of sensing integrity and of atrial arrhythmia episodes by highlighting sensing malfunction on atrial channels and leads which would otherwise result in a faulty assessment of arrhythmias.

The DISCOVERY study was an interventional non-randomized, longitudinal, prospective, multi-centric, diagnostic study. It was composed of two parts: Part One was a double-blind study, and analyzed data on genetic polymorphisms as prognostic of ventricular and atrial tachyarrhythmia. Part Two of the study evaluated the influence of ICD-based diagnostic information on long-term patient management and treatment. Subjects were enrolled for a period of approximately 24 months, and the total study duration was 48 months. The DISCOVERY study intended to determine the diagnostic value obtained from SNPs studied within the framework of Cardiac Compass and other diagnostic tools available in the commercially released Medtronic, Inc. produced ICD devices. The devices were manufactured in accordance with the provisions of the Active Medical Device Directive (90/385/EEC) and comply with all relevant legal requirements. The devices and leads were market released and used within labeling.

Subjects who were included in the study first had implantation of a market approved Medtronic, Inc. Dual-chamber ICD with long-term clinical trends as Cardiac Compass including, but not limited to, Marquis DR (7274), Maximo DR (7278), Intrinsic DR (7288), EnTrust DR (D153ATG), and Virtuoso DR (D164AWG). The components used were programmer 2090 and 2090W (Medtronic, Inc.), all market released leads, and all Medtronic, Inc. market released software. However, other leads and software known to those of skill in the art are contemplated. The 2090 and 2090W programmer and Medtronic, Inc. software was used to interrogate and program the parameters of the devices. The software was market released in Europe. Additional ICDs, leads, programmers, software and accessories were optionally incorporated into the study as they became commercially available. As part of the CE conformity assessment, a notified body evaluated the biocompatibility, clinical performance, and safety of all the devices and leads used in the DISCOVERY study.

The subjects had ICD indication for primary prevention of ventricular arrhythmia according to the current AHA/ACC/ESC guidelines (A report of the ACC/AHA Task Force and the ESC Committee for Practice Guidelines: ACC/AHA/ESC 2006 Guidelines For Management Of Patients With Ventricular Arrhythmia and the Prevention of Sudden Cardiac Death—Executive Summary, European Heart J., 2006; 27:2099-2140). Subjects were also willing and able to comply with the Clinical Investigation Plan, remained available for follow-up examinations, and signed an informed consent form within 10 days of receiving the implant.

Excluded subjects included pregnant women; women of childbearing potential who did not use a reliable form of birth control; subjects enrolled in a concurrent study that may confound the results of this study; minors; subjects with a life expectancy of less than two years; subjects who have had or were awaiting heart transplantation; subjects having syndromes known to be associated with Ion channelopathies such as Long- and Short-QT Syndrome, Brugada Syndrome, Catecholaminergic Polymorphic Ventricular Tachycardia (CPTV); and subjects otherwise deemed appropriate for exclusion based on an expectation of poor compliance.

The ICD devices used in the study were multi-programmable, Dual-chamber ICDs as previously described. All devices automatically detect and treat episodes of VT, VF, fast ventricular tachycardia and bradyarrhythmia. When a cardiac arrhythmia is detected, the implantable device delivers defibrillation, cardioversion, anti-tachycardia pacing or standard pacing therapy. The devices collect and store various types of data and provide a range of diagnostic tools to manage patient care.

A summary of the data and diagnostic tools, which are available during follow-up examination, is provided herein. The devices provided a Quick Look screen which supplies a summary of the episode data, device and lead status information, programmed bradycardia pacing parameters, conduction status, and device observations since the last patient session. The Quick Look screen is displayed after the software application is started. The Observations section of the Quick Look screen highlights significant device status events, lead status events, Patient Alert events, parameter programming, diagnostic data, and clinical status data.

The Cardiac Compass report provides up to 14 months of clinically significant data including arrhythmia episodes, therapies delivered, physical activity, heart rate, and bradycardia pacing activity. The report can be useful in correlating changes in data trends to changes in programmed parameters, drug regimen, or patient condition. The Cardiac Compass report provides an overall view based on the following daily checks or measurements: VT/VF episodes; indication of a cardioversion or defibrillation therapy delivered; ventricular rate during VF, FVT, or VT episodes; the number of VT-NS episodes per day; the total time in AT or AF (EnTrust and Virtuoso devices only); ventricular rate during AT or AF (EnTrust and Virtuoso devices only); percentage of atrial and ventricular pacing; average day and night ventricular rate; overall patient activity; heart rate variability; and OptiVol fluid index (Virtuoso device only).

The Cardiac Compass report also provides the following trend data. The “VT/VF episodes per day trend” provides a history of ventricular tachyarrhythmia and may reveal correlations between clusters of episodes and other clinical trends. Each day, the ICD records the total number of spontaneous VT and VF episodes. The episode counts are provided in histogram format on the report.

The device also records a shock indicator for any day on which it delivers an automatic defibrillation therapy, cardioversion therapy, or atrial shock therapy. The Cardiac Compass report displays an annotation for the day on which a defibrillation therapy, cardioversion therapy, or atrial shock therapy was delivered.

The Cardiac Compass displays a graph of the daily median ventricular rate for spontaneous VF, FVT and VT episodes, which may have occurred. This may provide an indication of the effects of anti-arrhythmic drugs on VF, FVT, and VT rates and a better understanding of the safety margins for detection.

The “non-sustained VT episodes” trend may reveal correlations between patient symptoms (such as palpitations) and VT-NS episodes and may indicate a need for further investigation of the status of the patient. Each day, the ICD records the total number of spontaneous VT-NS episodes. The episode counts are provided in histogram format on the report.

The “AT/AF total hour per day” trend for EnTrust and Virtuoso devices helps in the assessment of the need for anti-arrhythmic drugs or ablations to reduce AT/AF episode occurrences or for anti-coagulant drugs to reduce the risk of stroke. The device records a daily total for the time the patient spent in AT or AF.

The “ventricular rate during AT/AF” trend reveals correlations between patient symptoms and rapid ventricular responses to AT/AF. It is also useful in the assessment of the efficacy of an AV node ablation procedure or in the assessment VT/VF detection safety margins so that programming may be modified to avoid treating rapidly conducted AT/AF as VT/VF. The trend may be used further to prescribe or titrate anti-arrhythmic and rate control drugs. The device records average and maximum ventricular rates during episodes of AT and AF each day. The values are plotted on the Cardiac Compass report along with the average ventricular rates.

The percent pacing per day graph provides a view of pacing over time that can help identify pacing changes and trends. It displays the percentage of events occurring during each day that are atrial paces (AT and DR devices only) and ventricular paces. It can be useful to program the pacing parameters in a way that helps to avoid unnecessary ventricular stimulation in those patients that have no indication for ventricular pacing.

The “patient activity” trend can be evaluated and used for the following types of information. The trend can act as an early indicator of symptoms due to progressive diseases like heart failure, which causes fatigue and a consequent reduction in patient activity. Similarly, the trend allows monitoring of a patient's exercise regimen. The trend is an objective measurement of a patient's response to changes in therapy. The trend may also be used to study outcomes in ICD patients, along with additional parameters such as quality of life. The device uses activity count data derived from the rate response accelerometer signal to determine patient activity. The activity values are stored daily. For each seven days of stored data, the device calculates a seven-day average. This average is plotted for the Cardiac Compass report.

The night and day heart rate trend provides the following clinically useful information: gradual increase in heart rate, which may indicate cardiac decompensation as a symptom of heart failure; objective data that may be correlated with patient symptoms; indications of autonomic dysfunction or heart failure; and information regarding diurnal variations.

In AT and DR devices, the device measures the median atrial interval value every five minutes and calculates a variability value each day. The heart rate variability value, in milliseconds, is plotted on the Cardiac Compass report.

The “OptiVol” fluid index trend for Virtuoso devices displays the accumulation of the time and magnitude that the daily impedance is less than the reference impedance. If the daily impedance is less than the reference impedance, then the OptiVol fluid index trend increases. This may indicate that the patient's thoracic fluid has increased. The OptiVol fluid monitoring feature is an additional source of information for patient management.

The “rate histograms report” counts and collects atrial or ventricular events and classifies them by rate range and the percentage of time. The device automatically collects the histogram data without any programming by the clinician. This diagnostic is intended for ambulatory monitoring uses, such as monitoring rate distribution. Rate histograms also collect the ventricular rate during AT/AF. This diagnostic may be used to evaluate drug titration.

Flashback Memory allows analysis of heart rates leading to a VF, VT, or AT/AF episode and compares the pre-VF, pre-VT, and pre-AT/AF rhythms to the normal sinus rhythm and to other episodes. In AT and DR devices, Flashback Memory automatically records V-V and A-A intervals and stored marker data for the following events: the most recent VF episode, the most recent VT episode, the most recent AT/AF episode, and the most recent interrogation.

The ICD automatically and continuously monitors battery and lead status. The Battery and Lead Measurements screens after interrogation views and prints the following data: current battery voltage, last capacitor formation, last capacitor charge, sensing integrity counter data, last atrial lead position check (EnTrust and Virtuoso devices only), last lead impedance data, last sensing data, and last high-voltage therapy.

The automatically performed daily lead impedance and sensing measurements are used to generate lead performance graphs based on up to 82 weeks of measurements. A separate graph is provided for each of the following measurements: atrial placing lead impedance (AT and DR devices only), ventricular pacing lead impedance, defibrillation lead impedance, SVC lead impedance (if used), P-wave sensing amplitude (AT and DR devices only), and R-wave sensing amplitude.

Methods of stratifying patients into diagnostic groups based on a determination of risk of ventricular tachycardia are provided. Methods of evaluating ICD-based diagnostic information for the long-term treatment and management of primary prevention ICD patients are also provided. Subjects suitable for evaluation are first identified. A review of each subject is required to determine preliminary eligibility according to subject inclusion and exclusion criteria. Clinical data is collected via study electronic or paper case report forms (“eCRF” or “CRF”) at the time of subject's baseline, planned follow-up examinations, unscheduled follow-up examinations, system modification and subject exit, including deaths, as applicable.

The Baseline CRF is used to record the baseline data for all subjects. The information documented may include, for example, (1) verification of inclusion and exclusion criteria; (2) recording of the subject's demographic and medical history; (3) cardiovascular status and history, including arrhythmia history; (4) ICD implant indication; (5) physical assessment such as LV ejection fraction, 12-lead ECG; (6) New York Heart Association (NYHA) classification; (7) Recording of cardiovascular medications; and (8) date of blood test for the genetics analysis. A subject's cardiovascular history includes heart failure (“HF”) etiology, previous surgery and history of arrhythmia.

Cardiac medications are recorded at the baseline evaluation, but recordation of non-cardiac medications is not required. During every follow-up visit, however, only medication changes as a result of the use of patient-related diagnostics will be recorded, as described below. Cardiac medications include, but are not limited to, angiotensin-converting enzyme inhibitors and angiotensin receptor blockers, anti-arrhythmic medications, beta-blockers, diuretics, calcium channel blockers, anticoagulants, inotropes, nitrates, cardiac glycosides, and anti-lipidemics, e.g., statins.

New York Heart Association classification of functional capacity is based on a classification system originating in 1928, when the NYHA published a classification of subjects with cardiac disease based on clinical severity and prognosis. This classification has been updated in seven subsequent editions of Nomenclature and Criteria for Diagnosis of Diseases of the Heart and Great Vessels (Little, Brown & Co.). The ninth edition, revised by the Criteria Committee of the American Heart Association, New York City Affiliate, was released Mar. 4, 1994. These classifications are summarized below in Table 34.

TABLE 34
Functional Capacity

| | |
|---|---|
| Class I | Subjects with cardiac disease but without resulting limitation of physical activity. |
| | Ordinary physical activity does not cause undue fatigue, palpitation, dyspnea, or |
| | angina. |
| Class II | Subjects with cardiac disease resulting in slight limitation of physical activity. |
| | They are comfortable at rest. Ordinary physical activity results in fatigue, |
| | palpitation, dyspnea, or angina. |
| Class III | Subjects with cardiac disease resulting in marked limitation of physical activity. |
| | They are comfortable at rest. Less than ordinary activity causes fatigue, |
| | palpitation, dyspnea, or angina. |
| Class IV | Subjects with cardiac disease resulting in inability to carry on any physical activity |
| | without discomfort. |
| | Symptoms of HF or the anginal syndrome may be present even at rest. If any |
| | physical activity is undertaken, discomfort is increased. |

A left ventricular ejection fraction (“LVEF”) measurement is also performed at baseline if one has not been performed within 30 days prior to subject enrollment. The following information is collected: LVEF measurement, the method of LVEF measurement, radionucleotide entriculo-cardiography/MUGA, echocardiography, and ventricularcardiography via catheterization.

A 12-lead ECG is also performed during the baseline evaluation if one has not been performed within 30 days prior to subject enrollment. The QRS width and the lead used for measurement will be circled and maintained in the patient's file.

After a subject receives an IMD, the device is programmed. The programming is done according to the applicable Medtronic ICD System Reference Manual for the programs for detection and therapy parameters for bradycardia pacing and anti-tachycardia therapy. Table 35 outlines the required programming parameters and concern only the diagnostic quality of the data collected. Deviation from these settings must be recorded with the clinical evidence justifying deviation from the programming requirements.

TABLE 35

| Parameter | Feature | Value |
|---|---|---|
| VF | Detection | ON |
| | initial beats to detect (NID) | 18/24 |
| VT | Detection | ON or Monitor |
| | Initial beats to detect (NID) | 16 |
| | V Interval | >400 ms |
| | EGM 1 Source | Atip - Aring |
| | EGM 2 Source | Vtip - Vring |

Recommended programs are shown in Table 36 and may be subject to change by a subject's physician.

TABLE 36

| Parameter | Feature | Value |
|---|---|---|
| VF | V interval | 300 ms |
| | Redetect beats to detect | 9/12 |
| | Therapies | 6 × max. energy [J] |
| FVT | Detection | via VF |
| | V Interval | 240 ms |
| | Therapies | Burst (1 sequence)*, |
| | | 5 × max. energy [J] |
| VT | Redetect beats to detect | 8 |
| | Therapies | Burst (2)*, Ramp (1)**, 20 J, |
| | | 3 × max. energy [J] |
| SVT criteria | PR Logic: AFib/AFlutter | On |
| | PR Logic: Sinus Tach | On |
| | 1:1 VT-ST boundary | 66% (except EnTrust, Virtuoso) |
| | SVT Limit | 260 ms |
| Pacing | MVP | On (only InTrinsic, EnTrust, |
| | | Virtuoso) |
| | PAV (where applicable) | >230 ms |
| | SAV (where applicable) | >200 ms |

*Burst ATP: 8 intervals, R-S1 = 88%, 20 ms decrement
**Ramp ATP: 8 intervals, R-S1 = 81%, 10 ms decrement

At the conclusion of the implantation, as well as at the beginning and conclusion of every follow-up examination, the device is interrogated, and the data is collected via the device's Save-to-Disk function. A full interrogation is performed without clearing any episodes. If any Save-to-Disk files related to a follow-up visit are permanently missing, a Study Deviation form is completed. Subject data may be excluded from analysis if a sequential device-based arrhythmic history cannot be provided at a later time.

Clinical data is also collected at the time of the subject's planned follow-up, unscheduled follow-up, system modification and subject exit. Regular follow-up examinations take place at 6, 12, 18, and 24 months after device implantation. To obtain sufficient incidence of ventricular arrhythmia, a follow-up duration of 24 months per subject is required. If a follow-up visit falls outside the acceptable target day +/−30 days, the original follow-up schedule will be maintained for the remaining visits. Table 37 shows the method for determining appropriate follow-up visit scheduling. Medical treatment and device programming not included in Table 9 are left to the discretion of the examining physician during follow-up examinations.

TABLE 37

| | Days post Device-Implantation | | |
|---|---|---|---|
| Visits | Window start | Target day | Window end |
| 6 month follow-up | 153 | 183 | 213 |
| 12 month follow-up | 335 | 365 | 395 |
| 18 month follow-up | 518 | 548 | 578 |
| 24 month follow-up | 700 | 730 | 760 |

At every scheduled and unscheduled follow-up visit, the following information is recorded: (1) cardiac symptoms, (2) occurrence and classification of arrhythmia, (3) use of system-related diagnostics such as battery status, impedance, pacing threshold, and sensing, (4) use of patient-related diagnostics such as arrhythmia information, heart frequency and stimulation that may lead to a change in treatment, (5) programming changes of the device using the device diagnostic, (6) follow-up duration, (7) number of medication changes as a result of the use of patient-related diagnostics, and (8) NYHA classification. The procedures for collecting the subject demographic and medical history and NYHA classification and cardiac medication information are as previously described. A Save-to-Disk function is performed using the ICD.

If applicable, the following reports are also completed: a Study Deviation report and/or an Adverse Event report. An Adverse Event (“AE”) is any untoward medical occurrence in a subject. An Adverse Device Effect (ADE) is any untoward and unintended response to a medical device, including any event resulting from insufficiencies or inadequacies in the instructions for use or the deployment of the device, which also includes an event that is a result of user error. A Serious Adverse Event (SAE) is an AE that (1) leads to death, (2) leads to fetal distress, fetal death, or a congenital abnormality or birth defect, or (3) leads to a serious deterioration in the health of a subject that (i) resulted in a life-threatening illness or injury, (ii) resulted in a permanent impairment of a body structure or a body function, (iii) required in-patient hospitalization or prolongation of existing hospitalization, or (iv) resulted in medical or surgical intervention to prevent permanent impairment to a body structure or body function.

The Adverse Event report is only for serious adverse device effects (“SADE”) and serious procedure-related adverse events. An SADE is an event that has resulted in any of the consequences characteristic of a Serious Adverse Event or that might have led to any of those consequences if (i) suitable action had not been taken, (ii) intervention had not been made, or (iii) if circumstances had been less opportune. Serious procedure related adverse events are those that occur due to any procedure specific to the treatment and examination of the subject, including the implantation or modification of the system. Ventricular or supra-ventricular arrhythmias that are detected are not treated as adverse events, whether or not treated, because they constitute material events analysis.

Information reported on the Adverse Event form includes a description of the event, the diagnosis, the date of event onset, the relationship of the event to the procedure, the relationship of the event to the device or system, actions taken as a result of the event, and the outcome of the event. Adverse events are to be reported as soon as possible after the event occurs.

In the event that the device or leads require invasive modification (e.g., ICD or lead explants, ICD or lead replacement, or lead repositioning), a system modification CRF is completed. An Adverse Event CRF is likewise completed to document the underlying cause of the system modification. When possible, explanted ICD devices and/or leads are returned to Medtronic, Inc. for analysis.

The devices and other components contemplated by the invention are those described as being used in and intended for the DISCOVERY study. If the device or lead is not replaced with those not meeting these criteria, then the following steps are performed. First, prior to explant, the device is interrogated and the data is saved onto one or more diskettes as needed using the Save-to-Disk function. The subject is then followed over the next 30 days or until all Adverse Events associated with the initial system are either resolved or unresolved with no further action required, whichever occurs last. Finally, a study termination CRF is completed for the subject.

If the ICD or leads are replaced with those meeting the above criteria, then the following steps are performed. First, prior to explant, the device is interrogated and the data is saved onto one or more diskettes as needed using the Save-to-Disk function. The subject is then followed according to his or her regular examination schedule.

Each patient death is classified as follows: (1) cardiac, non-cardiac, or unknown; and (2) sudden, non-sudden, or unknown. All deaths are reported. In addition, deaths will be classified based on whether they are related to the device or lead system and whether they were arrhythmic, non-arrhythmic (vascular) or unknown. The following information will also be collected when a death occurs, if available: a medical report, EGM or IEGM related to the death, an autopsy report, and a full device interrogation using Save-to-Disk.

A cardiac death is defined as a death related to the electrical or mechanical dysfunction of the heart. The initiating event, which may be preventable, is differentiated from the terminal event. For this purpose, the initiating event of cardiac death thus requires further classification as either arrhythmic or vascular: (1) initiating event is arrhythmic, non-arrhythmic, or unknown; (2) initiating event is vascular, non-vascular or unknown. Non-cardiac deaths are all deaths with a known cause not classified as cardiac deaths. If insufficient information is available to classify a death as cardiac or non-cardiac, the death is classified as unknown.

Sudden death is a witnessed death within one hour after onset of acute symptoms, or un-witnessed death, that is unexpected and without other apparent cause, including death during sleep. Non-sudden death is a death that is not classified as sudden death, including cardiac death of hospitalized subjects on inotropic support.

Blood samples are collected from each subject and analyzed for seven single nucleotide polymorphisms in the genes GNB3, GNAQ, GNAS. Then, the subject data is analyzed to determine the existence of a correlation between these SNPs and the occurrence of arrhythmia. During the course of the evaluation, additional genetic factors related to other medical conditions, which may or may not be cardiac related, may also be revealed. The genetic profiles of the subjects may be used in additional research and analysis. This additional research involves medical research related to genetic effects on diseases and diagnostic and therapeutic applications of that research.

Statistical analysis is then performed on the data collected. For all analyses, a two-sided p-value or 0.05 is considered to be statistically significant. Groups of patients for which statistical analysis is performed must contain no fewer than four patients. No populations are defined for the statistical analyses. All data is analyzed as collected. There is no imputation of missing data. Continuous variables are reported using N,N missing, mean, standard deviation, minimum, median and maximum. Categorical variables are reported using N per category and percentages. In addition, for categorical variables where more than one category can be crossed, a report of how often a combination of categories has been checked is made. The analyses are performed after two years of follow-up have been completed for each subject. There is no correction for multiple testing.

After the baseline and follow-up patient data is collected, a determination is made of the value of a SNP in the genes GNB3, GNAS and GNAQ as a predictor for ventricular arrhythmia <400 ms. For the analysis of the predictive power of the various SNPs, sensitivity, specificity, and positive and negative predictive value are calculated as a predictor of the primary endpoint in patients without a history of spontaneous VT/VF (primary prevention indication for ICD implantation). Confidence intervals are calculated (95%, 2-sided). A primary endpoint is a ventricular arrhythmia which can be detected within a Tachycardia Detection Interval (TDI) programmed as follows: 18 out of 24 for ventricular fibrillation or 16 consecutive beats for ventricular tachycardia with a maximum cycle length of 400 ms. A cutoff value of 400 ms has been selected in order to capture a high amount of ventricular tachy-arrhythmias and at the same time avoid inappropriate ICD therapies. Sweeney et al. have shown in the PainFREE RX II trial (Appropriate and Inappropriate Ventricular Therapies, Quality of Life, and Mortality Among Primary and Secondary Prevention Implantable Cardioverter Defibrillator Patients: Results From the Pacing Fast VT Reduces Shock Therapies [PinFREE Rx II] Trial, Circulation, 2005; 111:2898-2905) that the mean cycle length of ventricular tachycardia in primary prevention patients is 351 msec. In the PainFree Rx II and in the EMPIRIC trial, (Wilkoff, B. L. et al., A Comparison of Empiric to Physician-Tailored Programming of Implantable Cardioverter-Defibrillators, J. Am. Coll. Cardiol., 2006; 48:330-339) a cutoff value of 400 ms was chosen, allowing heart rates as low as 150 beats per minute. A cutoff value of 400 ms lead to an amount of only 11.9% inappropriately treated SVTs in the EMPIRIC trial.

The sample size calculation is based upon a required accuracy of the estimate of the positive predictive value (PPV) of the potential risk stratifiers under study. A 95% confidence interval with a maximal width of ±5% is deemed appropriate. This level of accuracy of the estimated PPV requires a sample of 386 patients with a positive risk stratifier, assuming actual PPV is 40%. The bisection method is used along with the proportion confidence interval formulas found in Johnson and Kotz (Discrete Distributions, Houghton Mifflin Company, Boston, 1969, 58-60). This focus is on those markers that have incidence greater than one third of all patients. If 386 patients are required to reach the primary endpoint, 3×386=1158 patients are needed with a primary indication for ICD implantation. With an approximated 10% of the patients lost to follow-up, a total of 1287 patients are required. Alternatively, it may be acceptable to use a 95% confidence interval with a maximal width of ±7, 5%, requiring 583 patients in total.

The positive value of SNPs as predictor for death, cardiac death and atrial fibrillation or flutter in genes GNB3, GNAS and GNAQ is determined. A determination of the positive predictive value is also made for other SNPs having signal transduction components that impact on the activity of cardiac ion channels. To evaluate the predictive power of the various SNPs, sensitivity, specificity, positive and negative predictive value will be calculated as predictor of the endpoints: death, cardiac death, and atrial fibrillation or flutter.

Finally, a determination as to the most useful combination of genetic parameters, baseline data and follow-up data is made regarding the predictor of primary endpoint, all-cause mortality, cardiac death, and atrial arrhythmia. This determination involves analysis of the following parameters: (1) available genetic tests, (2) QRS width, (3) baseline medication, (4) age, (5) heart rate variability documented by the device diagnostics such as Cardiac Compass, (6) history of AT/AF, and (7) documented AT/AF by the device or Holter ECG. NYHA and EF data are related to ICD implantation indication and are assessed as eventual baseline correction. Usefulness is evaluated in terms of the greatest positive predictive value for the prognosis of sustained ventricular arrhythmia, non-cardiac death, cardiac death, and atrial arrhythmia.

For each combination of test and endpoint parameters, a univariate Cox proportional hazards model is used to assess the predictive value of the test. For each endpoint, the tests with a univariate correlation of p<0.05 are included in the multivariate Cox proportional hazard regression analysis. The best combination of these tests is selected by an automatic algorithm applied to a Cox proportional hazard model.

During performance of the methods of the invention, diagnostic data collected by the implanted devices is observed, compared, and analyzed. The results of such analyses may lead to conclusions and insights that, in turn, could result in device programming that might be more favorable for an individual patient or patient subgroups compared to the settings originally chosen. If new aspects regarding optimization of treatment such as the programming of the devices arise, appropriate changes in treatment may be made that will provide a benefit to the patient(s). The correlations made between the results of the genetic analyses and the amount of ventricular and atrial arrhythmia which are statistically significant benefit patients by providing for more appropriate patient selection for ICD therapy.

A method of determining the medical consequences of using ICD-based diagnostics is also provided. In the method, patient treatment using ICD-based diagnostics is evaluated along with the medical consequences of such treatment. Alternatively, the medical consequences of ICD-based rhythmic diagnostic data are evaluated. This analysis is performed using the subject follow-up CRFs. The determination is made by setting a value on diagnostic and treatment utility of the diagnostics based on the medical consequences.

A method of evaluating the frequency of programming changes involving AF-prevention and AF-therapy algorithms triggered by device diagnostics is also provided. Device interrogations documented at the beginning and end of each subject follow-up examination are used to identify changes in device programming regarding AF-prevention and AF-therapy. A determination is made as to the frequency of changes in device programming. Additionally, the frequency of pacing parameter programming changes and the resulting medical consequences is also evaluated. A determination is made of the medical consequences of such programming changes.

The medical consequences of the use of ICD systems and ICD-based diagnostics may include certain potential risks. These risks may include, but are not limited to, the following types of events and medical consequences. Adverse events associated with ICD systems include, but are not limited to: acceleration of arrhythmia (caused by the ICD), inappropriate detection of tachy-arrhythmia, inappropriate therapy for tachy-arrhythmia including shocks, potential sudden death due to failure to detect and/or inability to defibrillate or pace, air embolism, bleeding, chronic nerve damage, erosion, excessive fibrotic tissue growth, extrusion, fluid accumulation, hematoma or cysts, infection, body rejection, keloid formation, lead abrasion and discontinuity, lead migration/dislodgement, movement of the device from its original location, myocardial damage, pain, pneumothorax, seroma, thromboemboli, venous occlusion, venous or cardiac perforation, shunting current or insulating myocardium during defibrillation.

Patient conditions may also change. Even when there has been a satisfactory response to tachyarrhythmia therapies during clinically conducted electrophysiology studies, underlying or accompanying diseases or a change in anti-arrhythmic drug therapy may, over time, alter electrophysiologic characteristics of the heart. As a result of the changes, the programmed therapies may become ineffective and possibly dangerous, e.g., initiate an atrial tachyarrhythmia or accelerate a ventricular tachycardia to flutter or fibrillation. Changing patient conditions may also require modification of the ICD system due to factors such as increased defibrillation requirements, unacceptable sensing, elevated pacing thresholds, loss of pacing capture and diaphragmatic stimulation. Patients receiving frequent shocks despite anti-arrhythmic medical management could develop psychological intolerance to an ICD system that might include the following: dependency, depression, fear of premature battery depletion, fear of shocking while conscious, fear that shocking capability may be lost, imagined shocking, i.e., phantom shock.

Similarly, potential adverse events related to the use of leads include, but are not limited to, the following patient related conditions: cardiac perforation, cardiac tamponade, constrictive pericarditis, embolism, endocarditis, fibrillation or other arrhythmia, heart wall rupture, hemothorax, infection, pneumothorax, thrombosis and tissue necrosis. Other potential adverse events related to the lead include, but are not limited to, the following: insulation failure, lead conductor or electrode fracture, lead dislodgement, and poor connection to the ICD. These may lead to oversensing, undersensing, or loss of therapy.

In performing the methods of the invention, risks have been minimized by the careful assessment of each subject prior to, during, and after implantation of the ICD. The devices contemplated by the invention have independently selectable parameters available to maximize the detection and/or rejection of tachyarrhythmia. The efficacy of programmed detection and therapies for the treatment of episodes of ventricular tachycardia is routinely evaluated prior to permanent implantation and programming of any device. The risk of failure to terminate an arrhythmia by the ICD is minimized by demonstrating an adequate defibrillation safety margin at the time of implant and the ability to select and deliver up to six therapies for detected episodes of ventricular tachycardia or fibrillation.

Careful follow-up of patients receiving ICD systems also helps to minimize risks associated with the device (such as battery depletion) or associated with the patient (such as altered drug regimen). Patients are followed at regular intervals to confirm that the programmed parameters are appropriate and to monitor the implanted system. At each follow-up examination, the ICD is interrogated, and verification of an adequate pacing threshold margin is made as well as an evaluation of pacing and sensing characteristics.

Telemetry reports, such as device status data, episode counter data, therapy counter data, episode data reports, lead trend data, daily automatic lead measurements and Patient Alert™ reports provide information about the operation and status of the ICD system. Various programmable EGM recording sources can be useful for troubleshooting possible lead and connector problems.

Specific SNPs, either alone or in combination, can be used to predict SCA, or SCD, risk and to select to which drugs or device therapies a patients may be more or less likely to respond. Identification of therapies to which a subject is unlikely to respond allows for quicker access to a more appropriate drug or device therapy. The genetic information can be taken from a biological specimen containing the patient DNA to be used for SNP detection, or from a previously obtained genetic sequence specific to the given patient. Once it is determined that the given patient has a high risk for SCA, then evaluation of possible therapies can be performed. Specific anti-arrhythmic drugs and device therapies including ICD, cardiac resynchronization therapy, anti-tachycardiac pacing therapy and Subcutaneous ICD, or similar therapies can be assessed for their likely effect on the individual patient.

### EXAMPLES

### Bead-Based Genotyping and Haplotyping

A template can be generated by obtaining genomic DNA probes representing the SNPs of SEQ ID Nos. 1-849. Nested PCR can be used to generate a template for typing where amplifications could be performed using PCR Mastermix (Abgene, Inc., Rochester, N.Y.). Primary PCRs can be carried out with 20 ng genomic DNA in 10 μl 1×PCR Mastermix, 0.2 μM of primers, and 2 mM MgCl₂ with the following cycling conditions: 95° C. for 5 min; 40 cycles at 95° C. for 30 s, 58° C. for 30 s, 72° C. for 2 min 30 s; 72° C. for 10 min. The product can then be diluted 1:500 in 1×TE and re-amplified using asymmetric PCR. The amplified products can then be analyzed by gel electrophoresis and then used directly in a bead-based genotyping and haplotyping reaction.

### Allele-Specific Hybridization

For genotyping and haplotyping, allele-specific oligonucleotides (ASOs), representing the SNPs of SEQ ID Nos. 1-849 can be synthesized. The ASO can be 25 nucleotides long with a 5′ Uni-Link amino modifier where each ASO can be attached to a different colored bead. Genotyping can be performed in a 30 μl hybridization reaction containing 5 μl unpurified PCR product, 83 nM biotinylated sequence-specific oligonucleotide and beads corresponding to each allele of the SNPs of SEQ ID NO.'s 1-849 reacted in 1×TMAC buffer (4.5 M TMAC, 0.15% Sarkosyl, 75 mM Tris-HCl, pH 8.0 and 6 mM EDTA, pH 8.0). The reactions can then be denatured at 95° C. for 2 min and incubated at 54° C. for 30 min. An equal volume of 20 μg/ml streptavidin-R-phycoerythrin (RPE) (Molecular Probes, Inc., Eugene, Oreg.) in 1×TMAC buffer can be added and the reaction be incubated at 54° C. for 20 min prior to analysis on a Luminex 100. The data collection software can be set to analyze 100 beads from each set and the median relative fluorescent intensity can be used for analysis. Visual genotypes and haplotypes can be generated using the online software applications found at http://pga.gs.washington.edu/software.html.

It should be understood that the above-described embodiments and examples are merely illustrative of some of the many specific embodiments that represent the principles of the present invention. Numerous other versions can be readily devised by those skilled in the art without departing from the scope of the present invention.

We claim:

1. A diagnostic kit, comprising at least one probe that determines the presence or absence of one or more Single Nucleotide Polymorphism (SNP) associated with Sudden Cardiac Arrest (SCA) in a genetic sample, said one or more SNP being selected from any one of SEQ ID Nos. 1-858.

2. The diagnostic kit of claim 1, wherein the SNP is selected from the group of SEQ ID Nos. 850-855 and 858.

3. The diagnostic kit of claim 1, wherein the SNP is selected from the group of SEQ ID Nos. 844, 831, 825, 839 and 833.

4. The diagnostic kit of claim 1, wherein the SNP is selected from the group of SEQ ID Nos. 835, 832, 844, 846, 838, 848, 829, 842, 827, 828, 824, 836, 840, 845, 826, 837, 841, 843, 117, 535, 823, 834, 830, 847, and 849.

5. The diagnostic kit of claim 1, wherein the SNP is selected from the group of SEQ ID Nos. 535, 505, and 515.

6. The diagnostic kit of claim 1, wherein said at least one probe overlaps position 26 or 27 in any one of SEQ ID Nos. 850-855 and 858, where position 26 or 27 is flanked on either the 5′ and 3′ side by a single base pair, to any number of base pairs flanking the 5′ and 3′ side of position 26 or 27 sufficient to identify the SNP or result in a hybridization.

7. The diagnostic kit of claim 6, wherein said at least one probe is from 3 to 101 nucleotides in length.

8. The diagnostic kit of claim 7, wherein the length of the at least one probe has a length n for the lower bound, and a length (n+i) for the upper bound, where n={xε [symbol] |3≦x≦101} and i={yε [symbol] |0≦y≦(101−n)}.

9. The diagnostic kit of claim 7, wherein said at least one probe has a length selected from the group of from 25 to 35, 18 to 30, and 17 to 24 nucleotides.

10. The diagnostic kit of claim 1, further comprising a Polymerase Chain Reaction (PCR) primer set for amplifying nucleic acid fragments corresponding to any one of SEQ ID Nos. 850-855 and 858.

11. The diagnostic kit of claim 1, wherein said at least one probe has a label capable of being detected.

12. The diagnostic kit of claim 10, wherein the label is detected by electrical, fluorescent or radioactive means.

13. The diagnostic kit of claim 1, wherein said at least one probe is affixed to a substrate.

14. The diagnostic kit of claim 1, further comprising a computer processor programmed with software for extracting information of a hybridization of said at least one probe in the diagnostic kit.

15. The diagnostic kit of claim 1, wherein said at least one probe is an Allele Specific Oligomer (ASO).

16. The diagnostic kit of claim 1, wherein the SNP is bi-allelic.

17. The diagnostic kit of claim 1, wherein the SNP is multi-allelic.

18. The diagnostic kit of claim 1, wherein said at least one probe is selected from the group of sense, anti-sense, and naturally occurring mutants, of any one of SEQ ID Nos. 850-855 and 858.

19. A system for detecting one or more Single Nucleotide Polymorphisms (SNPs) associated with Sudden Cardiac Arrest (SCA), comprising a computer system, having a computer processor programmed with an algorithm, and one or more genetic databases that are in communication with the programmed processor, wherein the programmed computer processor is used to impute p-values for one or more known SNPs detected in DNA contained in one or more genetic samples obtained from a patient and/or from the one or more genetic databases, and the p-value is used to assess association with SCA.

20. An isolated nucleic acid molecule useful for predicting Sudden Cardiac Arrest (SCA), comprising a nucleotide sequence having a Single Nucleotide Polymorphism (SNP) selected using the system of claim 19.

21. A DNA microarray, comprising at least one probe that determines the presence or absence of a Single Nucleotide Polymorphism (SNP) associated with Sudden Cardiac Arrest (SCA) in a genetic sample in any one of SEQ ID Nos. 850-855 and 858.

22. The DNA microarray of claim 21, wherein the microarray comprises synthesized oligonucleotides.

23. The DNA microarray of claim 21, wherein the microarray consists of a randomly or non-randomly assembled bead-based array.

24. The DNA microarray of claim 21, wherein the microarray, wherein the microarray consists of mechanically assembled arrays of spotted material, said spotted material selected from the group of an oligonucleotide, a cDNA clone, and a Polymerase Chain Reaction (PCR) amplicon.

25. A method of distinguishing patients having an increased or decreased susceptibility to SCA using the DNA microarray of claim 21, comprising the steps of: providing a nucleic acid sample; performing a hybridization to form a double-stranded nucleic acid between the nucleic acid sample and a probe; and detecting the hybridization.

26. The method of claim 25, wherein hybridization is detected radioactively.

27. The method of claim 25, wherein hybridization is detected by fluorescence.

28. The method of claim 25, wherein hybridization is detected electrically.

29. The method of claim 25, wherein the nucleic acid sample comprises DNA.

30. The method of claim 25, wherein the nucleic acid sample comprises RNA.

31. The method of claim 25, wherein the nucleic acid sample is amplified.

32. The method of claim 31, wherein the nucleic acid sample is amplified using Polymerase Chain Reaction (PCR).

33. The method of claim 25, wherein hybridization occurs under stringent conditions.
